(19) United States
(12) Patent Application Publication
(10) Pub. No.: US 2022/0025403 A1
(43) Pub. Date: Jan. 27, 2022

(54) REDUCED AND MINIMAL MANIPULATION MANUFACTURING OF GENETICALLY-MODIFIED CELLS

(71) Applicant: Fred Hutchinson Cancer Research Center, Seattle, WA (US)

(72) Inventors: Jennifer E. Adair, Seattle, WA (US); Reza Shahbazi, Seattle, WA (US)

(73) Assignee: Fred Hutchinson Cancer Research Center, Seattle, WA (US)

(21) Appl. No.: 17/311,211

(22) PCT Filed: Dec. 5, 2019
(86) PCT No.: PCT/US19/64780

§ 371 Date: Jun. 4, 2021

Related U.S. Application Data
(60) Provisional application No. 62/775,721, filed on Dec. 5, 2018.

(51) Int. Cl. C12N 15/88; A61K 35/28; C07K 16/28; C12N 9/22; C12N 15/11; C12N 15/90; C12N 11/091

(52) U.S. Cl. CPC C12N 15/88; A61K 35/28; C07K 16/2812; C07K 16/2896; C07K 16/2803; C12N 2510/00; C12N 15/11; C12N 15/907; C12N 11/091; C12N 2800/80; C12N 2310/20; C12N 9/22

(57) ABSTRACT

Nanoparticles to genetically modify selected cell types within a biological sample that has been subjected to reduced or minimal manipulation are described. The nanoparticles deliver all components required for precise genome engineering and overcome numerous drawbacks associated with current clinical practices to genetically engineer cells for therapeutic purposes.

### CROSS-REFERENCE TO RELATED APPLICATION

This application claims priority to U.S. Provisional Patent Application No. 62/775,721 filed Dec. 5, 2018, which is incorporated herein by reference in its entirety as if fully set forth herein.

### STATEMENT REGARDING SEQUENCE LISTING

The Sequence Listing associated with this application is provided in text format in lieu of a paper copy and is hereby incorporated by reference into the specification. The name of the text file containing the Sequence Listing is F053-0091PCT_ST25.txt. The text file is 296 KB, was created on Dec. 5, 2019, and is being submitted electronically via EFS-Web.

### FIELD OF THE DISCLOSURE

The current disclosure provides nanoparticles to genetically modify selected cell types with reduced or minimal manipulation. The nanoparticles deliver all components required for precise genome engineering and overcome numerous drawbacks associated with current clinical practices to genetically engineer cells for therapeutic purposes.

### BACKGROUND OF THE DISCLOSURE

Patient-specific gene therapy has great potential to treat genetic, infectious, and malignant diseases. For example, retrovirus-mediated gene addition into hematopoietic stem cells (HSC) and hematopoietic stem cells and progenitor cells (HSPC) has demonstrated curative outcomes for several genetic diseases over the last 10 years including inherited immunodeficiencies (e.g., X-linked and adenosine deaminase deficient severe combined immunodeficiency (SCID)), hemoglobinopathies, Wiskott-Aldrich syndrome and metachromatic leukodystrophy. Additionally, this treatment approach has also improved outcomes for poor prognosis diagnoses such as glioblastoma. The use of gene-corrected autologous, or “self” cells, as opposed to cells from a donor, eliminates the risk of graft-host immune responses, negating the need for immunosuppressive drugs.

Current systems used in clinical medicine lack an optimal method to deliver gene-editing components to HSC and HSPC as well as other blood cell types. For example, the CRISPR-Cas9 platform is one approach being pursued in the clinical setting for gene editing in HSPC. If the goal is gene disruption, only electroporation is required to deliver gene editing components. However, electroporation is toxic to many cell types and this toxicity is especially problematic for therapies using HSC and/or HSPC where the starting cell numbers are low.

If the goal is to insert new genetic material, then a DNA template for homology directed repair must be included. This can be accomplished by electroporating in a single-stranded DNA (ssDNA) template if the new genetic material is small, but for larger templates, use of adeno-associated viral vectors (AAV) is the current gold standard in clinical practice. Whether electroporation alone or in combination with AAV is used, there is no guarantee that all of the separate gene-editing components to be delivered are delivered into the same cells. Moreover, electroporation relies on the mechanical disruption and permeabilization of cellular membranes, thus compromising the viability of cells, rendering them less than ideal for therapeutic use. Further, like virus-based methods, electroporation does not selectively deliver genes to specific cell types out of a heterogeneous pool, so it must be preceded by cell selection and purification process. Cell selection and purification processes are harsh processes leading to an undesirably high toxicity level. Finally, AAV treatment carries immunogenic potential when cells are reinfused.

Any improved method of delivering gene-editing components which can simplify the steps required and ensure that all components are delivered to intended cell types would be a significant improvement to the field of clinical medicine. Nanoparticles such as polyplexes and lipoplexes have been proposed, but these have been shown to be toxic, demonstrate limited efficiency of gene-editing component delivery and have limited gene-editing efficacy in HSC and HSPC.

### SUMMARY OF THE DISCLOSURE

The current disclosure provides nanoparticles (NP) that allow the selective genetic modification of selected cell types with reduced and minimal manipulation. Reduced manipulation means that the use of electroporation and viral vectors, such as AAV, are not required. Minimal manipulation means that the use of electroporation, viral vectors, and cell selection and purification processes are not required. Further, the current disclosure also provides NP specifically engineered to deliver all components required for genome editing. The NP can be used for therapies where a loss-of-function mutation is needed, but importantly, can also provide all components needed for gene addition or correction of a specific mutation. The described approaches are safe (i.e., no off-target toxicity), reliable, scalable, easy to manufacture, synthetic, and plug-and-play (i.e., the same basic platform can be used to deliver different therapeutic nucleic acids).

### BRIEF DESCRIPTION OF THE SEVERAL VIEWS OF THE DRAWINGS

Many of the drawings submitted herein are better understood in color. Applicant considers the color versions of the drawings as part of the original submission and reserves the right to present color images of the drawings in later proceedings.

FIGS. 1A-1C. (FIG. 1A) Current clinically used systems for ex vivo gene editing lack an optimal delivery method for HSC, HSPC, and other blood cells. As shown in (FIG. 1A), current clinically used protocols include 8 steps: (1) mobilization and apheresis; (2) immunomagnetic separation of the targeted cell type (e.g., CD34+ HSPC in FIG. 1A); (3) stimulation of the separated cells in culture media with recombinant growth factors (rhGFs); (4) electroporation of cells to deliver gene-editing components (e.g., CRISPR/Cas9 ribonucleoproteins in FIG. 1A); (5) incubation of cells in culture media and rhGFs following electroporation; (6) transduction with a viral vector (e.g., an adeno-associated viral vector (AAV) in FIG. 1A) carrying a gene-editing donor template; (7) further incubation of cells in culture media and rhGFs; and (8) cell harvest for reinfusion into the conditioned patient. A goal of clinical medicine is reduced and minimal manipulation manufacturing. (FIG. 1B) Reduced manipulation manufacturing does not require electroporation or viral vector delivery but may still utilize target cell purification processes. As shown in (FIG. 1B), NP disclosed herein can be used to reduce reliance on steps 3-6 of (FIG. 1A). (FIG. 1C) In some embodiments, minimal manipulation ex vivo manufacturing does not require separation of selected cell types, electroporation or viral-mediated gene-editing component delivery, thus greatly improving the efficiency of ex vivo cell manufacturing. NP disclosed herein with targeting ligands further reduce reliance on steps 2-7 of FIG. 1A and do not require use of cell selection and purification processes.

FIG. 2 (prior art). CD34+CD45RA−CD90+ cells are responsible for blood repopulation. Nonhuman primate CD34+ cells were separated by flow-sorting into fractions i (CD45RA-CD90+), ii (CD45RA−CD90−) and iii (CD45RA+CD90−), then transduced with LV encoding green fluorescent protein, mCherry or mCerulean and transplanted into myeloablated autologous recipients. In all cases, blood cell engraftment corresponded only to CD34+CD45RA−CD90+(fraction i) cells.

FIG. 3 (prior art). Logarithmic correlation of transplanted CD34highCD45RA− CD90+ cells/kg body weight with neutrophil and platelet engraftment (Spearman's rank correlation coefficient R2: 0.0-0.19=very weak, 0.20-0.39=weak, 0.4-0.59=moderate, 0.6-0.79=strong, 0.8-1.0=very strong). The linear regression and the 95% confidence interval are indicated by solid and dotted lines, respectively.

FIG. 4. AuNP size determines destination tissue/elimination pathway when administered to humans.

FIGS. 5A-5D. Schematics representing synthesis and structure of NP. (FIG. 5A) Schematic of early production scheme for gold nanoparticles (AuNPs), a scalable, synthetic delivery scaffold with established in vivo compatibility. (FIG. 5B) Schematic representation of a synthesis process for creating and loading AuNP with exemplary gene editing components. One depicted AuNP shows crRNA attached to an AuNP surface. Cpf1 nuclease and ssDNA are then attached to the crRNA. Another depicted AuNP shows crRNA linked to an 18-ethylene glycol spacer with a thiol modification that is attached to the surface of a 19 nm AuNP core. A CRISPR nuclease is attached to the cRNA to form an RNP. The AuNP is coated with a low molecular weight (MW (e.g., 2000)) polyethyleneimine (PEI). ssDNA is layered onto the PEI-coated surface. (FIG. 5C) Schematic representation of an Au/CRISPR NP assembly process. 1) AuNP cores are synthesized and purified. 2) crRNAs with a spacer arm and thiol group are conjugated to the surface of gold (Au) cores. 3) An RNP complex is formed on the surface by the interaction of the CRISPR nuclease with crRNA. 4) The RNP complex is coated with PEI of 2K MW. 5) ssDNA template is captured on the surface by electrostatic interaction with PEI. (FIG. 5D) Additional schematic depicting an AuNP described herein.

FIGS. 6A-6E. Exemplary AuNP with selected cell targeting ligands. (FIG. 6A) Depiction of an exemplary AuNP configured with all components for gene addition and cell targeting. Depicted components include crRNA, a Cpf1 nuclease, and single-stranded DNA (ssDNA) to provide a therapeutic nucleic acid sequence (e.g. a gene or corrected portion thereof). The targeting ligand includes an aptamer. (FIG. 6B) Schematic of an alternative formulated “layered” AuNP which can be used to deliver large oligonucleotides, such as donor templates including homology-directed repair templates (HDT), therapeutic DNA sequences, and other potential elements. Donor templates are located farther from the AuNP surface than the depicted ribonucleoprotein complex (RNP). An aptamer targeting ligand is also depicted. (FIG. 6C) The design represented in FIG. 5D with an aptamer targeting ligand attached to a nuclease through a direct amino acid link. (FIG. 6D) The design represented in FIG. 5D with an aptamer targeting ligand attached to a nuclease through a polyethylene glycol (PEG) tether. (FIG. 6E) The design represented in FIG. 5D with an antibody targeting ligand attached to a nuclease through an amine-to-sulfhydryl crosslinker or a direct amino acid link. Antibody targeting ligands attached through a PEG tether are also provided.

FIGS. 7A, 7B. Targeting locus on CCR5 gene. (FIG. 7A) The target locus has PAM sites for both Cpf1 and Cas9 with a 20 bp guide segment in the middle (SEQ ID NO: 1). (FIG. 7B) HDT were designed around the cut site with an 8 bp NotI recognition sequence insert and symmetrical homology arms of 40 bp length (SEQ ID NO: 2).

FIGS. 8A, 8B. Targeting locus within the γ-globin gene promoter. (FIG. 8A) The target locus has PAM sites for both Cpf1 and Cas9 with a 21 bp guide segment in the middle (SEQ ID NO: 3). (FIG. 8B) HDT were designed around the cut site with the 13 bp HPFH deletion and symmetrical homology arms of 30 bp length (SEQ ID NO: 4).

FIG. 9. Fully-loaded AuNPs are monodisperse and display good zeta potential.

FIGS. 10A-10D. Graphs and digital images showing the characteristic properties of synthesized AuNPs and optimal loading concentrations. (FIG. 10A) Localized surface plasmon resonance (LSPR) peaks of synthesized AuNPs. (FIG. 10B) LSPR peaks of the AuNP and Au/CRISPR NP. (FIG. 10C) Gel electrophoresis showing optimal AuNP/ssDNA w/w loading ratio. (FIG. 10D) Loading concentration of Au/CRISPR NP.

FIGS. 11A, 11B. Optimal loading concentrations. (FIG. 11A) AuNP/crRNA 50 nm (Ratio 6); AuNP/crRNA 15 nm (Ratio 1); and AuNP/crRNA/Cpf1/PEI/DNA 15 nm (Ratio 0.5). (FIG. 11B) Smaller AuNPs triple the available surface area with the same starting reagent amounts. By decreasing the size, surface area and conjugation ratio of the NPs increase.

FIGS. 12A-12E. (12A) Layer by layer conjugation of CRISPR components onto AuNP. (FIG. 12B) Dynamic light scattering characterization of AuNPs after each layering step. Sharp single peaks and shifts in size after adding each layer demonstrate precise attachment to the surface. (FIG. 12C) Average size (Z-Average, bar graphs plotted on the right axis) and polydispersity index (PDI, dots plotted on the left axis) of AuNPs after each layering step. PDI values <0.2 show high monodispersity without aggregation. Data are means±s.e (n=3). (FIG. 12D) Red shifts in LSPR of AuNPs after adding each component confirm cargo loading. (FIG. 12E) Zeta potential measurements after adding each layer changed from −26 mV for AuNPs to +27 mV for the final Au/CRISPR NP. Data are means±s.e (n=3).

FIGS. 13A-13D. Characterization of the optimal amounts of Cpf1 and ssDNA. (FIG. 13A) Size analysis of NP in different AuNP/Cpf1 w/w ratios. Measurements were done in triplicate. (FIG. 13B) Z-average and PDI values in different AuNP/Cpf1 w/w ratios. AuNP/Cpf1 w/w ratio of 0.6 was found to be optimal in terms of size and PDI. Measurements were done in triplicate. (FIG. 13C) Size analysis of NP in different AuNP/ssDNA w/w ratios. Measurements were done in triplicate. (FIG. 13D) Z-average and PDI values in different AuNP/ssDNA w/w ratios. The AuNP/ssDNA w/w ratio of 1 was found to be optimal in terms of size and PDI. Measurements were done in triplicate.

FIGS. 14A-14E. Au/CRISPR NP can deliver CRISPR components to the nucleus of HSPCs. (FIG. 14A) HSPC take up fully-loaded AuNPs in vitro. (FIG. 14B) Nucleus of primary human CD34+ HSPC following addition of Au/CRISPR NP to the culture (blue, Hoechst). (FIG. 14C) Fluorophore tagged crRNA (green, Alexa488) was used to track the cellular biodistribution in the cytoplasm and nucleus. (FIG. 14D) Fluorophore tagged ssDNA (Red, Alexa660) was also present both in the cytoplasm and nucleus. Visible small vesicles on the far left side of the image suggest passive uptake by endocytosis. (FIG. 14E) Overlay of all three stains showed colocalization of crRNA and ssDNA. Images were acquired by confocal microscope at Z-Stack mode and 60× magnification.

FIGS. 15A-15C. Au/CRISPR NP are non-toxic to primary human CD34+ HSPC. (FIGS. 15A, 15B) Live-Dead viability assay results after 24 h (upper panels) and 48 h (lower panels). Cell viabilities were above 70% for the Au/CRISPR NP treated group and were similar to the mock treated group. (FIG. 15C) Cell viabilities by trypan blue dye exclusion assay. Assay results were in close correlation with the live-dead assay results.

FIGS. 16A-16D. Graphs showing the gene cutting efficiency in K562 cells and CD34+ cells. (FIG. 16A) Percent viability after delivery with AuNPs and electroporation method. (FIG. 16B) Administration dose of CRISPR components. (FIGS. 16C,16D) Tracking Indels by Decomposition (TIDE) assay results showing percent cutting efficiency in K562 cells and CD34+ cells.

FIG. 17. Up to 10% gene editing and HDR was observed in vitro in primary CD34+ cells obtained from a G-CSF mobilized healthy adult donor. CD34+ cells were thawed using a rapid-thaw method and cultured overnight in Iscove's Modified Dulbecco's Medium (IMDM) containing 10% FBS and 1% Pen/Strep. The following morning, AuNPs were seeded and assembled as follows: seed; add crRNA with a PEG spacer to prevent electrostatic repulsions; add Cpf1 protein and allow RNPs to form; coat with 2K branched PEI and single-stranded oligonucleotide (ssODN).

In this example, there were no chemical modifications of crRNA other than terminal thiol additions to promote covalent bonding with the AuNP surface for attachment. SsODN was used as the HDT, here a 8 bp insert using a NotI site flanked by 40 nt of homology (symmetric) to the CCR5 target locus. Formulated AuNPs were added to cells and incubated for 48 hours with gentle plate mixing. After 48 hours, cells were harvested, washed, and genomic DNA (gDNA) was isolated for PCR amplification and analysis.

FIG. 18. TIDE assay results showing indels after editing with Au/CRISPR NP (15 nm, 50 nm, and 100 nm) in CD34+ cells.

FIGS. 19A-19C. In vitro analysis of cells transplanted into NSG mice. (FIG. 19A) 10% HDR was observed by TIDE without significant indels at the target locus in human CD34+ cells at the time of transplant. (FIG. 19B) Both T7 Endonuclease I (T7EI) and NotI restriction digest were only observed in cells that received fully-loaded AuNP. (FIG. 19C) Interestingly, increased colony-forming capacity for this donor was noted only when cells were treated with AuNPs. No significant differences were observed in the types of colonies formed across each condition.

FIG. 20. Early post-transplant analysis suggests gene edited cell engraftment. Peripheral blood was collected for gDNA analysis at 6 weeks after transplant. Across all mice treated with fully-loaded AuNPs, 7/10 displayed detectable editing ranging from 0.5-6% by TIDE. In one mouse (5% total editing), 1.7% HDR was observed by TIDE analysis.

FIGS. 21A-21D. Optimization of HDR conditions and optimal editing dosage. (FIG. 21A) HDT designed for the non-target strand display higher levels of NotI insertion. Data are means±s.e (n=3). (FIG. 21B) T7EI and NotI restriction enzyme digestions showing the related digestion bands. (FIG. 21C) effect of different Au/CRISPR NP concentrations on HDR in primary human HSPC. Data are means±s.e (n=3). (FIG. 21D) Concentrations over 20 μg/mL had toxic effects on CD34+ cells. Data are means±s.e (n=3). Statistical significance was determined by a two-sample t-test.

FIGS. 22A-22C. Effect of different serum conditions and transfection components on gene editing. (FIG. 22A) Cell viability after 48 h treatment in different conditions. Data are means±s.e (n=3). (FIG. 22B) Total editing levels by TIDE assay. Data are means±s.e (n=3). (FIG. 22C) HDR levels by TIDE assay. Data are means±s.e (n=3).

FIGS. 23A-23F. Au/CRISPR NP carrying Cpf1 outperform Cas9 in terms of HDR. (FIG. 23A) Total editing results by TIDE assay. Au/CRISPR NP improved Cas9 cutting efficiency at the CCR5 locus. Data are means±s.e (n=3). (FIG. 23B) HDR results by TIDE assay showed higher level of NotI insertion using Cpf1 as compared to Cas9. Levels of HDR observed for both Au/CRISPR NP-delivered Cpf1 and Cas9 were higher than electroporation. Data are means±s.e (n=3). Statistical significance was determined by a two-sample t-test. (FIG. 23C) Miseq analysis confirmed the observed trend with TIDE assay. Data are means±s.e (n=3). Statistical significance was determined by a two-sample t-test. (FIG. 23D) Cell viability of CD34+ cells after treatment with CRISPR Cpf1 and Cas9 using Au/CRISPR NP and electroporation methods. Cell viabilities were above 70% for all the study groups. Data are means±s.e (n=3). Statistical significance was determined by doing one-way ANOVA. (FIG. 23E) colony forming cell (CFC) assay results showing the total colony numbers. Data are means±s.e (n=3). (FIG. 23F) CFC assay results showing the percentage of different colonies. Data are means±s.e (n=3).

FIGS. 24A, 24B. Replated CFC assay showing the effect of treatment on colony forming potential of long-term progenitors. (FIG. 24A) CFC assay results showing the total colony numbers. Data are means±s.e (n=3). (FIG. 24B) CFC assay results showing the percentage of different colonies. Data are means±s.e (n=3).

FIG. 25. Targeting locus within the γ-globin gene promoter HDR results by Miseq analysis showed higher level of 13 bp deletion profile for Cpf1 in comparison to Cas9. Data are means±s.e (n=3).

FIG. 26. AuNP-treated CD34+ cells engraft in vivo. The same procedures were used as described in relation to FIG. 17, except that CD34+ cells were initially obtained from a different human donor. After 48 hours, cells were harvested, washed, and injected into sub-lethally irradiated adult (8-12 week) NSG mice. Cell reserves were used to assess plate colony assays and to isolate gDNA for PCR amplification and analysis.

FIGS. 27A-27G. AuNP treatment enhanced HSPC engraftment in NSG mice. (FIGS. 27A, 27B) Engraftment as measured by percentage of human CD45 expressing cells in peripheral blood of NSG recipients. AuNP- and Au/CRISPR-HDT-NP-treated cells engrafted better than mock-treated cells. Data are means±s.e (n=10 Au/CRISPR-HDT-NP, n=10 AuNP, n=5 Mock, n=4 un-injected). Statistical significance was determined by a two-sample t-test. (FIG. 27C) Human CD20+ B cell engraftment kinetics in the peripheral blood. (FIG. 27D) Human CD14+ monocyte engraftment kinetics in the peripheral blood. (FIG. 27E) Human CD3+ T cell engraftment kinetics in the peripheral blood. (FIG. 27F) CFC assay showing the total colony numbers for bone marrow samples. CFC results were in close correlation with engraftment results. Data are means±s.e (n=3). Statistical significance was determined by a two-sample t-test. (FIG. 27G) CFC assay results showing the frequency of different morphologies. Data are means±s.e (n=3).

FIG. 28. Mice weights were stable over the course of study. Tracking mice weights for different cohorts. Data are means±s.e (n=10 Au/CRISPR-HDT-NP, n=10 AuNP, n=5 mock, n=4 un-injected).

FIG. 29A-29D. Engraftment level of cell populations in the necropsy samples after treatment with Au/CRISPR NP. (FIG. 29A) Engraftment levels in the bone marrow. Data are means±s.e (n=10 Au/CRISPR-HDT-NP, n=10 AuNP, n=5 Mock). (FIG. 29B) Engraftment levels in the spleen. Data are means±s.e (n=10 Au/CRISPR-HDT-NP, n=10 AuNP, n=5 Mock). (FIG. 29C) Engraftment levels in the thymus. Data are means±s.e (n=10 Au/CRISPR-HDT-NP, n=10 AuNP, n=5 Mock). (FIG. 29D) Engraftment levels in the peripheral blood. Data are means±s.e (n=10 Au/CRISPR-HDT-NP, n=10 AuNP, n=5 Mock).

FIGS. 30A, 30B. (FIG. 30A) Colony forming potential of Au/CRISPR NP treated cells before engraftment. CFC assay showing the total colony numbers before engraftment. Data are means±s.e (n=3). Statistical significance was determined by a two-sample t-test. (FIG. 30B) CFC assay results showing the percentage of different colonies. Data are means±s.e (n=3).

FIG. 31. Representative colony morphologies after treatment with Au/CRISPR NP. Burst forming unit-erythroid (BFU-E), granulocyte monocyte (GM).

FIGS. 32A-32E. Persistent editing levels after engraftment. (FIG. 32A) TIDE assay results for total editing and HDR levels before engraftment. (FIG. 32B) Tracking of total editing levels.

Starting from 4 weeks after transplant, peripheral blood samples were collected every other week.

Data are means±s.e (n=10). (FIG. 32C) Tracking of HDR levels after engraftment. Data are means±s.e (n=10). (FIG. 32D) Total editing levels in peripheral blood, bone marrow and spleen at necropsy. Data are means±s.e (n=10). (FIG. 32E) HDR levels in peripheral blood, bone marrow and spleen at necropsy. Data are means±s.e (n=10).

FIG. 33. NotI and T7EI restriction enzyme digestion after treatment with Au/CRISPR NP.

FIG. 34. Sequences of crRNAs, HDT and primers (SEQ ID NOs: 5-19).

FIGS. 35A-35D. (FIG. 35A) Potential off target cutting sites for Cpf1 and Cas9 on CCR5 and γ-globin target sites (SEQ ID NOs: 20-27). (FIG. 35B) Cas9 and Cpf1 guide and HDR templates for hereditary persistence of fetal hemoglobin (HPFH) (SEQ ID NOs: 28-52 and 214-224). Each guide sequence spans a specific mutation. Target DNA sequences that can be used for crRNA synthesis are provided. (FIG. 35C) Transcribed RNA sequences (SEQ ID NOs: 225-262) from DNA target sites for genetic engineering (SEQ ID NOs: 20-22, 24-26, 28-32, 42, 43, 84-97, and 214-224). (FIG. 35D) Table provides complementary sets of DNA target sites, cRNA sequences, and HDT.

FIG. 36. Additional sequences supporting the disclosure (SEQ ID NOs: 112-138).

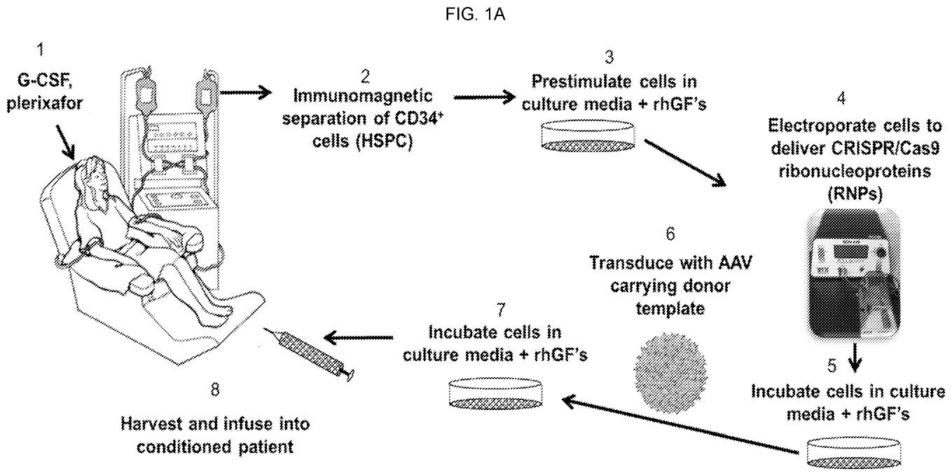

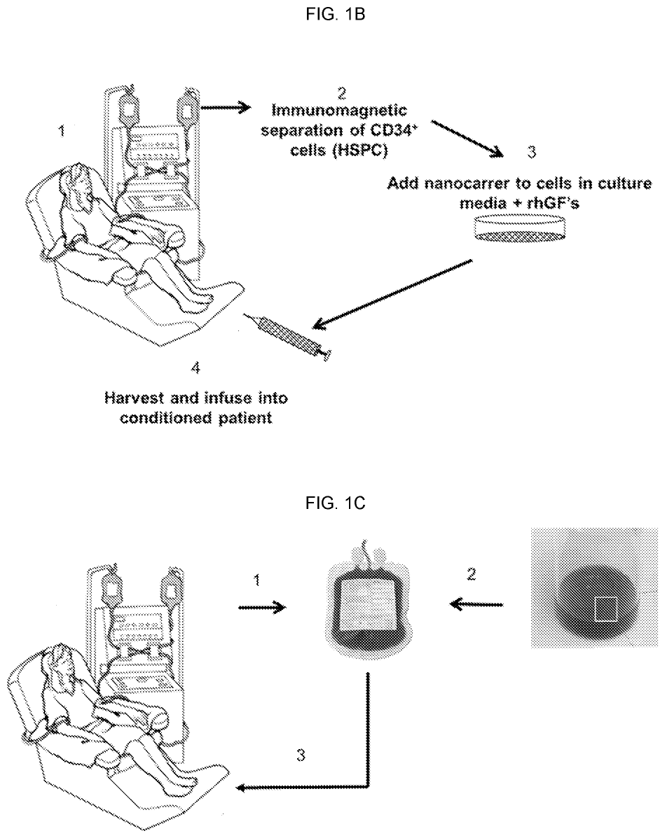

### DETAILED DESCRIPTION

Gene therapy has great potential to treat genetic, infectious, and malignant diseases. For example, retrovirus-mediated gene addition into hematopoietic stem cells (HSC) and hematopoietic stem cells and progenitor cells (HSPC) has demonstrated curative outcomes for several genetic diseases over the last 10 years including inherited immunodeficiencies (e.g., X-linked and adenosine deaminase deficient severe combined immunodeficiency (SCID)), hemoglobinopathies, Wiskott-Aldrich syndrome and metachromatic leukodystrophy. Additionally, this treatment approach has also improved outcomes for poor prognosis diagnoses such as glioblastoma. The use of gene-corrected autologous, or “self” cells, rather than cells from a donor, eliminates many risks of cell-based genetic therapies including graft-host immune responses, negating the need for immunosuppressive drugs.

Currently, clinical systems lack an optimal method to deliver gene-editing components to many cell types. For example, for hematopoietic stem cells (HSC) and hematopoietic stem and progenitor cells (HSPC), the current state-of-the-art includes the removal of cells from the patient via bone marrow aspirate or mobilized peripheral blood, sorting this bulk population for autologous HSPC by immunoselection of cells expressing the surface marker CD34, then culturing these cells in the presence of cytokines. If the goal is disruption of an existing problematic gene, electroporation is used to deliver gene editing components to the cells. Electroporation generally refers to applying an electric field to cells to increase the permeability of the cell's membrane to allow passage of molecules to be introduced into the cell. Electroporation is toxic to many cell types and this toxicity is especially problematic for therapies using HSC and/or HSPC where the starting cell numbers are low.

If the goal is to insert new genetic material into the cell, then a DNA template for homology directed repair must also be included. This can be accomplished by electroporation alone if the new genetic material is small, but for larger forms of genetic material, the additional use of adeno-associated viral vectors (AAV) is the current gold standard in clinical practice. There remains a known risk of genotoxicity and other limitations associated with the use of viral vectors for gene transfer. For example, risks of genotoxicity are evidenced by the development of malignancy due to insertional mutagenesis in patients treated with HSPC gene therapy. This adverse side effect stems from the semi-random nature of retroviral-mediated transgene delivery into the host cell genome. Dysregulation of nearby genes by the inserted transgene sequence has been the molecular basis for clonal expansion and malignant transformation observed in some gene therapy patients, but reciprocal interactions between the inserted transgene and the surrounding genomic context can also cause transgene attenuation or silencing, diminishing therapeutic effects. Other limitations associated with the use of particular viral vectors include induction of immune responses, a decreased efficacy over time in dividing cells (e.g., adeno-associated vectors), an inability to adequately target selected cell types in vivo (e.g., retroviral vectors), and, as indicated, an inability to control insertion site and number of insertions (e.g., lentiviral vectors).

The last several years have seen an explosion in gene editing as a safer alternative to retrovirus-mediated gene transfer, made possible by the development of engineered guide RNA and nucleases which target specific DNA sequences and predictably generate DNA double strand breaks (DSB) at the targeted sequence. To date, these programmable complexes have been most effective at providing promising therapies when removal or silencing of a problematic gene (i.e., generating a loss-of-function mutation) is needed. This is because DSBs are most commonly repaired by error-prone non-homologous end joining (NHEJ) which results in oligonucleotide insertions and deletions (indels) at the DSB site.

For gene addition or correction of a specific mutation, less common homology-directed repair (HDR) of the DSB is required. In this situation, a more complex payload including the engineered guide RNA and nuclease as well as a homology-directed repair template must be co-delivered. Proof-of-concept for this approach has been demonstrated in HSPC but also required either tandem electroporation of some gene editing components followed by transduction with non-integrating viral vectors, particularly recombinant adeno-associated viral (rAAV) vectors to deliver DNA templates, or simultaneous electroporation of defined concentrations of engineered nuclease components with chemically modified, single-stranded oligonucleotide template at specified cell concentrations. Moreover, each engineered guide RNA, nuclease and homology-directed repair template had to be uniquely engineered for each specified genetic target, requiring separate evaluation of delivery, activity and specificity in cell lines and HSPC.

Whether electroporation is used alone or in combination with AAV, there is no guarantee that all of the separate components required for gene editing are delivered into the same cells.

Further, electroporation and many viral vectors do not selectively deliver genes to specific cell types out of a heterogeneous pool, so these treatments must be preceded by cell selection and/or purification processes. Cell selection and purification processes are manipulations, which can lead to cell toxicity or loss of fitness. An example of this is blood stem cells which can start differentiating when manipulated leading to a loss of engraftment potential as more differentiated blood cells cannot support long-term blood production.

Thus, while there have been many exciting breakthroughs in the ability to perform genetic therapies at specific sites within the genome, the continued lack of a safe and potent delivery vehicle has hindered the clinical translation of gene editing systems, in particular, with HSC/HSPC.

Any improved method of delivering gene-editing components to cells which are less toxic and can simplify the steps required to ensure that all gene-editing components are delivered to cells would be a significant improvement to clinical medicine. From a logistical perspective, as well given the complex infrastructure required for manipulation of autologous cell products, having a more local and streamlined manufacturing process will decrease vein to vein times which may be important in certain disease contexts. Nanoparticles such as polyplexes and lipoplexes have been proposed, but these have been shown to be too toxic to cells and demonstrated limited efficiency of gene-editing component delivery to, for example, HSPC.

The current disclosure provides nanoparticles (NP) that allow the selective genetic modification of selected cell types with reduced and minimal manipulation. Reduced manipulation means that the use of electroporation and viral vectors, such as AAV, are not required. In particular embodiments, reduced manipulation means that electroporation and viral vectors, such as AAV, are not used. Minimal manipulation means that the use of electroporation, viral vectors, and cell selection and purification processes are not required. In particular embodiments, minimal manipulation means that electroporation, viral vectors, and cell selection and purification processes are not used. In particular embodiments, minimal manipulation means that a sample containing the selected blood cell type is only washed to remove platelets before being exposed to NP disclosed herein. As will be described in more detail elsewhere herein, whether the NP are used in reduced manipulation or minimal manipulation processes depends on whether a cell targeting ligand is associated with the NP.

Targeting ligands include, for example, antibodies, aptamers, ligands or other molecules that specify interaction of the NP with the cell type of interest. Selected cell targeting ligands can include surface-anchored targeting ligands that selectively bind the NP to selected cells and initiate cellular uptake. In particular embodiments, cellular uptake can be mediated by receptor-induced endocytosis. As disclosed in more detail elsewhere herein, selected cell targeting ligands can include antibodies, scFv proteins, DART molecules, peptides, and/or aptamers. Particular embodiments utilize antibodies, antibody binding fragments, or aptamers recognizing CD3, CD4, CD34, CD90, CD133, CD164, the luteinizing hormone-releasing hormone (LHRH) receptor, an aryl hydrocarbon receptor (AHR), or CD46 to target HSCs. Particular embodiments include as targeting ligands one or more of an anti-human CD3 antibody, an anti-human CD4 antibody, an anti-human CD34 antibody, an anti-human CD90 antibody, an anti-human CD133 antibody, an anti-human CD164 antibody, an anti-human CD133 aptamer, human luteinizing hormone, human chorionic gonadotropin (hCG, a ligand for LHRH receptor), degerelix acetate (an antagonist of the LHRH receptor), or StemRegenin 1 (a ligand for AHR).

When the disclosed NP are added to a heterogeneous mixture of cells (e.g., an ex vivo blood product), the engineered NP bind to selected cell populations and, are internalized into the target cell. This process provides entry for the genetic engineering components the NP carry, and consequently the selected cells become genetically modified. Provision of all components required for genetic engineering on a single particle ensures that a cell that takes up the particle receives all necessary components rather than a subset thereof. By targeting the NP to the desired cell population, cell selection (immunomagnetic or other) is no longer necessary.

Use of NP disclosed herein expedites the manufacturing of therapeutic cells ex vivo and results in less cellular harm during processing and genetic engineering. In particular embodiments, this method also reduces the amount of time from harvest of patient cells to re-infusion of a genetically modified blood cell product.

In particular embodiments, NP disclosed herein are gold nanoparticles (AuNP). AuNP particularly have been shown to be non-toxic to both non-dividing and dividing mammalian cells and have been applied for in vivo delivery of RNA therapeutics in clinical trials. Further, owing to their unique surface chemistry, AuNP can be loaded with all components required for gene editing. As described in more detail herein, the gene-editing components can be attached to the NP in a specifically designed layered configuration that optimizes the functionality and characterization of the NP in terms of, e.g., size, polydispersity index, and gene-editing efficiency.

Particular embodiments include a NP with components to provide a targeted loss-of-function mutation. These embodiments include a targeting element (e.g., guide RNA) and a cutting element (e.g. a nuclease) associated with the surface of the NP. In particular embodiments, the targeting element is conjugated to the surface of the NP through a thiol linker.

In particular embodiments, the targeting element and/or the cutting element are conjugated to the surface of the NP through a thiol linker. In particular embodiments, the targeting element is conjugated to the surface of the NP through a thiol linker and the cutting element is linked to the targeting element to form a ribonucleoprotein (RNP) complex. The targeting element targets the cutting element to a specific site for cutting and NHEJ repair.

Particular embodiments include a NP with components to provide a targeted gain-of-function mutation (e.g., gene addition or correction). In particular embodiments, these embodiments include a metal NP (e.g., AuNP) associated with a targeting element, a cutting element, a homology-directed repair template (HDT), and a therapeutic DNA sequence. The targeting element targets the cutting element to a specific site for cutting, the homology-directed repair template provides for HDR repair, wherein following HDR repair the therapeutic DNA sequence has been inserted within the target site. Together, homology-directed repair templates and therapeutic DNA sequences can be referred to herein as donor templates. In particular embodiments, the targeting element is conjugated to the surface of the NP through a thiol linker.

In particular embodiments, the targeting element and/or the cutting element are conjugated to the surface of the NP through a thiol linker. In particular embodiments, the targeting element is conjugated to the surface of the NP through a thiol linker and the cutting element is linked to the targeting element to form a ribonucleoprotein (RNP) complex. In these embodiments, the RNP complex is closer to the surface of the NP than donor template material. This configuration is beneficial when, for example, the targeting element and/or the cutting element are of bacterial origin. This is because many individuals who may receive NP described herein may have pre-existing immunity against bacterially-derived components such as bacterially-derived gene-editing components. Including bacterially-derived gene-editing components on an inner layer of the fully formulated NP allows non-bacterially-derived components (e.g., donor templates) to shield bacterially-derived components (e.g. targeting elements and/or cutting elements) from the patient's immune system. This protects the bacterially-derived components from attack and also avoids or reduces unwanted inflammatory responses against the NP following administration. In addition, this may allow for repeated administration of the NP in vivo without inactivation by the host immune response.

Particular embodiments can utilize an AuNP associated with at least four layers wherein the first layer includes CRISPR (Clustered Regularly Interspaced Short Palindromic Repeats) guide RNA (crRNA), the second layer includes a nuclease, the third layer includes ssDNA, and the fourth layer includes a targeting ligand, wherein the first layer is closest to the surface of the NP core, the second layer is second closest to the surface of the NP core, the third layer is third closest to the nanoparticle core, and the fourth layer is the farthest from the NP core. In particular embodiments, an layer refers to a layer associated with a NP that includes components that are used in genetic modification of selected cell populations including crRNA, nuclease, donor template, targeting ligand, and/or components that are used to create the layers including linkers and polymers (e.g., polyethylene glycol (PEG), and polyethyleneimine (PEI)).

Particular embodiments utilize CRISPR gene editing. In particular embodiments, CRISPR gene editing can occur with CRISPR guide RNA (crRNA) and/or a CRISPR nuclease (e.g., Cpf1 (also referred to as Cas12a) or Cas9).

Particular embodiments adopt features that increase the efficiency and/or accuracy of HDR. For example, Cpf1 has a short single crRNA and cuts target DNA in staggered form with 5′ 2-4 nucleotide (nt) overhangs called sticky ends. Sticky ends are favorable for HDR, Kim et al. (2016) Nat Biotechnol. 34(8): 863-8. Moreover, donor templates should be released from the NP before the genome cut by the RNP occurs to promote HDR. Accordingly, in particular embodiments disclosed herein donor templates are found farther from the surface of the NP than targeting elements and cutting elements. The current disclosure also unexpectedly found that delivery of gene-editing components on a AuNP increases the efficiency and/or accuracy of HDR.

Accordingly, particular embodiments deliver gene-editing components utilizing AuNP.

The specific cargo for genetic engineering is tailored to the individual patient based on the treatment outcome desired. When targeting ligands are not included as a component of the NP, the NP provide for reduced manipulation manufacturing removing the need to utilize electroporation and viral vector delivery. The inclusion of targeting ligands allows for minimal manipulation manufacturing removing the need to perform cell selection and purification processes.

Following addition of the NP to a reduced or minimally-manipulated blood cell product, a period of incubation occurs. Following this, optionally cell products may be washed to remove excess NP and re-administered to the patient. In particular embodiments, cells can be stored.

Storage can include room temperature, refrigeration (2-8° C.), or cryopreservation (<−20° C. including storage in liquid nitrogen or vapor phase) conditions depending on the length of time required for patient preparation for reinfusion. The biological sample can be cryo-preserved before and/or after exposure to the NP before re-infusion to a patient.

Aspects of the Disclosure are now described in additional detail and options as follows: (I) Gene Editing Systems and Components; (II) Nanoparticles and their Conjugation with Gene-Editing Components; (Ill) Gene Editing Efficiency; (IV) Selected Cells and Selected Cell Targeting Ligands; (V) Sources & Processing of Cell Populations; (VI) Formulation and Cryopreservation of Cells; (VII) Nanoparticle Formulations; (VIII) Kits; (IX) Exemplary Methods of Use; (X) Exemplary Manufacturing Protocols & Comparisons; (XI) Assays to Asses Nanoparticle Performance; (XII) Exemplary Embodiments; (XIII) Experimental Examples; and (XIV) Closing Paragraphs.

### (I) GENE EDITING SYSTEMS AND COMPONENTS

Within the teachings of the current disclosure, any gene editing system capable of precise sequence targeting and modification can be used. These systems typically include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. Alternatively, a targeting element and a cutting element can be linked together into one dual purpose molecule. When insertion of a therapeutic nucleic acid sequence is intended, the systems also include a HDR template (which can include homology arms) associated with the therapeutic nucleic acid sequence. As detailed further below, however, different gene editing systems can adopt different components and configurations while maintaining the ability to precisely target, cut, and modify selected genomic sites.

In particular embodiments, sites for genetic engineering can be targeted using CRISPR gene editing systems. The CRISPR nuclease system is a prokaryotic immune system that confers resistance to foreign genetic elements such as plasmids and phages and provides a form of acquired immunity. CRISPRs are DNA loci containing short repetitions of base sequences. In the context of a prokaryotic immune system, each repetition is followed by short segments of spacer DNA belonging to foreign genetic elements that the prokaryote was exposed to. This CRISPR array of repeats interspersed with spacers can be transcribed into RNA. The RNA can be processed to a mature form and associate with a Cas (CRISPR-associated) nuclease. A CRISPR-Cas system including an RNA having a sequence that can hybridize to the foreign genetic elements and Cas nuclease can then recognize and cut these exogenous genetic elements in the genome.

A CRISPR-Cas system does not require the generation of customized proteins to target specific sequences, but rather a single Cas enzyme can be programmed by a short guide RNA molecule (crRNA) to recognize a specific DNA target. The CRISPR-Cas systems of bacterial and archaeal adaptive immunity show extreme diversity of protein composition and genomic loci architecture. The CRISPR-Cas system loci have more than 50 gene families and there are no strictly universal genes, indicating fast evolution and extreme diversity of loci architecture. So far, adopting a multi-pronged approach, there is comprehensive Cas gene identification of 395 profiles for 93 Cas proteins. Classification includes signature gene profiles plus signatures of locus architecture. A classification of CRISPR-Cas systems is proposed in which these systems are broadly divided into two classes, Class1 with multi-subunit effector complexes and Class 2 with single-subunit effector modules exemplified by the Cas9 protein. Efficient gene editing in human CD34+ cells using electroporation of CRISPR/Cas9 mRNA and single-stranded oligodeoxyribonucleotide (ssODN) as a donor template for HDR has been demonstrated. De Ravin et al. Sci Transl Med. 2017; 9(372): eaah3480. Novel effector proteins associated with Class2 CRISPR-Cas systems may be developed as powerful genome engineering tools and the prediction of putative novel effector proteins and their engineering and optimization is important.

In addition to the Class 1 and Class 2 CRISPR-Cas systems, more recently a putative Class2, Type V CRISPR-Cas class exemplified by Cpf1 has been identified Zetsche et al) 0.2015 (Cell 163)3(: 759-771.

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation, potentially increasing the efficiency of HDR. Successful HDR results in mutation of the PAM sequence such that no further cutting occurs. Moreover, staggered DSBs with sticky ends permit orientation-specific donor template insertion, which is advantageous in non-dividing cells.

As indicated previously, particular embodiments adopt features that increase the efficiency and/or accuracy of HDR. For example, Cpf1 has a short single crRNA and cuts target DNA in staggered form with 5′ 2-4 nucleotide (nt) overhangs called sticky ends. Sticky ends are favorable for HDR, Kim et al. (2016) Nat Biotechnol. 34(8): 863-8. Moreover, donor templates should be released from the NP before the genome cut by the RNP occurs to promote HDR. Accordingly, in particular embodiments disclosed herein donor templates are found farther from the surface of the NP than targeting elements and cutting elements. The current disclosure also unexpectedly found that delivery of gene-editing components on a AuNP increases the efficiency and/or accuracy of HDR. Accordingly, particular embodiments deliver gene-editing components utilizing AuNP.

Particular embodiments can utilize engineered variant Cpf1s. For example, US 2018/0030425 describes engineered Cpf1 nucleases from Lachnospiraceae bacterium ND2006 and Acidaminococcus sp. BV3L6 with altered and improved target specificity. Particular variants include Lachnospiraceae bacterium ND2006 with mutations (i.e., replacement of the native amino acid with a different amino acid, e.g., alanine, glycine, or serine), at one or more of the following positions: S203, N274, N278, K290, K367, K532, K609, K915, Q962, K963, K966, K1002, and/or S1003. Particular Cpf1 variants can also include Acidaminococcus sp. BV3L6 Cpf1 (AsCpf1) with mutations (i.e., replacement of the native amino acid with a different amino acid, e.g., alanine, glycine, or serine (except where the native amino acid is serine)), at one or more of the following positions: N178, S186, N278, N282, R301, T315, S376, N515, K523, K524, K603, K965, Q1013, Q1014, and/or K1054. In particular embodiments, engineered Cpf1 variants include eCfp1. Other Cpf1 variants are described in US 2016/0208243 and WO/2017/184768.

Particular embodiments utilize zinc finger nucleases (ZFNs) as gene editing agents. ZFNs are a class of site-specific nucleases engineered to bind and cleave DNA at specific positions.

ZFNs are used to introduce double strand breaks (DSBs) at a specific site in a DNA sequence which enables the ZFNs to target unique sequences within a genome in a variety of different cells.

Moreover, subsequent to double-stranded breakage, HDR or NHEJ takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, FokI endonuclease. The FokI domain functions as a dimer requiring two constructs with unique DNA binding domains for sites on the target sequence. The FokI cleavage domain cleaves within a five or six base pair spacer sequence separating the two inverted half-sites.

For additional information regarding ZFNs, see Kim, et al. Proceedings of the National Academy of Sciences of the United States of America 93, 1156-1160 (1996); Wolfe, et al. Annual review of biophysics and biomolecular structure 29, 183-212 (2000); Bibikova, et al. Science 300, 764 (2003); Bibikova, et al. Genetics 161, 1169-1175 (2002); Miller, et al. The EMBO journal 4, 1609-1614 (1985); and Miller, et al. Nature biotechnology 25, 778-785 (2007)].

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant FokI endonucleases. For additional information regarding TALENs, see Boch, et al.

Science 326, 1509-1512 (2009); Moscou, & Bogdanove, Science 326, 1501 (2009); Christian, et al. Genetics 186, 757-761 (2010); and Miller, et al. Nature biotechnology 29, 143-148 (2011).

Particular embodiments utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Exemplary crRNAs for relevant genetic engineering targets include:

(SEQ ID NO: 80, chr11-gsh-gRNA 1)
UAAUUUCUACUCUUGUAGAUUUCGGACCCGUGCUACAACUU;
(SEQ ID NO: 81, chr11-gsh-gRNA 2)
UAAUUUCUACUCUUGUAGAUAUAGAAUAGCCUCAUAUUUUA;
(SEQ ID NO: 82, chr11-gsh-gRNA 3)
UAAUUUCUACUCUUGUAGAUGAGCUGUUGGCAUCAUGUUCCUG;
(SEQ ID NO: 83, chr11-gsh-gRNA 4)
UAAUUUCUACUCUUGUAGAUUCCAAACCUCCUAAAUGAUAC;
and
(SEQ ID NO: 5, chr11-gsh-gRNA 5)
UAAUUUCUACUCUUGUAGAUCACCCGAUCCACUGGGGAGCA.

Relevant target sites for genetic engineering include (with PAM sites italicized):

(SEQ ID NO: 84, chr11-gsh-target 1)
TTTGTGTCCCCGTTTTGGTTGGTAAAC;
(SEQ ID NO: 85, chr11-gsh-target 2)
TTTAAAAATCAATACCGATAATAATGA;
(SEQ ID NO: 86, chr11-gsh-target 3)
TTTCTTAATATGAATATTAATATCGGT;
(SEQ ID NO: 87, chr11-gsh-target 4)
TTTCCGTATCTGGAAGGGGCATCTTGG;
(SEQ ID NO: 88, chr11-gsh-target 5)
TTTCCTTAGGACCGGAAGGATTACAGC;
(SEQ ID NO: 89, chr11-gsh-target 6)
TTTGCCTAAAAGGCACTATGTCAAATG;
(SEQ ID NO: 90, chr11-gsh-target 7)
TTTGGAGCTGTTGGCATCATGTTCCTG;
(SEQ ID NO: 91, chr11-gsh-target 8)
TTTGATTCTTTTCTATCTCAGGACAGA;
(SEQ ID NO: 92, chr11-gsh-target 9)
TTTATAGACATCCCACACTGTAGTTCT;
(SEQ ID NO: 93, chr11-gsh-target 10)
TTTATTAATTTGAGAACCAACATAAGG;
(SEQ ID NO: 94, chr11-gsh-target 11)
TTTATTTTCTTTTTGGTAAGAAGGAAC;
(SEQ ID NO: 95, chr11-gsh-target 12)
TTTCACACACACACACACACACACACA;
(SEQ ID NO: 96, chr11-gsh-target 13)
TTTATCCAAACCTCCTAAATGATAC;
(SEQ ID NO: 21, chr11-gsh-target 14)
TTTACACCCGATCCACTGGGGAGCA;
and
(SEQ ID NO: 97, chr11-gsh-target 15)
TTTTTGATTCTTTTCTATCTCAGGACA.

These target sites reflect genomic safe harbors (GSH) within HSPC. In particular embodiments, these GSH sites are SEQ ID NOs: 21 and 84-97 (chr11-gsh-target 1-15) reflected above but with 1, 2, 3, or 4 nucleotide substitutions to account for typical genetic variations across populations.

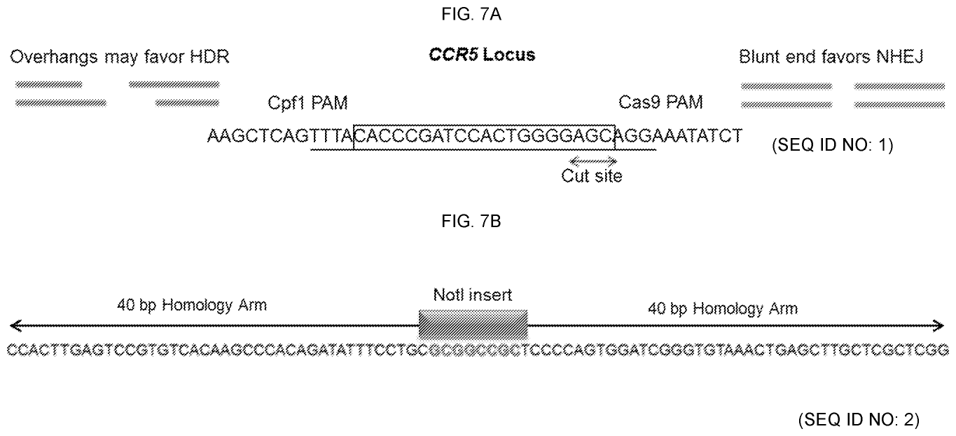

The current disclosure also provides target sites and targeting sequences for loci useful in the treatment of other disorders, such as hemoglobinopathies and human immunodeficiency virus (HIV) (see, e.g., FIGS. 7A, 7B, 8A, 8B, 34 and 35A-35D).

In particular embodiments, NP can deliver factors that promote the desired DNA repair pathway of interest. The first step in any pathway to repair a double-stranded DNA break is stabilization of the free ends of the DNA at the break site. DNA stabilizing proteins specific to the repair pathway of interest can be incorporated to promote that specific DNA repair pathway. For NHEJ, two proteins are involved in stabilizing the free ends of the DNA: Ku70 and Ku80. For HDR, a three-protein complex known as MRN consisting of MRE11, Nbs1 and RAD50 is required.

These molecules can include oligos (mRNA) or proteins for any of the factors involved to ensure that cells receiving gene editing machinery also have these factors present. Alternatively, or in combination, small interfering RNAs (siRNAs, short-hairpin RNAs or microRNAs) that would reduce expression of NHEJ pathways could also be included.

Templates for HDR can be symmetric or asymmetric homology arms as described by Richardson et al., Nat Biotechnol. 2016; 34(3):339-44. Each donor template can include homology arms (HDR template) flanking a 20 bp random DNA barcode element for clone tracking, upstream of a human phosphoglycerate kinase (PGK) promoter driving expression of therapeutic DNA sequence in clinical use. Humanized Cpf1 protein can be synthesized by a commercial manufacturer (Aldevron), and guide RNA with two modifications, an atom oligoethylene glycol spacer and a 3′ terminal thiol can also be obtained from a commercial source (Integrated DNA Technologies, Coralville, Iowa). Single-stranded homology template DNA (ssODN) can also be synthesized by a commercial manufacturer (Integrated DNA Technologies, Coralville, Iowa). For examples of such sequences, see FIGS. 7A, 7B, 8A, 8B, 34, 35B, and 35D.

As indicated, in particular embodiments, gene editing systems to provide a genetic therapy will include guide RNA and a nuclease. In particular embodiments, donor templates can be used, especially when performing a gain-of-function therapy or a precise loss-of-function therapy. In particular embodiments, gene editing systems include an HDR template and a therapeutic nucleic acid sequence.

All nucleic acid-based components of gene editing systems can be single stranded, double stranded, or may have a mix of single stranded and double stranded regions. For example, guide RNA or a donor template may be a single-stranded DNA, a single-stranded RNA, a double-stranded DNA, or a double-stranded RNA. In particular embodiments utilizing NP described herein, the end of a nucleic acid farthest from the NP surface may be protected (e.g., from exonucleolytic degradation) by methods known to those of skill in the art. For example, one or more dideoxynucleotide residues can be added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include addition of terminal amino group(s) and the use of modified internucleotide linkages such as, for example, phosphorothioates, phosphoramidates, and O-methyl ribose or deoxyribose residues. Chemically modified mRNA can be used to increase intracellular stability, while asymmetric homology arms and phosphorothioate modification can be incorporated into the ssODN to improve HDR efficiency. In particular embodiments utilizing NP described herein, nucleic acids may be protected from electrostatic (charge-based) repulsions by, for example, addition of a charge shielding spacer. In particular embodiments, a charge shielding spacer can include an 18 atom oligoethylene glycol (OEG) spacer added to one or both ends. In particular embodiments, a charge shielding spacer can include a 10⁻²⁶ atom oligoethylene glycol (OEG) spacer added to one or both ends.

Donor templates can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

In particular embodiments, a HDR template (HDT) is designed to serve as a template in homologous recombination, such as within or near a target sequence nicked or cleaved by an enzyme (e.g., nuclease) of a gene editing system. A HDR template polynucleotide may be of any suitable length, such as 10, 15, 20, 25, 50, 75, 100, 150, 200, 500, 1000, 2000, 3000, 4000, 5000, or more nucleotides. In particular embodiments, the HDR template polynucleotide is complementary to a portion of a polynucleotide including the target sequence. When optimally aligned, a HDR template polynucleotide overlaps with one or more nucleotides of a target sequence (e.g., 1, 5, 10, 15, 20, 25, 30, 35, 40, 45, 50, 60, 70, 80, 90, 100 or more nucleotides).

In particular embodiments, the HDR template can include sufficient homology to a genomic sequence at the cleavage site, e.g. 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between it and the genomic sequence to which it bears homology. 25, 50, 100, or 200 nucleotides, or more than 200 nucleotides of sequence homology between a HDR template and a targeted genomic sequence (or any integral value between 10 and 200 nucleotides, or more) can support HDR. Homology arms or flanking sequences are generally identical to the genomic sequence, for example, to the genomic region in which the double stranded break (DSB) occurs. However, absolute identity is not required.

In particular embodiments, the donor template includes a heterologous therapeutic nucleic acid sequence flanked by two regions of homology, such that HDR between the target DNA region and the two flanking sequences results in insertion of the heterologous therapeutic nucleic acid sequence at the target region. In some examples, homology arms or flanking sequences of HDR templates are asymmetrical.

As indicated, in particular embodiments, donor templates include a therapeutic nucleic acid sequence. Therapeutic nucleic acid sequences can include a corrected gene sequence; a complete gene sequence and/or one or more regulatory elements associated with expression of the gene. A corrected gene sequence can be a portion of a gene requiring correction or can provide a complete replacement copy of a gene. A corrected gene sequence can provide a complete copy of a gene, without necessarily replacing an existing defective gene. One of ordinary skill in the art will recognize that removal of a defective gene when providing a corrected copy may or may not be required. When inserting a gene within a genetic safe harbor, a therapeutic nucleic acid sequence should include a coding region and all regulatory elements required for its expression.

Examples of therapeutic genes and gene products include skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C90RF72, α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, FancI, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT−1, YES, zac1, iduronidase, IDS, GNS, HGSNAT, SGSH, NAGLU, GUSB, GALNS, GLB1, ARSB, HYAL1, F8, F9, HBB, CYB5R3, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.

In particular embodiments, a therapeutic gene includes a coding sequence for a therapeutic expression product (e.g., protein, RNA) and all associated regulatory elements (e.g., promoters, etc.) to result in expression of the gene product.

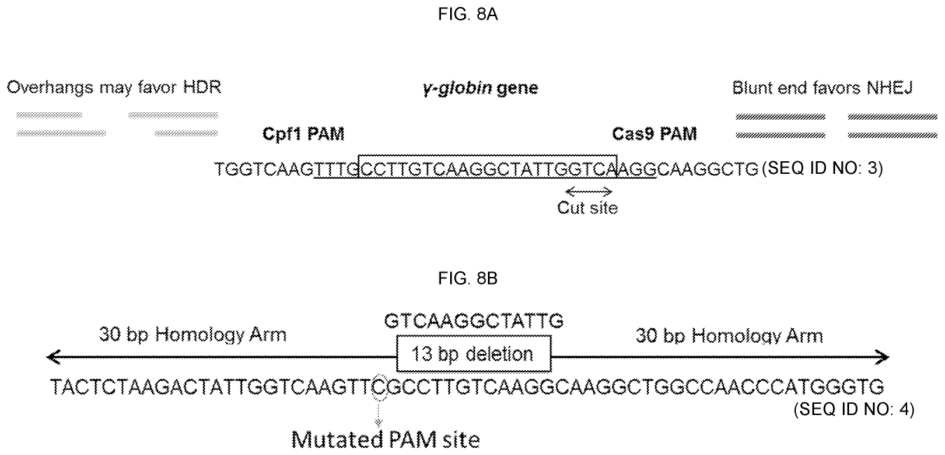

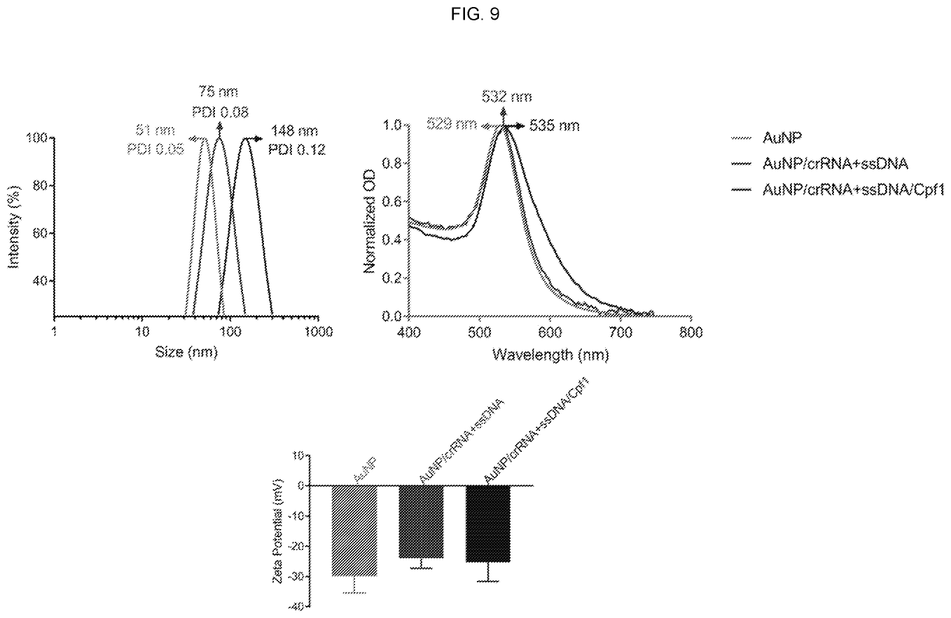

In particular embodiments, therapeutic genetic engineering disrupts a genetic site to prevent binding. See, for example, FIG. 8A, 8B. In particular embodiments, genetic engineering is based on gene-editing components including Cpf1 and guide RNA targeting a single nucleotide polymorphism (SNP) or 13 nucleotide deletion overlapping a BCL11a binding site in the γ globin locus on chromosome 11 or a SNP within an erythroid-specific enhancer element in the second intron of the BCL11a gene on chromosome 2. In particular embodiments, genetic engineering is based on gene-editing components including Cpf1 and guide RNA targeting a mutation located within a 5 bp BCL11a binding site of the γ-globin locus on chromosome 11 or one of two SNP mutations located in the BCL11a gene on chromosome 2 in an erythroid-specific enhancer region selected from rs1427407 and rs7569946. See also FIGS. 8A, 8B, 34 and 35A-35D.

In particular embodiments, a therapeutic nucleic acid sequence (e.g., a gene) can be selected for incorporation into a genetic site to provide for in vivo selection of the genetically modified cell. For example, in vivo selection using a cell-growth switch allows a minor population of genetically modified cells to be inducibly amplified. A strategy to achieve in vivo selection has been to employ drug selection while coexpressing a transgene that conveys chemoresistance, such as 06-methylguanine-DNA-methyltransferase) MGMT. (An alternate approach is to confer an enhanced proliferative potential upon gene-modified HSC through the delivery of the homeobox transcription factor HOXB4. In particular embodiments, a suicide gene can be incorporated into the genetically modified cell so that such population of cells can be eliminated, for example, by administration of a drug that activities the suicide gene. See, for example, Cancer Gene Ther. 2012 August; 19(8):523-9; PLoS One. 2013; 8(3):e59594. and Molecular Therapy—Oncolytics (2016) 3, 16011.

Particular embodiments include contacting a blood cell with a gene editing system capable of inserting a donor template at a target site. In particular embodiments, the gene editing system includes crRNA capable of hybridizing to a target sequence, and a nucleic acid encoding a nuclease enzyme such as Cpf1 or Cas9.

Particular embodiments include contacting a blood cell with a gene editing system capable of inserting a donor template at a target site. In particular embodiments, the gene editing system includes crRNA capable of hybridizing to a target sequence and a nucleic acid encoding a nuclease enzyme such as Cpf1 or Cas9. In particular embodiments, Cas9 or Cpf1 coding sequences can include SEQ ID NOs: 112-124. In particular embodiments, Cas9 or Cpf1 amino acid sequences can include SEQ ID NOs: 125-138.

### (II) NANOPARTICLES AND THEIR CONJUGATION WITH GENE-EDITING COMPONENTS

As indicated, delivery methods of gene editing systems that do not rely on electroporation, viral vectors, and/or cell selection or purification processes are needed.

The current disclosure provides engineered NP that allow delivery of the gene editing components without the need to rely on electroporation or viral vector delivery of gene-editing components. When a therapeutic use need only de-activate a problematic gene, the NP need only be associated with a targeting element and a cutting element (although other components may be included as necessary or helpful for a particular purpose). When a therapeutic use adds or corrects a gene, the NP are associated with a targeting element, a cutting element, and a donor template. To further avoid cell selection or purification processes, targeting ligands can be attached to the NP to result in selective delivery of the NP to a selected cell population within a heterogenous pool of cells.

Particular embodiments utilize colloidal metal NP. A colloidal metal includes any water-insoluble metal particle or metallic compound dispersed in liquid water. A colloid metal can be a suspension of metal particles in aqueous solution. Any metal that can be made in colloidal form can be used, including Au, silver, copper, nickel, aluminum, zinc, calcium, platinum, palladium, and iron. In particular embodiments, AuNP are used, e.g., prepared from HAuCI4. In particular embodiments, the NP are non-Au NP that are coated with Au to make Au-coated NP.

Methods for making colloidal metal NP, including Au colloidal NP from HAuCI4, are known to those having ordinary skill in the art. For example, the methods described herein as well as those described elsewhere (e.g., US 2001/005581; 2003/0118657; and 2003/0053983) can be used to make NP.

In particular exemplary embodiments, AuNP cores were synthesized in three different size ranges (15, 50, 100 nm) by an optimized Turkevich and seeding-growth methods (Shahbazi, et al., Nanomedicine (Lond), 2017. 12(16): p. 1961-1973; Shahbazi, et al., Nanotechnology, 2017. 28(2): p. 025103; Turkevich, et al. Discussions of the Faraday Society, 1951. 11(0): p. 55-75; Perrault & Chan, Journal of the American Chemical Society, 2009. 131(47): p. 17042-17043). In the first step, seed AuNPs of 15 nm were synthesized by bringing 100 mL of 0.25 mM Au (Ill) chloride trihydrate solution to the boiling point and adding 1 mL of 3.33% trisodium citrate dehydrate solution. Synthesis of NP was carried out in high stirring speeds over 10 min. Prepared NP were cooled down to 4° C. and used in the following growth step.

In order to prepare AuNPs in 50 nm and 100 nm size ranges, two different 100 mL of 0.25 mM Au (Ill) chloride trihydrate solutions were prepared and in mild stirring conditions 2440 μL and 304 μL of seed AuNPs were added separately to synthesize 50 nm and 100 nm AuNPs, respectively. To these solutions was added 1 mL of 15 mM trisodium citrate dehydrate solution and the mixture was brought to the highest stirring speed. Then, 1 mL of 25 mM hydroquinone solution was added and synthesis was continued over 30 min for 50 nm AuNPs and 5 h for 100 nm AuNPs. Finally, synthesized NP were purified by centrifuging at 5000×g and dispersing in ultra-pure water. In particular embodiments NP cores are >100 nm; >90 nm; >80 nm; >70 nm; >60 nm; >50 nm; >40 nm; >30 nm; or 20 nm.

While AuNPs are particularly described, NP encompassed in the present disclosure may be provided in different forms, e.g., as solid NP (e.g., metal such as silver, Au, iron, titanium), non-metal, lipid-based solids, polymers, suspensions of NP, or combinations thereof. Metal, dielectric, and semiconductor NP may be prepared, as well as hybrid structures (e.g., core-shell NP). NP made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure.

As indicated, a variety of active components can be conjugated to the NP disclosed herein for targeted gene editing. For example, nucleic acids that are gene editing system components can be conjugated directly or indirectly, and covalently or noncovalently, to the surface of the NP.

For example, a nucleic acid may be covalently bonded at one end of the nucleic acid to the surface of the NP.

Nucleic acids conjugated to the NP can have a length of from 10 nucleotides (nt)-1000 nt, e.g., 1 nt-25 nt, 25 nt-50 nt, 50 nt-100 nt, 100 nt-250 nt, 250 nt-500 nt, 500 nt-1000 nt or greater than 1000 nt. In particular embodiments, nucleic acids modified by conjugation to a linker do not exceed 50 nt or 40 nt in length.

When conjugated indirectly through, for example, an intervening linker, any type of molecule can be used as a linker. For example, a linker can be an aliphatic chain including at least two carbon atoms (e.g., 3, 4, 5, 6, 7, 8, 9, 10, 11, 12, 13, 14, or more carbon atoms), and can be substituted with one or more functional groups including a ketone, ether, ester, amide, alcohol, amine, urea, thiourea, sulfoxide, sulfone, sulfonamide, and/or disulfide.

In particular embodiments the linker includes a disulfide at the free end (e.g. the end not conjugated to the guide RNA) that couples the NP surface. In particular embodiments, the disulfide is a C2-C10 disulfide, that is it can be an aliphatic chain terminating in a disulfide that includes 2, 3, 4, 5, 6, 7, 8, 9, or 10 carbon atoms, although it is envisioned that longer aliphatic chains can be used. In particular embodiments, the disulfide is a 3 carbon disulfide (C3 S-S).

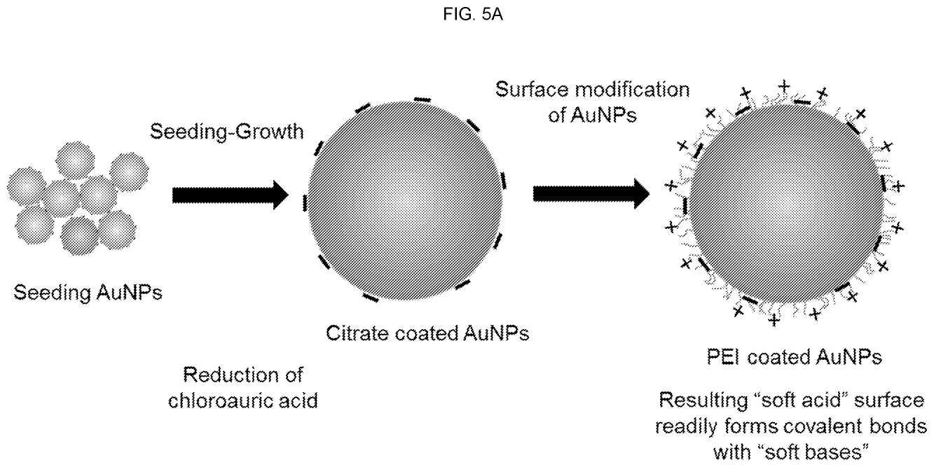

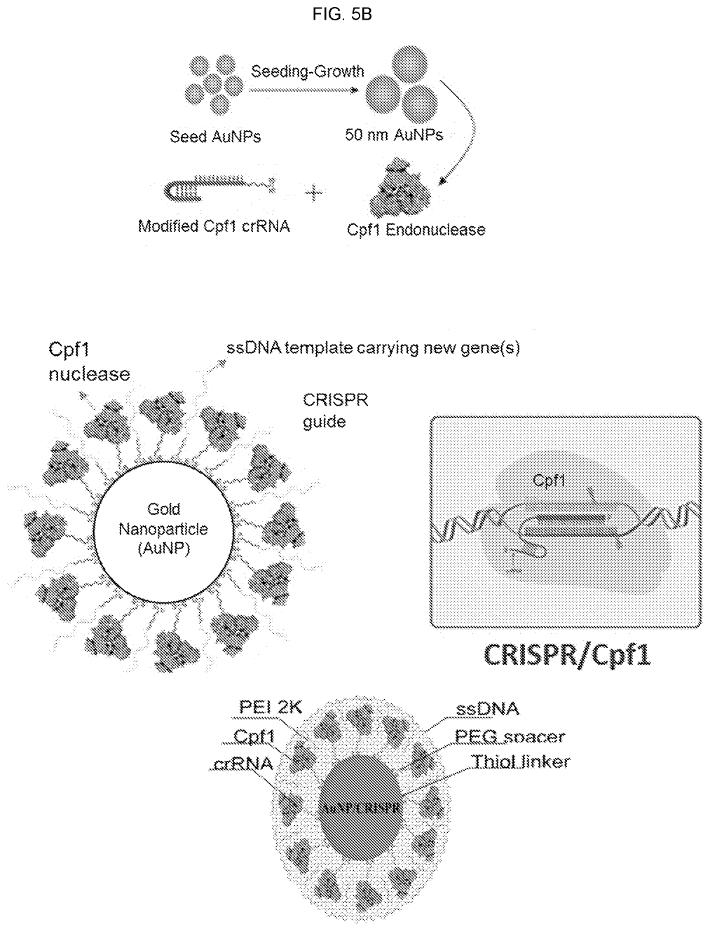

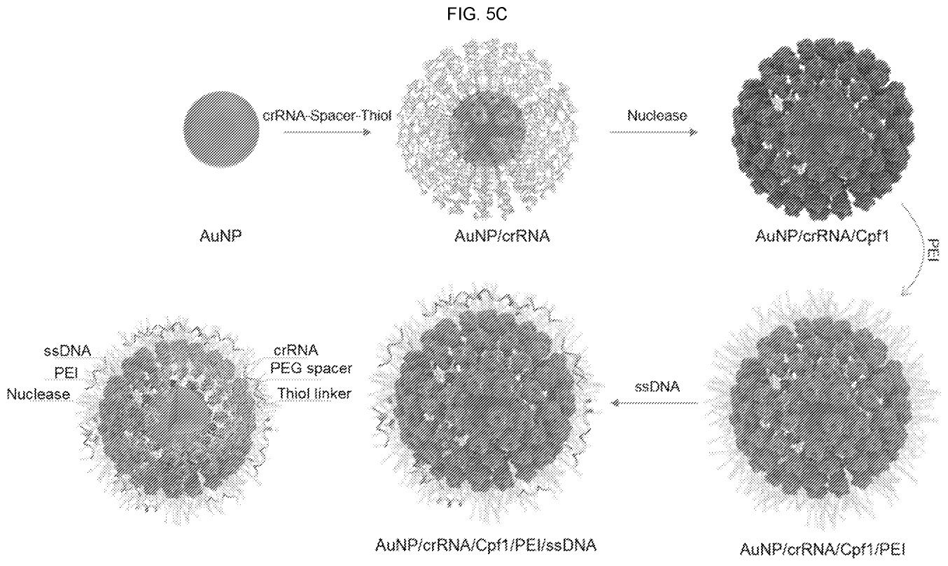

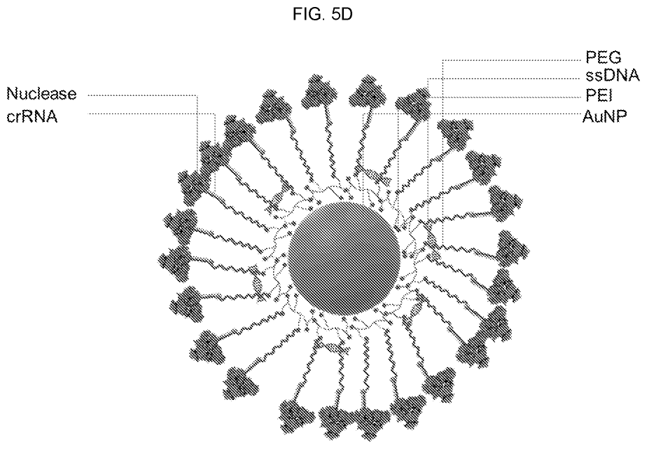

Linkers can have either sulfhydryl groups (SH) or disulfide groups (S-S) or a different number of sulfur atoms. In particular embodiments, a thiol modification can be introduced without using a linker. In particular embodiments, a nuclease enzyme is delivered as a protein pre-conjugated with its guide RNA (a ribonucleoprotein (RNP) complex). In this formulation, the guide RNA molecule is bound to the NP and the nuclease enzyme, by default, can be also bound (see, for example, FIG. 5B).

One advance disclosed herein is the ability to modify CRISPR components for linkage to a NP. This is because most of the modifications in CRISPR components can compromise cutting efficiency. For example, Li et al. (Engineering CRISPR-Cpf1 crRNAs and mRNAs to maximize genome editing efficiency. 2017. 1: p. 0066) indicated that the 5′ end of Cpf1 crRNA is not safe for any modification because such modifications result in the abrogation of the crRNA binding to Cpf1 nuclease. Disclosed herein is a modification to the 3′ end of crRNA that does not compromise cutting efficiency. In particular embodiments, in the first step of conjugation to a NP the 3′ end of the crRNA is modified with an 18-atom hexa-ethyleneglycol spacer (18 spacer) and 3 carbon disulfide (C3 S-S) to attach the crRNA to the surface of AuNPs.

Based on the foregoing, in particular embodiments, for example when the NP includes Au, a linker can be any thiol-containing molecule. Reaction of a thiol group with Au results in a covalent sulfide (—S—) bond. AuNPs have high affinity to thiol (-SH) and dithiol (S-S) groups and semi-covalent bonds occur between the surface of AuNP and sulfur groups (Hakkinen, Nat Chem, 2012. 4(6): p. 443-455). In particular embodiments, thiol groups can be added to nucleic acids to facilitate attachment to the surface of AuNPs. This approach can improve nucleic acid uptake and stability (see, e.g., Mirkin, et al., A Nature, 1996. 382(6592): p. 607-609).

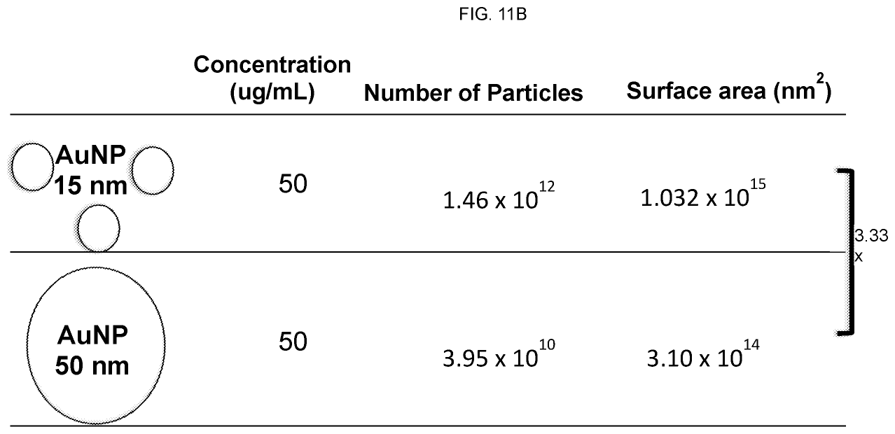

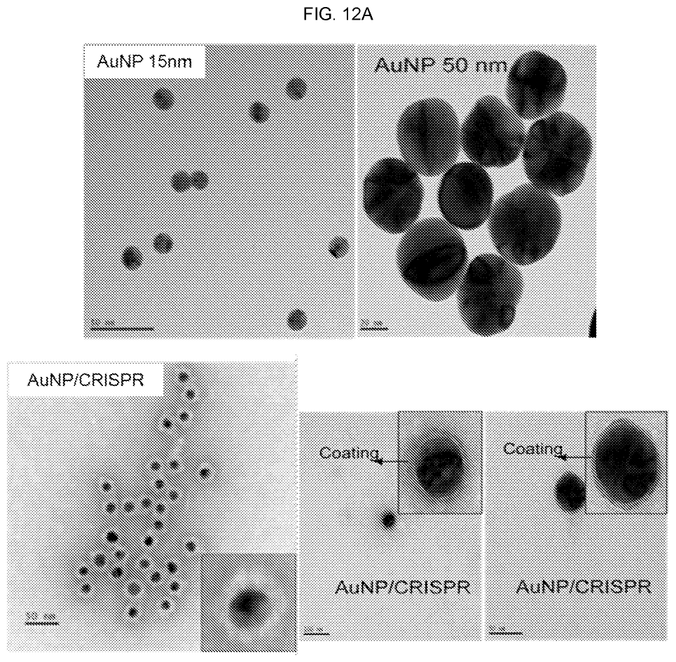

Using an optimized two step method of seeding-growth, highly monodisperse AuNPs were synthesized in 3 different size ranges (15 nm, 50 nm, 100 nm) and conjugated with Cpf1 crRNA and endonuclease (FIGS. 5B and 11B). Because of the strong electrostatic repulsion between the negatively charged surface and negatively charged crRNA it is difficult to attach the crRNA to the surface of AuNPs without, for example, the thiol modification. In particular embodiments, in the second step, after purification of the crRNA conjugated AuNPs, Cpf1 endonuclease is added and incubated with crRNA conjugated AuNPs to facilitate its binding to the 5′ handle of the crRNA (Dong, et al., Nature, 2016. 532(7600): p. 522-526). The compact structure of the designed NP containing both crRNA and Cpf1 endonuclease results in a conformation which increases the stability against degrading agents and facilitates the uptake of the Au/CRISPR NP by cells owing to an overall neutral charge (i.e., zeta potential). While special relevance was given to optimizing the disclosed NP for CRISPR/Cpf1, the same concept may be applied to other CRISPR classes.

Also, along with the crRNA and Cpf1 endonuclease, 18 spacer thiol modified single stranded DNA (ssDNA) can be attached to the surface of AuNPs to obtain a novel NP with the aim of being used in homology directed repair (HDR).

In particular embodiments, a spacer-thiol linker can be added to either of the Cpf1 or Cas9 proteins themselves or engineered variants of the foregoing (e.g., as described below), by addition of a cysteine residue on either the N- or C-terminus. The nuclease protein can then be added as a first layer on the AuNP core's surface. This spacer-thiol linker can increase the stability of the protein and increase cutting efficiency. In particular embodiments, an RNA complex is formed between crRNA and nuclease and then attached to the surface of AuNP core's surface through a spacer-thiol linker.

As indicated previously, adding gene-editing components of a bacterial origin as a first loading step can provide beneficial shielding of these components following administration to a subject with pre-existing immunity to the component. The shielding can be due to other gene-editing components (e.g., donor templates) and need not rely on a protective polymer shell. In particular embodiments, a polymer shell is excluded. In particular embodiments, the shielding may permit serial in vivo administration.

In particular embodiments, crRNAs can be added to AuNPs in different AuNP/crRNA w/w ratios (0.25, 0.5, 1, 1.5, 2, 3, 4, 5, 6) and mixed. Citrate buffer with the pH of 3 can be added to the mixture in 10 mM concentration to screen the negative repulsion between negatively charged crRNA and AuNP. After stirring for 5 min, NP can be centrifuged down and the unbound crRNA can be visualized by agarose gel electrophoresis. After determining the optimal conjugation concentration, 1 μL of 63 μM Cpf1 nuclease can be added to AuNP/crRNA solution and incubated for 20 min.

Importantly, the use of a citrate buffer provides significant advantages in manufacturing.

Previous methods have relied on the use of NaCl to screen the negatively-charged NP surface and reduce repulsion of similarly negatively-charged DNA. However, NaCl can cause irreversible aggregation of AuNP, so it must be added gradually over time with incremental changes in concentration. Generally, NaCl must be added over a 48-hour time period to avoid aggregation.

When citrate buffer is used with a pH of 3, this binding can happen with higher efficiency in less than 3 minutes. Zhang, et al. (2012). Journal of the American Chemical Society 134(17): 7266-7269 reducing the cost of goods and time in the GMP manufacturing facility.

Size and morphology of prepared Au/CRISPR NP can be characterized by imaging under transmission electron microscope (TEM). AuNPs (4 μL) can be added to copper grids and allowed to dry out overnight. Imaging is carried out at 120 kV.

Coating with gene-editing components can be visualized by negative staining electron microscopy. For example, NP can be stained with 0.7% uranyl formate and 2% uranyl acetate, respectively. Stained sample (4 μL) can be added to carbon-coated copper grid and incubated for 1 min and blotted with a piece of filter paper. After three washing cycles with 20 μl stain solution, 4 μl stain solution can be added to the grids and blotted and air dried.

NP can also be characterized by Nanodrop UV-visible spectrophotometer by analyzing the shifts in localized surface plasmon resonance (LSPR) peak of the N P before and after conjugation with gene-editing components.

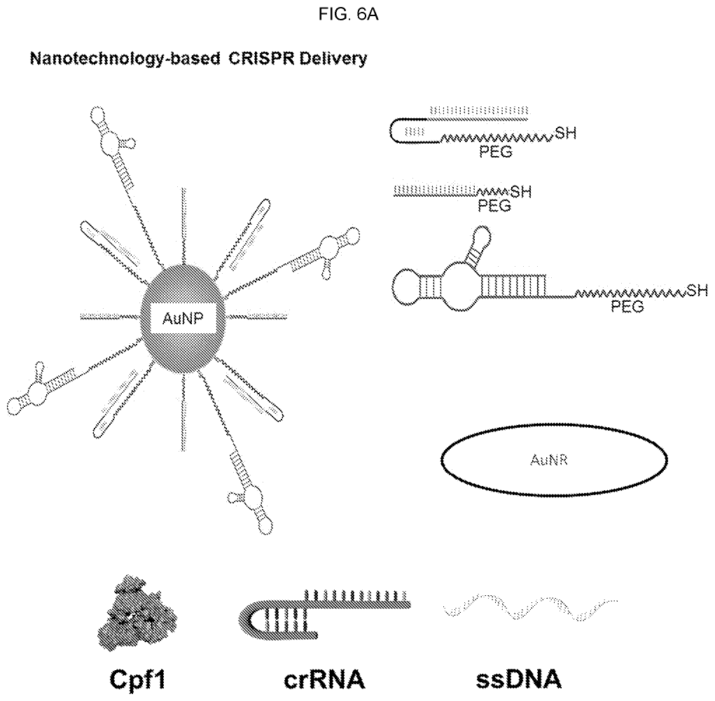

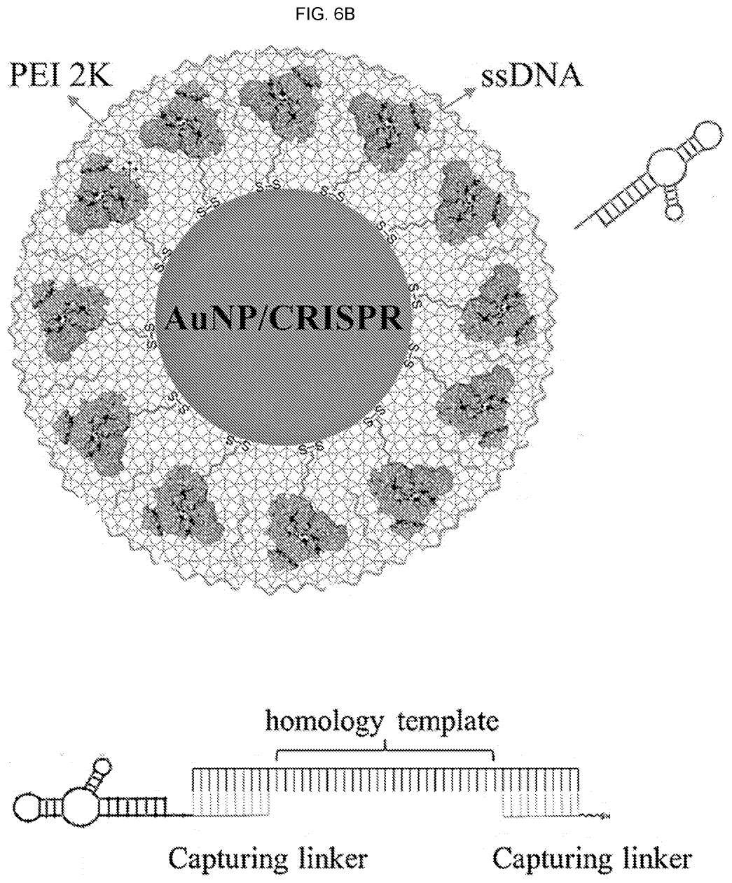

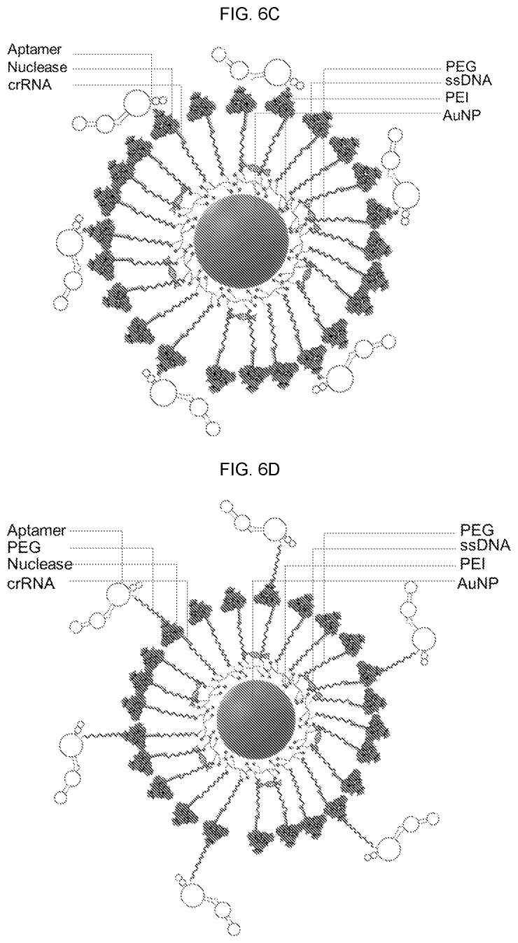

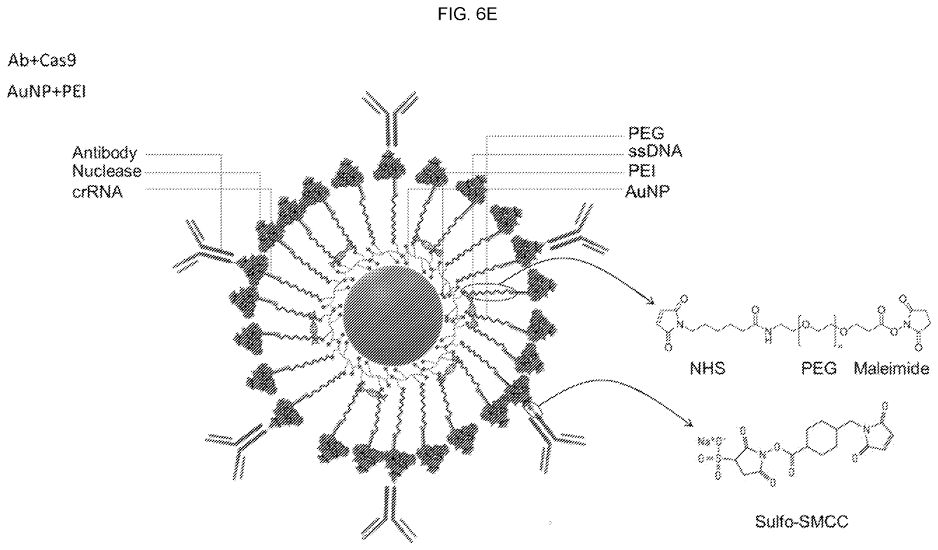

In particular embodiments, a NP is layered, such as during synthesis to include PEI or other positively charged polymers for increasing surface area and conjugating larger ssDNA or other molecules, such as targeting ligands and/or large donor templates (see, for example, FIG. 6B). This NP can be prepared in a layer by layer form and positively charged polymers (such as; PEI in different molecular weights and forms) can be used to coat the negatively charged surface of either AuNP or gene-editing component coated AuNP to attach either gene editing components and other components (such as antibody binding domains). Layering essentially increases the surface area of the NP available for conjugating molecules such as large oligonucleotides with or without other proteins.

Particular embodiments utilize a positively charged polymer with a molecular weight between 1,000-3,000 daltons (e.g., 1,000; 1,200; 1,400; 1,600; 1,800; 2,000; 2,200; 2,400; 2,600; 2,800; or 3,000 daltons). Examples of positively-charged polymers include polyamines; polyorganic amines (e.g., polyethyleneimine (PEI), polyethyleneimine celluloses); poly(amidoamines) (PAMAM); polyamino acids (e.g., polylysine (PLL), polyarginine); polysaccharides (e.g, cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins.

Blends of polymers (and optionally lipids) in any concentration and in any ratio can also be used. Blending different polymer types in different ratios using various grades can result in characteristics that borrow from each of the contributing polymers. Various terminal group chemistries can also be adopted.

In particular embodiments, a positively-charged polymer (e.g., PEI) can be added as a coating on already-formed portions of an NP and ssDNA can be added concurrently or thereafter.

Alternatively, the conjugation steps can be changed by adding ssDNA as a layer followed by addition of a positively-charged polymer as a subsequent layer. In particular embodiments, positively-charged polymers, and ssDNA are not included as a first layer, as this layer can be reserved for RNP complexes coupled to linkers.

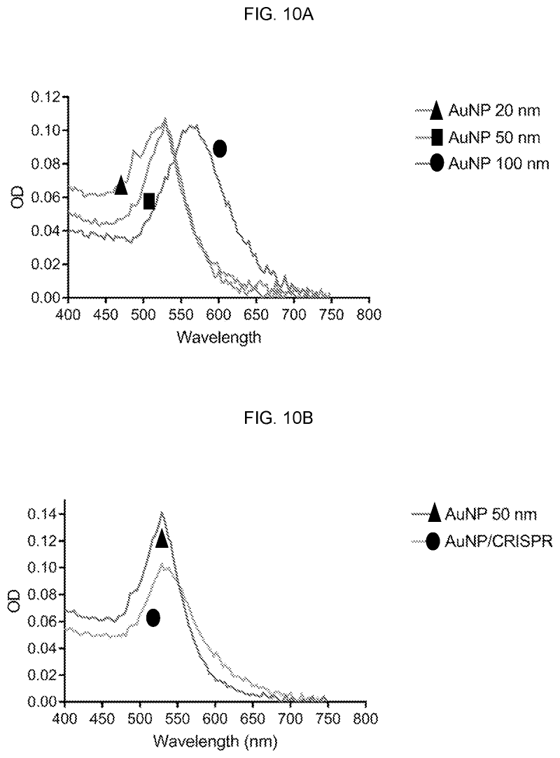

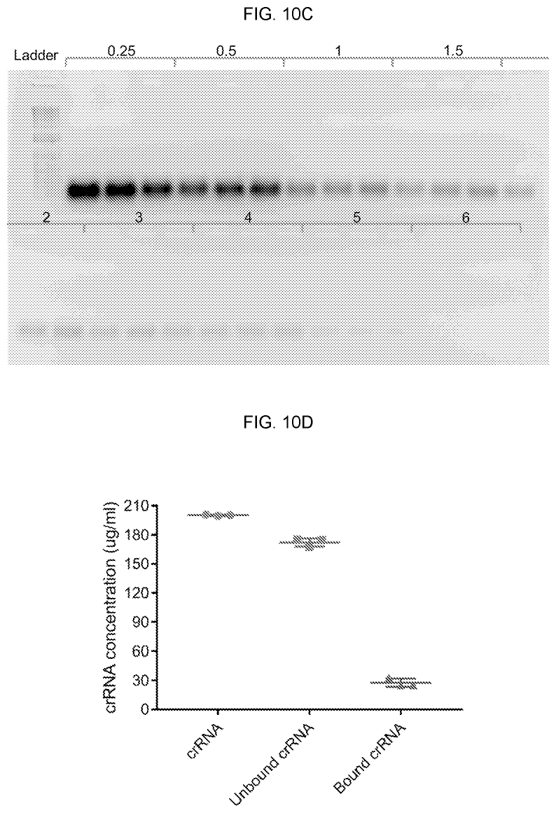

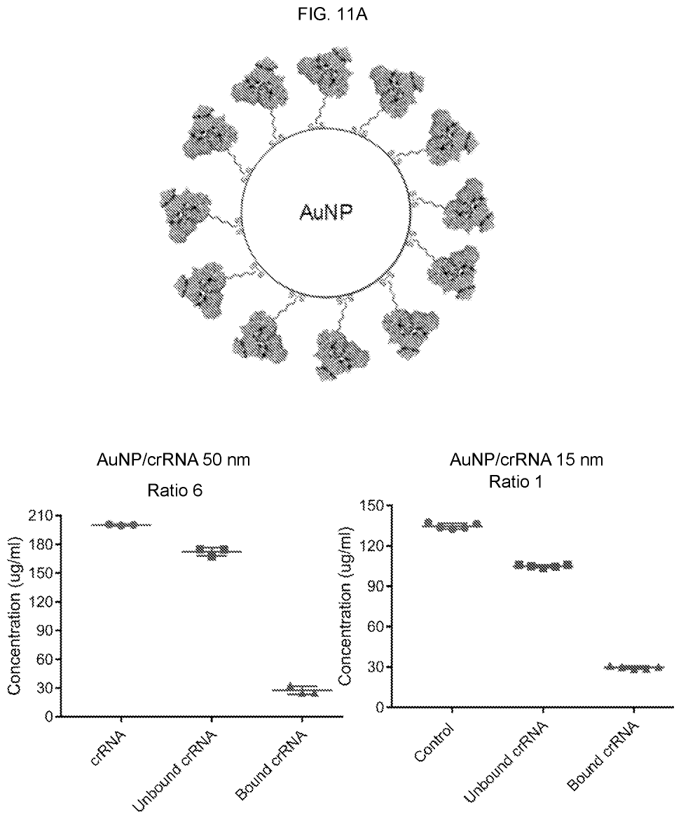

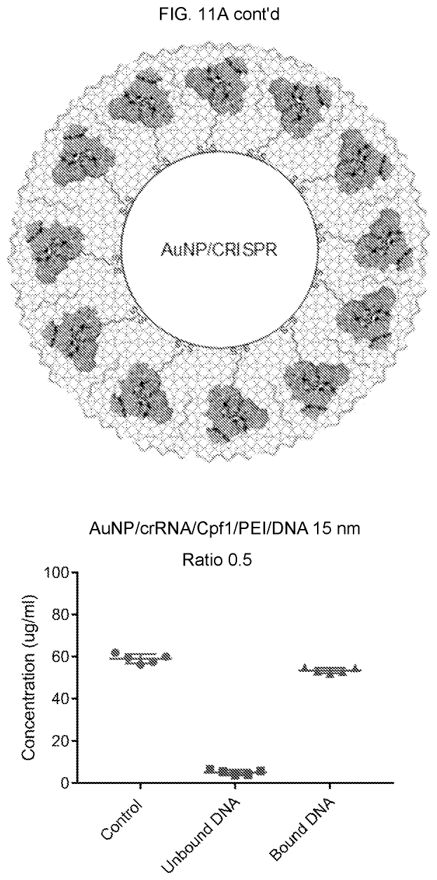

In particular embodiments, a multilayered NP of the disclosure has an average size of 25-70 nm and is highly monodisperse. Transmission electron microscope images (TEM) and LSPR of AuNP showed a uniform surface coating without any aggregation (FIGS. 10A, 10B). Given the synthetic nature of the entire delivery system, all components can be assembled within a few hours, as opposed to previous approaches which required multiple days due to, for example, use of NaCl as a charge screen.

As shown in FIG. 10A, synthesized NP were highly monodisperse and successful 4 nm coating without any aggregation was achieved which increased the size of the NP to 54 nm after coating for 50 nm AuNPs. Also, decrease in the intensity and red shifting of the LSPR of AuNPs showed the successful conjugation with gene-editing components without any aggregation (FIG. 10A). Each layer will have a different optimal loading ratio. The first layer consists of RNA, however to test the optimal ratio for loading this layer, a single stranded DNA test nucleotide was used (ssDNA). This test oligonucleotide was modified with the same 18 spacer C3 S-S used to modify crRNA. In loading studies, different AuNP/crRNA w/w ratios showed that the ratio of 6 particle core:ssDNA (and by inference, crRNA) is optimal to carry out the conjugation (FIG. 10C). Using this optimal loading ratio crRNA was loaded on the surface of AuNPs in 30 μg/mL concentration (FIG. 10D). These data help calculate the exact application dosage for gene editing studies.

As will be understood by one of ordinary skill in the art, the provided ratios are iterative, because as each layer is added, the ratio for optimal loading is slightly different. Characteristics of the NP as a whole, as well as the last layer added, and the properties of the new layer to be added all influence the ratio. In particular embodiments, for crRNA (first layer), a ratio of 6:1 is optimal. In particular embodiments, for the Cpf1 protein, a ratio of 0.6 is optimal for loading onto a NP core+crRNA layer, and the final HDT layer has an optimal loading ratio of 1. Modifications to the Cpf1 protein or changes to the length or chemical modification of the HDT can impact these ratios.

Particularly useful ratios of particle core to gene-editing components include weight/weight (w/w) ratios of 0.5; 0.6; or 0.7 particle core: Cpf1 and 0.9; 1.0; or 1.1 particle core: HDT.

The described approaches resulted in a highly potent, loaded, gene-editing NP capable of delivering both synthetic, non-chemically modified ribonucleoproteins along with a ssDNA homology template for insertion of new DNA, without the need for electroporation or viral vector delivery. In particular embodiments, the hydrodynamic size of a fully loaded AuNP is 150-190 nm, 160-185 nm, 170-180 nm or 176 nm.

An additional particle design includes the following components extending from proximal to distal of a NP core's surface in the following order: thiolated PEI, a linker, a targeting element, and a cutting element. In particular embodiments, the linker is a polyethylene glycol linker. In particular embodiments, a water-soluble, amine-to-sulfhydryl crosslinker that contains NHS-ester and maleimide reactive groups at opposite ends of a medium-length cyclohexane spacer arm can be used to link a cutting element with a targeting ligand. In particular embodiments, the amine-to-sulfhydryl crosslinker includes sulfosuccinimidyl 4-[N-maleimidomethyl]cyclohexane-1-carboxylate (sulfo-SMCC, FIG. 6E). In particular embodiments, ssDNA is within a layer surrounding the NP's core that is co-extensive with the linker's layer. This configuration is depicted in, for example, FIGS. 5D and 6C-6E.

Linkers include polymer linkers. In particular embodiments, a linker can be an amino acid sequence having from one up to 500 amino acids, which can provide flexibility and room for conformational movement between two regions, domains, motifs, cassettes or modules connected by the linker. In particular embodiments, linkers can be flexible, rigid, or semi-rigid, depending on the desired function or structure of components joined by the linker. In particular embodiments, a linker can be direct when it connects two molecules, regions, domains, motifs, cassettes or modules. In particular embodiments, a linker can be indirect when two molecules, regions, domains, motifs, cassettes or modules are not connected directly by a single linker but by linkers from both sides to yet a third linker or domain. Exemplary linker sequences include those having from one to ten repeats of GlyₓSer_{y}, wherein x and y are independently an integer from 0 to 10 provided that x and y are not both 0 (e.g., (Gly₄Ser)₃ (SEQ ID NO: 98), (Gly₃Ser)₂ (SEQ ID NO: 99), Gly₂Ser, or a combination thereof such as (Gly₃Ser)₂Gly₂Ser) (SEQ ID NO: 100)).

Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

### (III) GENE EDITING EFFICIENCY

The optimal concentrations of crRNA, hAsCpf1 RNA and ssODN for electroporation were determined in K562 cells. The optimal concentration displays the highest viability and GFP expression. K562 cells were cultured in 24 well plates in 1×10⁵ cells/well concentration. Iscove's Modified Dulbecco's Medium (IMDM) with 10% FBS and 1% PenStrep was used to culture the cells. CD34+ cells were cultured in 24 well plates in 5×10⁵ cells/well concentration. Culture conditions for CD34+ cells were the same as K562 cells with required growth factors. Au/CRISPR NP were added in 25 nM concentration to the wells and editing efficiency was evaluated after 48 h incubation. In particular embodiments, AuNP/CRISPR can be incubated with cell populations for 1-48 h, 1-36 h, 1-24 h, or 1-12 h. In particular embodiments, AuNP/CRISPR can be incubated with cell populations for 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 25 h, 26 h, 27 h, 28 h, 29 h, 30 h, 31 h, 32 h, 33 h, 34 h, 35 h, 36 h, 37 h, 38 h, 39 h, 40 h, 41 h, 42 h, 43 h, 44 h, 45 h, 46 h, 47 h, 48 h, or more. Electroporation of the cells was performed with a Harvard Apparatus ECM 830 Square Wave Electroporation System using BTX Express Solution (USA) in 1 mm cuvettes in 250 V and 5 ms pulse duration. 1 mm BTX cuvettes with a 2 mm gap width were used to electroporate 1-3 million K562 cells at 250V for 5 milliseconds. Cells were resuspended in culture media and analyzed following electroporation. In the context of minimal manipulation embodiments, 1-24, 1-48 or 1-72 hours are preferred for clinical logistics or disease context. In certain instances, it could take 2 days to condition a cancer patient for reinfusion, but in a genetic disease setting the patient might not be conditioned and limiting the time of manipulation outside the body is preferred.

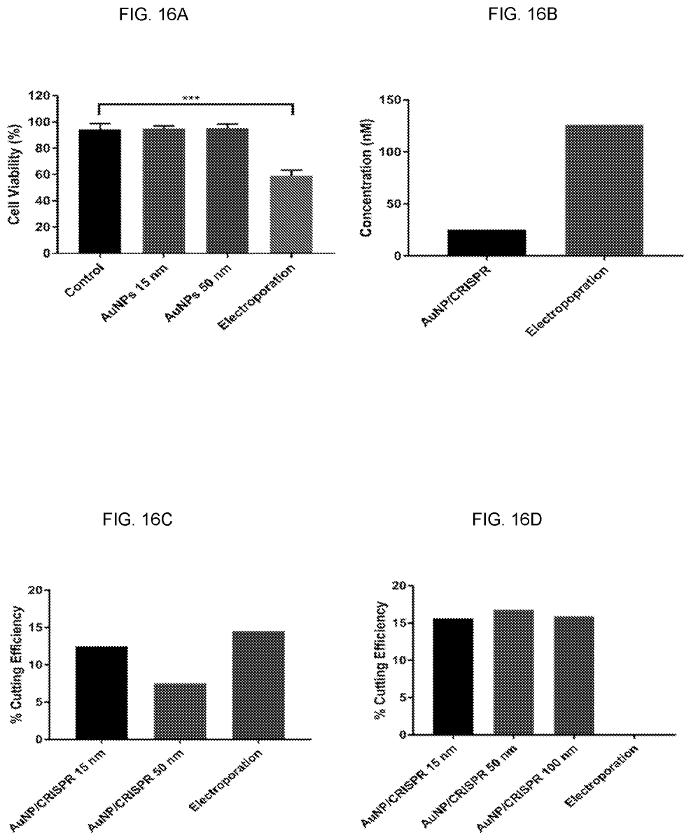

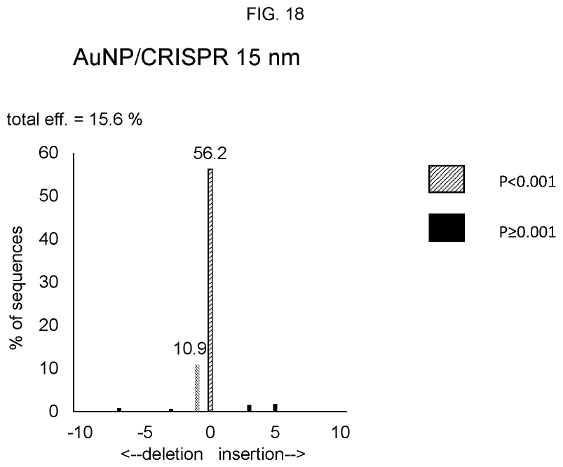

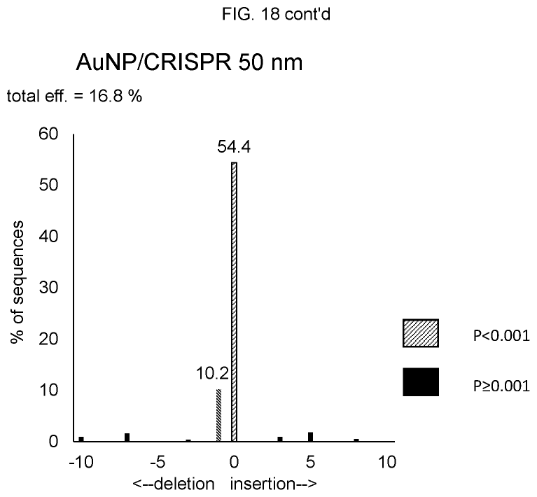

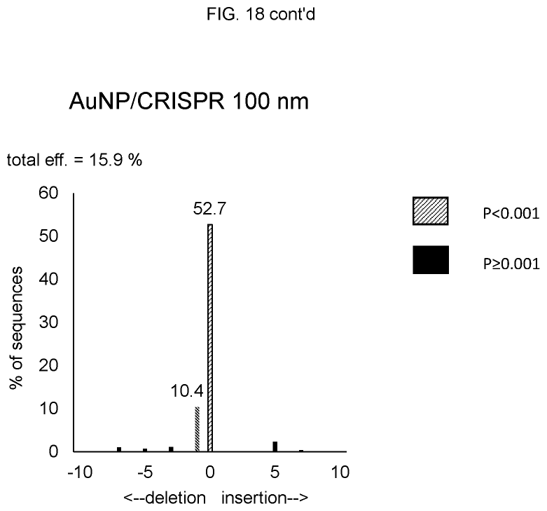

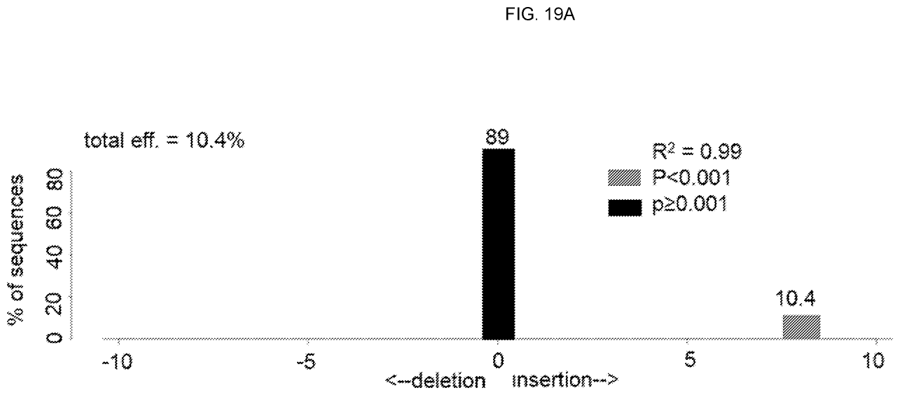

AuNP/CRISPR targeting the chr11:67812349-67812375 location were able to successfully cut the target site in very low crRNA and Cpf1 endonuclease concentrations (25 nM) in comparison to electroporation method in which a higher amount of crRNA and Cpf1 was used (126 nM) (FIG. 16C) to achieve the same efficiency of cutting. Cutting efficiency for this site was low due to the A>T mutation 15 bp after the PAM site. In the next test, the same location was targeted in primary CD34+ cells and it was shown that Au/CRISPR NP were able to target the site in a very low crRNA and Cpf1 endonuclease concentrations with very good cutting efficiency without raising any toxic effects (FIGS. 16A, 16D, and 18). Unfortunately, electroporation of the primary CD34+ cells adversely affected the viability of the cells and no cutting was seen for electroporated cells. Calculated concentration for AuNP/CRISPR was 5-fold lower than required concentration for electroporation method (FIG. 16B). As previously mentioned by Kim et al. (Nat Biotechnol, 2016. 34(8): p. 863-8), the rate of deletions to insertions was higher with the CRISPR Cpf1 gene editing system (FIG. 18).

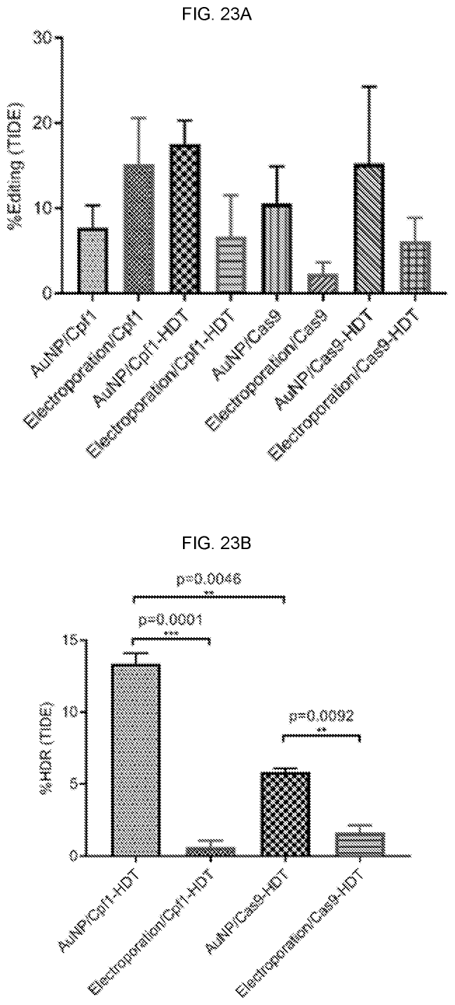

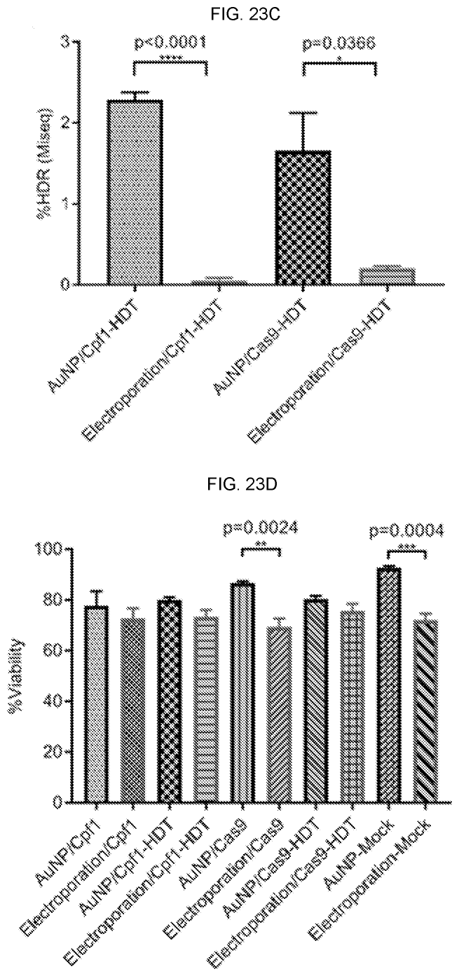

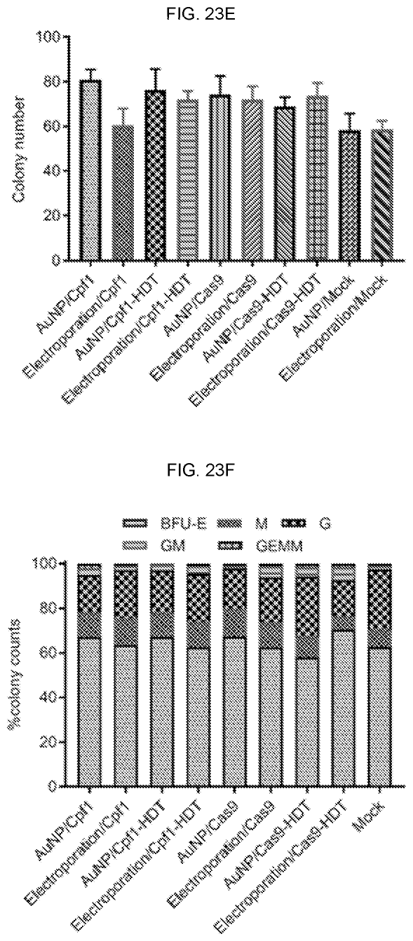

As shown in FIGS. 23A-23C, AuNP-mediated gene delivery improves Cas9 performance, however, Cpf1 is better for HDR. AuNP treated cells demonstrated higher viability compared to electroporated cells. For Cas9, AuNP mediated delivery improved total editing and HDR, relative to electroporation. For Cpf1 delivered without a homology-directed repair template (HDT), electroporation resulted in higher total gene editing (insertions and deletions, indels). This suggests that electroporation itself may impact the repair pathway used or the frequency of Cpf1 cutting at the target site. Addition of HDT to the Cpf1 formulation improved total editing and resulted in the highest HDR rates. Together, these data suggest that the fully-loaded formulation of AuNP+Cpf1/crRNA+HDT results in the highest rates of HDR with minimal indel formation. This is ideal for a number of target loci for gene editing.

In particular embodiments, a number of assays known in the art can be used to detect gene editing and/or the level (percent) or rate of gene editing. In particular embodiments, deletion or introduction of an enzyme restriction site as a result of gene editing can be assessed by restriction enzyme digestion of amplified genomic DNA flanking a gene editing target site and visualization of digestion products by gel electrophoresis. In particular embodiments, a T7 Endonuclease I (T7EI) assay can be used. In a T7EI assay, genomic DNA from cells that had been targeted for genetic modification can be isolated, and genomic regions flanking a gene editing target site can be PCR amplified. Amplified products can be annealed and digested with T7EI. T7EI recognizes and cleaves non-perfectly matched DNA, so any gene editing can be detected as mismatches in annealed heteroduplexes, which are then cut by T7EI. Percent gene modification in a T7EI assay can be calculated as follows: Percent gene modification=100×(1−(1−fraction cleaved)^{1/2}). T7EI assay kits can be obtained from, e.g., New England Biolabs, Ipswich, Mass.

In particular embodiments, gene editing or the level (percent) of gene editing can be detected by Tracking of Indels by Decomposition (TIDE) assay. A genomic region flanking a gene editing target site can be PCR amplified and amplification products can be purified. Sanger sequencing on the purified products can be carried out with fluorescently labeled terminating dideoxynucleoside triphosphates (sequencing kits available from e.g., Thermo Fisher Scientific, Waltham, Mass.). After cycle sequencing, obtained sequences can be run on TIDE software. Results can be reported as percent gene modification (Brinkman et al., Nucleic Acids Research, 42(22): e168-e168 (2014)).

In particular embodiments, gene editing or the level (percent) of gene editing can be detected by sequencing. A genomic region flanking a gene editing target site can be PCR amplified and amplification products can be purified. A second PCR can be performed to add adapters and/or other sequences needed for a given sequencing platform. Any sequencing method can be utilized, including sequencing by synthesis, pyrosequencing, sequencing by ligation, rolling circle amplification sequencing, single molecule real time sequencing, sequencing based on detection of released protons, and nanopore sequencing.

In particular embodiments, use of a therapeutic formulation including NP described herein can yield a mean total gene editing of 5% to 100%, 5% to 90%, 5% to 80%, 5% to 70%, 5% to 60%, 5% to 50%, 5% to 40%, 5% to 30%, or 5% to 20%, in target cells. In particular embodiments, use of a therapeutic formulation including NP described herein can yield a mean total gene editing of 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more in target cells.

Confocal microscopy demonstrated that disclosed NP avoided lysosomal entrapment and successfully localized to the nucleus of CD34+ primary hematopoietic cells from healthy donors.

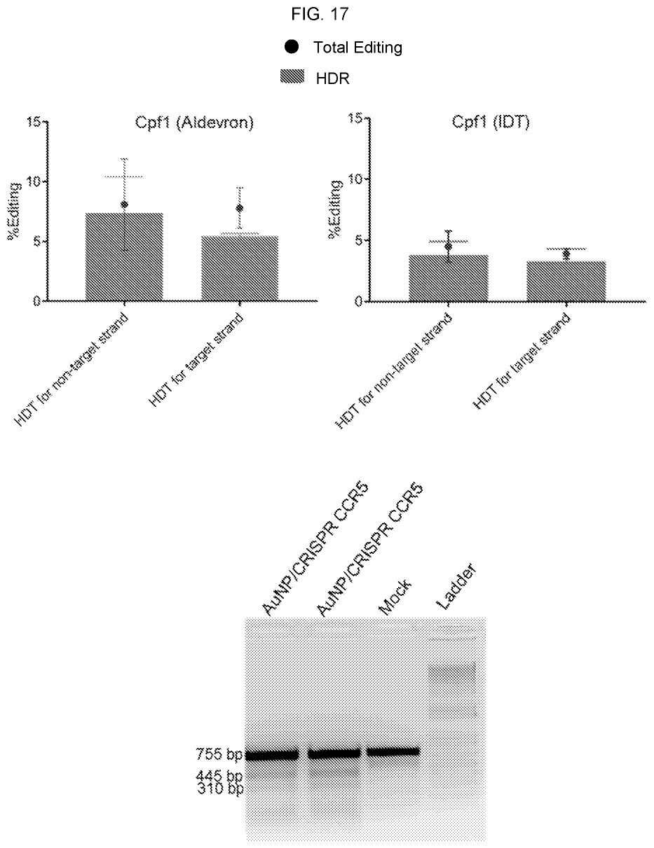

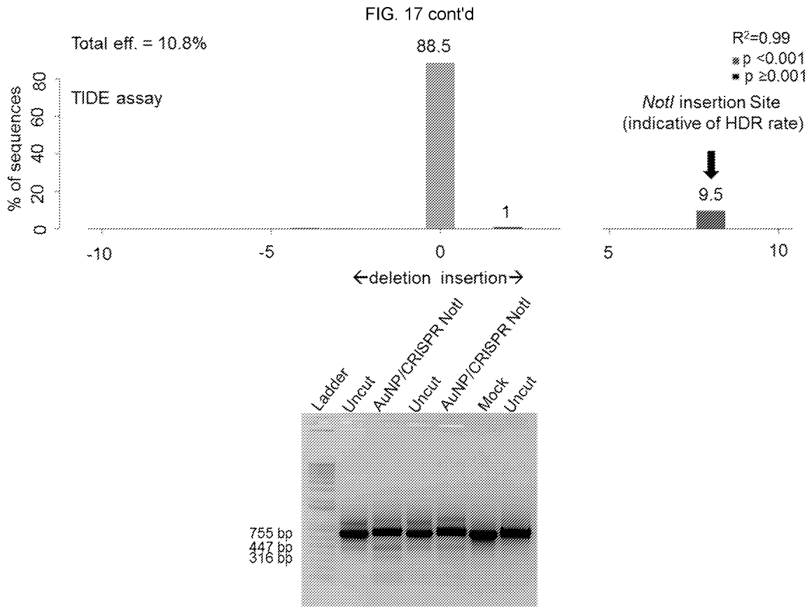

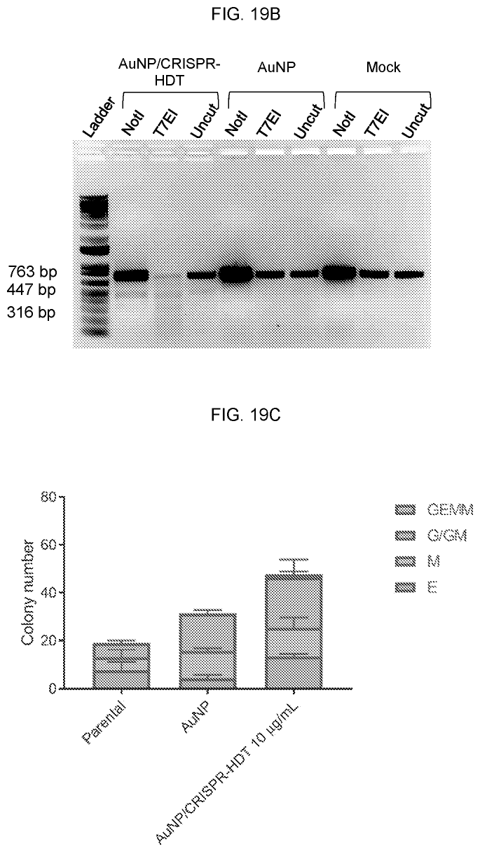

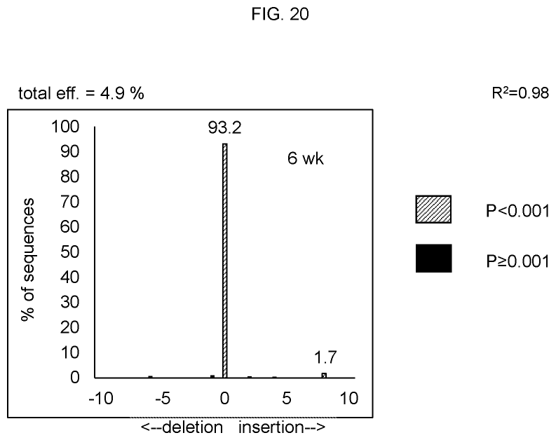

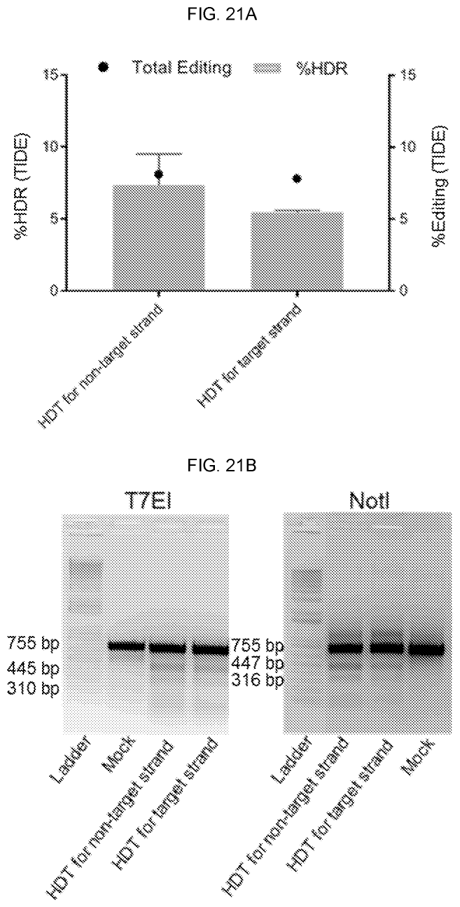

Knock-in frequencies of up to 10% were demonstrated using a NotI restriction enzyme template with homology arm lengths of ±40 nucleotides to a CCR5 locus without cytotoxicity. Designing template to the non-target DNA strand yielded a higher homology directed repair (HDR) efficiency (FIG. 17), with clear 447 bp and 316 bp cut bands following digestion with NotI and T7EI enzymes (FIG. 19B). Direct comparison of Cpf1 and Cas9 nuclease activity at the same CCR5 target site demonstrated a Cpf1 bias for HDR and template knock-in over Cas9, which preferentially generated indels. Xenotransplantation of CRISPR Cpf1 NP-treated human CD34+ cells into immune deficient mice demonstrated an early increased trend in engraftment compared to non-treated cells, suggesting an unknown benefit of NP-treated HSPCs. The frequency of CCR5 genetically modified cell engraftment was the same as observed in culture, with 10% of human cells displaying NotI template addition in vivo.

In particular embodiments, 1, 2, 3, 4, 5, 8, 10, 12, 15, or 20 μg/mL NP are added per mL of a minimally-manipulated blood cell product for an incubation period. The incubation period can be, e.g., 40 minutes to 48 hours long (in particular embodiments, 1 hour). In particular embodiments, the incubation period is 1 hour, 2 hours, 3 hours, 4 hours, 5, hours, and every integer up to 48 hours. Incubation can occur at 2-8 degrees C. (refrigeration), 23-28 degrees Celsius (room temp), or 37 degrees Celsius (body temperature). Mild rocking or rotating of the product can occur during the incubation at any temperature.

### (IV) SELECTED CELLS AND SELECTED CELL TARGETING LIGANDS

Cell populations (i.e., cell types) to target for genetic modification include HSC, HSPC, hematopoietic progenitor cells (HPC), T cells, B cells, natural killer (NK) cells, macrophages, monocytes, mesenchymal stem cells (MSC), white blood cells (WBC), mononuclear cells (MNC), endothelial cells (EC), stromal cells, and/or a bone marrow fibroblasts. A selected cell population can refer to a cell population that is to be targeted or has been targeted for genetic modification by NP of the present disclosure.

HSCs are pluripotent and ultimately give rise to all types of terminally differentiated blood cells. HSC can self-renew, or it can differentiate into more committed progenitor cells, which progenitor cells are irreversibly determined to be ancestors of only a few types of blood cell. For instance, the HSC can differentiate into (i) myeloid progenitor cells which ultimately give rise to monocytes and macrophages, neutrophils, basophils, eosinophils, erythrocytes, megakaryocytes/platelets, dendritic cells, or (ii) lymphoid progenitor cells which ultimately give rise to T-cells, B-cells, and NK-cells. Once the stem cell differentiates into a myeloid progenitor cell, its progeny cannot give rise to cells of the lymphoid lineage, and, similarly, lymphoid progenitor cells cannot give rise to cells of the myeloid lineage. For a general discussion of hematopoiesis and hematopoietic stem cell differentiation, see Chapter 17, Differentiated Cells and the Maintenance of Tissues, Alberts et al., 1989, Molecular Biology of the Cell, 2nd Ed., Garland Publishing, New York, N.Y.; Chapter 2 of Regenerative Medicine, Department of Health and Human Services, August 2006, and Chapter 5 of Hematopoietic Stem Cells, 2009, Stem Cell Information, Department of Health and Human Services.

Particular HSC populations include HSC1 (Lin-CD34+CD38-CD45RA−CD90+CD49f+) and HSC2 (CD34+CD38-CD45RA−CD90−CD49f+). For example, in particular embodiments, human HSC1 can be identified by the following profile: CD34+/CD38−/CD45RA−/CD90+ or CD34+/CD45RA−/CD90+ and mouse LT-HSC can be identified by Lin−Sca1+ckit+CD150+CD48−Flt3−CD34− (where Lin represents the absence of expression of any marker of mature cells including CD3, Cd4, CD8, CD11b, CD11c, NK1.1, Gr1, and TER119). Thus, HSC1 can include the marker profile: LHR+/CD34+/CD38−/CD45RA−/CD90+. In addition to expression of LHR, in particular embodiments, HSC1 can be identified by the following profile: Lin−/CD34+/CD38−/CD45RA−/CD90+/CD49f+. Thus, HSC1 can include the marker profile: LHR+/Lin−/CD34+/CD38−/CD45RA−/CD90+/CD49f+. In addition to expression of LHR, in particular embodiments, HSC2 can be identified by the following profile: CD34+/CD38-/CD45RA−/CD90−/CD49f+. Thus, HSC2 can include the marker profile: LHR+/CD34+/CD38-/CD45RA−/CD90−/CD49f+. Based on the foregoing profiles, expression of LHR can be combined with presence or absence of the following one or more markers to identify HSC1 and/or HSC2 cell populations: Lin/CD34/CD38/CD45RA/CD90/CD49f as well as CD133. Various other combinations may also be used so long as the marker combination reliably identifies HSC1 or HSC2. In particular embodiments, HSC are identified by a CD133+ profile. In particular embodiments, HSC are identified by a CD34+/CD133+ profile. In particular embodiments, HSC are identified by a CD164+ profile. In particular embodiments, HSC are identified by a CD34+/CD164+ profile.

HSPC refer to hematopoietic stem cells and/or hematopoietic progenitor cells. HSPC can self-renew or can differentiate into myeloid progenitor cells or lymphoid progenitor cells as described above for HSC. HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. Preferably, the HSPC are CD34+ cells.

In particular embodiments, ‘HSC/HSPC’ can refer to either HSC, HSPC, or both.

Lymphocytes include T cells and B cells. T cells are a key part of an immune system, helping to control immune responses as well as to kill cells such as virus-infected cells and cancer cells. There are several T cell types, including helper T cells, cytotoxic T cells, central memory T cells, effector memory T cells, regulatory T cells, and naïve T cells. B cells participate in the adaptive immune system, including producing antibodies against invaders such as bacteria, viruses, and other organisms.

Several different subsets of T-cells have been discovered, each with a distinct function. In particular embodiments, selected cell targeting ligands achieve selective direction to particular lymphocyte populations through receptor-mediated endocytosis. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the as T-cells.

CD3 is expressed on all mature T cells. Accordingly, selected cell targeting ligands disclosed herein can bind CD3 to achieve selective delivery of nucleic acids to all mature T-cells.

Activated T-cells express 4-1BB (CD137), CD69, and CD25. Accordingly, selected cell targeting ligands disclosed herein can bind 4-1BB, CD69 or CD25 to achieve selective delivery of nucleic acids to activated T-cells. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response. S

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) as used herein refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

Regulatory T cells (“TREG”) are a subpopulation of T cells, which modulate the immune system, maintain tolerance to self-antigens, and abrogate autoimmune disease. TREG express CD25, CTLA-4, GITR, GARP and LAP.

“Naive” T-cells as used herein refers to a non-antigen experienced T cell that expresses CD62L and CD45RA and does not express CD45RO as compared to central or effector memory cells. In particular embodiments, naive CD8+T lymphocytes are characterized by the expression of phenotypic markers of naive T-cells including CD62L, CCR7, CD28, CD127, and CD45RA.

B cells can be distinguished from other lymphocytes by the presence of the B cell receptor (BCR). The principal function of B cells is to make antibodies. B cells express CD5, CD19, CD20, CD21, CD22, CD35, CD40, CD52, and CD80. Selected cell targeting ligands disclosed herein can bind CD5, CD19, CD20, CD21, CD22, CD35, CD40, CD52, and/or CD80 to achieve selective delivery of nucleic acids to B-cells. Also antibodies targeting the B-cell receptor isotype constant regions (IgM, IgG, IgA, IgE) can be used to target B-cell subtypes.

Natural killer cells (also known as NK cells, K cells, and killer cells) are activated in response to interferons or macrophage-derived cytokines. NK cells can induce apoptosis or cell lysis by releasing granules that disrupt cellular membranes and can secrete cytokines to recruit other immune cells. They serve to contain viral infections while the adaptive immune response is generating antigen-specific cytotoxic T cells that can clear the infection. NK cells express NKG2D, CD8, CD16, CD56, KIR2DL4, KIR2DS1, KIR2DS2, KIR3DS1, NKG2C, NKG2E, NKG2D, and several members of the natural cytotoxicity receptor (NCR) family. Examples of NCRs include NKp30, NKp44, NKp46, NKp80, and DNAM-1.

Macrophages (and their precursors, monocytes) reside in every tissue of the body (in certain instances as microglia, Kupffer cells and osteoclasts) where they engulf apoptotic cells, pathogens and other non-self-components. Examples of proteins expressed on the surface of macrophages (and their precursors, monocytes) include CD11b, CD11c, CD64, CD68, CD119, CD163, CD206, CD209, F4/80, IFGR2, Toll-like receptors (TLRs) 1-9, IL-4Ra, and MARCO.

The selected cell targeting ligands that can be attached to NP disclosed herein selectively bind cells of interest within a heterogeneous cell population. “Selective delivery” to a selected cell type within a heterogenous mixture of cells means that at least 20%, 25%, 30%, 35%, 40%, 45%, 50%, 55%, 60%, 65%, 70%, 75%, 80%, 85%, 90%, 95%, or 99% of administered NP are proportionately taken up in the targeted cells versus the cells in the population that do not express the target marker. In particular embodiments, 50% or more of the selected cell population within a sample take up NPs and less than 20% of any one non-target cell population take up NP.

In particular embodiments, binding domains of selected cell targeting ligands include cell marker ligands, receptor ligands, antibodies, peptides, peptide aptamers, nucleic acids, nucleic acid aptamers, spiegelmers or combinations thereof. Within the context of selected cell targeting ligands, binding domains include any substance that binds to another substance to form a complex capable of mediating endocytosis.

“Antibodies” are one example of targeting ligands and include whole antibodies or binding fragments of an antibody, e.g., Fv, Fab, Fab′, F(ab′)2, and single chain Fv fragments (scFvs) or any biologically effective fragments of an immunoglobulin that bind specifically to a motif expressed by a selected cell type. Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions.

Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242 (1988) 423-426; Huston, et al., Proc. Natl. Acad. Sci. USA 85 (1988) 5879-5883; Plueckthun, in The Pharmacology of Monoclonal Antibodies, vol. 113, Rosenburg and Moore (eds.), Springer-Verlag, New York), (1994) 269-315; WO1993/16185; U.S. Pat. Nos. 5,571,894; and 5,587,458.

A Fab fragment is a monovalent antibody fragment including V_{L}, V_{H}, CL and CH1 domains.

A F(ab′)₂ fragment is a bivalent fragment including two Fab fragments linked by a disulfide bridge at the hinge region. Diabodies include two epitope-binding sites that may be bivalent. See, for example, EP 0404097; WO1993/01161; and Holliger, et al., Proc. Natl. Acad. Sci. USA 90 (1993) 6444-6448. Dual affinity retargeting antibodies (DART™; based on the diabody format but featuring a C-terminal disulfide bridge for additional stabilization (Moore et al., Blood 117, 4542-51 (2011))) can also be formed. Antibody fragments can also include isolated CDRs. For a review of antibody fragments, see Hudson, et al., Nat. Med. 9 (2003) 129-134.

Antibodies from human origin or humanized antibodies have lowered or no immunogenicity in humans and have a lower number of non-immunogenic epitopes compared to non-human antibodies. Antibodies and their fragments will generally be selected to have a reduced level or no antigenicity in human subjects.

Antibodies that specifically bind a motif expressed by a selected cell type can be prepared using methods of obtaining monoclonal antibodies, methods of phage display, methods to generate human or humanized antibodies, or methods using a transgenic animal or plant engineered to produce antibodies as is known to those of ordinary skill in the art (see, for example, U.S. Pat. Nos. 6,291,161 and 6,291,158). Phage display libraries of partially or fully synthetic antibodies are available and can be screened for an antibody or fragment thereof that can bind to a selected cell type motif. For example, binding domains may be identified by screening a Fab phage library for Fab fragments that specifically bind to a target of interest (see Hoet et al., Nat. Biotechnol. 23:344, 2005). Phage display libraries of human antibodies are also available.

Additionally, traditional strategies for hybridoma development using a target of interest as an immunogen in convenient systems (e.g., mice, HuMAb Mouse®, TC Mouse™, KM-Mouse®, llamas, chicken, rats, hamsters, rabbits, etc.) can be used to develop targeting ligand binding domains. In particular embodiments, antibodies specifically bind to motifs expressed by a selected lymphocyte and do not cross react with nonspecific components or unrelated targets. Once identified, the amino acid sequence or nucleic acid sequence coding for the antibody can be isolated and/or determined.

Aptamers may be designed to facilitate selective delivery, including delivery across the cellular membrane, to intracellular compartments, or into the nucleus. Methods of making aptamers and conjugating such aptamers to the surface of NP are described in, for example, Huang et al. Anal. Chem., 2008, 80 (3), pp 567-572. In particular embodiments, an aptamer of the present disclosure binds CD133.

In particular embodiments, peptide aptamers refer to a peptide loop (which is specific for a target protein) attached at both ends to a protein scaffold. This double structural constraint greatly increases the binding affinity of the peptide aptamer to levels comparable to an antibody.

The variable loop length is typically 8 to 20 amino acids (e.g., 8 to 12 amino acids), and the scaffold may be any protein which is stable, soluble, small, and non-toxic (e.g., thioredoxin-A, stefin A triple mutant, green fluorescent protein, eglin C, and cellular transcription factor Spl).

Peptide aptamer selection can be made using different systems, such as the yeast two-hybrid system (e.g., Gal4 yeast-two-hybrid system) or the LexA interaction trap system.

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. In particular embodiments, aptamers are small (15 KD; or between 15-80 nucleotides or between 20-50 nucleotides). Aptamers are generally isolated from libraries consisting of 10¹⁴-10¹⁵ random oligonucleotide sequences by a procedure termed SELEX (systematic evolution of ligands by exponential enrichment; see, for example, Tuerk et al., Science, 249:505-510, 1990; Green et al., Methods Enzymology. 75-86, 1991; and Gold et al., Annu. Rev. Biochem., 64: 763-797, 1995). Further methods of generating aptamers are described in, for example, U.S. Pat. Nos. 6,344,318; 6,331,398; 6,110,900; 5,817,785; 5,756,291; 5,696,249; 5,670,637; 5,637,461; 5,595,877; 5,527,894; 5,496,938; 5,475,096; and 5,270,16. Spiegelmers are similar to nucleic acid aptamers except that at least one β-ribose unit is replaced by β-D-deoxyribose or a modified sugar unit selected from, for example, β-D-ribose, α-D-ribose, β-L-ribose.

In particular embodiments, an RNA aptamer sequence has binding affinity for an aptamer ligand on or in the cell. In particular embodiments, the aptamer ligand is on the cell, for example so that it is at least partially available on the extracellular face or side of the cell membrane. For example, the aptamer ligand may be a cell-surface protein. The aptamer ligand may therefore be one part of a fusion protein, one other part of the fusion protein having a membrane anchor or membrane-spanning domain. In particular embodiments, the aptamer ligand is in the cell. For example, the aptamer ligand may be internalized within a cell, i.e. within (beyond) the cell membrane, for example in the cytoplasm, within an organelle (including mitochondria), within an endosome, or in the nucleus. In particular embodiments, an aptamer can include a donor template sequence, which can include a homology-directed repair (HDR) template and a therapeutic nucleic acid sequence.

Selected cell targeting ligands disclosed herein can bind CD34, CD46, CD90, CD133, CD164, Sca-1, CD117, LHRH receptor, and/or AHR to achieve selective delivery of NP to HSCs.

As indicated previously, particular embodiments include as targeting ligands one or more of a CD34 antibody, a CD90 antibody, a CD133 antibody, a CD164 antibody, an aptamer, human luteinizing hormone, human chorionic gonadotropin, degerelix acetate (an antagonist of the LHRH receptor), or StemRegenin 1.

In particular embodiments, the targeting ligand that binds CD34 is a human or humanized antibody. In particular embodiments, the targeting ligand that binds CD34 is antibody clone: 581; antibody clone: 561; antibody clone: REA1164; or antibody clone: AC136; or a binding fragment derived therefrom.

In particular embodiments, the binding domain that binds CD34 includes a variable light chain including a CDRL1 sequence including RSSQTIVHSNGNTYLE (SEQ ID NO: 139), a CDRL2 sequence including QVSNRFS (SEQ ID NO: 140), a CDRL3 sequence including FQGSHVPRT (SEQ ID NO: 141), a CDRH1 sequence including GYTFTNYGMN (SEQ ID NO: 142), a CDRH2 sequence including WINTNTGEPKYAEEFKG (SEQ ID NO: 143), and a CDRH3 sequence including GYGNYARGAWLAY (SEQ ID NO: 144). For more information regarding binding domains that bind CD34, see WO2008CN01963. Additional CD34 binding domains are also commercially available. For example, Invitrogen offers CD34 Monoclonal Antibody (QBEND/10; Clone: QBEND/10; Catalog #: MA1-10202).

In particular embodiments, the binding domain that binds CD90 is antibody clone: 5E10; antibody clone: DG3; antibody clone: REA897; or a binding fragment derived therefrom.

In particular embodiments, the binding domain that binds CD90 is a single chain antibody including the sequence CMASASQVQLVQSGAEVKKPGASVKVSCKASGYTFTGYYVHWVRQAPGQGLEWMGWVNPN SGDTNYAQKFQGRVTMTRDTSISTAYMELSGLRSDDTAVYYCARDGDEDWYFDLWGRGTPV TVSSGILGSGGGGSGGGGSGGGGSDIRLTQSPSSLSASIGDRVTITCRASQGISRSLVWYQQK PGKAPRLLIYAASTLQSGVPSRFSGSGSGTDFTLTISSLQPEDFATYYCLQHNTYPFTFGPGTK VDIKSGIPEQKL (SEQ ID NO: 145). In particular embodiments, the binding domain is human or humanized. For more information regarding binding domains that bind CD90, see WO2017US35989. CD90 binding domains are also commercially available. For example, Abcam offers Anti-CD90/Thy1 antibody ([EPR3133]; Clone: EPR3133; Catalog #: ab133350).

In particular embodiments, the binding domain that binds CD133 is antibody clone: REA820; antibody clone: REA753; antibody clone: REA816; antibody clone: 293C3; antibody clone: AC141; antibody clone: AC133; antibody clone: 7; or a binding fragment derived therefrom.

In particular embodiments, the binding domain that binds CD133 is derived from C178ABC-CD133MAb. In particular embodiments, the binding domain includes a variable light chain of NIVMTQSPKSMSMSLGERVTLSCKASENVDTYVSWYQQKPEQSPKVLIYGASNRYTGVPDRF TGSGSATDFSLTISNVQAEDLADYHCGQSYRYPLTFGAGTKLELKR (SEQ ID NO: 146) and a variable heavy chain of EIQLQQSGPDLMKPGASVKISCKASGYSFTNYYVHWVKQSLDKSLEWIGYVDPFNGDFNYNQ KFKDKATLTVDKSSSTAYMHLSSLTSEDSAVYYCARGGLDWYDTSYWYFDVWGAGTAV (SEQ ID NO: 147).

In particular embodiments, the binding domain includes a variable light chain including a CDRL1 sequence including QSSQSVYNNNYLA (SEQ ID NO: 148), a CDRL2 sequence including RASTLAS (SEQ ID NO: 149), a CDRL3 sequence including QGEFSCDSADCAA (SEQ ID NO: 150), a CDRH1 sequence including GIDLNNY (SEQ ID NO: 151), a CDRH2 sequence including FGSDS (SEQ ID NO: 152), and a CDRH3 sequence including GGL.

In particular embodiments, the binding domain is human or humanized. For more information regarding binding domains that bind CD133, see WO2011089211, U.S. Pub. No. 2018/0105598, and/or U.S. Pub. No. 2013/0224202. CD133 binding domains are also commercially available. For example, Abcam offers Anti-CD133 antibody ([EPR20980-45; Clone: EPR20980-45; Catalog #: ab226355).

In particular embodiments, the binding domain that binds CD133 is an aptamer. The aptamer can be Aptamer A15 or B19 from Tocris Biosciences. In particular embodiments, aptamer A15 refers to an RNA aptamer with 15 bases and the formula C₁₈₂H₂₁₉F9N₅₈O₁₀₄P₁₆. This aptamer has a molecular weight of 5549.58, and sequence modifications: 2-fluoropyrimidines, 3′-inverted deoxythymidine cap, 5′-fluorescent DY647 tag. See also Shigdar et al (2013) RNA aptamers targeting cancer stem cell marker CD133. Cancer Lett. 330 84 PMID: 23196060. In particular embodiments, aptamer 19 refers to an RNA apatamer with 19 bases and the formula C221H263F10N730131P20. This aptamer has a molecular weight of 6847.32, and sequence modifications: 2-fluoropyrimidines, 3′-inverted deoxythymidine cap, 5′-fluorescent DY647 tag. See also Shigdar et al (2013) RNA aptamers targeting cancer stem cell marker CD133. Cancer Lett. 330 84 PMID: 23196060

In particular embodiments, the RNA aptamer includes a consensus sequence including CCCUCCUACAUAGGG (SEQ ID NO: 153). In particular embodiments the RNA aptamer includes a consensus sequence including GAGACAAGAAUAAACGCUCAACCCACCCUCCUACAUAGGGAGGAACGAGUUACUAUAGA GCUUCGACAGGAGGCUCACAAC (SEQ ID NO: 154); GAGACAAGAAUAAACGCUCAACCCACCCUCCUACAUAGGGAGGAACGAGUUACUAUAG (SEQ ID NO: 155); GCUCAACCCACCCUCCUACAUAGGGAGGAACGAGU (SEQ ID NO: 111); CCACCCUCCUACAUAGGGUGG (SEQ ID NO: 156); CAGAACGUAUACUAUUCUG (SEQ ID NO: 157); AGAACGUAUACUAUU (SEQ ID NO: 158); or GAGACAAGAAUAAACGCUCAAGGAAAGCGCUUAUUGUUUGCUAUGUUAGAACGUAUACU AUUUCGACAGGAGGCUCACAACAGGC (SEQ ID NO: 159). For additional information regarding CD133 aptamers, see EP2880185.

Particular embodiments using targeting ligands that bind luteinizing hormone receptor (LHR). Particular embodiments can utilize the LH alpha subunit and the LH beta subunit. In particular embodiments, the alpha subunit includes

(SEQ ID NO: 53)
DCPECTLQENPFFSQPGAPILQCMGCCFSRAYPTPLRSKKTMLVQKNVT
SESTCCVAKSYNRVTVMGGFKVENHTACHCSTCYYHKS (human)
or
(SEQ ID NO: 54)
GCPECKLKENKYFSKLGAPIYQCMGCCFSRAYPTPARSKKTMLVPKNIT
SEATCCVAKAFTKATVMGNARVENHTECHCSTCYYHKS (mouse).

In particular embodiments, the LH beta subunit includes

(SEQ ID NO: 55)
SREPLRPWCHPINAILAVEKEGCPVCITVNTTICAGYCPTMMRVLQAVL
PPLPQVVCTYRDVRFESIRLPGCPRGVDPVVSFPVALSCRCGPCRRSTS
DCGGPKDHPLTCDHPQLSGLLFL (human)
or
(SEQ ID NO: 56)
SRGPLRPLCRPVNATLAAENEFCPVCITFTTSICAGYCPSMVRVLPAAL
PPVPQPVCTYRELRFASVRLPGCPPGVDPIVSFPVALSCRCGPCRLSSS
DCGGPRTQPMACDLPHLPGLLLL (mouse).

Numerous antibodies that bind LHR or other HSC1/HSC2 markers are commercially available. For example, anti-LHR antibodies are commercially available from Abcam, Invitrogen, Alomone Labs, Novus Biologicals, Origene Technologies, Bio-Rad, Abbexa, St. John's Laboratory, Millipore Sigma (Burlington, Mass.), LifeSpan Biosciences, etc.

In particular embodiments, an anti-LHR binding agent includes a CDRH1 including GYSITSGYG (SEQ ID NO: 57); a CDRH2 including IHYSGST (SEQ ID NO: 58); a CDRH3 including ARSLRY (SEQ ID NO: 59); and a CDRL1 including SSVNY (SEQ ID NO: 60); a CDRL2 including DTS; and a CDRL3 including HQWSSYPYT (SEQ ID NO: 61).

In particular embodiments, an anti-LHR binding agent includes a CDRH1 including GFSLTTYG (SEQ ID NO: 62); a CDRH2 including IWGDGST (SEQ ID NO: 63); and a CDRH3 including AEGSSLFAY (SEQ ID NO: 64); and a CDRL1 including QSLLNSGNQKNY (SEQ ID NO: 65); a CDRL2 including WAS; and a CDRL3 including QNDYSYPLT (SEQ ID NO: 66).

In particular embodiments, an anti-LHR binding agent includes a CDRH1 including GYSFTGYY (SEQ ID NO: 67); a CDRH2 including IYPYNGVS (SEQ ID NO: 68); and a CDRH3 including ARERGLYQLRAMDY (SEQ ID NO: 69); and a CDRL1 including QSISNN (SEQ ID NO: 70); a CDRL2 including NAS; and a CDRL3 including QQSNSWPYT (SEQ ID NO: 71).

In particular embodiments, an anti-LHR binding agent includes a heavy chain including EVQLQESGPDLVKPSQSLSLTCTVTGYSITSGYGWHRQFPGNKLEWMGYIHYSGSTTYNPSLK SRISISRDTSKNQFFLQLNSVTTEDTATYYCARSLRYWGQGTTLTVSS (SEQ ID NO: 72) and a light chain including DIVMTQTPAIMSASPGQKVTITCSASSSVNYMHWYQQKLGSSPKLWIYDTSKLAPGVPARFSG SGSGTSYSLTISSMEAEDAASYFCHQWSSYPYTFGSGTKLEIK (SEQ ID NO: 73).

In particular embodiments, an anti-LHR binding agent includes a heavy chain including QVQLKESGPGLVAPSQSLSrrCTVSGFSLTTYGVSWVRQPPGKGLEWLGVIWGDGSTYYHSAL ISRLSISKDNSKSQVFLKLNSLQTDDTATYYCAEGSSLFAYWGQGTLVTVS A (SEQ ID NO: 74) and a light chain including DIVMTQSPSSLTVTAGEKVTMSCKSSQSLLNSGNQKNYLTWYQQKPGQPPKLLIYWASTRQS GVPDRFTGSGSGTDFTLTISSVQAEDXAVYYCQNDYSYPLTFGSGTKLEIK (SEQ ID NO: 75).

In particular embodiments, an anti-LHR binding agent includes a heavy chain including EVQLEQSGGGLVQPGGSRKLSCAASGFTFSSFGMHWVRQAPEKGLEWVAYISSGSSTLHYA DTVKGRFTISRDNPKNTLFLQMKLPSLCYGLLGSRNLSHRLL (SEQ ID NO: 76) and a light chain including DIVLTQTPSSLSASLGDTITITCHASQNINVWLFWYQQKPGNIPKLLIYKASNLLTGVPSRFSGSG SGTGFTLTISSLQPEDIATYYCQQGQSFPWTFGGGTKLEIK (SEQ ID NO: 77).

In particular embodiments, an anti-LHR binding agent includes a heavy chain including QVKLQQSGPELVKPGASVKISCKASGYSFTGYYMHWVKQSHGNILDWIGYIYPYNGVSSYNQK FKGKATLTVDKSSSTAYMELRSLTSEDSAVYYCARERGLYQLRAMDYWGQGTSVTVSS (SEQ ID NO: 78) and a light chain including DIVLTQTPATLSVTPGDSVSLSCRASQSISNNLHWYQQKSHESPRLLIKNASQSISGIPSKF SGSGSGTDFTLRINSVETEDFGMYFCQQSNSWPYTFGSGTKLEIK (SEQ ID NO: 79).

In particular embodiments, an anti-LHR binding agent includes subunit beta 3 of human choriogonadotropin (CGB3; UniProt ID PODN86) including

(SEQ ID NO: 160)
SKEPLRPRCRPINATLAVEKEGCPVCITVNTTICAGYCPTMTRVLQGVL
PALPQVVCNYRDVRFESIRLPGCPRGVNPVVSYAVALSCQCALCRRSTT
DCGGPKDHPLTCDDPRFQDSSSSKAPPPSLPSPSRLPGPSDTPILPQ.

Particular embodiments include using targeting ligands that bind an aryl hydrocarbon receptor (AHR). AHR is a member of the family of basic helix-loop-helix transcription factors. AHR regulates the function of xenobiotic-metabolizing enzymes and the toxicity and carcinogenic properties of several compounds. AHR also plays an important role in the regulation of pluripotency and stemness of HSCs. Inhibition of AHR by StemRegenin 1 (SR1) has been shown to lead to an increase in cells expressing CD34 and an increase in cells that retain the ability to engraft immunodeficient mice.

In particular embodiments, SR1, also known as 4-(2-((2-(benzo[b]thiophen-3-yl)-9-isopropyl-9H-purin-6-yl)amino)ethyl)phenol, has a chemical formula of C₂₄H₂₃N₅OS and the following structure:

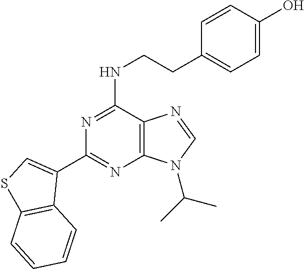 [SMILES: CC(C)n1cnc2c(NCCc3ccc(O)cc3)nc(-c3csc4ccccc34)nc21]

SR1 is commercially available from vendors such as Cayman Chemical Company, Ann Arbor, Mich.; STEMCELL™ Technologies, Vancouver, Calif.; and Abcam, Cambridge, Mass.

In particular embodiments, binding domains of selected cell targeting ligands include T-cell receptor motif antibodies; T-cell α chain antibodies; T-cell β chain antibodies; T-cell γ chain antibodies; T-cell δ chain antibodies; CCR7 antibodies; CD1a antibodies; CD1b antibodies; CD1c antibodies; CD1d antibodies; CD3 antibodies; CD4 antibodies; CD5 antibodies; CD7 antibodies; CD8 antibodies; CD11b antibodies; CD11c antibodies; CD16 antibodies; CD19 antibodies; CD20 antibodies; CD21 antibodies; CD22 antibodies; CD25 antibodies; CD28 antibodies; CD34 antibodies; CD35 antibodies; CD39 antibodies; CD40 antibodies; CD45RA antibodies; CD45RO antibodies; CD46 antibodies; CD52 antibodies; CD56 antibodies; CD62L antibodies; CD68 antibodies; CD80 antibodies; CD86 antibodies CD90 antibodies; CD95 antibodies; CD101 antibodies; CD117 antibodies; CD127 antibodies; CD137 (4-1BB) antibodies; CD148 antibodies; CD163 antibodies; CD164 antibodies; F4/80 antibodies; IL-4Ra antibodies; Sca-1 antibodies; CTLA-4 antibodies; GITR antibodies; GARP antibodies; LAP antibodies; granzyme B antibodies; LFA-1 antibodies; or transferrin receptor antibodies.

Targeting ligands that result in selective NP delivery to T cells can include a binding domain that binds CD3 derived from at least one of OKT3 (described in U.S. Pat. No. 5,929,212), otelixizumab, teplizumab, visilizumab, 20G6-F3, 4B4-D7, 4E7-C9, 18F5-H10, or TR66. In particular embodiments, the binding domain includes a variable light chain of EIVLTQSPATLSLSPGERATLSCRASQSVSSYLAWYQQKPGQAPRLLIYDASNRATGIPARFSG SGSGTDFTLTISSLEPEDFAVYYCQQRSNWPPLTFGGGTKVEIK (SEQ ID NO: 161) and a variable heavy chain of QVQLVESGGGVVQPGRSLRLSCAASGFKFSGYGMHWVRQAPGKGLEWVAVIWYDGSKKYY VDSVKGRFTISRDNSKNTLYLQMNSLRAEDTAVYYCARQMGYWHFDLWGRGTLVTVSS (SEQ ID NO: 162).

In particular embodiments, the binding domain includes a variable light chain of EIVLTQSPATLSLSPGERATLSCRASQSVSSYLAWYQQKPGQAPRLLIYDASNRATGIPARFSG SGSGTDFTLTISSLEPEDFAVYYCQQRSNWPPLTFGGGTKVEIK (SEQ ID NO: 161) and a variable heavy chain of QVQLVQSGGGVVQSGRSLRLSCAASGFKFSGYGMHWVRQAPGKGLEWVAVIWYDGSKKYY VDSVKGRFTISRDNSKNTLYLQMNSLRGEDTAVYYCARQMGYWHFDLWGRGTLVTVSS (SEQ ID NO: 163).

In particular embodiments, the binding domain includes a variable light chain including a CDRL1 sequence including SASSSVSYMN (SEQ ID NO: 164), a CDRL2 sequence including RWIYDTSKLAS (SEQ ID NO: 165), a CDRL3 sequence including QQWSSNPFT (SEQ ID NO: 166), a CDRH1 sequence including KASGYTFTRYTMH (SEQ ID NO: 167), a CDRH2 sequence including INPSRGYTNYNQKFKD (SEQ ID NO: 168), and a CDRH3 sequence including YYDDHYCLDY (SEQ ID NO: 169).

In particular embodiments, the binding domain includes a variable light chain including a CDRL1 sequence including QSLVHNNGNTY (SEQ ID NO: 170), a CDRL2 sequence including KVS, a CDRL3 sequence including GQGTQYPFT (SEQ ID NO: 171), a CDRH1 sequence including GFTFTKAW (SEQ ID NO: 172), a CDRH2 sequence including IKDKSNSYAT (SEQ ID NO: 173), and a CDRH3 sequence including RGVYYALSPFDY (SEQ ID NO: 174).

In particular embodiments, the binding domain includes a variable light chain including a CDRL1 sequence including QSLVHDNGNTY (SEQ ID NO: 175), a CDRL2 sequence including KVS, a CDRL3 sequence including GQGTQYPFT (SEQ ID NO: 171), a CDRH1 sequence including GFTFSNAW (SEQ ID NO: 175), a CDRH2 sequence including IKARSNNYAT (SEQ ID NO: 176), and a CDRH3 sequence including RGTYYASKPFDY (SEQ ID NO: 177).

In particular embodiments, the binding domain includes a variable light chain including a CDRL1 sequence including QSLEHNNGNTY (SEQ ID NO: 179), a CDRL2 sequence including KVS, a CDRL3 sequence including GQGTQYPFT (SEQ ID NO: 171), a CDRH1 sequence including GFTFSNAW (SEQ ID NO: 176), a CDRH2 sequence including IKDKSNNYAT (SEQ ID NO: 180), and a CDRH3 sequence including RYVHYGIGYAMDA (SEQ ID NO: 181).

In particular embodiments, the binding domain includes a variable light chain including a CDRL1 sequence including QSLVHTNGNTY (SEQ ID NO: 182), a CDRL2 sequence including KVS, a CDRL3 sequence including GQGTHYPFT (SEQ ID NO: 183), a CDRH1 sequence including GFTFTNAW (SEQ ID NO: 184), a CDRH2 sequence including KDKSNNYAT (SEQ ID NO: 185), and a CDRH3 sequence including RYVHYRFAYALDA (SEQ ID NO: 186).

In particular embodiments, the binding domain is human or humanized. For more information regarding binding domains that bind CD3, see U.S. Pat. No. 8,785,604, PCT/US 17/42264, and/or WO02051871. CD3 binding domains are also commercially available. For example, LSBio offers PathPlus™ CD3 Antibody Monoclonal IHC LS-B8669 (Clone: SP7; Catalog #: LS-B8669-100).

CD4-expressing T cells can be targeted for selective NP delivery with a binding domain that binds CD4 is an antibody. In particular embodiments, the binding domain includes a variable light chain of DIVMTQSPDSLAVSLGERVTMNCKSSQSLLYSTNQKNYLAWYQQKPGQSPKLLIYWASTRES GVPDRFSGSGSGTDFTLTISSVQAEDVAVYYCQQYYSYRTFGGGTKLEIK (SEQ ID NO: 187) and a variable heavy chain of QVQLQQSGPEVVKPGASVKMSCKASGYTFTSYVIHWVRQKPGQGLDWIGYINPYNDGTDYDE KFKGKATLTSDTSTSTAYMELSSLRSEDTAVYYCAREKDNYATGAWFAYWGQGTLVTVSS (SEQ ID NO: 188). In particular embodiments, the binding domain includes a variable light chain including a CDRL1 sequence including KSSQSLLYSTNQKNYLA (SEQ ID NO: 189), a CDRL2 sequence including WASTRES (SEQ ID NO: 190), a CDRL3 sequence including QQYYSYRT (SEQ ID NO: 191), a CDRH1 sequence including GYTFTSYVIH (SEQ ID NO: 192), a CDRH2 sequence including YINPYNDGTDYDEKFKG (SEQ ID NO: 193), and a CDRH3 sequence including EKDNYATGAWFAY (SEQ ID NO: 194). In particular embodiments, the binding domain is human or humanized. For more information regarding binding domains that bind CD4, see PCT App NO. WO2008US05450. CD4 binding domains are also commercially available. For example, R&D Systems offers Human CD4 Antibody (Clone: 34930; Catalog #: MAB379).

CD28 is a surface glycoprotein present on 80% of peripheral T-cells in humans and is present on both resting and activated T-cells. CD28 binds to B7-1 (CD80) and B7-2 (CD86). In particular embodiments, a CD28 binding domain (e.g., scFv) is derived from CD80, CD86 or the 9D7 antibody. Additional antibodies that bind CD28 include 9.3, KOLT-2, 15E8, 248.23.2, and EX5.3D10. Further, 1YJD provides a crystal structure of human CD28 in complex with the Fab fragment of a mitogenic antibody (5.11A1). In particular embodiments, antibodies that do not compete with 9D7 are selected.

In particular embodiments, a CD28 binding domain is derived from TGN1412. In particular embodiments, the variable heavy chain of TGN1412 includes: QVQLVQSGAEVKKPGASVKVSCKASGYTFTSYYIHWVRQAPGQGLEWIGCIYPGNVNTNYNE KFKDRATLTVDTSISTAYMELSRLRSDDTAVYFCTRSHYGLDWNFDVWGQGTTVTVSS (SEQ ID NO: 195) and the variable light chain of TGN1412 includes: DIQMTQSPSSLSASVGDRVTITCHASQNIYVWLNWYQQKPGKAPKLLIYKASNLHTGVPSRFS GSGSGTDFTLTISSLQPEDFATYYCQQGQTYPYTFGGGTKVEIK (SEQ ID NO: 196).

In particular embodiments, the CD28 binding domain includes a variable light chain including a CDRL1 sequence including HASQNIYVWLN (SEQ ID NO: 197), CDRL2 sequence including KASNLHT (SEQ ID NO: 198), and CDRL3 sequence including QQGQTYPYT (SEQ ID NO: 199), a variable heavy chain including a CDRH1 sequence including GYTFTSYYIH (SEQ ID NO: 200), a CDRH2 sequence including CIYPGNVNTNYNEK (SEQ ID NO: 201), and a CDRH3 sequence including SHYGLDWNFDV (SEQ ID NO: 202).

In particular embodiments, the CD28 binding domain including a variable light chain including a CDRL1 sequence including HASQNIYVWLN (SEQ ID NO: 197), a CDRL2 sequence including KASNLHT (SEQ ID NO: 198), and a CDRL3 sequence including QQGQTYPYT (SEQ ID NO: 199) and a variable heavy chain including a CDRH1 sequence including SYYIH (SEQ ID NO: 203), a CDRH2 sequence including CIYPGNVNTNYNEKFKD (SEQ ID NO: 204), and a CDRH3 sequence including SHYGLDWNFDV (SEQ ID NO: 202).

Activated T-cells express 4-1BB (CD137). In particular embodiments, the 4-1BB binding domain includes a variable light chain including a CDRL1 sequence including RASQSVS (SEQ ID NO: 205), a CDRL2 sequence including ASNRAT (SEQ ID NO: 206), and a CDRL3 sequence including QRSNWPPALT (SEQ ID NO: 207) and a variable heavy chain including a CDRH1 sequence including YYWS (SEQ ID NO: 208), a CDRH2 sequence including INH, and a CDRH3 sequence including YGPGNYDWYFDL (SEQ ID NO: 209).

In particular embodiments, the 4-1BB binding domain includes a variable light chain including a CDRL1 sequence including SGDNIGDQYAH (SEQ ID NO: 210), a CDRL2 sequence including QDKNRPS (SEQ ID NO: 211), and a CDRL3 sequence including ATYTGFGSLAV (SEQ ID NO: 212) and a variable heavy chain including a CDRH1 sequence including GYSFSTYWIS (SEQ ID NO: 213), a CDRH2 sequence including KIYPGDSYTNYSPS (SEQ ID NO: 101) and a CDRH3 sequence including GYGIFDY (SEQ ID NO: 102).

Particular embodiments disclosed herein include targeting ligands that bind epitopes on CD8. In particular embodiments, the CD8 binding domain (e.g., scFv) is derived from the OKT8 antibody. For example, in particular embodiments, the CD8 binding domain is a human or humanized binding domain (e.g., scFv) including a variable light chain including a CDRL1 sequence including RTSRSISQYLA (SEQ ID NO: 103), a CDRL2 sequence including SGSTLQS (SEQ ID NO: 104), and a CDRL3 sequence including QQHNENPLT (SEQ ID NO: 10⁵). In particular embodiments, the CD8 binding domain is a human or humanized binding domain (e.g., scFv) including a variable heavy chain including a CDRH1 sequence including GFNIKD (SEQ ID NO: 106), a CDRH2 sequence including RIDPANDNT (SEQ ID NO: 107), and a CDRH3 sequence including GYGYYVFDH (SEQ ID NO: 108). These reflect CDR sequences of the OKT8 antibody.

Examples of commercially available antibodies with binding domains that bind to an NK cell receptor include: 5C6 and 1D11 (available from BioLegend® San Diego, Calif.); mAb 33, which binds KIR2DL4 (available from BioLegend®); P44-8, which binds NKp44 (available from BioLegend®); SK1, which binds CD8; and 3G8 which binds CD16. A binding domain that binds KIR2DL1 and KIR2DL2/3 includes a variable light chain region of the sequence: EIVLTQSPVTLSLSPGERATLSCRASQSVSSYLAWYQQKPGQAPRLLIYDASNRATGIPARFSG SGSGTDFTLTISSLEPEDFAVYYCQQRSNWMYTFGQGTKLEIKRT (SEQ ID NO: 109) and a variable heavy chain region of the sequence: QVQLVQSGAEVKKPGSSVKVSCKASGGTFSFYAISWVRQAPGQGLEWMGGFIPIFGAANYAQ KFQGRVTITADESTSTAYMELSSLRSDDTAVYYCARIPSGSYYYDYDMDVWGQGTTVTVSS (SEQ ID NO: 110). Additional NK binding antibodies are described in WO/2005/0003172 and U.S. Pat. No. 9,415,104.

Commercially available antibodies that bind to proteins expressed on the surface of macrophages include M1/70, which binds CD11b (available from BioLegend); KP1, which binds CD68 (available from ABCAM, Cambridge, United Kingdom); and ab87099, which binds CD163 (available from ABCAM).

The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); AI-Lazikani et al. (1997) J Mol Biol 273: 927-948 (Chothia numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); Lefranc M P et al. (2003) Dev Comp Immunol 27(1): 55-77 (IMGT numbering scheme); and Honegger and Pluckthun (2001) J Mol Biol 309(3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering.

In particular embodiments, when a gain of function genetic modification is intended, selective delivery can be enhanced by including regulatory elements that restrict expression of inserted constructs to the intended/selected cell type. For example, selective delivery can be enhanced by using the CD45 promoter, Wiskott-Aldrich syndrome (WASP) promoter or interferon (IFN)-beta promoter for HSCs; the murine stem cell virus promoter or the distal Ick promoter for HSCs or T cells; or the B29 promoter for B cells.

Other agents that can also facilitate internalization by and/or transfection of lymphocytes, such as poly(ethyleneimine)/DNA (PEI/DNA) complexes can also be used.

In particular embodiments, targeting ligands can be linked to a nuclease, for example, using amine-to-sulfhydryl, or sulfhydryl to sulfhydryl crosslinkers with various PEG spacers and/or Gly-Ser spacers. The addition of spacers allows flexibility to bind cognate receptors or cell surface proteins. In particular embodiments, spacers can have between 1-50; 10-50; 20-50; 30-50; 1-500; 10-250; 20-200; 30-150; 40-100; 50-75; or 5-75 repeating units or residues.

### (V) SOURCES & PROCESSING OF CELL POPULATIONS

Sources of HSC, HSPC and other lymphocytes include umbilical cord blood, placental blood, bone marrow, peripheral blood, embryonic cells, aortal-gonadal-mesonephros derived cells, lymph, liver, thymus, and spleen from age-appropriate donors. Methods regarding collection and processing, etc. of biological samples including blood samples are known. See, for example, Alsever et al., 1941, N.Y. St. J. Med. 41:126; De Gowin, et al., 1940, J. Am. Med. Ass. 114:850; Smith, et al., 1959, J. Thorac. Cardiovasc. Surg. 38:573; Rous and Turner, 1916, J. Exp. Med. 23:219; and Hum, 1968, Storage of Blood, Academic Press, New York, pp. 26-160; Kodo et al., 1984, J. Clin Invest. 73:1377-1384), All collected samples can be screened for undesirable components and discarded, treated, or used according to accepted current standards at the time. In particular embodiments, a biological sample includes any biological fluid, tissue, blood cell product, and/or organ that contains cell populations of interest.

A source of or biological sample including cell populations of interest can be obtained from a subject using any procedure generally known in the art. In particular embodiments, HSC/HSPC in peripheral blood are mobilized prior to collection. Peripheral blood HSC/HSPC can be mobilized by any method. Peripheral blood HSC/HSPC can be mobilized by treating the subject with any agent(s), described herein or known in the art, that increase the number of HSC/HSPC circulating in the peripheral blood of the subject. For example, in particular embodiments, peripheral blood is mobilized by treating the subject with one or more cytokines or growth factors (e.g., G-CSF, kit ligand (KL), IL-I, IL-7, IL-8, IL-11, Flt3 ligand, SCF, thrombopoietin, or GM-CSF (such as sargramostim)). Different types of G-CSF that can be used in the methods for mobilization of peripheral blood include filgrastim and longer acting G-CSF-pegfilgrastim. In particular embodiments, peripheral blood is mobilized by treating the subject with one or more chemokines (e.g., macrophage inflammatory protein-1a (MIP1α/CCL3)), chemokine receptor ligands (e.g., chemokine receptor 2 ligands GROβ and GROβ_{Δ4}), chemokine receptor analogs (e.g., stromal cell derived factor-la (SDF-1a) protein analogs such as CTCE-0021, CTCE-0214, or SDF-1a such as Met-SDF-1p), or chemokine receptor antagonists (e.g., chemokine (C-X-C motif) receptor 4 (CXCR4) antagonists such as AMD3100).

In particular embodiments, peripheral blood is mobilized by treating the subject with one or more anti-integrin signaling agents (e.g., function blocking anti-very late antigen 4 (VLA-4) antibody, or anti-vascular cell adhesion molecule 1 (VCAM-1)).

Peripheral blood can be mobilized by treating the subject with one or more cytotoxic drugs such as cyclophosphamide, etoposide or paclitaxel.

In particular embodiments, peripheral blood can be mobilized by administering to a subject one or more of the agents listed above for a certain period of time. For example, the subject can be treated with one or more agents (e.g., G-CSF) via injection (e.g., subcutaneous, intravenous or intraperitoneal), once daily or twice daily, for 1, 2, 3, 4, 5, 6, 7, 8, 9, 10, 11, 12, 13 or 14 days prior to collection of HSC/HSPC. In specific embodiments, HSC/HSPC are collected within 1, 2, 3, 4, 5, 6, 7, 8, 12, 14, 16, 18, 20 or 24 hours after the last dose of an agent used for mobilization of HSC/HSPC into peripheral blood. In particular embodiments, HSC/HSPC are mobilized by treating the subject with two or more different types of agents described above or known in the art, such as a growth factor (e.g., G-CSF) and a chemokine receptor antagonist (e.g., CXCR4 receptor antagonist such as AMD3100), or a growth factor (e.g., G-CSF or KL) and an anti-integrin agent (e.g., function blocking VLA-4 antibody). Different types of mobilizing agents can be administered concurrently or sequentially. For additional information regarding methods of mobilization of peripheral blood see, e.g., Craddock et al., 1997, Blood 90(12):4779-4788; Jin et al., 2008, Journal of Translational Medicine 6:39; Pelus, 2008, Curr. Opin. Hematol. 15(4):285-292; Papayannopoulou et al., 1998, Blood 91(7):2231-2239; Tricot et al., 2008, Haematologica 93(11):1739-1742; and Weaver et al., 2001, Bone Marrow Transplantation 27(2):S23-S29).

HSC/HSPC from peripheral blood can be collected from the blood through a syringe or catheter inserted into a subject's vein. For example, in particular embodiments, the peripheral blood can be collected using an apheresis machine. Blood flows from the vein through the catheter into an apheresis machine, which separates the white blood cells, including HSC/HSPC from the rest of the blood and then returns the remainder of the blood to the subject's body. Apheresis can be performed for several days (e.g., 1 to 5 days) until enough selected cell types (e.g., HSC, T cells) have been collected.

In particular embodiments, no further collection or isolation of selected cell types is needed before exposing the acquired sample to NP disclosed herein because the NP selectively target selected cell types within a heterogeneous cell population. In particular embodiments, the acquired sample has undergone no other manipulation aside from NP addition.

In some embodiments, blood cells collected from a subject are washed, e.g., to remove the plasma fraction and to place the cells in an appropriate buffer or media for subsequent exposure to NP. In particular embodiments, the cells are washed with phosphate buffered saline (PBS). In some embodiments, the wash solution lacks calcium and/or magnesium and/or many or all divalent cations. Washing can be accomplished using a semi-automated “flow-through” centrifuge (for example, the Cobe 2991 cell processor, Baxter) according to the manufacturer's instructions. Tangential flow filtration (TFF) can also be performed. In particular embodiments, cells can re-suspended in a variety of biocompatible buffers after washing, such as, Ca++/Mg++ free PBS.

In particular embodiments, it may be beneficial to engage in some limited further cell collection and isolation before exposure to NP disclosed herein. In particular embodiments, selected cell types can be collected and isolated from a sample using any appropriate technique.

Appropriate collection and isolation procedures include magnetic separation; fluorescence activated cell sorting (FACS; Williams et al., 1985, J. Immunol. 135:1004; Lu et al., 1986, Blood 68(1):126-133); affinity chromatography; agents joined to a monoclonal antibody or used in conjunction with a monoclonal antibody; “panning” with antibody attached to a solid matrix (Broxmeyer et al., 1984, J. Clin. Invest. 73:939-953); selective agglutination using a lectin such as soybean (Reisner et al., 1980, Proc. Natl. Acad. Sci. U.S.A. 77:1164); etc. Particular embodiments can utilize limited isolation. Limited isolation refers to crude cell enrichment, for example, by removal of red blood cells and/or adherent phagocytes.

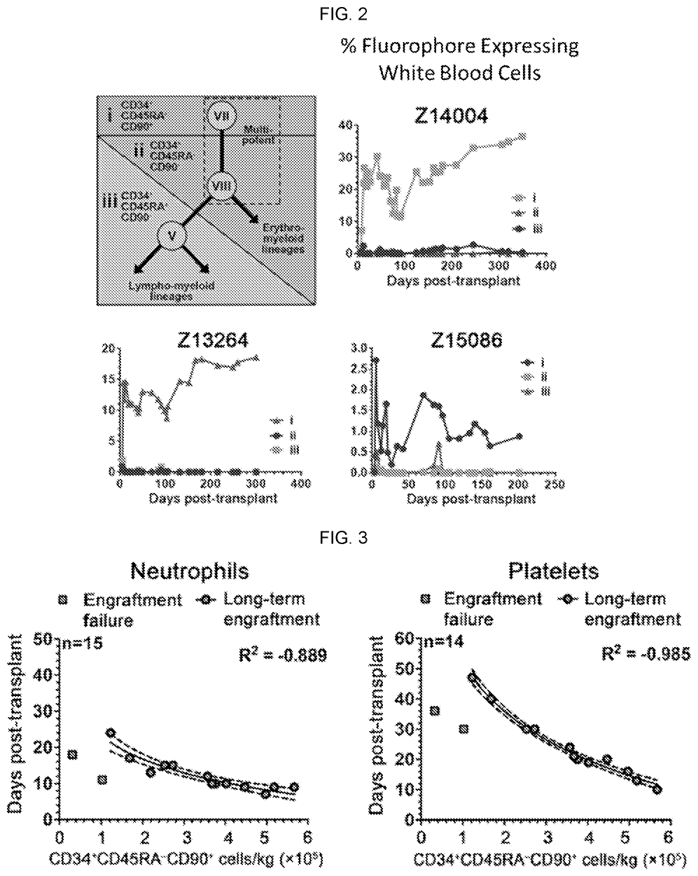

In particular embodiments, a subject sample (e.g., a blood sample) can be processed to select/enrich for the cellular profiled described in relation to FIG. 2, using, for example, CD34+ HSPC using antibodies directly or indirectly conjugated to magnetic particles in connection with a magnetic cell separator, for example, the CliniMACS@ Cell Separation System (Miltenyi Biotec, Bergisch Gladbach, Germany). In particular embodiments, where some limited cell enrichment is performed, cells within samples can be enriched for based on CD34 alone; CD133+ alone; CD90+ alone; CD164+ alone; CD46+ alone; or LH+ alone. In particular embodiments, cells can be enriched for and/or isolated based on one or more of CD34; CD133+; CD90+; CD164+; CD46+; AHR+; or LH+ in various combinations. In particular embodiments, LH+ means that a cell expresses the LHRH receptor. In particular embodiments, AHR+ means that a cell expresses the aryl hydrocarbon receptor.

When reduced, but not minimal manufacturing is practiced, it can be useful to expand HSC/HSPC. Expansion can occur in the presence of one more growth factors, such as: angiopoietin-like proteins (Angptls, e.g., Angpt12, Angpt13, Angpt17, Angpt15, and Mfap4); erythropoietin; fibroblast growth factor-1 (FGF-1); Flt-3 ligand (Flt-3L); granulocyte colony stimulating factor (G-CSF); granulocyte-macrophage colony stimulating factor (GM-CSF); insulin growth factor-2 (IFG-2); interleukin-3 (IL-3); interleukin-6 (IL-6); interleukin-7 (IL-7); interleukin-11 (IL-11); stem cell factor (SCF; also known as the c-kit ligand or mast cell growth factor); thrombopoietin (TPO); and analogs thereof (wherein the analogs include any structural variants of the growth factors having the biological activity of the naturally occurring growth factor; see, e.g., WO 2007/1145227 and U.S. Patent Publication No. 2010/0183564).

In particular embodiments, the amount or concentration of growth factors suitable for expanding HSC/HSPC or lymphocytes is the amount or concentration effective to promote proliferation. Lymphocyte populations are preferably expanded until a sufficient number of cells are obtained to provide for at least one infusion into a human subject, typically around 10⁴ cells/kg to 10⁹ cells/kg.

The amount or concentration of growth factors suitable for expanding HSC/HSPC or lymphocytes depends on the activity of the growth factor preparation, and the species correspondence between the growth factors and lymphocytes, etc. Generally, when the growth factor(s) and lymphocytes are of the same species, the total amount of growth factor in the culture medium ranges from 1 ng/ml to 5 μg/ml, from 5 ng/ml to 1 μg/ml, or from 5 ng/ml to 250 ng/ml. In particular embodiments, the amount of growth factors can be in the range of 5-1000 or 50-100 ng/ml.

In particular embodiments, growth factors are present in an expansion culture condition at the following concentrations: 25-300 ng/ml SCF, 25-300 ng/ml Flt-3L, 25-100 ng/ml TPO, 25-100 ng/ml IL-6 and 10 ng/ml IL-3. In particular embodiments, 50, 100, or 200 ng/ml SCF; 50, 100, or 200 ng/ml of Flt-3L; 50 or 100 ng/ml TPO; 50 or 100 ng/ml IL-6; and 10 ng/ml IL-3 can be used.

HSC/HSPC or lymphocytes can be expanded in a tissue culture dish onto which an extracellular matrix protein such as fibronectin (FN), or a fragment thereof (e.g., CH-296 (Dao et. al., 1998, Blood 92(12):4612-21)) or RetroNectin® (a recombinant human fibronectin fragment; (Clontech Laboratories, Inc., Madison, Wis.) is bound.

Notch agonists can be particularly useful for expanding HSC/HSPC. In particular embodiments, HSC/HSPC can be expanded by exposing the HSC/HSPC to an immobilized Notch agonist, and 50 ng/ml or 100 ng/ml SCF; to an immobilized Notch agonist, and 50 ng/ml or 100 ng/ml of each of Flt-3L, IL-6, TPO, and SCF; or an immobilized Notch agonist, and 50 ng/ml or 100 ng/ml of each of Flt-3L, IL-6, TPO, and SCF, and 10 ng/ml of IL-11 or IL-3.

For additional general information regarding appropriate culturing and/or expansion conditions, see U.S. Pat. No. 7,399,633; U.S. Patent Publication No. 2010/0183564; Freshney Culture of Animal Cells, Wiley-Liss, Inc., New York, N.Y. (1994)); Vamum-Finney et al., 1993, Blood 101:1784-1789; Ohishi et al., 2002, J. Clin. Invest. 110:1165-1174; Delaney et al., 2010, Nature Med. 16(2): 232-236; WO 2006/047569A2; WO 2007/095594A2; U.S. Pat. No. 5,004,681; WO 2011/127470 A1; WO 2011/127472A1; and See Chapter2 of Regenerative Medicine, Department of Health and Human Services, August 2006, and the references cited therein.

When reduced, but not minimal manipulation manufacturing is performed, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, a selected T cell type can be enriched for and/or isolated based on cell-marker based positive and/or negative selection. Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques.

CD3⁺, CD28⁺ T cells can be positively selected for and expanded using anti-CD3/anti-CD28 conjugated magnetic beads (e.g., DYNABEADS® M-450 CD3/CD28 T Cell Expander).

In particular embodiments, a CD8⁺ or CD4⁺ selection step is used to separate CD4⁺ helper and CD8⁺ cytotoxic T cells. Such CD8⁺ and CD4⁺ populations can be further sorted into sub-populations by positive or negative selection for markers expressed or expressed to a relatively higher degree on one or more naive, memory, and/or effector T cell subpopulations.

In some embodiments, enrichment for central memory T (T_{CM}) cells is carried out. In particular embodiments, memory T cells are present in both CD62L subsets of CD8⁺ peripheral blood lymphocytes. PBMC can be enriched for or depleted of CD62L, CD8 and/or CD62L⁺CD8⁺ fractions, such as by using anti-CD8 and anti-CD62L antibodies.

In some embodiments, the enrichment for central memory T (T_{CM}) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CCR7, CD45RO, and/or CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or RORI, and positive selection based on a marker characteristic of central memory T cells, such as CCR7, CD45RO, and/or CD62L, where the positive and negative selections are carried out in either order.

In particular embodiments, cell enrichment results in a bulk CD8+ FACs-sorted cell population.

T cell populations can be incubated in a culture-initiating composition to expand T cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

Exemplary culture media for culturing T cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10⁻⁴-0.75×10⁻⁴M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, Md.) supplemented with 5% human AB serum (Gemcell, West Sacramento, Calif.), 1% HEPES (Gibco, Grand Island, N.Y.), 1% Pen-Strep (Gibco), 1% GlutaMax (Gibco), and 2% N-acetyl cysteine (Sigma-Aldrich, St. Louis, Mo.). T cell culture media are also commercially available from Hyclone (Logan, Utah). Additional T cell activating components that can be added to such culture media are described in more detail below.

In some embodiments, the T cells are expanded by adding to the culture-initiating composition feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can include gamma-irradiated PBMC feeder cells. In some embodiments, the PBMC are irradiated with gamma rays in the range of 3000 to 3600 rads to prevent cell division. In some aspects, the feeder cells are added to culture medium prior to the addition of the populations of T cells.

Optionally, the incubation may further include adding non-dividing EBV-transformed lymphoblastoid cells (LCL) as feeder cells. LCL can be irradiated with gamma rays in the range of 6000 to 10,000 rads. The LCL feeder cells in some aspects is provided in any suitable amount, such as a ratio of LCL feeder cells to initial T lymphocytes of at least 10:1.

In some embodiments, the stimulating conditions include temperature suitable for the growth of human T lymphocytes, for example, at least 25° C., at least 30° C., or 37° C.

The activating culture conditions for T cells include conditions whereby T cells of the culture-initiating composition proliferate or expand.

### (VI) FORMULATION AND CRYOPRESERVATION OF CELLS

Cells genetically modified using minimal manipulation manufacturing processing can be directly administered to a subject following the genetic modification. In particular embodiments, genetically-modified cells can be formulated into cell-based compositions for administration to the subject. A cell-based composition refers to cells prepared with a pharmaceutically acceptable carrier for administration to a subject.

Exemplary carriers and modes of administration of cells are described at pages 14-15 of U.S. Patent Publication No. 2010/0183564. Additional pharmaceutical carriers are described in Remington: The Science and Practice of Pharmacy, 21st Edition, David B. Troy, ed., Lippicott Williams & Wilkins (2005).

In particular embodiments, cells can be harvested from a culture medium, and washed and concentrated into a carrier in a therapeutically-effective amount. Exemplary carriers include saline, buffered saline, physiological saline, water, Hanks' solution, Ringer's solution, Nonnosol-R (Abbott Labs), Plasma-Lyte A® (Baxter Laboratories, Inc., Morton Grove, Ill.), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, cell-based compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells, for example, within cell-based compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 101. If a patient is conditioned, product equivalent to a minimum of 2 million CD34+ cells/kg of body weight infused is preferred. In a non-conditioned patient, a minimum of 1 million CD34+ cells/kg of body weight can be acceptable.

In cell-based compositions disclosed herein, cells are generally in a volume of a liter or less, 500 mL or less, 250 mL or less, or 100 mL or less. Hence the density of administered cells is typically greater than 10⁴ cells/mL, 10⁷ cells/mL, or 10⁸ cells/mL.

The cells or cell-based compositions disclosed herein can be prepared for administration by, for example, injection, infusion, perfusion, or lavage. The cells or cell-based compositions can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

In particular embodiments, cells or cell-based compositions are administered to a subject in need thereof as soon as is reasonably possible following the completion of genetic modification and/or formulation for administration. In particular embodiments, it can be necessary or beneficial to cryopreserve a cell. The terms “frozen/freezing” and “cryopreserved/cryopreserving” can be used interchangeably. Freezing includes freeze drying. In particular embodiments, cryo-preserving fresh cells can reduce non-desired cell populations. Accordingly, particular embodiments include cryo-preserving a biological sample before NP are administered to the sample. In particular embodiments, biological samples are washed to remove platelets before cryopreservation.

As is understood by one of ordinary skill in the art, the freezing of cells can be destructive (see Mazur, P., 1977, Cryobiology 14:251-272) but there are numerous procedures available to prevent such damage. For example, damage can be avoided by (a) use of a cryoprotective agent, (b) control of the freezing rate, and/or (c) storage at a temperature sufficiently low to minimize degradative reactions. Exemplary cryoprotective agents include dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al.., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), and inorganic salts (Phan The Tran and Bender, 1960, Proc. Soc. Exp. Biol. Med. 104:388; Phan The Tran and Bender, 1961, in Radiobiology, Proceedings of the Third Australian Conference on Radiobiology, Ilbery ed., Butterworth, London, p. 59). In particular embodiments, DMSO can be used. Addition of plasma (e.g., to a concentration of 20-25%) can augment the protective effects of DMSO. After addition of DMSO, cells can be kept at 0° C. until freezing, because DMSO concentrations of 1% can be toxic at temperatures above 4° C.

In the cryopreservation of cells, slow controlled cooling rates can be critical and different cryoprotective agents (Rapatz et al., 1968, Cryobiology 5(1): 18-25) and different cell types have different optimal cooling rates (see e.g., Rowe and Rinfret, 1962, Blood 20:636; Rowe, 1966, Cryobiology 3(1):12-18; Lewis, et al., 1967, Transfusion 7(1):17-32; and Mazur, 1970, Science 168:939-949 for effects of cooling velocity on survival of stem cells and on their transplantation potential). The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling.

In particular embodiments, DMSO-treated cells can be pre-cooled on ice and transferred to a tray containing chilled methanol which is placed, in turn, in a mechanical refrigerator (e.g., Harris or Revco) at −80° C. Thermocouple measurements of the methanol bath and the samples indicate a cooling rate of 1° to 3° C./minute can be preferred. After at least two hours, the specimens can have reached a temperature of −80° C. and can be placed directly into liquid nitrogen (−196° C.).

After thorough freezing, the cells can be rapidly transferred to a long-term cryogenic storage vessel. In particular embodiments, samples can be cryogenically stored in liquid nitrogen (−196° C.) or vapor (−1° C.). Such storage is facilitated by the availability of highly efficient liquid nitrogen refrigerators.

Further considerations and procedures for the manipulation, cryopreservation, and long term storage of cells, can be found in the following exemplary references: U.S. Pat. Nos. 4,199,022; 3,753,357; and 4,559,298; Gorin, 1986, Clinics In Haematology 15(1):19-48; Bone-Marrow Conservation, Culture and Transplantation, Proceedings of a Panel, Moscow, July 22-26, 1968, International Atomic Energy Agency, Vienna, pp. 107-186; Livesey and Linner, 1987, Nature 327:255; Linner et al., 1986, J. Histochem. Cytochem. 34(9):1123-1135; Simione, 1992, J. Parenter. Sci. Technol. 46(6):226-32).

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

In particular embodiments, methods can be used to prevent cellular clumping during thawing. Exemplary methods include: the addition before and/or after freezing of DNase (Spitzer et al., 1980, Cancer 45:3075-3085), low molecular weight dextran and citrate, hydroxyethyl starch (Stiff et al., 1983, Cryobiology 20:17-24), etc.

As is understood by one of ordinary skill in the art, if a cryoprotective agent that is toxic to humans is used, it should be removed prior to therapeutic use. DMSO has no serious toxicity.

### (VII) NANOPARTICLE FORMULATIONS

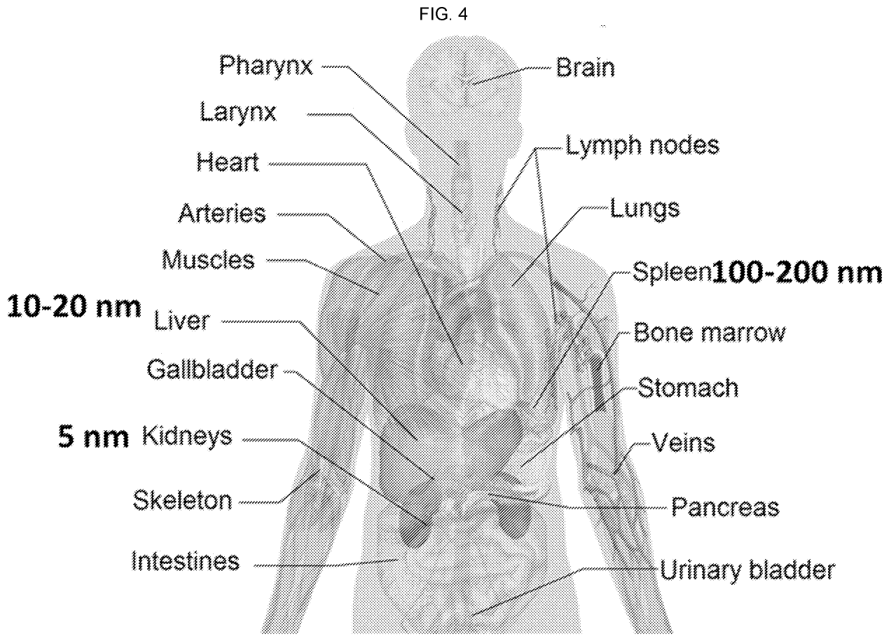

NP disclosed herein can also be formulated for direct administration to subject. As depicted in FIG. 4, the size of an AuNP can be selected to affect biodistribution within the human body. NP suitable for use in the present disclosure can be any shape and can range in size from 5 nm-1000 nm in size, e.g., from 5 nm-10 nm, 5-50 nm, 5 nm-75 nm, 5 nm-40 nm, 10 nm-30, or 20 nm-30 nm. NP can also have a size in the range of from 10 nm-15 nm, 15 nm-20 nm, 20 nm-25 nm, 25 nm-30 nm, 30 nm-35 nm, 35 nm-40 nm, 40 nm-45 nm, or 45 nm-50 nm, 50 nm-55 nm, 55 nm-60 nm, 60 nm-65 nm, 65 nm-70 nm, 70 nm-75 nm, 75 nm-80 nm, 80 nm-85 nm, 85 nm-90 nm, 90 nm-95 nm, 95 nm-100 nm, 100 nm-10⁵ nm, 10⁵ nm-110 nm, 110 nm-115 nm, 115 nm-120 nm, 120 nm-125 nm, 125 nm-130 nm, 130 nm-135 nm, 135 nm-140 nm, 140 nm-145 nm, 145 nm-150 nm, 100 nm-500 nm, 100 nm-150 nm, 150 nm-200 nm, 200 nm-250 nm, 250 nm-300 nm, 300 nm-350 nm, 350 nm-400 nm, 400 nm-450 nm, or 450 nm-500 nm. In particular embodiments, NP greater than 550 nm are excluded. This is because particles or aggregated particles of >600 nm are not amenable to cellular uptake.

Therapeutically effective amounts of NP within a composition can include at least 0.1% w/v or w/w particles; at least 1% w/v or w/w particles; at least 10% w/v or w/w particles; at least 20% w/v or w/w particles; at least 30% w/v or w/w particles; at least 40% w/v or w/w particles; at least 50% w/v or w/w particles; at least 60% w/v or w/w particles; at least 70% w/v or w/w particles; at least 80% w/v or w/w particles; at least 90% w/v or w/w particles; at least 95% w/v or w/w particles; or at least 99% w/v or w/w particles.

### (VIII) KITS

The disclosure also provides kits containing any one or more of the elements disclosed herein. In particular embodiments, a kit can include NP as described herein including guide RNA and a nuclease capable of cutting a target sequence. The kit may additionally include one or more HDT, targeting ligands, and/or polymers (e.g., PEG, PEI). Elements may be provided individually or in combinations, and may be provided in any suitable container, such as a vial, a bottle, a bag or a tube. In some embodiments, the kit includes instructions in one or more languages.

In particular embodiments, a kit includes one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from 7 to 10. In some embodiments, the kit includes a guide RNA (e.g., cRNA), a nuclease (e.g., Cpf1), an Au core, and/or a homologous recombination template polynucleotide.

Kits may also include one or more components to collect, process, modify, and/or formulate cells for administration. Kits can be provided with components to perform reduced or minimal manipulation ex vivo cell manufacturing. Articles of manufacture and/or instructions for clinical staff can also be included.

### (IX) EXEMPLARY METHODS OF USE

As indicated, selected cell types can be obtained from a subject. In particular embodiments, the cells are re-introduced into the same subject from whom the original sample was derived in a therapeutically effective amount. In particular embodiments, the cells are administered to a different subject in a therapeutically effective amount.

The compositions and formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). In particular embodiments, subjects are human patients.

Examples of diseases that can be treated using the NP compositions or cell formulations manufactured with reduced or minimal manipulation described herein include monogenetic blood disorders, hemophilia, Grave's Disease, rheumatoid arthritis, pernicious anemia, Multiple Sclerosis (MS), inflammatory bowel disease, systemic lupus erythematosus (SLE), Wiskott-Aldrich syndrome (WAS), chronic granulomatous disease (CGD), Battens disease, adrenoleukodystrophy (ALD) or metachromatic leukodystrophy (MLD), muscular dystrophy, pulmonary aveolar proteinosis (PAP), pyruvate kinase deficiency, Shwachmann-Diamond-Blackfan anemia, dyskeratosis congenita, cystic fibrosis, Parkinson's disease, Alzheimer's disease, amyotrophic lateral sclerosis (Lou Gehrig's disease), acute lymphoblastic leukemia (ALL), acute myelogenous leukemia (AML), agnogenic myeloid metaplasia, amegakaryocytosis/congenital thrombocytopenia, ataxia telangiectasia, β-thalassemia major, CLL, chronic myelogenous leukemia (CML), chronic myelomonocytic leukemia, common variable immune deficiency (CVID), complement disorders, congenital (X-linked) agammaglobulinemia, familial erythrophagocytic lymphohistiocytosis, Hodgkin's lymphoma, Hurler's syndrome, hyper IgM, IgG subclass deficiency, juvenile myelomonocytic leukemia, mucopolysaccharidoses, multiple myeloma, myelodysplasia, non-Hodgkin's lymphoma, paroxysmal nocturnal hemoglobinuria (PNH), primary immunodeficiency diseases with antibody deficiency, pure red cell aplasia, refractory anemia, selective IgA deficiency, severe aplastic anemia, SCD, and/or specific antibody deficiency.

### (X) EXEMPLARY MANUFACTURING EMBODIMENTS & COMPARISONS

| Parameter | Disclosed Embodiment |
|---|---|
| Size of AuNP Core | 15 nm |
| AuNP Synthesis Method | Turkevich (1951) |
| Starting solution | 0.25 mM chloroauric acid (HAuCl₄) |
| 1st synthesis step | Bring above solution to boiling point and reduce by adding 3.33% |
| | sodium citrate (Na3C6H5O7) while stirring vigorously (700 rpm) |
| | under a reflux system |
| 2nd synthesis step | Reduce by adding 3.33% sodium citrate (Na₃C₆H₅O₇) while |
| | stirring vigorously (700 rpm) under a reflux system |
| Cleanup step | Wash AuNPs 3X |
| Initial Resuspension | Rnase free molecular grade water (H₂O) |
| First Loading Step | 10 micrograms/mL AuNP added to crRNA (Cpf1/Cas12a) or |
| | crRNA + tracrRNA (Cas9) solution at a weight/weight ratio of 0.5 |
| Second Loading Step | 10 mM Citrate buffer (pH 3.0) added and mixed for 5 |
| | min. Nanoconjugates are centrifuged at 20000 x g for 20 minutes |
| | at room temperature and re-dispersed in 0.9% sodium choloride. |
| Third Loading Step | Add nuclease protein (Cpf1/Cas12a or Cas9) to nanoconjugate |
| | solution at a weight/weight ratio of 0.6 |
| Fourth Loading Step | Add 0.005% branched polyethylenimine (2000 MW) and mix by |
| | pipetting. |
| Fifth Loading Step | Add single stranded DNA template (ssODN) to nanoconjugates in |
| | a weight to weight ratio of 1.0 |
| Final Resuspension | RNase free water |
| Guide RNA Loaded | Guide RNA (crRNA) with the following modifications: For Cpf1 |
| | (Cas12a): 1. 3′ 18-atom oligo ethylene glycol (OEG) spacer |
| | (iSp18) 2. 3′ terminal thiol For Cas9: (unmodified tracrRNA) 1. 5′ |
| | 18-atom oligo ethylene glycol (OEG) spacer (iSp18) 2. 5′ terminal |
| | thiol |
| Nuclease Loaded | Cpf1 (Cas12a), Cas9, or Mega-TAL |
| ssODN Loaded | Unmodified homology-directed template with symmetric or |
| | asymmetric homology arms of any length, up to a total of 3 |
| | kilobases in total |
| Final actual size of fully | 25-30 nm |
| loaded AuNP | |
| Final hydrodynamic size | 176 nm |
| of fully loaded AuNP | |

Comparison of Exemplary Manufacturing Protocols.

| | Synthesis Protocol to | Synthesis Protocol to | |
|---|---|---|---|
| | Generate NP as Depicted | Generate NP as Depicted | |
| Parameter | in FIGs. 5B and 6B | in FIGs. 5D and 6C-6E | Notes |
| AuNP | Turkevich (1951) | Turkevich (1951) | |
| Synthesis | | | |
| Method | | | |
| Size of AuNP | 15 nm | | |
| Core | | | |
| Starting | 0.25 mM chloroauric acid | 0.25 mM chloroauric acid | |
| solution | (HAuCl₄) | (HAuCl₄) | |
| 1st synthesis | Bring above solution to | Bring above solution to | |
| step | boiling point and reduce by | boiling point and reduce by | |
| | adding 3.33% sodium | adding 3.33% sodium | |
| | citrate (Na3C6H5O7) while | citrate (Na3C6H5O7) while | |
| | stirring vigorously (700 | stirring vigorously (700 | |
| | rpm) under a reflux system | rpm) under a reflux system | |
| 2nd synthesis | Reduce by adding 3.33% | Reduce by adding 3.33% | |
| step | sodium citrate (Na₃C₆H₅O₇) | sodium citrate (Na₃C₆H₅O₇) | |
| | while stirring vigorously | while stirring vigorously | |
| | (700 rpm) under a reflux | (700 rpm) under a reflux | |
| | system | system | |
| 3rd synthesis | | Seeding-growth for 50 and | |
| step | | 100 nm NP. Add 2.44 mL, | |
| | | and 304 uL of 15 nm AuNP | |
| | | to 100 mL of 0.25 mM | |
| | | HAuCl4 solution for 50 nm | |
| | | and 100 nm NP | |
| | | respectively and mix with 1 | |
| | | mL of 15 mM sodium | |
| | | citrate solution. Finally, | |
| | | while stirring 1 mL of 25 | |
| | | mM hydroquinone solution | |
| | | is added and mixed for 30 | |
| | | min to make NP. | |
| 4th synthesis | | Coat the surface of NP by | |
| step | | adding thiolated PEI in | |
| | | 0.005% concentration and | |
| | | mixing for 15 min. | |
| Cleanup step | Wash AuNPs 3X | Wash AuNPs 3X | |
| Initial | Rnase free molecular | Rnase free molecular | |
| Resuspension | grade water (H₂O) | grade water (H₂O) | |
| First Loading | 10 micrograms/mL AuNP | Fully loading the surface of | |
| Step | added to crRNA | NP with ssDNA template in | |
| | (Cpf1/Cas12a) or crRNA + | AuNP/ssDNA w/w ratio of | |
| | tracrRNA (Cas9) solution | 0.5. | |
| | at a weight/weight ratio of | | |
| | 0.5 | | |
| Second | 10 mM Citrate buffer (pH | Thilation of CRISPR | NaCl screens the |
| Loading Step | 3.0) added and mixed for 5 | nuclease by 2- | negative charge |
| | min. Nanoconjugates are | iminothiolane and | on the surface of |
| | centrifuged at 20000 x g | purification. Maleimide | the AuNP so that |
| | for 20 minutes at room | activation of the targeting | negatively charged |
| | temperature and re- | moeity by SM(PEG)24 | DNA is not |
| | dispersed in 0.9% sodium | linker and following | repelled. Citrate |
| | choloride. | purification conjugation to | buffer performs |
| | | CRISPR nuclease. | the same function |
| | | | in 3-5 minutes, |
| | | | whereas sodium |
| | | | chloride must be |
| | | | added gradually in |
| | | | incremental |
| | | | concentrations |
| | | | over 48 hours. |
| Third Loading | Add nuclease protein | Maleimide activation of | RNP has a |
| Step | (Cpf1/Cas12a or Cas9) to | crRNA by Sulfo-SMCC and | negative charge |
| | nanoconjugate solution at | following purification | so it cannot bind to |
| | a weight/weight ratio of 0.6 | making RNP complex with | the negative |
| | | conjugated CRISPR | surface of AuNP |
| | | nuclease. | conjugated with |
| | | | DNA. In these |
| | | | methods the RNP |
| | | | complex is formed |
| | | | by specific |
| | | | interaction of the |
| | | | Cas9 or Cpf1 with |
| | | | the crRNA on the |
| | | | surface of AuNP. |
| Fourth | Add 0.005% branched | Conjugation of targeting | |
| Loading Step | polyethylenimine (2000 | moeity/CRISPR | |
| | MW) and mix by pipetting. | nuclease/crRNA complex | |
| | | to ssDNA loaded NP | |
| | | through available thiol | |
| | | groups of PEI. | |
| Fifth Loading | Add single stranded DNA | none | |
| Step | template (ssODN) to | | |
| | nanoconjugates in a | | |
| | weight to weight ratio of | | |
| | 1.0 | | |
| Sixth Loading | None | none | |
| Step | | | |
| Final | RNase free water | PBS | |
| Resuspension | | | |
| Final actual | 25-30 nm | 30-130 nm | |
| size of fully | | | |
| loaded AuNP | | | |
| Final | 176 nm | 50-200 nm | |
| hydrodynamic | | | |
| size of fully | | | |
| loaded AuNP | | | |
| Target cell | Dividing and Nondividing | Dividing cells: Blood cells | |
| population | cells: Blood cells (HSC, | (HSC, HSPC) Stem Cells. | |
| | HSPC, T cells, NK Cells, | | |
| | Monocytes, Lymphocytes, | | |
| | Macrophages, | | |
| | Megakaryocytes); Central | | |
| | Nervous System | | |
| | (Astrocytes, Neurons, Glial | | |
| | cells, Microglia); Stromal | | |
| | cells (Mesenchymal stem | | |
| | cells, fibroblasts); Epithelial | | |
| | cells, Stem Cells. | | |
| Guide RNA | Guide RNA (crRNA) with | Guide RNA (crRNA) with | Cpf1 (Cas12a) |
| Loaded | the following modifications: | the following modifications: | only requires |
| | ForCpfl (Cas12a): 1. 3′ | ForCpfl (Cas12a): 1. 3′ | crRNA, which is |
| | 18-atom oligo ethylene | Amine or thiol 2. 3′ Internal | 40 nt in length. |
| | glycol (OEG) spacer | PEG and terminal | Cas9 requires two |
| | (iSp18) 2. 3′ terminal thiol | maleimide or NHS ester | RNAs, the crRNA |
| | For Cas9: (unmodified | For Cas9: (unmodified | guide (40 nt) and a |
| | tracrRNA) 1. 5′ 18-atom | tracrRNA) 1. 5′ Amine or | tracrRNA. If the |
| | oligo ethylene glycol | thiol 2. 5′ Internal PEG and | single-guide |
| | (OEG) spacer (iSp18) 2. 5′ | terminal maleimide or NHS | method is used for |
| | terminal thiol | ester | Cas9, the single |
| | | | crRNA must be |
| | | | 100 nt in length, |
| | | | which is not |
| | | | suitable for |
| | | | chemical |
| | | | modification. |
| Nuclease | Cpf1 (Cas12a), Cas9, or | Cpf1 (Cas12a), Cas9, or | Mega-TAL is |
| Loaded | Mega-TAL (see notes) | Mega-TAL (see notes) | engineered to |
| | | | include a terminal |
| | | | cysteine residue |
| | | | for thiol-mediated |
| | | | covalent binding |
| | | | directly to the |
| | | | surface of the |
| | | | AuNP (no guide |
| | | | RNA required). |
| | | | The same |
| | | | procedure can be |
| | | | done with Cpf1 or |
| | | | Cas9 to make a |
| | | | different form of n |
| | | | NP. |
| ssODN | Unmodified homology- | Modified and unmodified | |
| Loaded | directed template with | homology-directed | |
| | symmetric or asymmetric | template with symmetric or | |
| | homology arms of any | asymmetric homology | |
| | length, up to a total of 3 | arms of any length, up to a | |
| | kilobases in total | total of 3 kilobases in total | |
| Targeting | None | Antibody (CD34, CD133, | |
| Moiety | | CD164, CD90); aptamer | |
| Loaded | | (CD133) and/or ligand | |
| | | (luteinizing hormone or | |
| | | degerelix acetate). These | |
| | | can be loaded alone or in | |
| | | combination with one | |
| | | another. | |

### (XI) ASSAYS TO ASSESS NANOPARTICLE PERFORMANCE

Assays known in the art can be used to assess effectiveness of NP described herein including: effectiveness of NP uptake by cell populations, effect on cell viability from NP uptake, and any residual presence of NP in minimally manipulated blood cell products including cell populations genetically modified using NP described herein. The presence, level, or rate of gene editing of selected cell populations can also be determined, as described above. Assays can also be used to determine whether a therapeutic formulation including NP described herein and/or whether a minimally manipulated blood cell product including cell populations genetically modified using NP described herein are selected for further development.

NP uptake by cell populations can be assessed by a number of methods known in the art including confocal microscopy, fluorescence activated cell sorting (FACS), and inductively coupled plasma (ICP) techniques including: ICP-mass spectrometry (ICP-MS), ICP-atomic emission spectroscopy (ICP-AES), and ICP-optical emission spectroscopy (ICP-OES). In particular embodiments, crRNA and/or donor template can be labeled with dyes and assessed for uptake by cells using confocal microscopy. In particular embodiments, FACS using fluorescently labeled antibodies recognizing cell surface markers can be used in conjunction with confocal microscopy to test whether cell populations of interest have been targeted by the labeled NP. In particular embodiments, labeled antibodies recognizing cell surface markers are on small magnetized particles, and immunomagnetic bead-based sorting can be performed to determine what cell populations have been targeted by the labeled NP. In particular embodiments, ICP techniques allow for qualitative and quantitative trace element detection. Particular embodiments of ICP uses plasma to atomize or excite samples for detection. In particular embodiments, an ICP can be generated by directing the energy of a radio frequency generator into a suitable gas such as ICP argon, helium, or nitrogen. In particular embodiments, ICP-MS can be used to detect any residual NP in minimally manipulated blood cell products including cell populations genetically modified using NP described herein.

In particular embodiments, 50% to 100%, 50% to 90%, or 50% to 80%, of target cells take up NP described herein. In particular embodiments, 50%, 55%, 60%, 65%, 70%, 75%, 80%, 85%, 90%, 95%, or 100% of target cells take up NP described herein. In particular embodiments, target cells are cells that are targeted by NP described herein for genetic modification. In particular embodiments, target cells are cells that are targeted by NP by a targeting ligand on the NP that binds to a cell surface marker on the cells. In particular embodiments, non-target cells are cells that are not targeted by NP described herein for genetic modification. In particular embodiments, non-target cells are cells that are not targeted by NP described herein because they do not express the cell surface marker recognized by a targeting ligand on the NP.

Cell viability after treatment with Au/CRISPR NP can be analyzed at different time points using trypan blue, a stain that labels dead cells exclusively and thus can be used to discriminate between viable and dead cells. Trypan blue is available from a commercial distributor such as Invitrogen (Carlsbad, Calif.). Counting of cells can be performed using a cell counter such as the Countess II FL Automated Cell Counter from ThermoFisher Scientific (Waltham, Mass.). Percent cell viability of each sample can be recorded and reported as mean±SD.

Cell viability can also be analyzed using fluorescence-based assays such as the LIVE/DEAD® assay kit from Invitrogen (Carlsbad, Calif.). In a LIVE/DEAD® assay, two compounds can distinguish between live and dead cells. First, a cell-impermeant dye (e.g., ethidium homodimer-1) only binds to the surface of live cells and yields very dim fluorescence, while the dye can penetrate the cell membrane in dead cells and bind to internal molecules, yielding very bright fluorescence. Second, a non-fluorescent cell-permeant dye (e.g., calcein AM) can be converted to an intensely fluorescent version (e.g., calcein) by an esterase activity in live cells. Labeled cells can be imaged under a fluorescence microscope using appropriate excitation and emission values. Live and dead cells can be counted and imaged using appropriate software.

In particular embodiments, 70% to 100%, 70% to 90%, or 70% to 80%, of target cells are viable after treatment with a therapeutic formulation including NP described herein. In particular embodiments, 70%, 75%, 80%, 85%, 90%, 95%, or 100% of target cells are viable after treatment with a therapeutic formulation including NP described herein.

In particular embodiments, the fitness of HSC/HSPC treated with NP described herein can be assessed by a colony forming cell (CFC) assay (also known as a methylcellulose assay). In a CFC assay, the ability of HSC/HSPC to proliferate and differentiate into colonies in a semi-solid media in response to cytokine stimulation can be assessed. Cells can be plated in methylcellulose containing recombinant human growth factors and incubated for a specified period of time. Resulting colonies can be counted and scored for morphology on a stereo microscope to determine the number of colony-forming cells for every number of cells plated (e.g., 100,000 cells plated).

In particular embodiments, the fitness of HSC/HSPC treated with NP described herein can be assessed by in vivo studies using sub-lethally irradiated immunodeficient (NOD/SCID gamma −/−; NSG) mice. These studies can assess the fitness of HSC/HSPC by the cells' ability to reconstitute a myelosuppressed host. In particular embodiments, a specified number of cells can be infused into NSG mice, and the mice are followed for a number of weeks to assess engraftment of the HSC/HSPC.

Engraftment of HSC/HSPC and/or other cell populations can be assessed by collecting biological samples (e.g., blood, bone marrow, spleen) from the mice and performing FACS using fluorescently labeled antibodies binding cell surface markers. In particular embodiments, FACS can detect the level of CD45 expressing cells (HSC/HSPC), CD20 expressing cells (B cells), CD14 expressing cells (monocytes), CD3 expressing cells (T cells), CD4 expressing cells (T cells), and CD8 expressing cells (T cells). In particular embodiments, immunomagnetic bead-based sorting including small magnetized particles containing antibodies binding cell surface markers can be used.

In particular embodiments, a therapeutic formulation including NP described herein can undergo release testing to determine suitability of the therapeutic formulation for reinfusion testing in vivo. In particular embodiments, release testing includes gram stain, 3 day sterility, 14 day sterility, mycoplasma, endotoxin, and cell viability by trypan blue. In particular embodiments, a therapeutic formulation can be advanced for further development if the release testing yields: negative results for gram stain, 3 day sterility, 14 day sterility, and mycoplasma; ≤0.5 EU/mL endotoxin; and ≥70% viability by trypan blue.

In particular embodiments, performance of a minimally manipulated blood cell product including cell populations genetically modified using NP described herein can be assessed in vivo using NSG mice. In particular embodiments, engraftment of HSC/HSPC and/or other cell populations can be assessed as described above.

Mice infused with a minimally manipulated blood cell product including cell populations genetically modified using NP described herein can be monitored visually for any effects of the infusion on health (e.g., grooming, weight, activity level) following protocols as described in Burkholder et al. Health Evaluation of Experimental Laboratory Mice. Current Protocols in Mouse Biology, 2012; 2:145-165. In particular embodiments, presence of NP in the infused blood cell product can be assessed by ICP-MS. In particular embodiments, presence of NP in urine and feces of the mice can be assessed by ICP-MS at a given time after infusion (e.g., 72 hours) to determine whether all NP have been cleared (mass balance). In particular embodiments, the minimum threshold in urine/feces over 72 hours is 0, and the maximum threshold cannot exceed total mass injected. If bioaccumulation is indicated, micro computed tomography (CT) imaging of live mice can be performed to assess the location of accumulation. In particular embodiments, ICP-MS and/or necropsy can also be performed to determine sites for bioaccumulation. In particular embodiments, micro CT, necropsy, and/or trace element analysis (e.g., ICP-MS) can be combined with histopathology to assess potential toxicity of NP in infused mice. In particular embodiments, organ toxicity in infused mice is compared relative to untreated controls from all donors. In particular embodiments, for histopathology, the minimum threshold is no toxicity, and the maximum threshold is graded using adverse event criteria as published for each target organ.

### (XII) EXEMPLARY EMBODIMENTS

1. A method of genetically modifying a selected cell population in a biological sample that has undergone reduced or minimal manipulation including adding a nanoparticle (NP) disclosed herein to the biological sample.
2. The method of embodiment 1, wherein the NP is a gold NP (AuNP).
3. The method of embodiment 1 or 2, wherein the NP includes guide RNA (gRNA) wherein one end of the gRNA is conjugated to a linker, and the other end of the gRNA is conjugated to a nuclease, and wherein the linker allows covalent linkage of the gRNA to the surface of the NP.
4. The method of embodiment 3, wherein the gRNA includes a Clustered Regularly Interspaced Short Palindromic Repeat (CRISPR) guide RNA (crRNA).
5. The method of embodiment 4, wherein the 3′ end of the crRNA is conjugated to the linker.
6. The method of embodiment 4, wherein the 5′ end of the crRNA is conjugated to the linker.
7. The method of embodiments 4 or 5, wherein the 5′ end of the crRNA is conjugated to the nuclease.
8. The method of embodiment 4 or 6, wherein the 3′ end of the crRNA is conjugated to the nuclease.
9. The method of any of embodiments 3-8, wherein the linker includes a spacer with a thiol modification.
10. The method of embodiment 9, wherein the spacer is an oligoethylene glycol spacer.
11. The method of embodiment 10, wherein the oligoethylene glycol spacer is a 10⁻²⁶ atom oligoethylene glycol spacer.
12. The method of embodiment 10 or 11, wherein the oligoethylene glycol spacer is an 18 atom oligoethylene glycol spacer.
13. The method of any of embodiments 3-12, wherein the crRNA includes a sequence set forth in SEQ ID NO: 5; SEQ ID NO: 6; SEQ ID NO: 13; SEQ ID NO: 14; or SEQ ID NO: 225-264.
14. The method of any of embodiments 3-13, wherein the NP further includes a donor template farther from the surface of the NP than the gRNA and the nuclease.
15. The method of embodiment 14, wherein the donor template includes a therapeutic gene.
16. The method of embodiment 15, wherein the therapeutic gene includes or encodes skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C90RF72, a2R1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, FancI, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT−1, YES, zac1, iduronidase, IDS, GNS, HGSNAT, SGSH, NAGLU, GUSB, GALNS, GLB1, ARSB, HYAL1, F8, F9, HBB, CYB5R3, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.
17. The method of any of embodiments 14-16, wherein the donor template includes a homology-directed repair template (HDT) including sequences having homology to genomic sequences undergoing modification.
18. The method of embodiment 18, wherein the HDT comprises a sequence set forth in SEQ ID NO: 2; SEQ ID NO: 4; SEQ ID NO: 8; SEQ ID NO: 15; SEQ ID NO: 33-41; or SEQ ID NO: 44-52.
19. The method of any of embodiments 14-18, wherein the donor template includes single-stranded DNA (ssDNA).
20. The method of any of embodiments 1-19, wherein the NP is a AuNP associated with at least three layers, wherein the first layer includes single-stranded DNA (ssDNA), the second layer includes a Clustered Regularly Interspaced Short Palindromic Repeat (CRISPR) guide RNA (crRNA), and the third layer includes a nuclease, and wherein the first layer is the closest layer to the surface of the AuNP core, the second layer is the second closest layer to the surface of the AuNP core, and the third layer is the third closest layer to the surface of the AuNP core.
21. The method of embodiment 20, wherein the first layer further includes polyethylene glycol (PEG).
22. The method of any of embodiments 1-21, wherein the adding is in an amount of 1, 2, 3, 4, 5, 8, 10, 12, 15, or 20 μg of NP per milliliter (mL) of biological sample.
23. The method of any of embodiments 1-22, wherein the biological sample and the added NP are incubated for 1-48 hours.
24. The method of any of embodiments 1-22, wherein the biological sample and the added NP are incubated until testing confirms the uptake of the NP into cells.
25. The method of embodiment 24, wherein the testing includes confocal microscopy imaging or inductively coupled plasma (ICP) techniques.
26. The method of embodiment 24 or 25, wherein the testing includes ICP-mass spectrometry (ICP-MS), ICP-atomic emission spectroscopy (ICP-AES) or ICP-optical emission spectroscopy (ICP-OES).
27. The method of any of embodiments 1-26, wherein the NP is associated with a positively-charged polymer (e.g, polyethyleneimine (PEI)) coating.
28. The method of embodiment 27, wherein the positively-charged polymer coating creates a surface of the NP, wherein the surface optionally includes donor template.
29. The method of any of embodiments 1-28, wherein the NP includes a targeting ligand.
30. The method of embodiment 29, wherein the targeting ligand includes an antibody or antigen binding fragment thereof, an aptamer, a protein, and/or a binding domain.
31. The method of embodiment 29 or 30, wherein the targeting ligand extends beyond the surface of the NP.
32. The method of any of embodiments 29-31, wherein the targeting ligand is a binding molecule that binds CD3, CD4, CD34, CD46, CD90, CD133, CD164, a luteinizing hormone-releasing hormone (LHRH) receptor, or an aryl hydrocarbon receptor (AHR) (as examples, antibody clone: 581; antibody clone: 561; antibody clone: REA1164; antibody clone: AC136; antibody clone: 5E10; antibody clone: DG3; antibody clone: REA897; antibody clone: REA820; antibody clone: REA753; antibody clone: REA816; antibody clone: 293C3; antibody clone: AC141; antibody clone: AC133; antibody clone: 7; aptamer A15; aptamer B19; HCG (Protein/Ligand); Luteinizing hormone (LH Protein/Ligand); or a binding fragment derived from any of the foregoing).
33. The method of any of embodiments 29-32, wherein the targeting ligand is an anti-human CD3 antibody or antigen binding fragment thereof, an anti-human CD4 antibody or antigen binding fragment thereof, an anti-human CD34 antibody or antigen binding fragment thereof, an anti-human CD46 antibody or antigen binding fragment thereof, an anti-human CD90 antibody or antigen binding fragment thereof, an anti-human CD133 antibody or antigen binding fragment thereof, an anti-human CD164 antibody or antigen binding fragment thereof, an anti-human CD133 aptamer, a human luteinizing hormone, a human chorionic gonadotropin, degerelix acetate, or StemRegenin 1.
34. The method of any of embodiments 29-33, wherein the nuclease and targeting ligand are linked.
35. The method of embodiment 34, wherein the nuclease and targeting ligand are linked through an amino acid linker (e.g., a direct amino acid linker, a flexible amino acid linker, or a tag-based amino acid linker (e.g., Myc Tag or Strep Tag)).
36. The method of embodiments 34 or 35, wherein the nuclease and targeting ligand are linked through polyethylene glycol.
37. The method of any of embodiments 34-36, wherein the nuclease and targeting ligand are linked through an amine-to-sulfhydryl crosslinker.
38. The method of any of embodiments 3-37, wherein the nuclease is selected from Cpf1, Cas9, or Mega-TAL.
39. The method of any of embodiments 3-38, wherein the nuclease is Cpf1.
40. The method of any of embodiments 34-39, wherein the targeting ligand linked to the nuclease is farther from the surface of the NP than ssDNA associated with the NP.
41. The method of any one of embodiments 1-40, wherein the NP is associated with crRNA targeting a site described herein.
42. The method of any of embodiments 1-41, wherein the method targets a genomic site including a sequence selected from a sequence including SEQ ID NO: 1; SEQ ID NO: 3; SEQ ID NO: 20-32; SEQ ID NO: 42; SEQ ID NO: 43; SEQ ID NO: 84-97; or SEQ ID NO: 214-224.
43. The method of any of embodiments 1-42, wherein the method includes targeting a genomic site for genetic modification with a sequence selected from SEQ ID NO: 5; SEQ ID NO: 6; SEQ ID NO: 13; SEQ ID NO: 14; or SEQ ID NO: 225-264.
44. The method of any of embodiments 1-43, wherein the selected cell population includes a blood cell selected from a hematopoietic stem cell (HSC), a hematopoietic progenitor cell (HPC), a hematopoietic stem and progenitor cell (HSPC), a T cell, a natural killer (NK) cell, a B cell, a macrophage, a monocyte, a mesenchymal stem cell (MSC), a white blood cell (WBC), a mononuclear cell (MNC), an endothelial cell (EC), a stromal cell, and/or a bone marrow fibroblast.
45. The method of embodiment 44, wherein the blood cell includes a CD34⁺CD45RA⁻CD90⁺ HSC.
46. The method of embodiment 44 or 45, wherein the blood cell includes a CD34⁺/CD133⁺ HSC.
47. The method of any of embodiments 44-46, wherein the blood cell includes an LH⁺ HSC.
48. The method of any of embodiments 44-47, wherein the blood cell includes a CD34⁺CD90⁺ HSPC.
49. The method of any of embodiments 44-48, wherein the blood cell includes a CD34⁺CD90⁺CD133⁺ HSPC.
50. The method of any of embodiments 44-49, wherein the blood cell includes an AHR⁺ HSPC.
51. The method of any of embodiments 44-50, wherein the blood cell includes a CD3⁺ T cell.
52. The method of any of embodiments 44-51, wherein the blood cell includes a CD4⁺ T cell.
53. The method of any of embodiments 44-52, wherein the blood cell is a human blood cell.
54. The method of any of embodiments 1-53, wherein the biological sample includes peripheral blood and/or bone marrow.
55. The method of any of embodiments 1-54, wherein the biological sample includes granulocyte colony stimulating factor (GCSF) mobilized peripheral blood, and/or plerixa for mobilized peripheral blood.
56. The method of any of embodiments 1-55, wherein the method yields a mean total gene editing rate of 5% to 50%.
57. The method of any of embodiments 1-56, wherein the method yields greater than 60% cell viability in the selected cell population.
58. A cell modified according to a method of any one of embodiments 1-57.
59. A cell of embodiment 58, wherein the cell has not undergone electroporation.
60. A cell of embodiment 58 or 59, wherein the cell has not been exposed to a viral vector.
61. A cell of any of embodiments 58-60, wherein the cell has not been exposed to a viral vector encoding a donor template or an HDT.
62. A cell of any of embodiments 58-61, wherein the cell has not undergone a cell separation process intended to separate the cell from a biological sample.
63. A cell of any of embodiments 58-62, wherein the cell has not undergone a magnetic cell separation process.
64. A therapeutic formulation including a cell of any of embodiments 58-63.
65. A method of providing a therapeutic nucleic acid sequence to a subject in need thereof including administering a cell of any of embodiments 58-63 or a therapeutic formulation of embodiment 64 to the subject thereby providing a therapeutic nucleic acid sequence to the subject.
66. A nanoparticle (NP) including
a core that is less than 30 nm in diameter;
a guide RNA-nuclease ribonucleoprotein (RNP) complex wherein the gRNA includes a 3′ end and a 5′ end, wherein the 3′ end is conjugated to a spacer with a chemical modification, and the 5′ end is conjugated to the nuclease, and wherein the chemical modification is covalently linked to the surface of the core;
a positively-charged polymer coating wherein the positively-charged polymer has a molecular weight of less than 2500 daltons, surrounds the RNP complex, and contacts the surface of the core; and
a donor template (e.g., optionally including a homology-directed repair template (HDT)) on the surface of the positively-charged polymer coating.
67. The NP of embodiment 66, wherein the core includes gold (Au).
68. The NP of embodiment 66 or 67, wherein the weight/weight (w/w) ratio of core to nuclease is 0.6.
69. The NP of any of embodiments 66-68, wherein the w/w ratio of core to HDT is 1.0.
70. The NP of any of embodiments 66-69, wherein the NP is less than 70 nm in diameter.
71. The NP of any of embodiments 66-70, wherein the NP has a polydispersity index (PDI) of less than 0.2.
72. The NP of any of embodiments 66-71, wherein the gRNA includes a Clustered Regularly Interspaced Short Palindromic Repeat (CRISPR) crRNA.
73. The NP of embodiment 72, wherein the crRNA includes a sequence as set forth in SEQ ID NO: 5; SEQ ID NO: 6; SEQ ID NO: 13; SEQ ID NO: 14; or SEQ ID NO: 225-264.
74. The NP of any of embodiments 66-73, wherein the nuclease includes Cpf1 or Cas9.
75. The NP of any of embodiments 66-74, wherein the positively-charged polymer coating includes polyethyleneimine (PEI), polyamidoamine (PAMAM); polylysine (PLL), polyarginine; cellulose, dextran, spermine, spermidine, or poly(vinylbenzyl trialkyl ammonium).
76. The NP of any of embodiments 66-75, wherein the positively-charged polymer has a molecular weight of 1500-2500 daltons.
77. The NP of any of embodiments 66-76, wherein the positively-charged polymer has a molecular weight of 2000 daltons.
78. The NP of any of embodiments 66-77, wherein the chemical modification includes a free thiol, amine, or carboxylate functional group.
79. The NP of any of embodiments 66-78, wherein the spacer includes an oligoethylene glycol spacer.
80. The NP of embodiment 79, wherein the oligoethylene glycol spacer includes an 18 atom oligoethylene glycol spacer.
81. The NP of any of embodiments 66-80, wherein the HDT includes sequences having homology to genomic sequences undergoing modification.
82. The NP of embodiment 81, wherein the HDT includes a sequence as set forth in SEQ ID NO: 2; SEQ ID NO: 4; SEQ ID NO: 8; SEQ ID NO: 15; SEQ ID NO: 33-41; or SEQ ID NO: 44-52.
83. The NP of any of embodiments 66-82, wherein the HDT includes single-stranded DNA (ssDNA).
84. The NP of any of embodiments 66-83, wherein the donor template includes a therapeutic gene.
85. The NP of embodiment 84, wherein the therapeutic gene encodes skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72, α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, FancI, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT−1, YES, zac1, iduronidase, IDS, GNS, HGSNAT, SGSH, NAGLU, GUSB, GALNS, GLB1, ARSB, HYAL1, F8, F9, HBB, CYB5R3, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.
86. The NP of any of embodiments 66-85, wherein the NP further includes a targeting ligand linked to the nuclease.
87. The NP of embodiment 86, wherein the targeting ligand includes a binding molecule that binds CD3, CD4, CD34, CD46, CD90, CD133, CD164, a luteinizing hormone-releasing hormone (LHRH) receptor, or an aryl hydrocarbon receptor (AHR).
88. The NP of embodiments 86 or 87, wherein the targeting ligand includes an anti-human CD3 antibody or antigen binding fragment thereof, an anti-human CD4 antibody or antigen binding fragment thereof, an anti-human CD34 antibody or antigen binding fragment thereof, an anti-human CD46 antibody or antigen binding fragment thereof, an anti-human CD90 antibody or antigen binding fragment thereof, an anti-human CD133 antibody or antigen binding fragment thereof, an anti-human CD164 antibody or antigen binding fragment thereof, an anti-human CD133 aptamer, a human luteinizing hormone, a human chorionic gonadotropin, degerelix acetate, or StemRegenin 1.
89. The NP of any of embodiments 86-88, wherein the targeting ligand includes antibody clone: 581; antibody clone: 561; antibody clone: REA1164; antibody clone: AC136; antibody clone: 5E10; antibody clone: DG3; antibody clone: REA897; antibody clone: REA820; antibody clone: REA753; antibody clone: REA816; antibody clone: 293C3; antibody clone: AC141; antibody clone: AC133; antibody clone: 7; aptamer A15; aptamer B19; HCG (Protein/Ligand); Luteinizing hormone (LH Protein/Ligand); or a binding fragment derived from any of the foregoing.
90. The NP of any of embodiments 86-89, wherein the nuclease and targeting ligand are linked.
91. The NP of embodiments 90, wherein the nuclease and targeting ligand are linked through an amino acid linker (e.g., a direct amino acid linker, a flexible amino acid linker, and/or a tag-based amino acid linker).
92. The NP of any of embodiments 86-91, wherein the nuclease and targeting ligand are linked through polyethylene glycol (PEG).
93. The NP of any of embodiments 86-92, wherein the nuclease and targeting ligand are linked through an amine-to-sulfhydryl crosslinker.
94. A composition including a NP of claim 66-93 and a biological sample.
95. The composition of embodiment 94, wherein the biological sample includes a selected cell population.
96. The composition of embodiment 95, wherein the selected cell population includes a blood cell selected from a hematopoietic stem cell (HSC), a hematopoietic progenitor cell (HPC), a hematopoietic stem and progenitor cell (HSPC), a T cell, a natural killer (NK) cell, a B cell, a macrophage, a monocyte, a mesenchymal stem cell (MSC), a white blood cell (WBC), a mononuclear cell (MNC), an endothelial cell (EC), a stromal cell, and/or a bone marrow fibroblast.
97. The composition of embodiment 95, wherein the blood cell includes a CD34⁺CD45RA⁻CD90⁺ HSC; a CD34⁺/CD133⁺ HSC; an LH⁺ HSC; a CD34⁺CD90⁺ HSPC; a CD34⁺CD90⁺CD133⁺ HSPC; and/or an AHR⁺ HSPC.
98. The composition of embodiment 95, wherein the blood cell includes a CD3⁺ T cell and/or a CD4⁺ T cell.
99. The composition of any of embodiments 94-98, wherein the biological sample includes peripheral blood, bone marrow, granulocyte colony stimulating factor (GCSF) mobilized peripheral blood, and/or plerixa for mobilized peripheral blood.
100. The composition of any of embodiments 94-99, wherein NP is within the biological sample in an amount of 1, 2, 3, 4, 5, 8, 10, 12, 15, or 20 μg of NP per milliliter (mL) of biological sample.
101. A kit including one or more components described in any of the preceding embodiments.

### (XIII) EXPERIMENTAL EXAMPLES

### Example 1. Synthesizing Gold Nanoparticle Cores

Gold nanoparticles (AuNPs) of 15 nm size range were synthesized by Turkevich's method with slight modification. Turkevich, et al., (1951). Discussions of the Faraday Society 11(0): 55-75.). 0.25 mM Chloroauric acid solution was brought to the boiling point and reduced by adding 3.33% sodium citrate solution and stirred vigorously under reflux system for 10 min. Synthesized NP were washed three times and re-dispersed in highly pure water.

Cpf1 and Cas9 Guide RNA Structures. Single Cpf1 guide RNA was ordered from commercial source, Integrated DNA Technologies; IDT), with two custom modifications on the 3′ end. The first modification included an 18-atom oligo ethylene glycol (OEG) spacer (iSp18), and the second modification included a thiol modification. The OEG spacer (e.g. polyethylene glycol (PEG) or hexaethylene glycol (HEG), etc.), was at a ratio of 1 per oligonucleotide and served to prevent electrostatic repulsion between oligonucleotides. While an 18-atom spacer was used, other lengths are also appropriate. The thiol modification was also added at a ratio of 1 per oligonucleotide and served as the basis for covalent interactions to bind the oligonucleotide to the surface of the AuNP.

5′-/AltR1/rUrA rArUrU rUrCrU rArCrU rCrUrU rGrUrA rGrArU rCrArC rCrCrG rArUrC rCrArC rUrGrG rGrGrA rGrCrA/iSp18//3ThioMC3-D/-3′ (SEQ ID NO: 5)
For cas9, a two-part guide system including tracrRNA and crRNA was used. crRNA for Cas9 was ordered from IDT with the same 18 spacer-thiol modifications as above, but on the 5′ end.
5′-/5ThioMC6-D//iSp18/rCrA rCrCrC rGrArU rCrCrA rCrUrG rGrGrG rArGrC rGrUrU rUrUrA rGrArG rCrUrA rUrGrC rU/AltR2/-3′ (SEQ ID NO: 6)
The accompanying tracrRNA was unmodified. In these sequences, “r” stands for RNA and spaces are provided for ease of reading.

Preparing the Au/CRISPR NP. crRNAs with 18 spacer-thiol modifications were used. AuNPs in 10 μg/mL concentration was added to crRNA solution in AuNP/crRNA w/w ratio of 0.5. Following that, citrate buffer with the pH of 3 was added in 10 mM concentration and mixed for 5 min. Prepared AuNP/crRNA nanoconjugates were centrifuged down and re-dispersed in PBS. Then, Cpf1 nuclease was added in AuNP/Cpf1 w/w ratio of 0.6. Polyethylenimine (PEI) of 2000 MW was added in 0.005% concentration and mixed thoroughly. In the final step, ssDNA template was added in the AuNP/ssDNA w/w ratio of 1.

### Example 2. Targeted Homology Directed Repair in Blood Stem and Progenitor Cells with Highly Potent Gene-Editing Nanoparticles

Abstract. Ex vivo CRISPR gene editing in hematopoietic stem and progenitor cells has corrected genetic diseases, protected from infectious diseases and provided new treatments for cancer. While the current process for gene editing with homologous recombination, electroporation followed by non-integrating virus transduction, has resulted in high levels of gene editing at some genetic loci, this complex manipulation has resulted in cellular toxicity and compromised fitness of transplanted blood cells. Here, a highly potent gene-editing NP was developed using colloidal AuNP. To ensure delivery of all required machinery upon uptake of a single NP, a loading design was developed which is capable of passive cellular entry without the need for electroporation or viruses. This small, highly monodisperse NP avoided lysosomal entrapment, and successfully localized to the nucleus in primary human hematopoietic stem and progenitor cells without observable toxicity. NP-mediated gene editing was efficient and sustained with different gene-editing nucleases at multiple loci of therapeutic interest. Engraftment kinetics of NP-treated primary cells in humanized mice were better relative to non-treated cells, with no observable differences in differentiation in vivo. This is the first demonstration of efficient, passive delivery of an entire gene editing payload into primary human blood stem and progenitor cells.

Introduction. Retrovirus-mediated gene correction in hematopoietic stem and progenitor cells (HSPC) has demonstrated curative outcomes for various genetic, infectious and malignant disorders (Hacein-Bey-Abina et al., N Engl J Med, 371(15): 1407-1417 (2014); Cicalese et al., Blood, 128(1): 45-54 (2016); Sessa et al., Lancet, 388(10043): 476-487 (2016); Hacein-Bey et al., JAMA, 313(15): 1550-1563 (2015); and Dunbar et al., Science, 359(6372) (2018)). The use of gene-modified autologous, or “self”, HSPC eliminates the risk of graft-host immune responses, negating the need for immunosuppressive drugs required in allogeneic hematopoietic stem cell transplant. However, effective implementation of HSPC gene therapy faces several major challenges. Currently, limited quantities of therapeutic retrovirus vector can be produced at Good Manufacturing Practices (GMP) quality, creating a major bottleneck to widespread use of this technology. In addition to the challenges of manufacturing sufficient vector quantities, there is a known risk of genotoxicity associated with the use of retrovirus vectors for gene transfer evidenced by the development of malignancy due to insertional mutagenesis (Hacein-Bey-Abina et al., Science, 302(5644): 415-419 (2003); Hacein-Bey-Abina et al., N Engl J Med, 348(3): 255-256 (2003); Ott et al., Nat Med, 12(4): 401-409 (2006); and Stein et al., Nat Med, 16(2): 198-204 (2010)). All of these challenges have inspired the development of non-viral means for genetic modification.

Most prominently, gene editing has been proposed as a safer alternative to retrovirus-mediated gene transfer, made possible by the development of engineered nucleases such as clustered regularly interspaced short palindromic repeat (CRISPR)-Cas nucleases (Cornu et al., Nat Med, 23(4): 415-423 (2017)). These programmable nucleases incorporate one or more RNA molecules to target specific sequences in the DNA for cutting by the nuclease protein component. Of these, Cas9 nuclease is the most well studied. This nuclease complexes with two RNA molecules, a guide RNA (crRNA) and a tracer RNA (tracrRNA), to recognize a cognate protospacer adjacent motif (PAM) site consisting of an NGG sequence and then makes a blunt-end double strand break in the DNA. This break can be repaired by several cellular mechanisms, but the two most common are non-homologous end joining (NHEJ) and homology-directed repair (HDR) (Chang et al., Nature reviews Molecular cell biology, 18(8): 495-506 (2017)). For the latter to occur, an intact template sequence homologous to the cut site must be present. The sister chromatid can serve as a template, but synthetic template molecules can also be provided in surplus to enhance HDR efficiency. While the flanking regions of this template must significantly or completely match the flanking regions of the cut site, new genetic code can be inserted within, permitting precise editing of or addition of new DNA to the genome when HDR occurs, whereas with NHEJ, insertions and/or deletions (indels) are the most likely outcome (Chang et al., Nature reviews Molecular cell biology, 18(8): 495-506 (2017)). Recently, Cpf1 (or Cas12a), has also demonstrated utility in genome editing. This nuclease differs from Cas9 in that it recognizes a different protospacer adjacent motif (PAM) site (e.g. TTTN, where N can be either A, C, G or T), requires a single guide RNA and results in staggered cutting of the DNA with 5′ overhangs (Zetsche et al., Cell, 163(3): 759-771 (2015)). The smaller size and staggered cutting of Cpf1 are postulated to enhance the ease of delivery and likelihood of HDR when template oligonucleotides are provided.

For the most utility in HSPC gene therapy, a delivery platform including the designer nuclease of choice, with or without a DNA template, which performs efficiently and reliably without cytotoxicity would be ideal. The current clinical state of the art for this approach in HSPC requires electroporation of engineered nuclease components as mRNA or ribonucleoprotein (RNP) complexes. If HDR is preferred, the most effective method has been electroporation followed by transduction with non-integrating virus vectors (Dever et al., Nature, 539(7629): 384-389 (2016)), or simultaneous electroporation of defined concentrations of engineered nuclease components with chemically modified, single-stranded oligonucleotide (ssODN) template at specified cell concentrations (De Ravin et al., Sci Transl Med, 9(372) (2017)). Electroporation is known to induce toxicity and moreover, there is no means to control the number of cells which take up each component of the payload or the concentrations of each component that are successfully delivered by electroporation (Lefesvre et al., BMC molecular biology, 3: 12-12 (2002)). Finally, where non-integrating viruses are used as templates, the systems still depend on GMP-grade viral particles to be available. Thus, NP-based delivery is being actively pursued for the delivery of gene-editing components (Li et al., Human gene therapy, 26(7): 452-462 (2015)).

In this regard, lipid-based, polymer-based and AuNP carry great potential for the delivery of gene-editing components to cells (Finn et al., Cell Reports, 22(9): 2227-2235 (2018); Lee et al., Nature Biomedical Engineering, 1(11): 889-901 (2017); and Lee et al., Nature Biomedical Engineering, 2(7): 497-507 (2018)). While polymer and lipid nanoparticles represent “encapsulating” or “entrapping” delivery vehicles, the unique surface loading of AuNP facilitates precise modification and functionalization by different molecules, such as RNA, DNA and proteins (Rosi et al., Science, 312(5776): 1027-1030 (2006)). Because the surface area is known, controlled loading of payload components ensures uniformity of AuNP preparations, leading to more predictable delivery (Ding et al., Molecular Therapy, 22(6): 1075-1083 (2014)). Finally, AuNP are considered relatively nontoxic compared to lipid and polymer nanocarriers (Pan et al., Small (Weinheim an der Bergstrasse, Germany), 3(11): 1941-1949 (2007); Alkilany et al., Journal of Nanoparticle Research, 12(7): 2313-2333 (2010); and Lewinski et al., Small (Weinheim an der Bergstrasse, Germany), 4(1): 26-49 (2008)), which is critical for nonmalignant dividing somatic cells such as HSPC. Indeed, Lee et al. have demonstrated the utility of a polymer-encapsulated AuNP design in the delivery of CRISPR Cas9 and Cpf1 to non-dividing somatic tissues such as muscle and brain (Lee et al., Nature Biomedical Engineering, 1(11): 889-901 (2017) and Lee et al., Nature Biomedical Engineering, 2(7): 497-507 (2018)), but these carriers have not demonstrated efficacy in HSPC or with accompanying oligonucleotide templates. Moreover, the combination of polymer encapsulation with a Au nanocore greatly increases the overall NP size and alters the cytotoxicity profile of the NP.

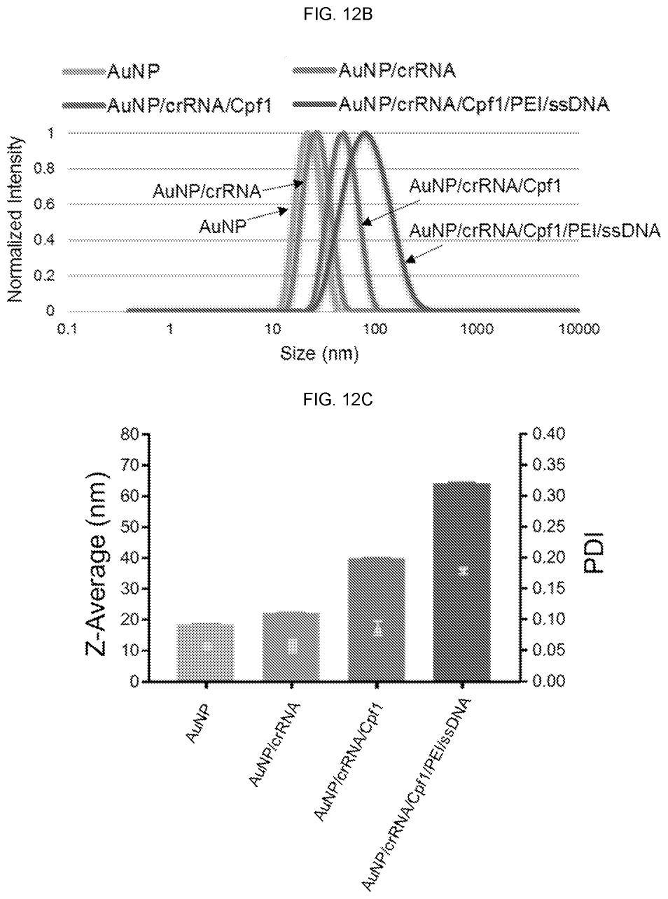

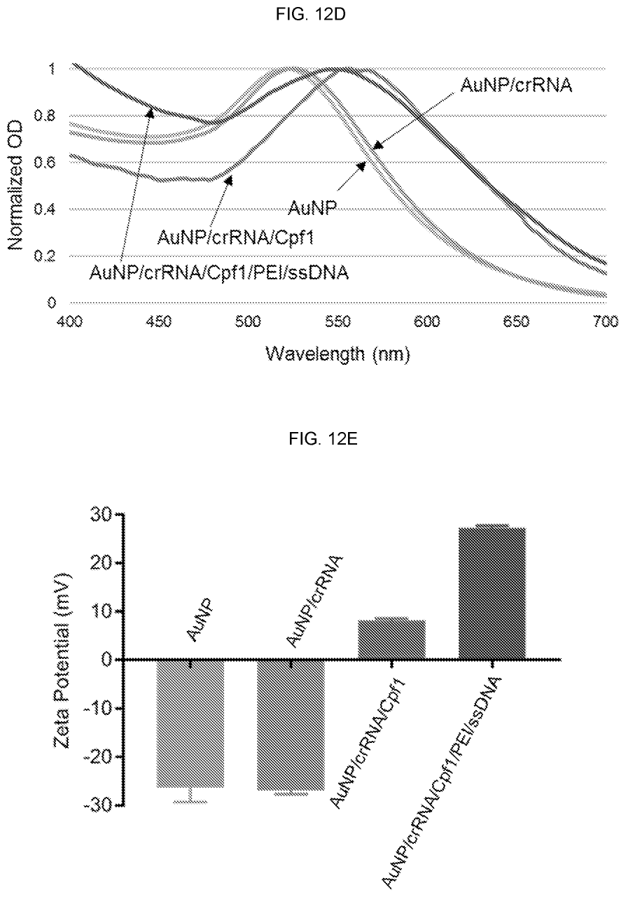

A simple Au-based gene-editing NP (e.g., Au/CRISPR NP) was designed with layer by layer conjugation of the gene-editing components (guide RNA and nuclease) on the surface of AuNP with or without a single stranded DNA template to support HDR (HDT), which does not require polymer encapsulation (FIGS. 5C and 12A).

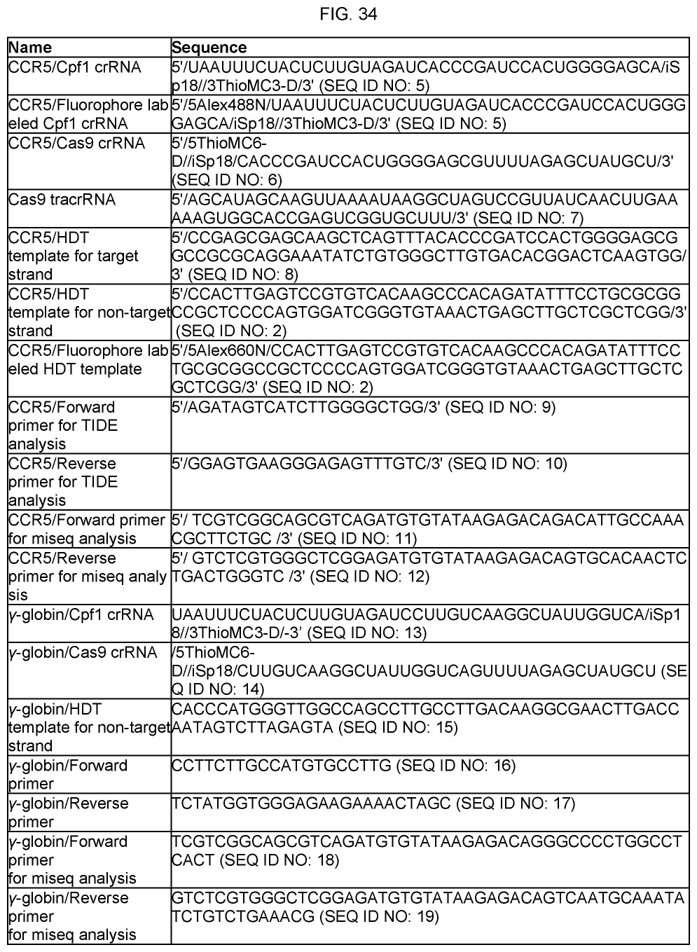

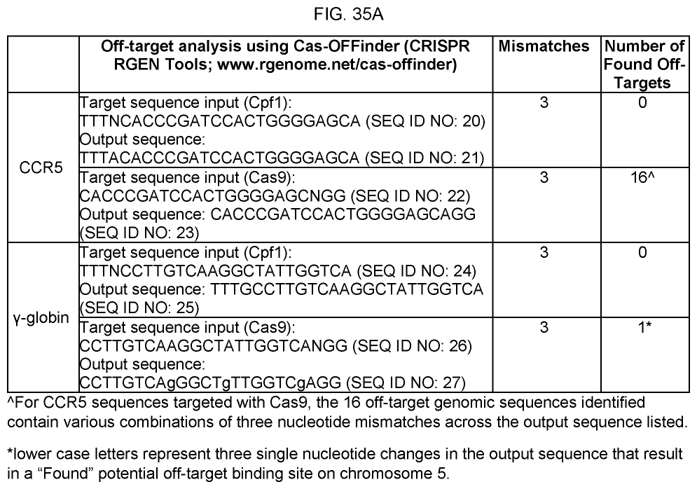

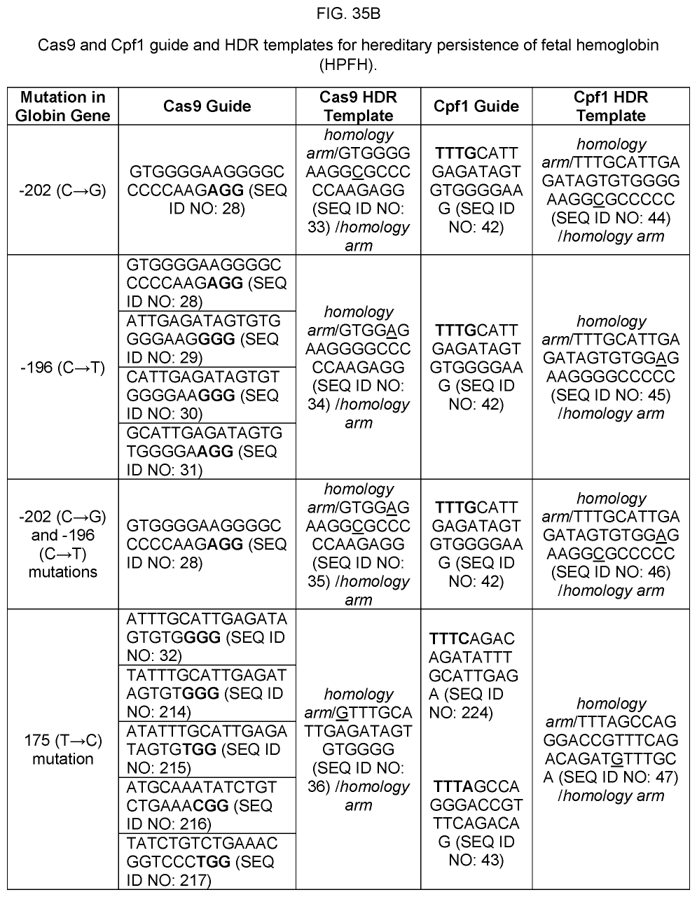

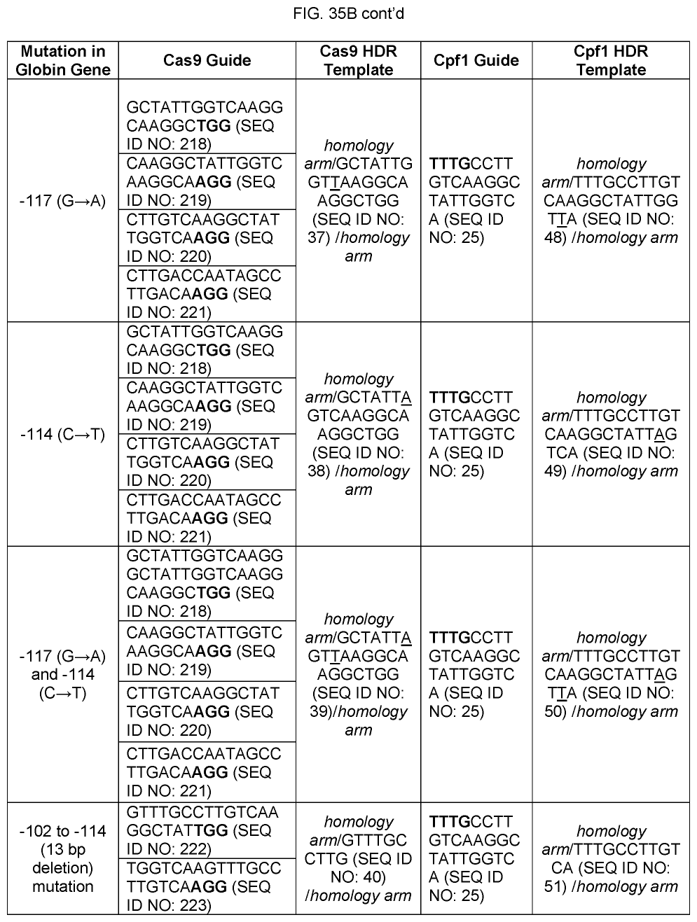

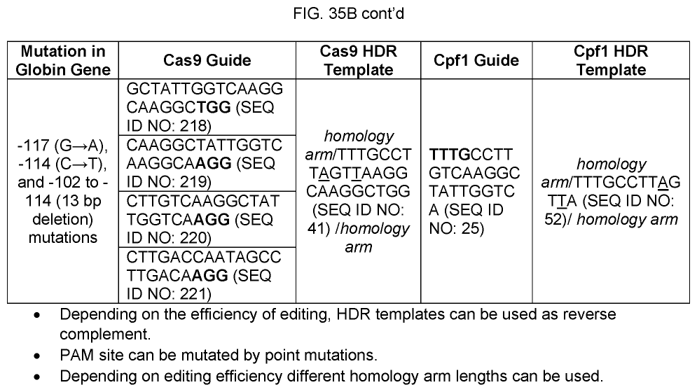

An AuNP core of 19 nm was synthesized using the citrate reduction method (Turkevich et al., Discussions of the Faraday Society, 11(0): 55-75 (1951)). Synthesized NP were highly monodisperse with an observed polydispersity index (PDI) of 0.05 (FIGS. 12B and 12C). The process for the preparation and the conjugation of the different layers can be found in FIG. 5C. In the first layer, CRISPR RNA (crRNA) for Cpf1 or Cas9 synthesized with an 18-nucleotide oligo ethylene glycol (OEG) spacer and a terminal thiol linker (crRNA-18 spacer-SH) was attached to the surface of Au by semi covalent Au-thiol interaction (sequence information can be found in FIG. 34). Analysis of the published crystal structures of these Cas nucleases with crRNA and/or tracrRNA and double-stranded DNA suggested that adding a spacer-thiol linker to the crRNA would not have any effect on the recognition of the guide segment and nuclease activity (Yamano T et al., Cell, 165(4): 949-962 (2016) and Lee et al., eLife, 6: e25312 (2017)). The inclusion of the OEG spacer arm reduced electrostatic repulsion between the strands of crRNA to increase the loading capacity on the surface of AuNP. As shown in FIG. 12B, the AuNP core with crRNA resulted in a NP size of 22 nm with a PDI of 0.05. Nuclease proteins were then attached to the 5′ handle of surface-loaded crRNA by the natural affinity of nuclease to the 3D structure of crRNA. Nuclease attachment increased the size of NP to 40 nm with PDI of 0.08 for Cpf1. This RNP-loaded AuNP served as a basis for comparison of nuclease activity without HDT present. For HDT loading, RNP-loaded AuNP were further coated with branched low molecular weight (2000) polyethylenimine (PEI) to prepare the base for electrostatic conjugation of HDT in the outermost layer. This “fully loaded” AuNP demonstrated a size of 64 nm and remained highly monodisperse with an observed PDI of 0.17 (FIGS. 12A-12C). Uniform morphology without any aggregation was inferred from transmission electron microscope images and looking at fine localized surface plasmon resonance (LSPR) shifts after each attachment step (FIGS. 12A, 12D). Zeta potential of the NP changed from −26 mV to +27 mV with complete layering (FIG. 12E). This positive charge of the final NP likely prevented precipitation and aggregation over time, as these were not observed over a period of 48 hours following formulation.

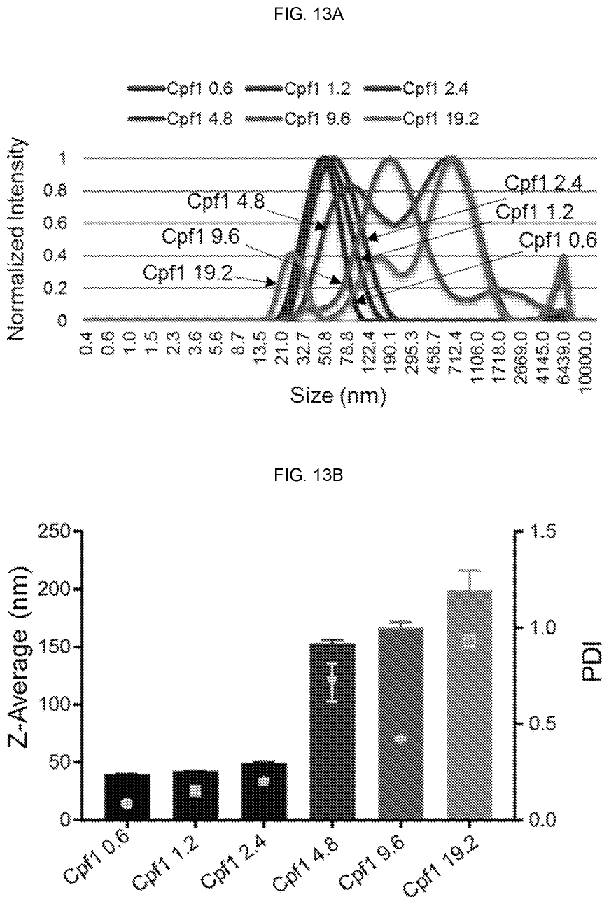

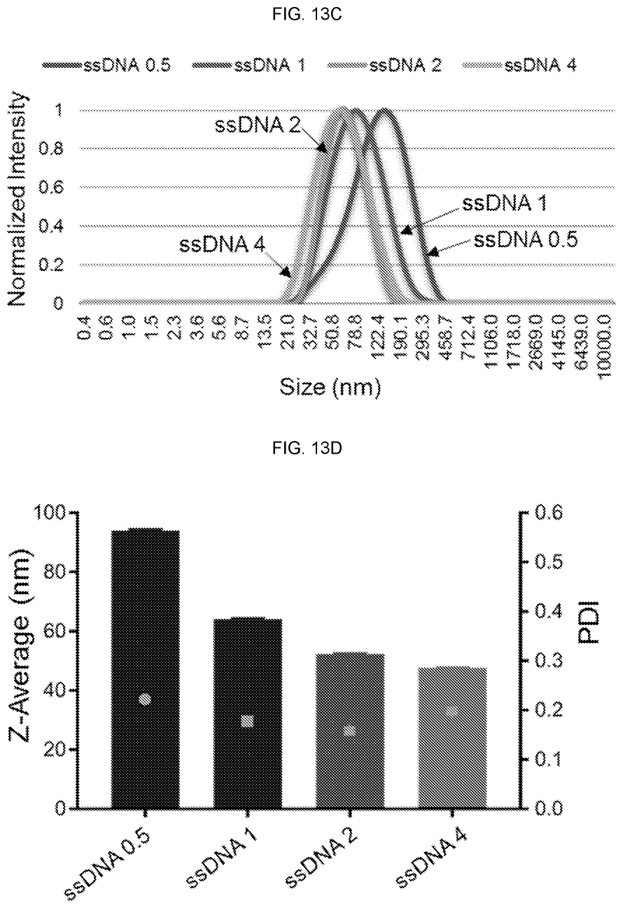

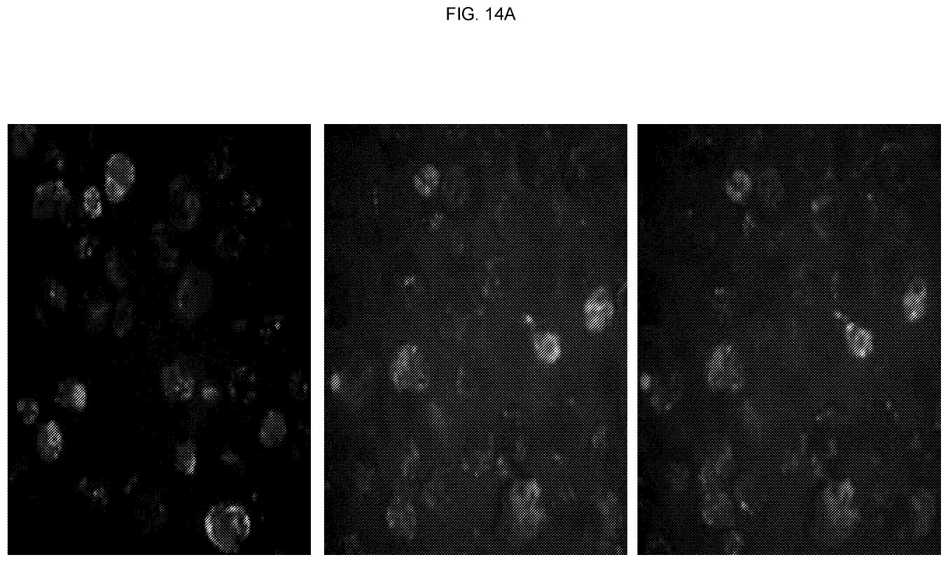

This highly stable and monodisperse structure is owed to the adjustment of weight/weight (w/w) ratios between AuNP and gene-editing components. Analysis of different w/w ratios between AuNP and Cpf1 demonstrated that lower ratios of Cpf1 can trigger aggregation with an optimal w/w ratio of 0.6 (FIGS. 13A, 13B). The loading capacity of Cpf1 was found to be 8.8 μg/mL in this ratio. In contrast to Cpf1, lower w/w ratio between AuNP and HDT lead to aggregation with an optimal w/w ratio of 1 (FIGS. 13C, 13D).

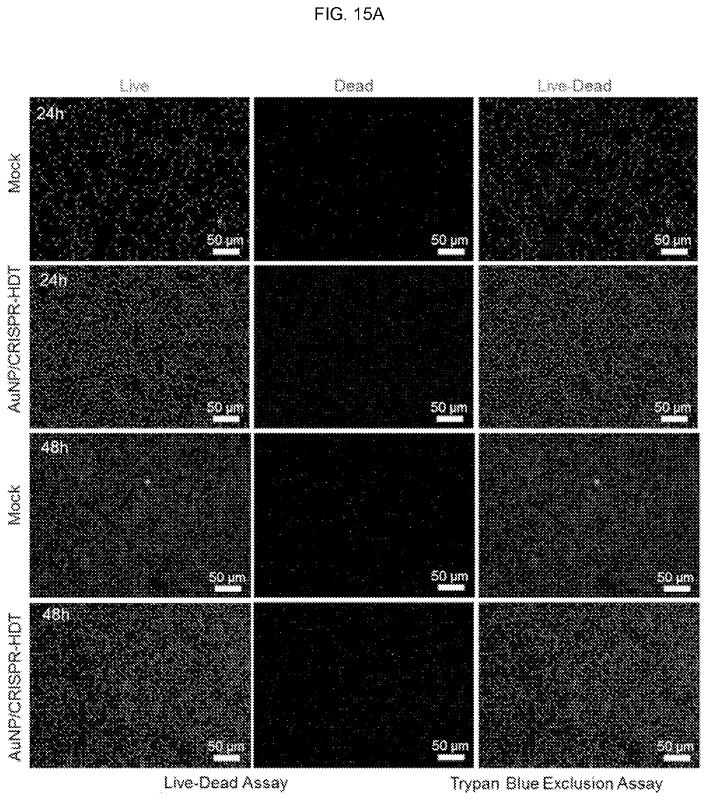

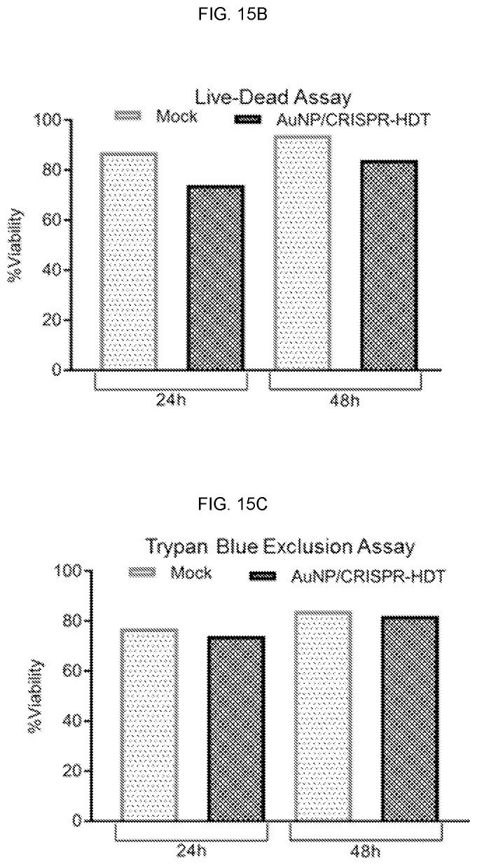

To determine the impact of this NP on primary HSPC, HSPC were isolated from leukapheresis products on the basis of CD34 expression from granulocyte colony stimulating factor (G-CSF) mobilized healthy adult volunteers. Cells were cultured in supportive media and AuNP formulations were added to culture at a concentration of 10 μg/mL. Potential toxicity in CD34⁺ cells was analyzed by both live-dead staining, and trypan blue dye exclusion assays after 24 h and 48 h incubations with Au/CRISPR NP (FIGS. 15A-15C). Au/CRISPR NP treated samples demonstrated more than 80% viability in both assays, with no variation between treated and untreated cells by trypan blue assay.

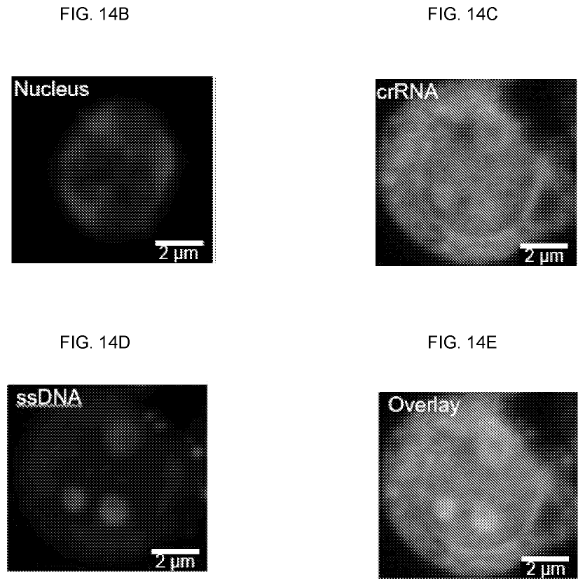

Although HSPCs are known to be very difficult to transfect, within 6 h after treatment with Au/CRISPR NP confocal microscopy imaging showed good uptake and localization of the gene editing components in the nucleus of primary HSPC (FIGS. 14A-14E). Here cellular biodistribution of both fluorescently labeled crRNA and HDT were tracked in z-series and in both cases clear nuclear localization was observed (FIG. 14E).

To test the utility of Au/CRISPR NP for gene editing, two different genomic loci were targeted with demonstrated therapeutic value in HSPC: (1) the chemokine receptor 5 (CCR5) gene on chromosome 3, and (2) the gamma globin (γ-globin) gene promoter on chromosome 11. Disruption of CCR5 has been associated with resistance to human immunodeficiency virus (HIV) infection by eliminating the attachment and entry of the virus through the expressed CCR5 co-receptor (Lopalco et al., Viruses, 2(2): 574-600 (2010)). Targeting this disruption in HSPC renders future T cell progeny resistant to HIV infection. Alternatively, introduction of a specific deletion within the γ-globin promoter recapitulates a naturally-occurring phenomenon known as hereditary persistence of fetal hemoglobin (HPFH), which has been shown to be useful for the treatment of hemoglobinopathies such as sickle cell disease and β-thalassemia (Akinsheye et al., Blood, 118(1): 19 (2011)).

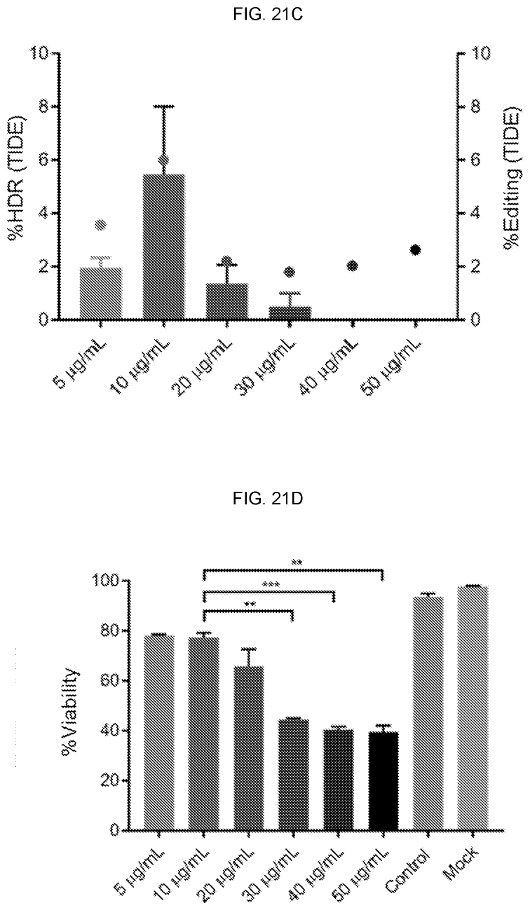

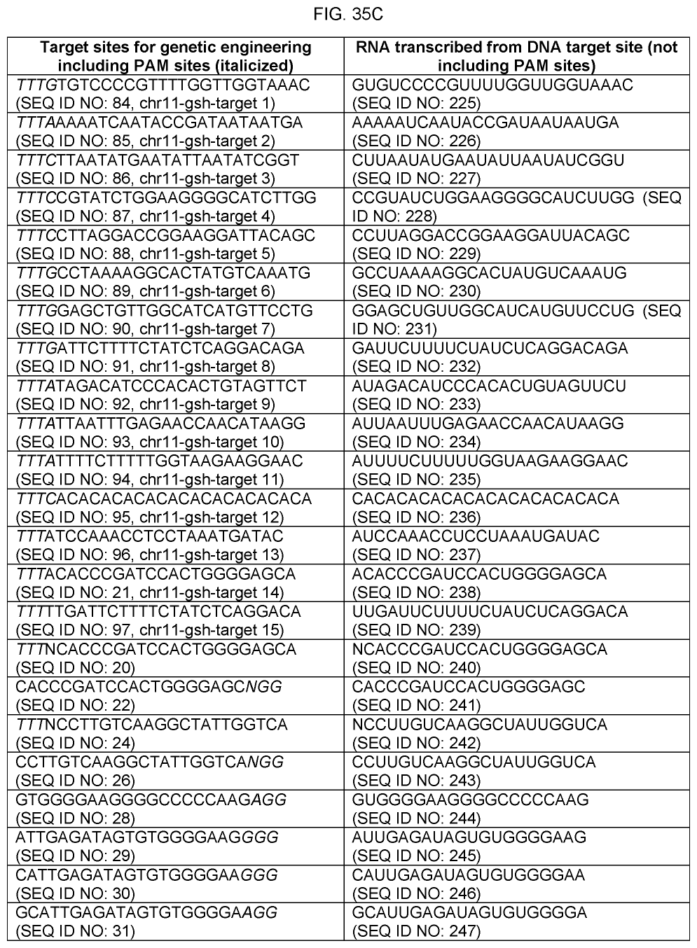

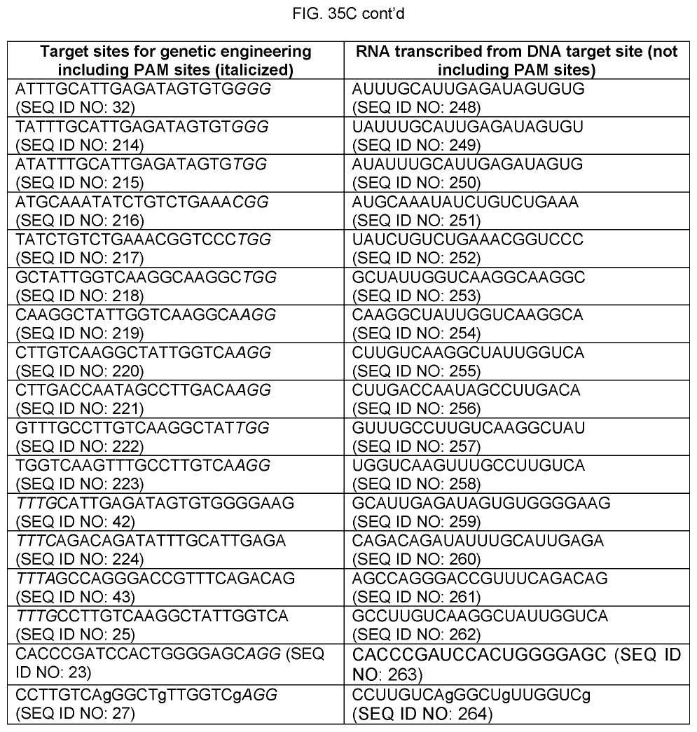

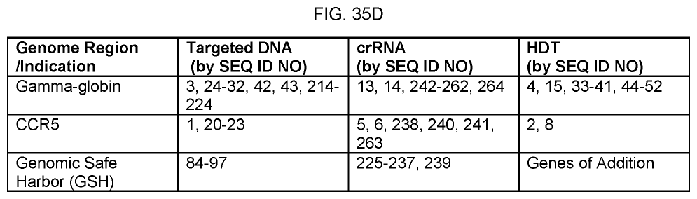

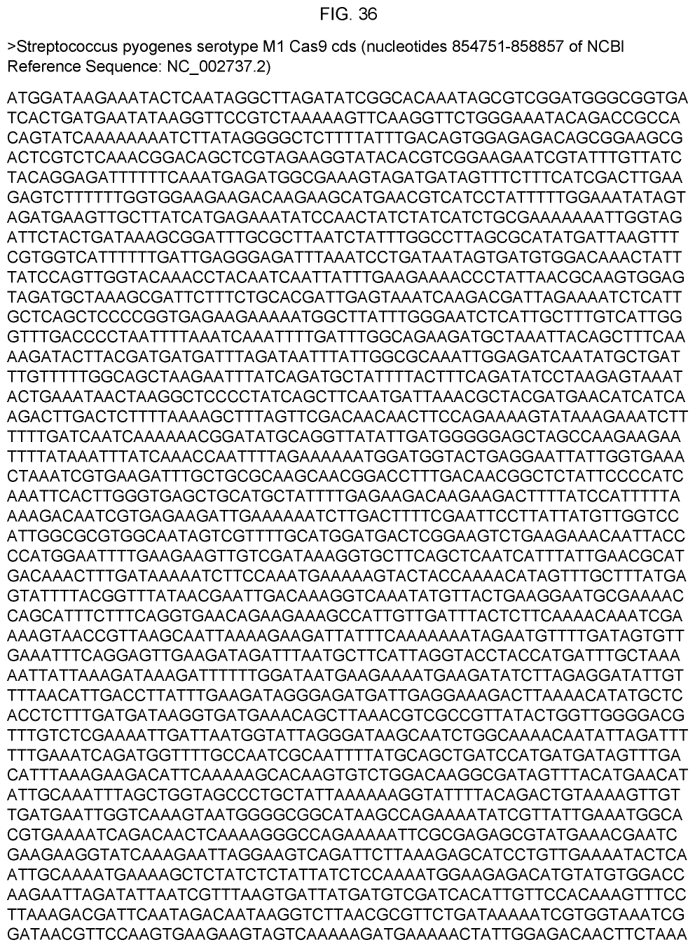

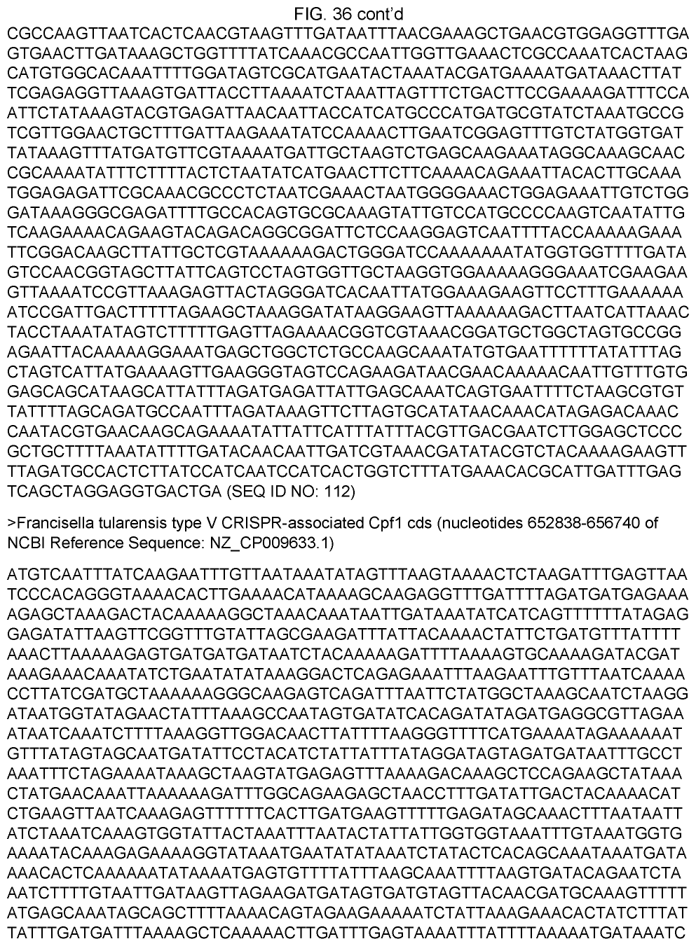

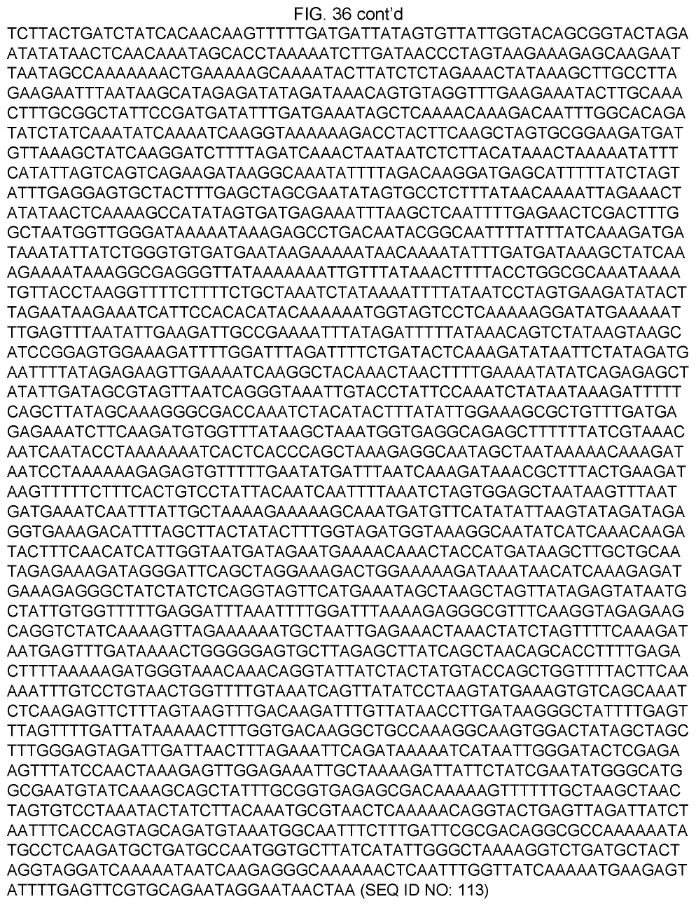

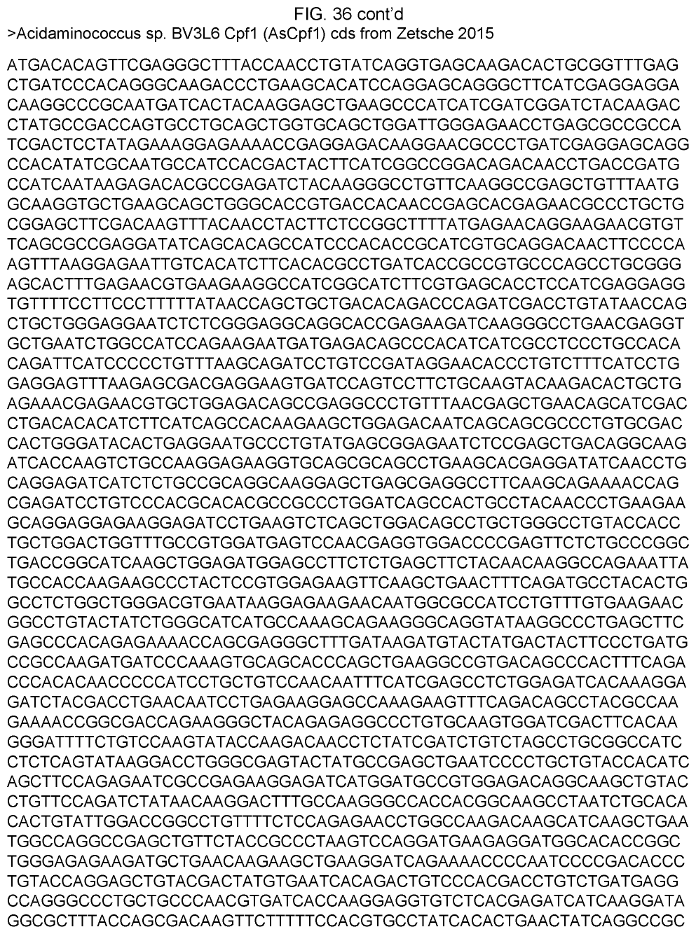

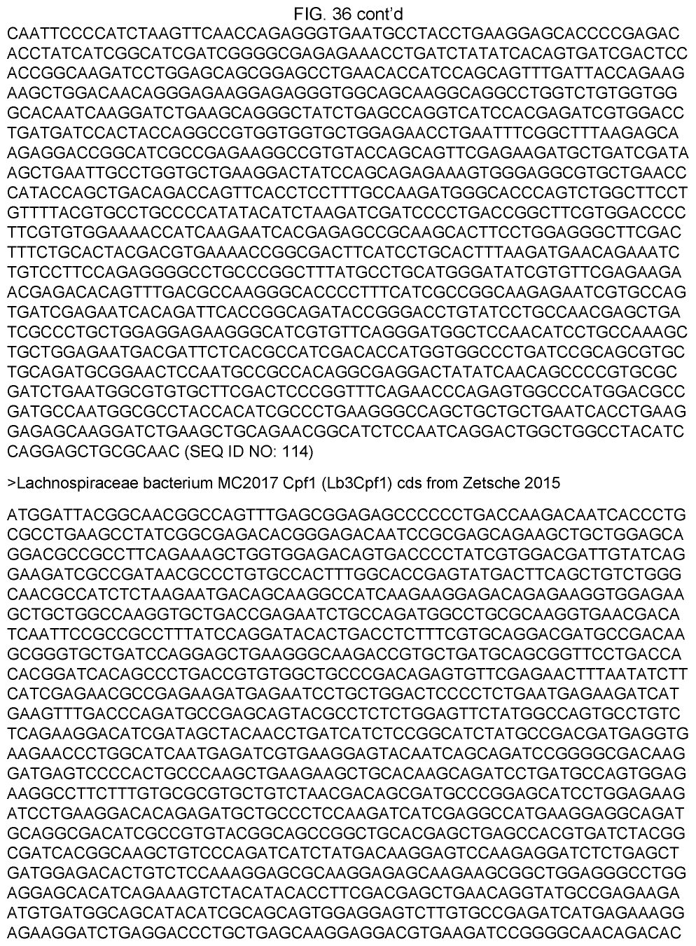

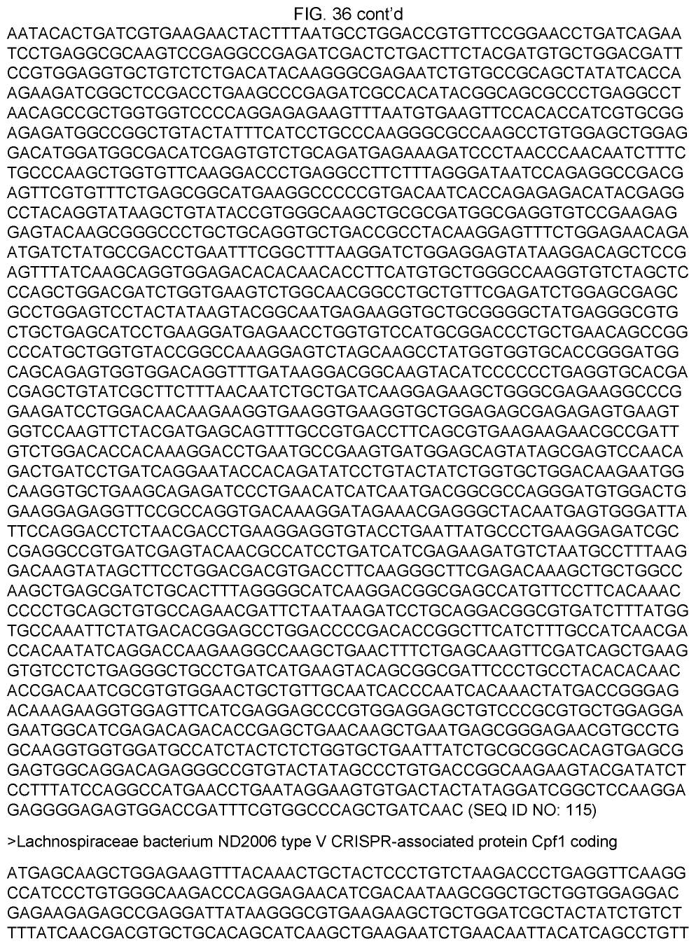

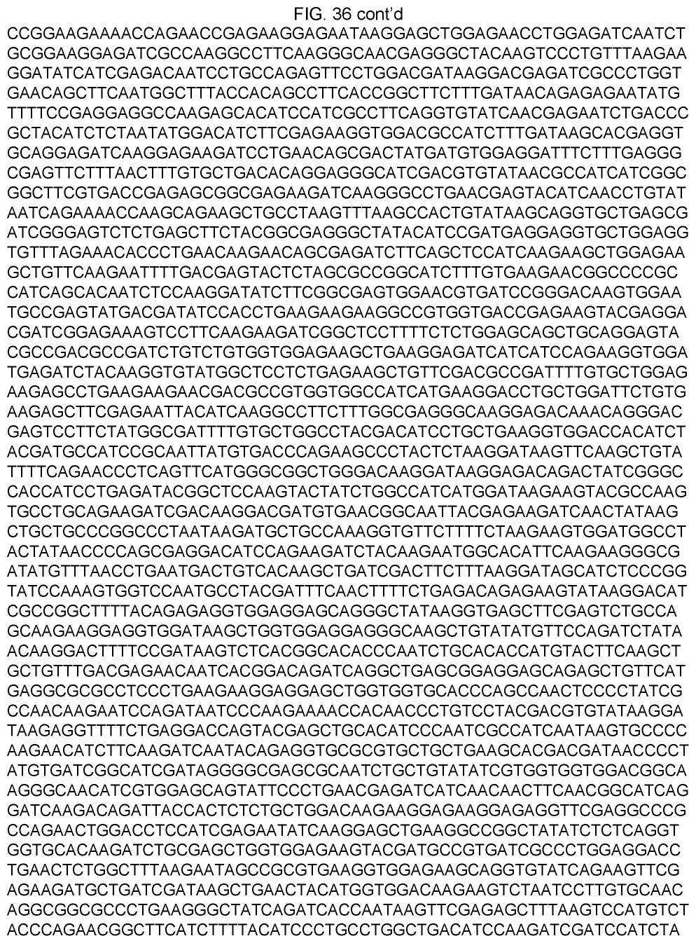

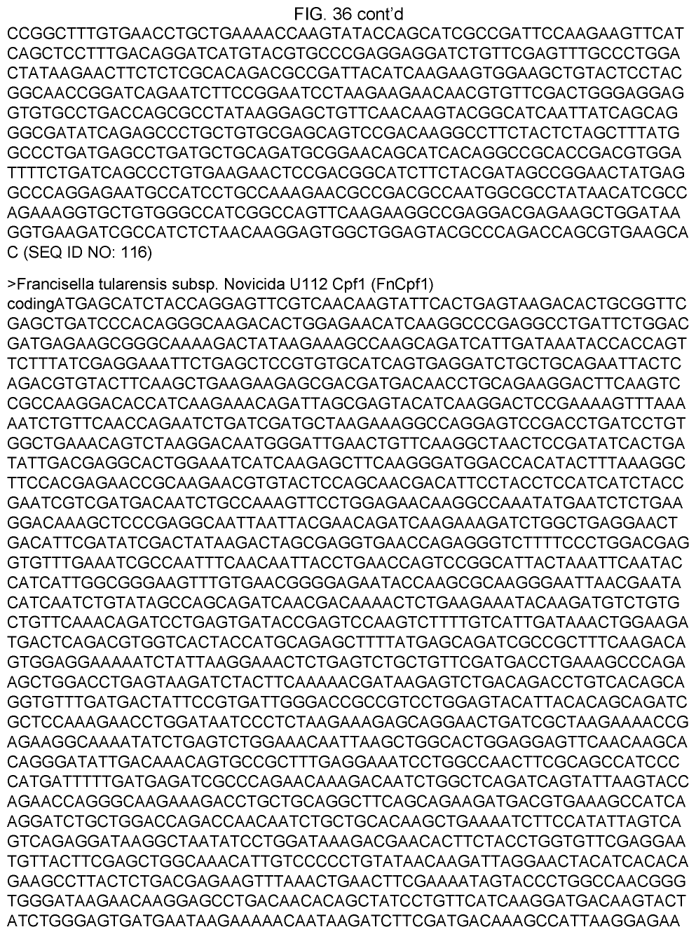

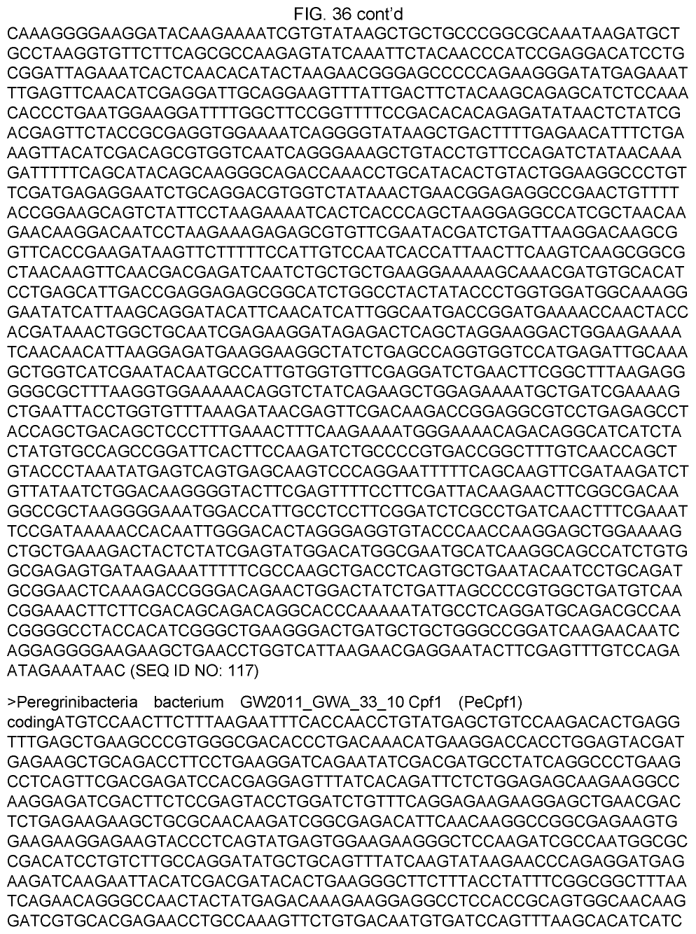

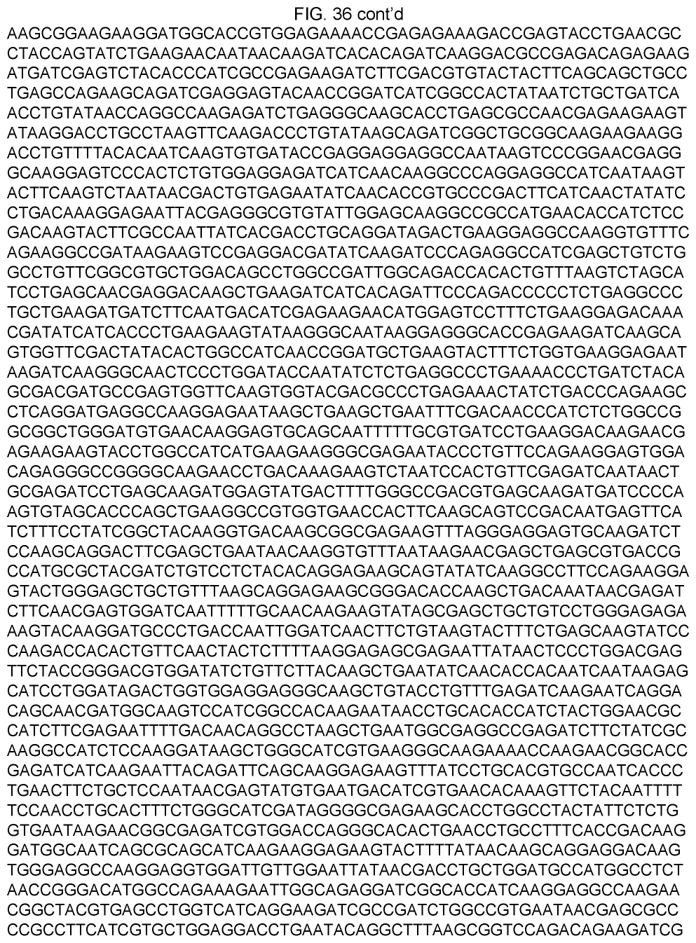

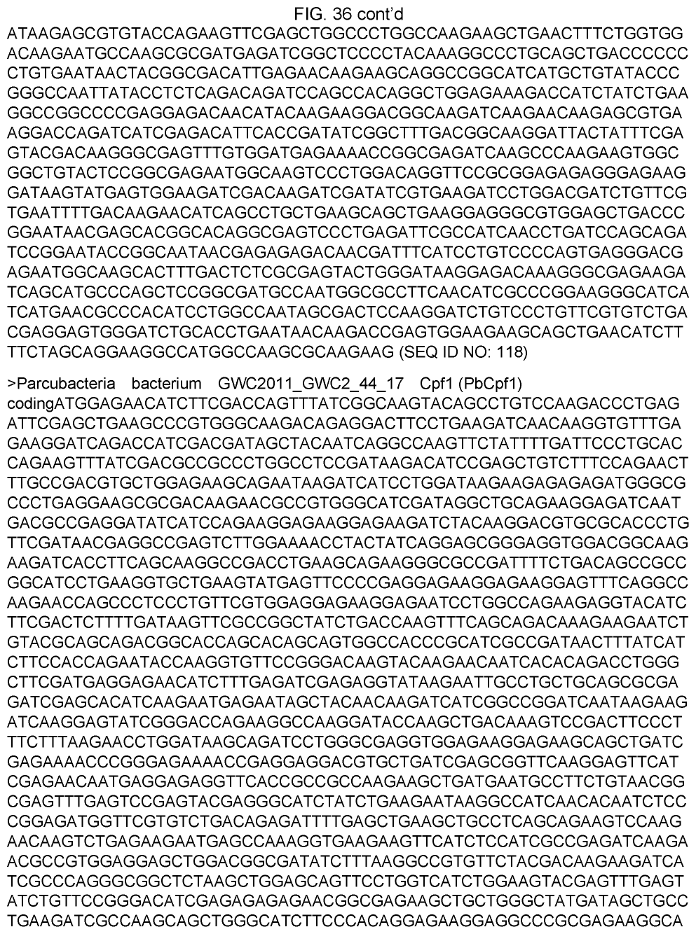

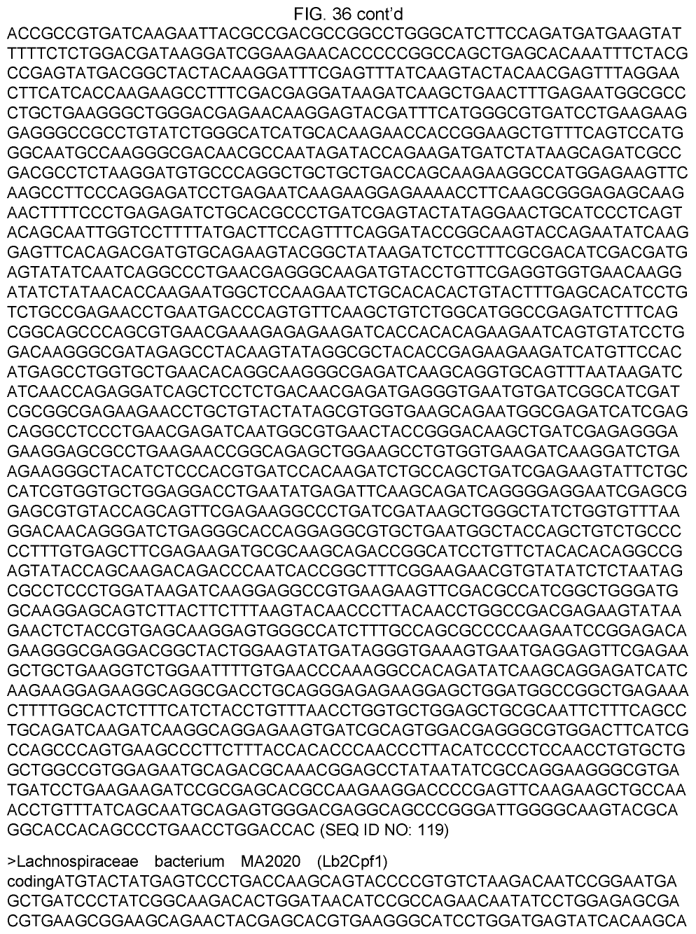

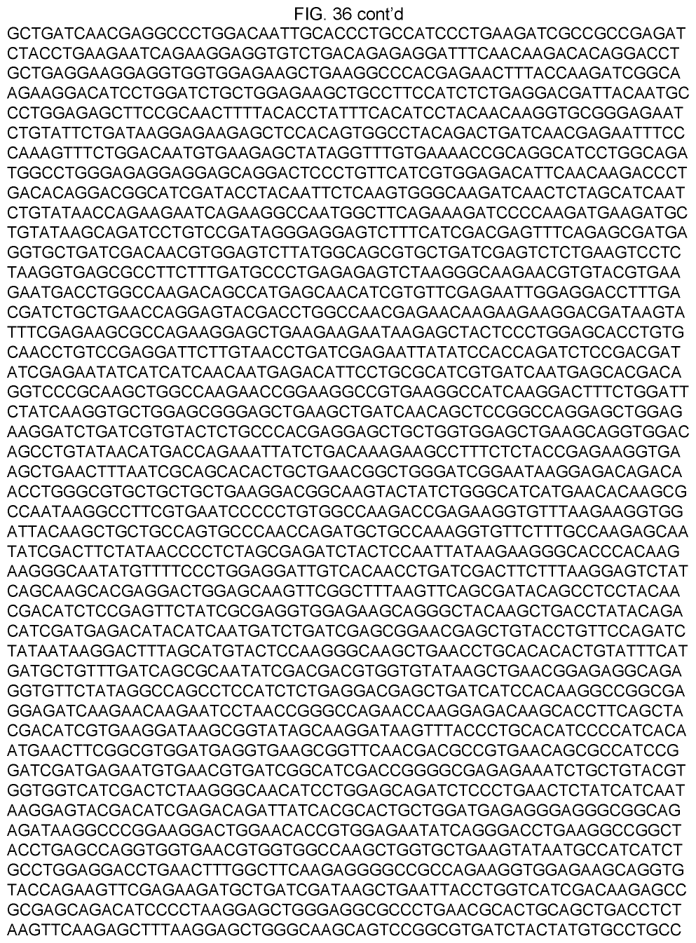

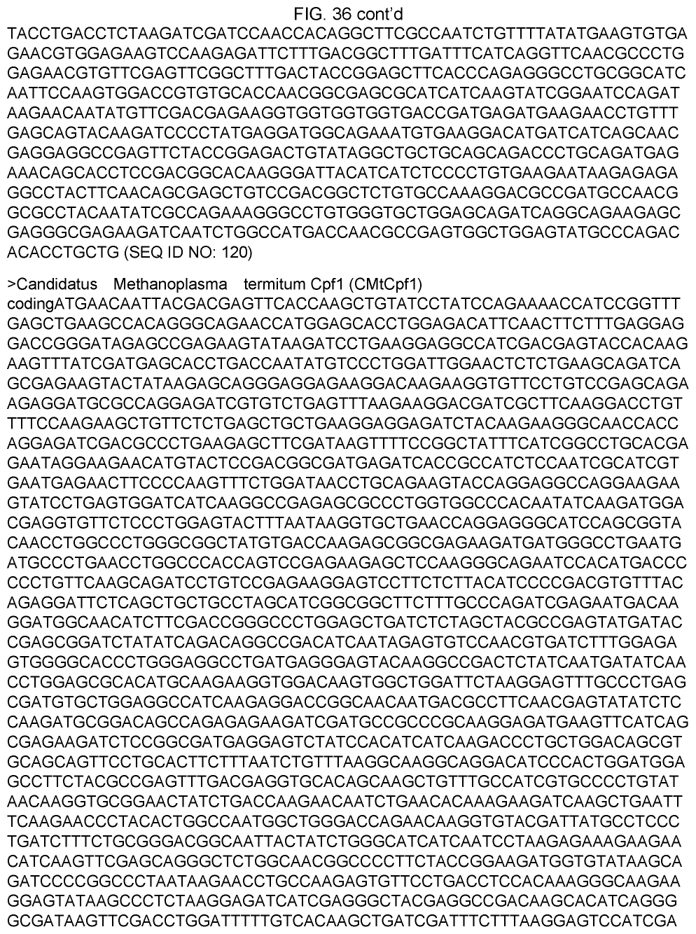

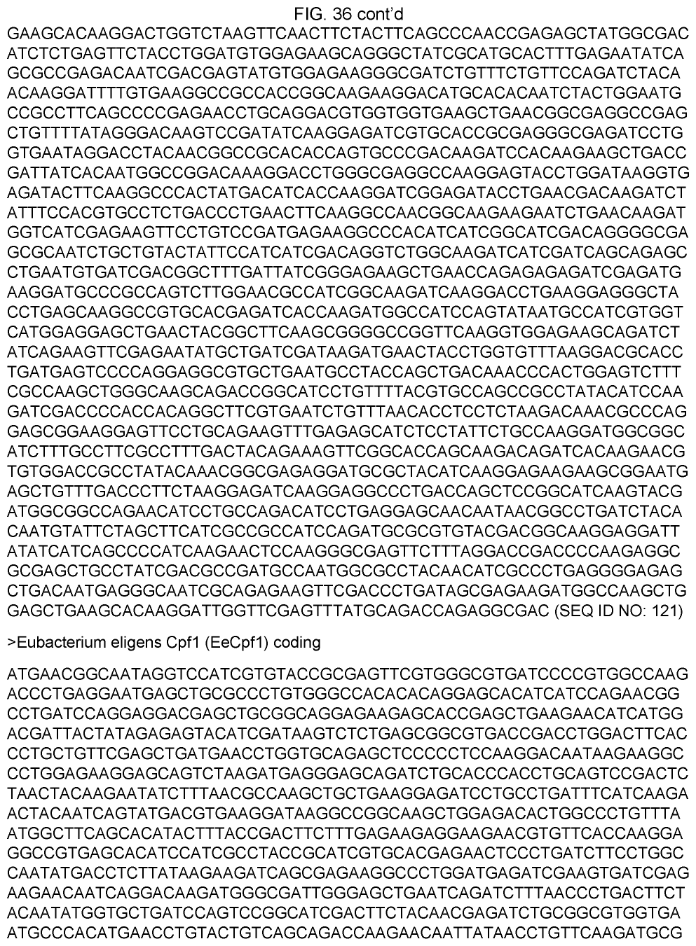

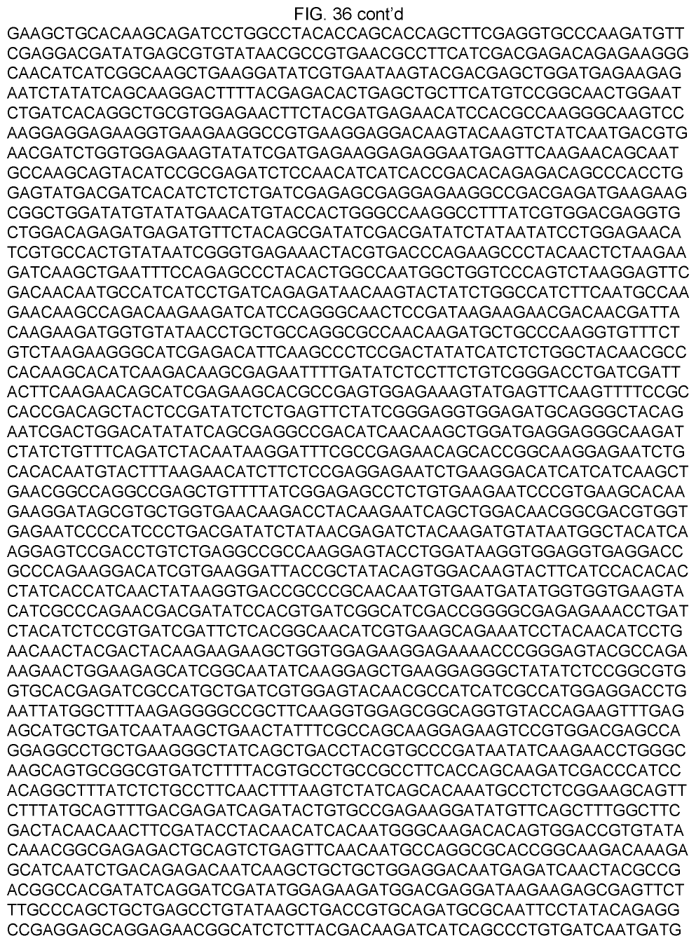

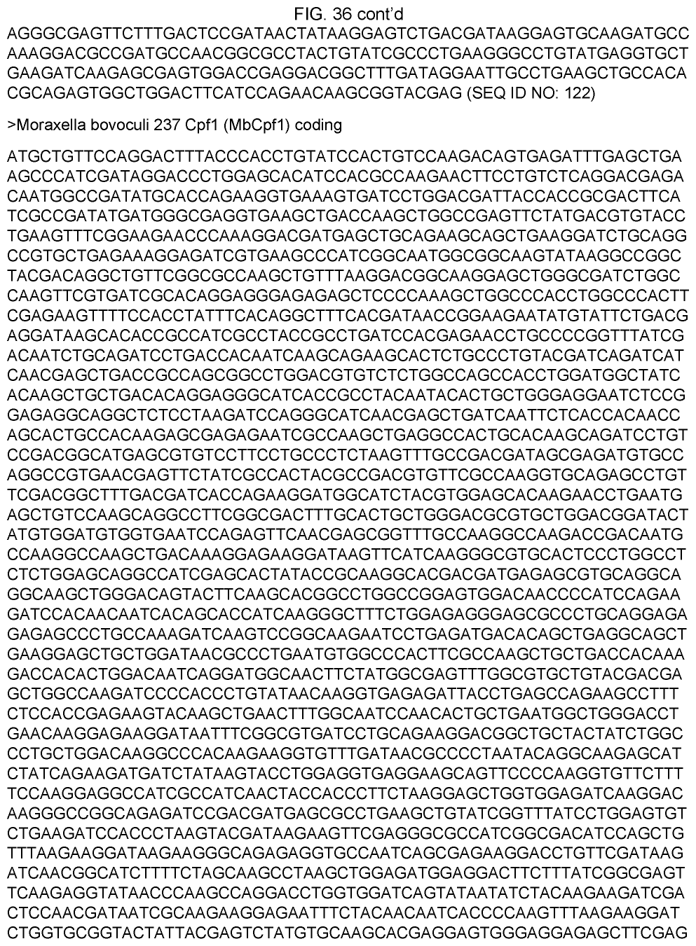

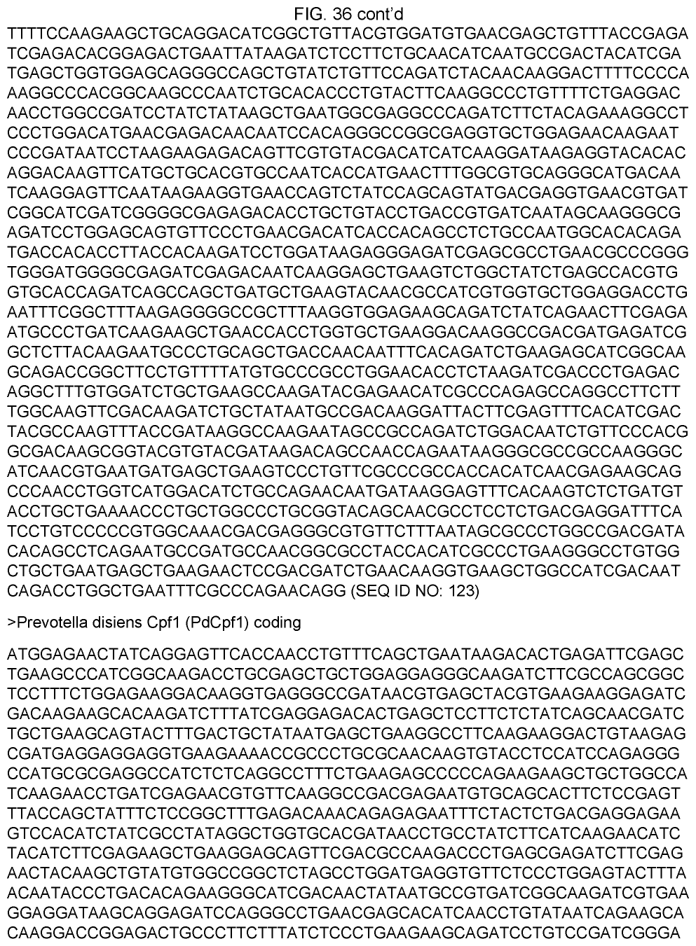

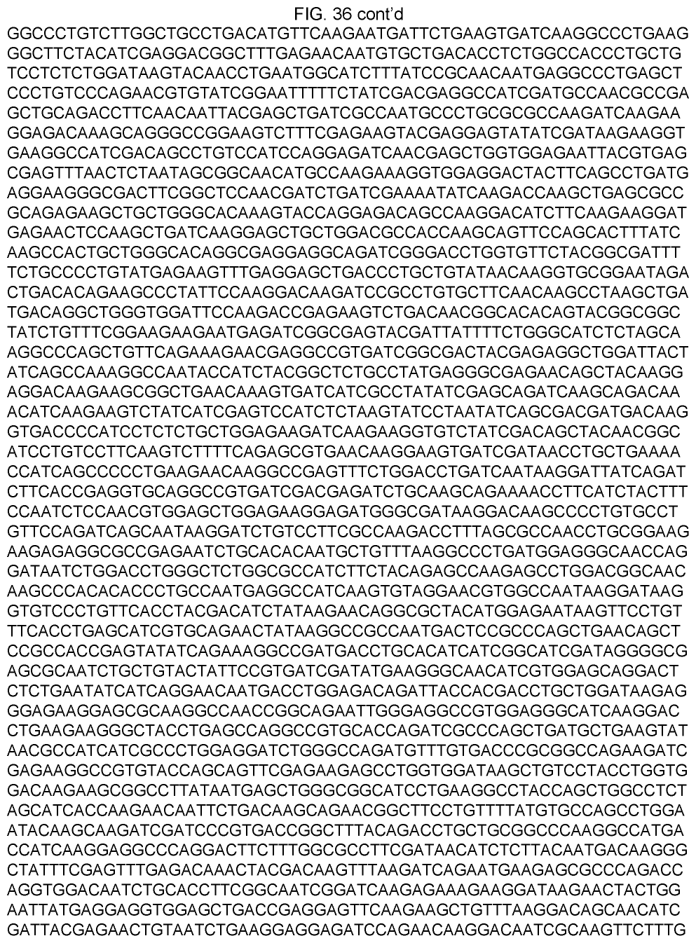

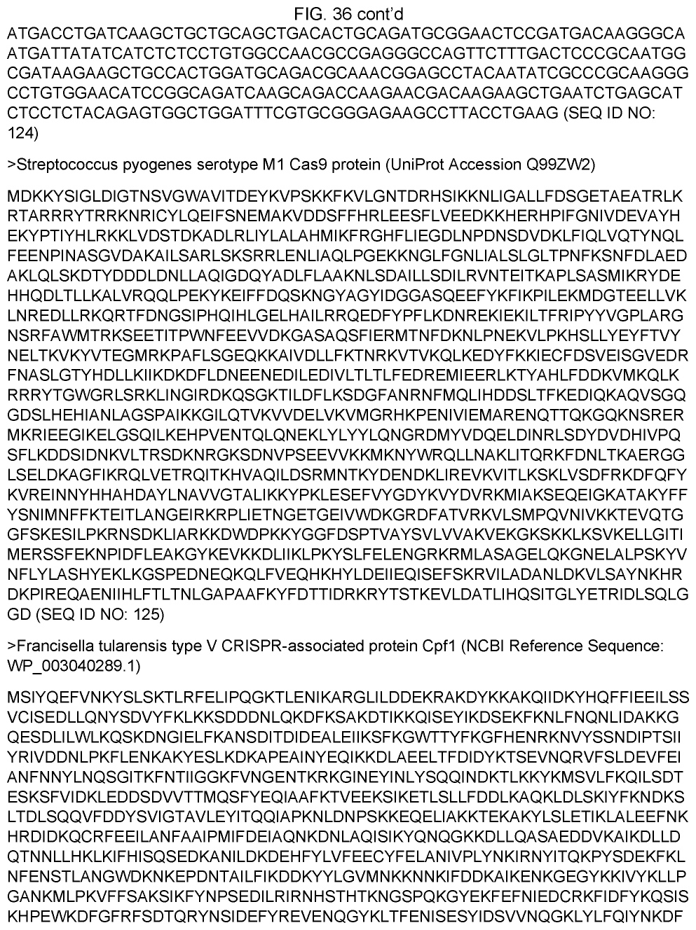

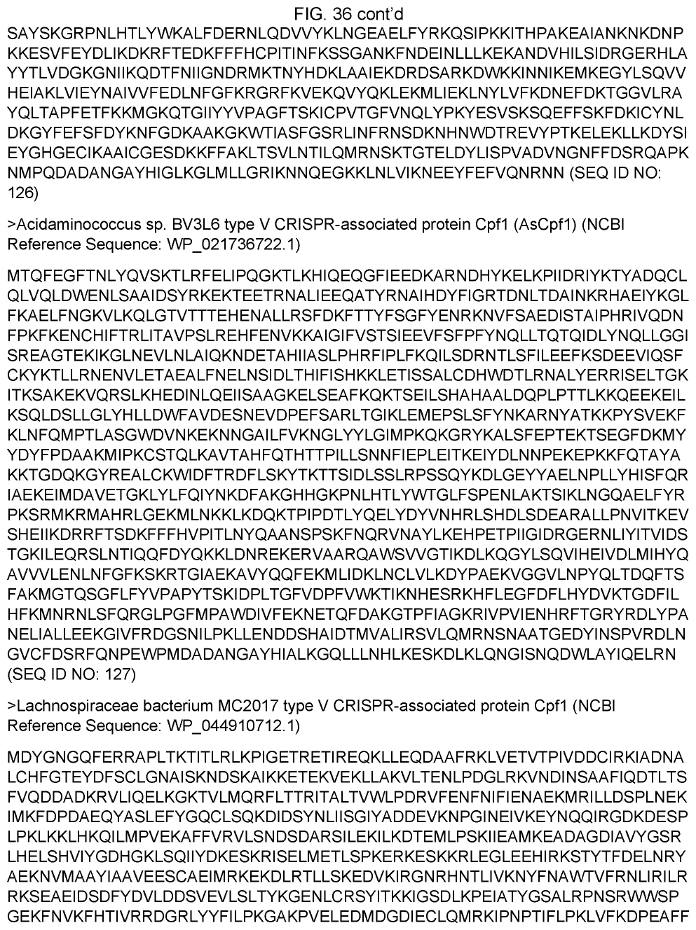

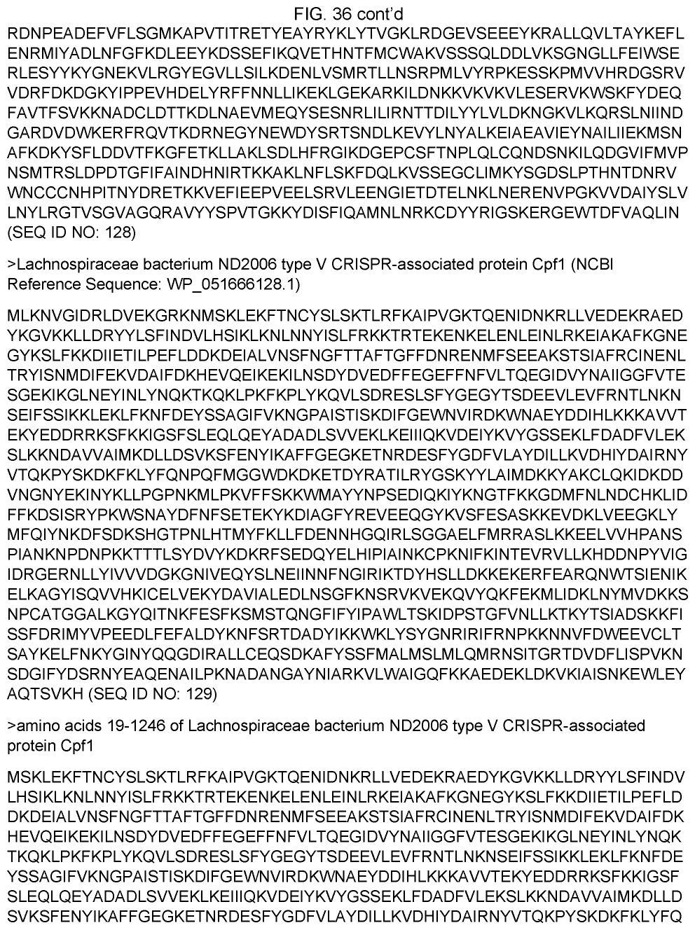

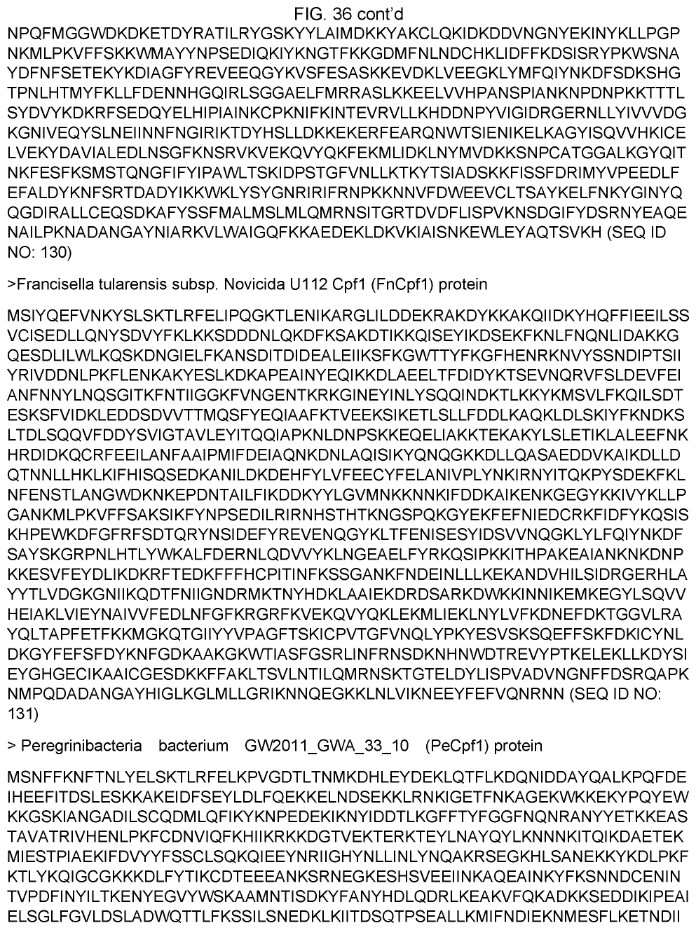

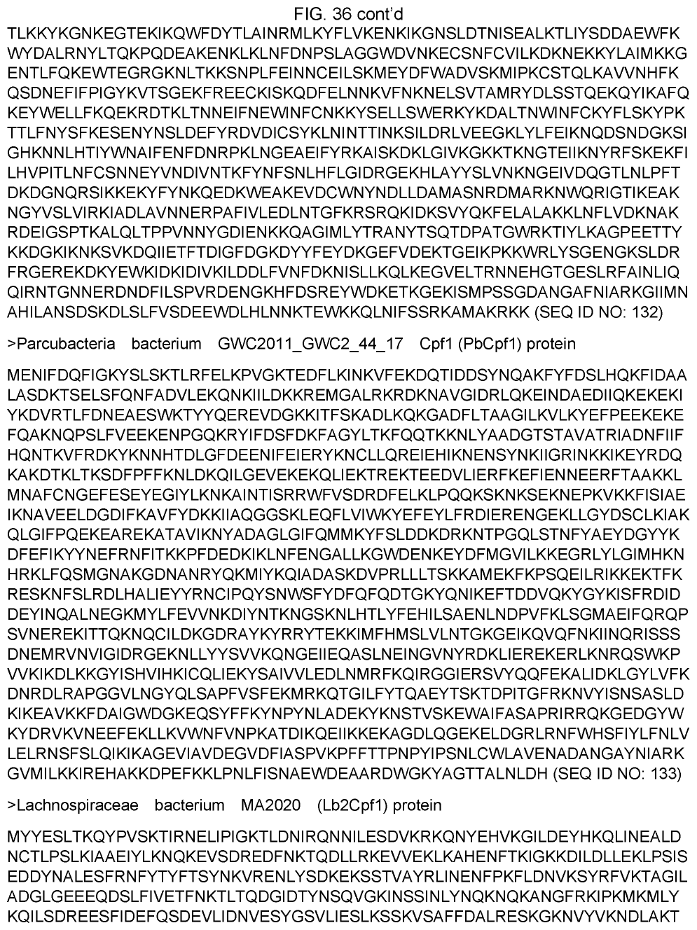

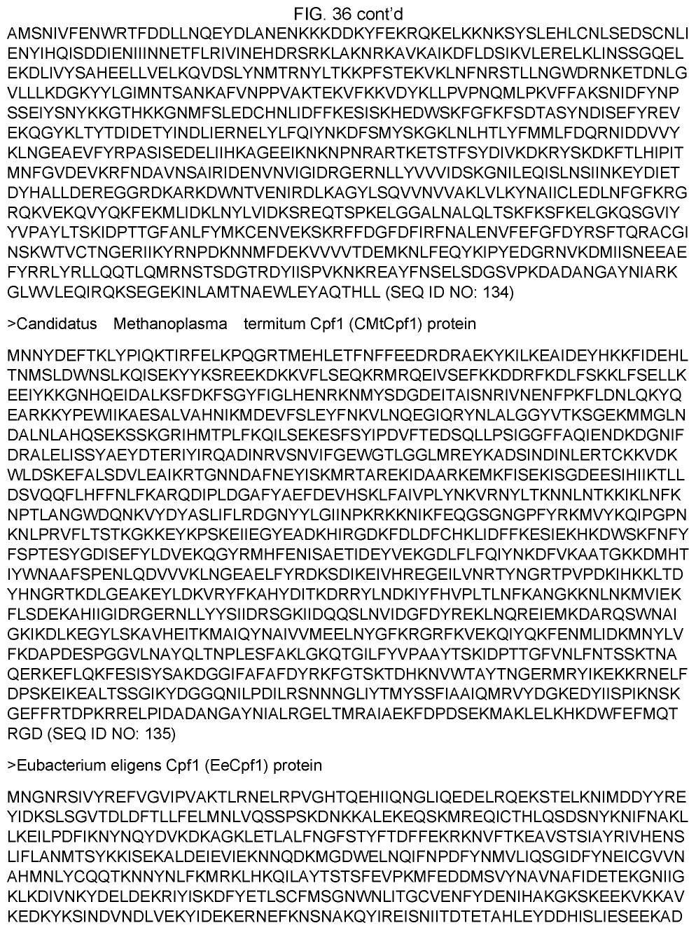

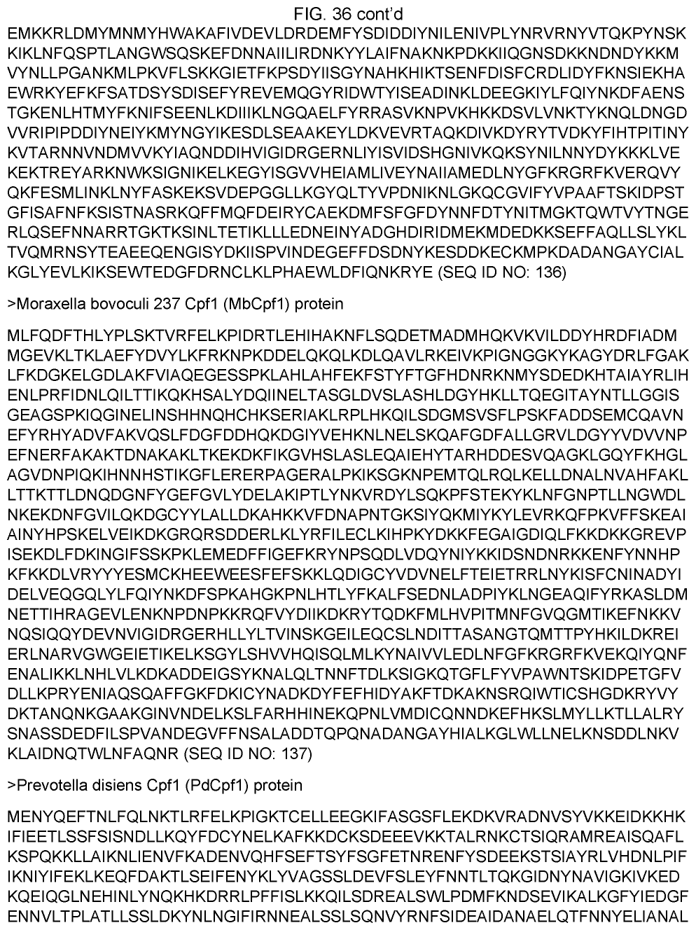

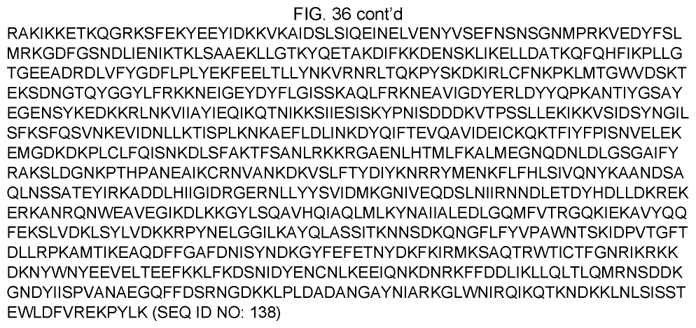

In silico off target analysis of the CCR5 target by CasOFFinder software demonstrated no homologous sites in the human genome with fewer than 3 bp mismatches for Cpf1 (FIG. 35A-35D) (Bae et al., Bioinformatics, 30(10): 1473-1475 (2014)). A target site was chosen encoding both Cpf1 and Cas9 PAM sites accessible with a single guide RNA, enabling direct comparison of these two CRISPR nucleases (FIGS. 7A, 7B). However, before testing began, HDT was optimized for Cpf1. Previous data demonstrated cleavage of the non-target strand by the RuvC domain is a prerequisite for the target strand cleavage by the Nuc domain (Yamano T et al., Cell, 165(4): 949-962 (2016)). Therefore, HDTs designed for the DNA target and non-target strands were tested. This HDT was comprised of 40 bp homology arms flanking the Cpf1 cut site (17 bp downstream from the PAM), on each end with 8 bp of NotI restriction enzyme cut site in the middle to disrupt CCR5 expression and enable HDR analysis. Using tracking of indels by decomposition (TIDE), a total editing rate of 8.1% was observed for the non-target strand and 7.8% for the target strand, with 7.3% HDR when HDT designed against the non-target strand was used, compared to 5.4% HDR when HDT designed against the target strand was used (FIG. 21A). These results were confirmed by T7EI and NotI restriction enzyme digestion assays (FIG. 21B), and were in close correlation with the previously published data by Yamano T et al., Cell, 165(4): 949-962 (2016).

The efficiency of HDR in primary HSPC was next optimized by preparing Au/CRISPR-HDT-NP in different concentrations (5 μg/mL-50 μg/mL) based on the amount of AuNP core suspended in molecular grade water. A concentration of 10 μg/mL demonstrated the highest total editing and HDR rate, with increasing concentrations demonstrating increased cytotoxicity and lower rates of HDR (FIGS. 21C, 21D).

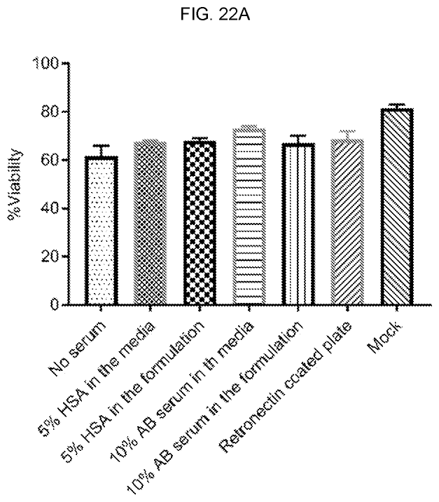

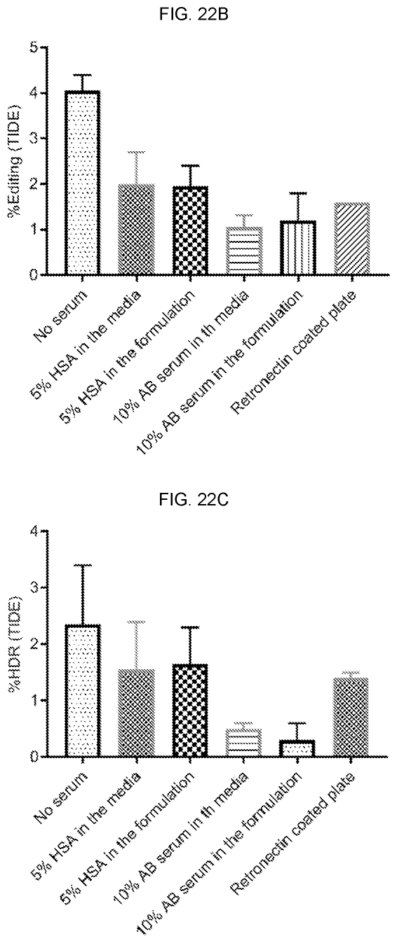

Typically, during clinical manipulation for ex vivo gene transfer, HSPC are cultured in serum-free media containing recombinant human growth factors on a layer of recombinant fibronectin fragment (RetroNectin®). Final formulations for infusion into patients consist of harvested HSPC suspended in nonpyrogenic isotonic solution such as Plasma-Lyte containing 2% human serum albumin (HSA). To determine the impact of these reagents, gene editing by Au/CRISPR-HDT NP were tested in the presence of HSA, RetroNectin® or pooled human A/B serum. No change in cytotoxicity was observed for any of the reagents (FIG. 22A), but all reagents reduced the total editing and HDR rates (FIGS. 22B, 22C). Thus, for all subsequent experiments, HDT (where included in the formulation) was designed against the non-target DNA strand, all formulations are added to HSPC in culture at a concentration of 10 μg/mL in molecular grade water, and HSPC were cultured in serum-free, supportive media without RetroNectin® or HSA.

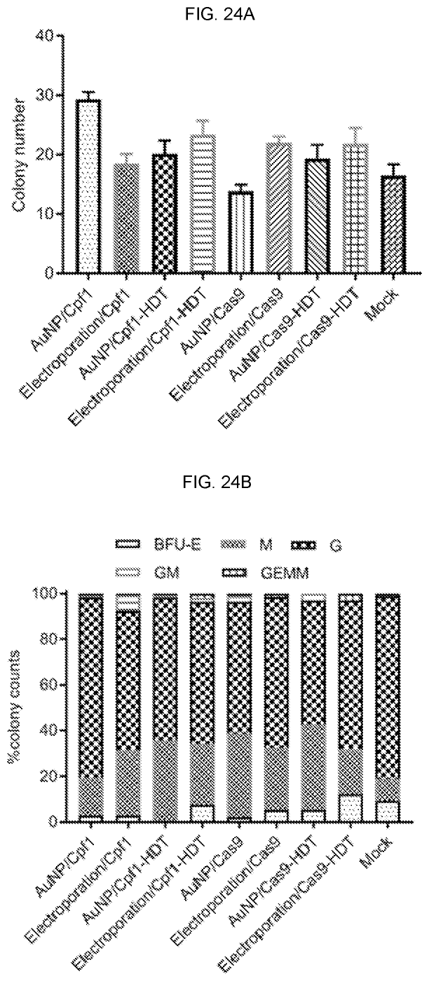

It was hypothesized that staggered cuts with 5′ overhangs made by Cpf1 would favor HDR more so than blunt ended cuts by Cas9 in HSPC. To test this hypothesis, Au/CRISPR NP were prepared targeting the CCR5 locus with and without HDT for both Cpf1 and Cas9. For comparison, the delivery was performed side by side with electroporation at identical concentrations of each component. Notably, additional chemical modifications were not included to the guide RNA, such as 2′ O-methyl ribonucleotide, 2′-deoxy-2′-fluoro-ribonucleotide and phosphorothioates (Yin et al., Nature Biotechnology, 35: 1179 (2017)), in any condition. TIDE analysis demonstrated a range of total editing between 2% and 25% with minimal significance (FIG. 23A). However, increased NotI restriction site incorporation was observed indicative of HDR in HSPC treated with Cpf1 or Cas9 delivered by the Au/CRISPR NP compared to electroporation by both TIDE and next generation sequencing, with Cpf1 outperforming Cas9 (FIGS. 23A-23C). All cell viabilities for all the samples were above 70%, but with higher viability observed in samples treated with AuNP, and in particular, significantly higher viability when Cas9 was delivered by AuNP rather than electroporation (FIG. 23D). HSPC fitness in these samples was analyzed by a colony-forming cell (CFC) assay with no observed differences in CFC potential or morphology (FIGS. 23E, 23F). This standard CFC assay is representative of more short-term blood progenitors [Wognum B., Yuan N., Lai B., Miller C. L. (2013) Colony Forming Cell Assays for Human Hematopoietic Progenitor Cells. In: Helgason C., Miller C. (eds) Basic Cell Culture Protocols. Methods in Molecular Biology (Methods and Protocols), vol 946. Humana Press, Totowa, N.J.], thus as a measure of long-term repopulating capacity, colonies from the original assay were re-plated. No significant differences in number or type of secondary CFCs were observed relative to the mock (untreated) control sample, but the pattern of higher CFC numbers in AuNP treated samples relative to electroporated samples was not observed (FIGS. 24A, 24B).

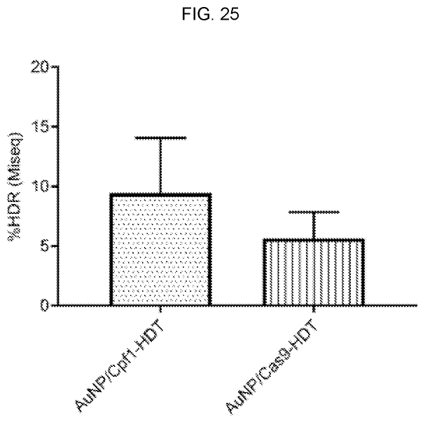

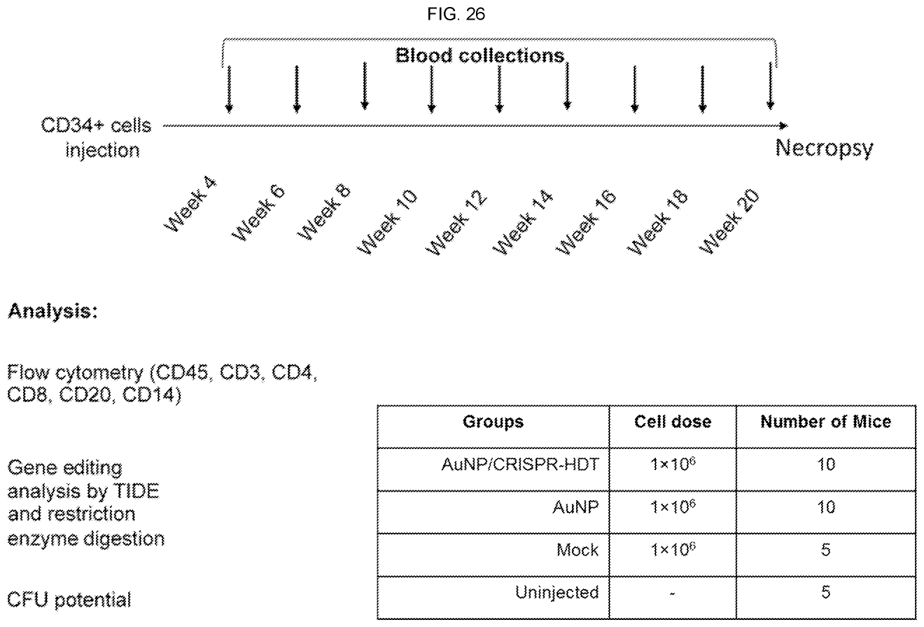

The same hypothesis was tested at the γ-globin promoter locus to affirm the Cpf1 preference for HDR. Here again, both Cpf1 and Cas9 PAM sequences were identified with an identical target cut site and no predicted off-target cutting (FIGS. 8A, 8B; FIG. 35A-35D). An HDT to insert a documented HPFH-associated, 13-bp deletion overlapping a repressor binding site in this promoter (Akinsheye et al., Blood, 118(1): 19 (2011)) was used. Obtained results in primary HSPC showed the same trend at this locus, with higher levels of HDR for Cpf1-containing Au/CRISPR NP as compared to Cas9-containing NP (FIG. 25).

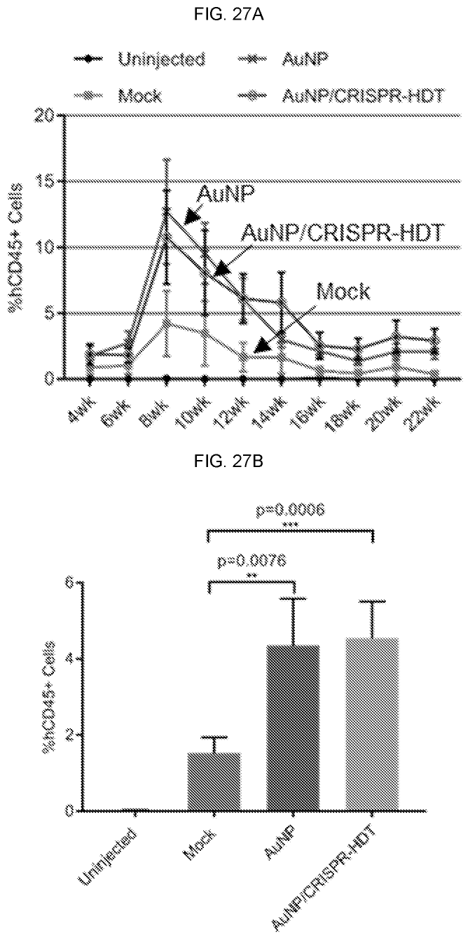

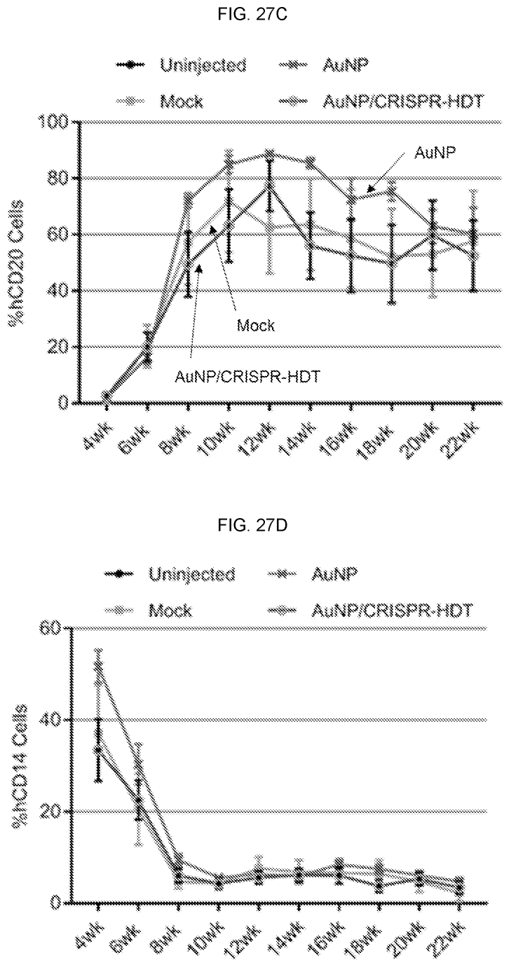

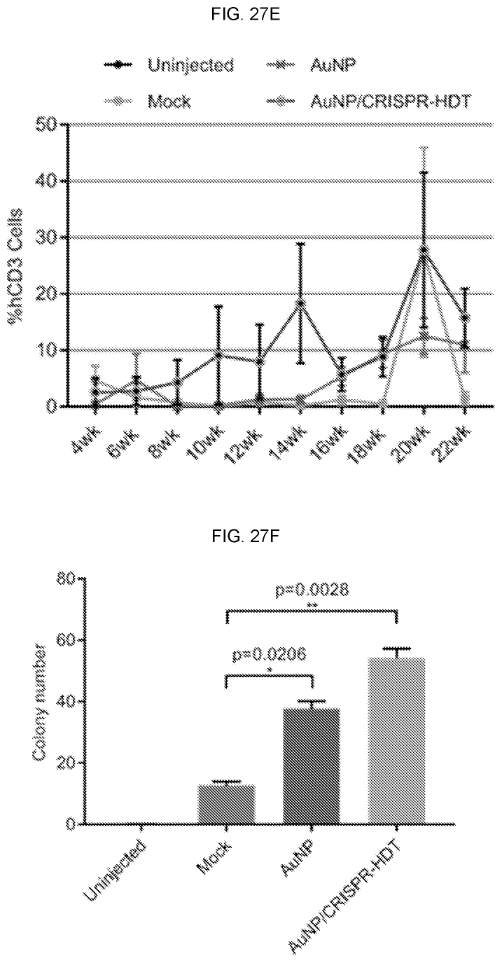

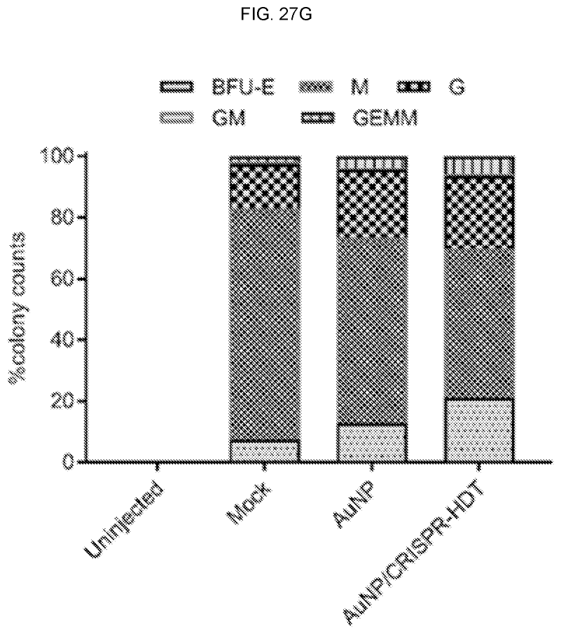

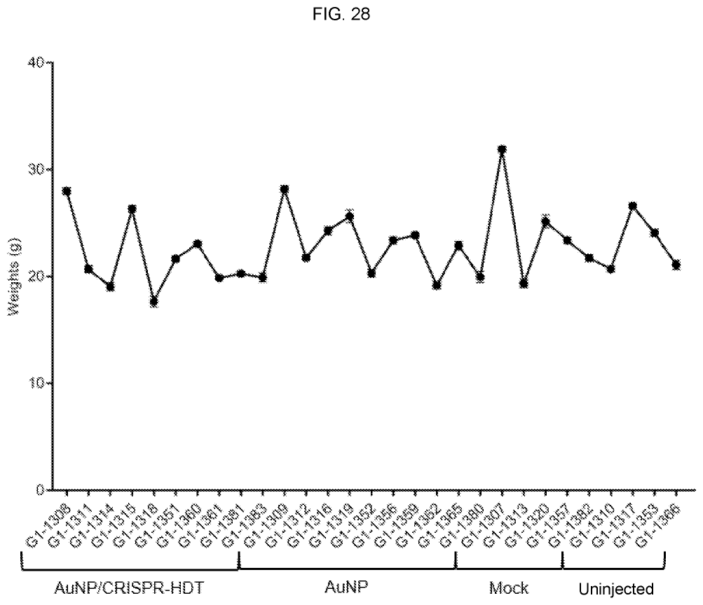

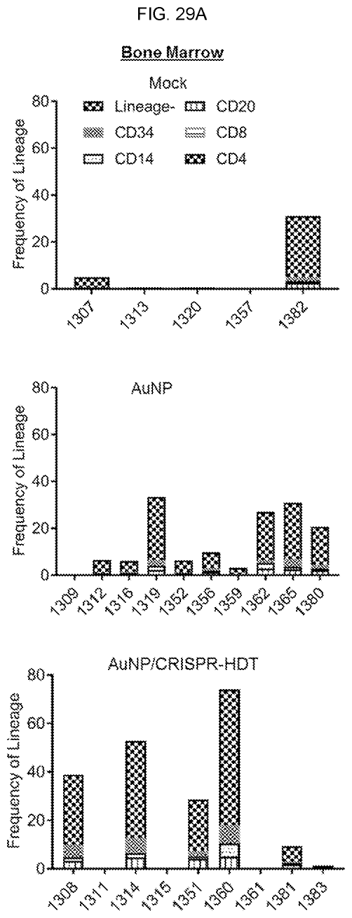

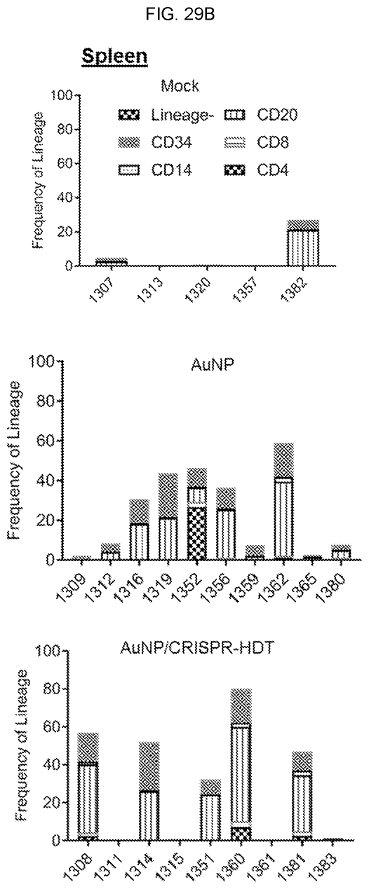

The next step was to determine whether NP treatment ex vivo compromised HSPC fitness following reinfusion. The best measure of HSPC fitness is ability to reconstitute a myelosuppressed host. Thus, primary human CD34⁺ HSPC were treated with Au/CRISPR-HDT-NP ex vivo and infused into sub-lethally irradiated immunodeficient (NOD/SCID gamma−/−; NSG) mice at 10⁶ cells/per mouse. Mice were followed for 22 weeks, with maximum engraftment observed at 8 weeks following transplant and stable engraftment establishing around week 16 after transplant (FIG. 27A). Mouse weights were monitored over the course of study and were stable over time (FIG. 28). Surprisingly, HSPC treated with Au/CRISPR-HDT-NP or AuNP alone engrafted at higher levels than mock (untreated) cells, but with similar kinetics (FIG. 27B). Different blood cell lineages were analyzed. Reconstitution of B cells reached peak at 10 weeks after transplant and then started to level-off through week 22 (FIG. 27C). Initial monocyte engraftment was high but decreased over the first 8 weeks and stabilized (FIG. 27D). Low levels of T cells were observed until week 16, which then increased for all the study groups (FIG. 27E). No significant differences in the proportion of B cells, monocytes or T cells were observed relative to the ex vivo HSPC treatment administered.

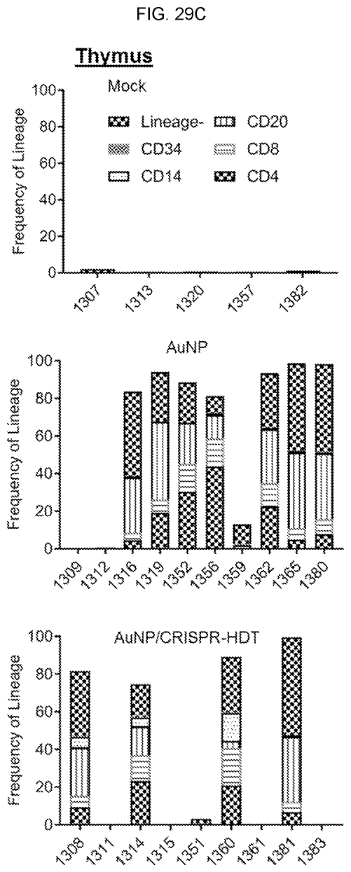

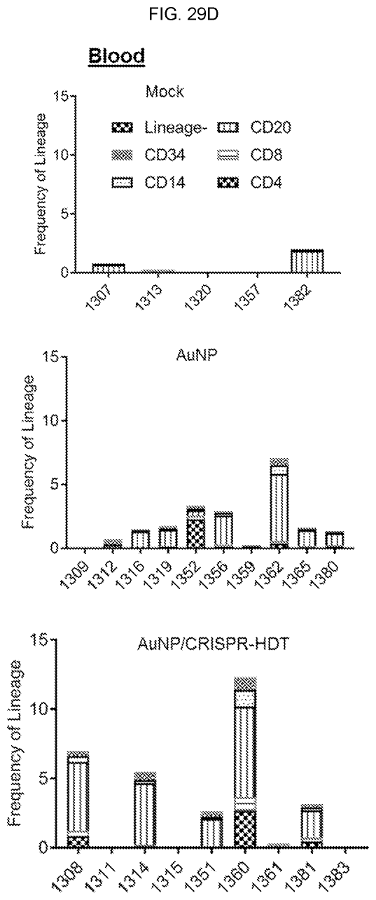

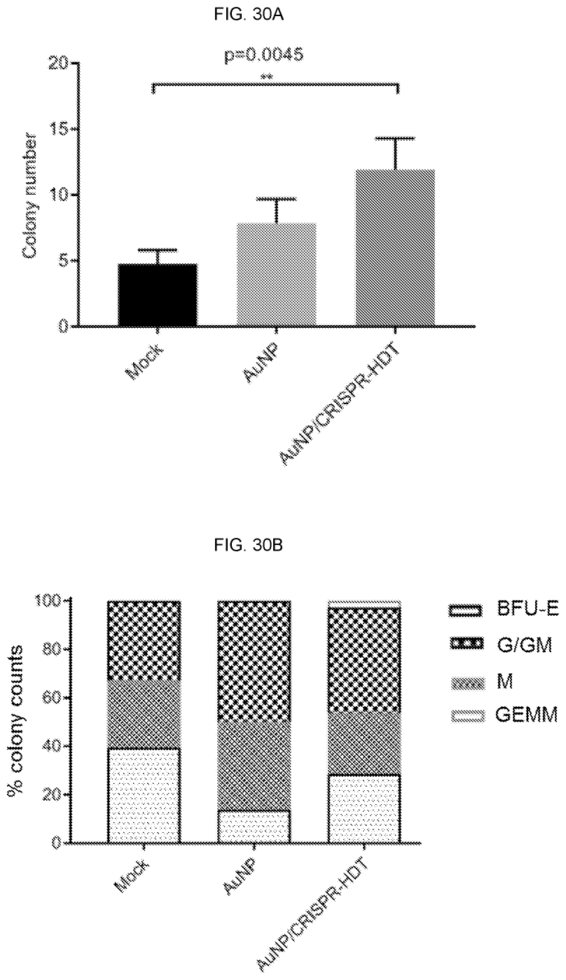

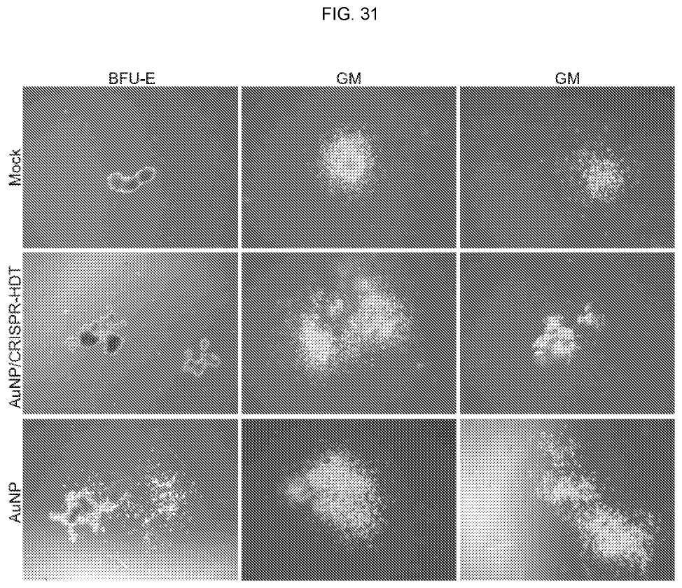

Mice were sacrificed after 22 weeks and bone marrow, spleen, thymus, and peripheral blood samples were retrieved. Flow cytometry analysis of the necropsy samples showed that in comparison to the mock group, AuNP and Au/CRISPR-HDT-NP treated groups were associated with higher levels of engraftment (FIGS. 29A-29D). Importantly, the frequency of multipotent CD34+ cells was higher in bone marrow, spleen, and peripheral blood of AuNP-treated animals (FIGS. 29A, 29B, 29D), and the frequency of CD20-expressing cells was higher in the spleen, thymus and peripheral blood (FIGS. 29B, 29C, 29D). A human-specific CFC assay of the bone marrow samples was in close correlation with the engraftment results and showed that AuNP and Au/CRISPR-HDT-NP treated groups had significantly higher colony numbers compared to the mock treated group (FIG. 27F). This was closely related with the higher number of multipotential progenitor cells in these groups (FIG. 27G). These results were also in close correlation with the CFC assay results observed in the treated HSPC infusion product before the transplantation suggesting a positive effect of AuNP treatment in ex vivo cultured HSPC (FIGS. 30A-30B). Colony morphologies for all the treated samples are shown in FIG. 31.

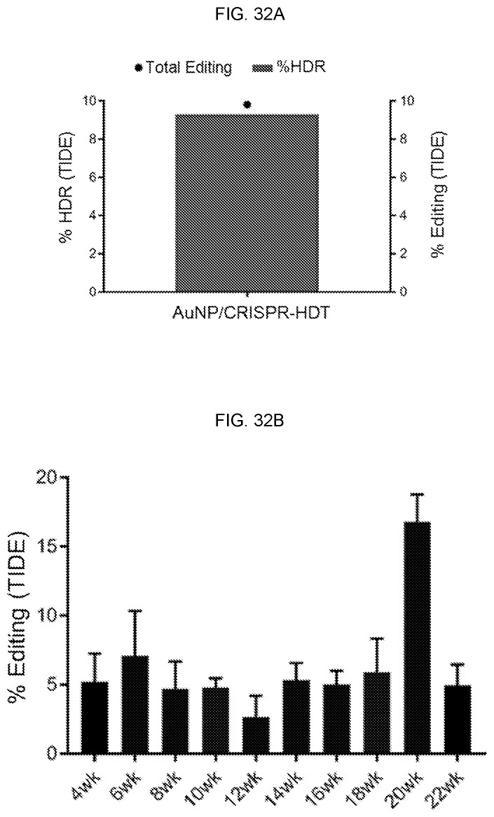

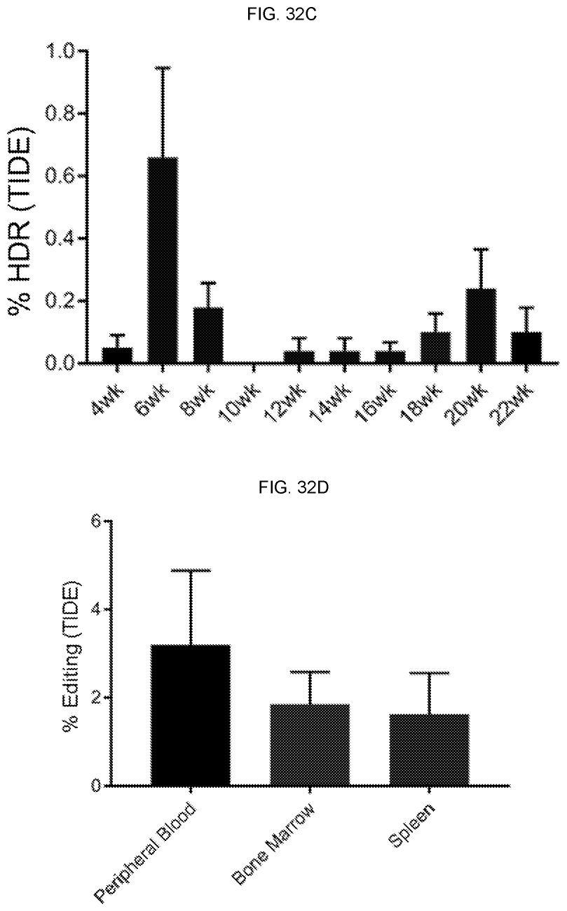

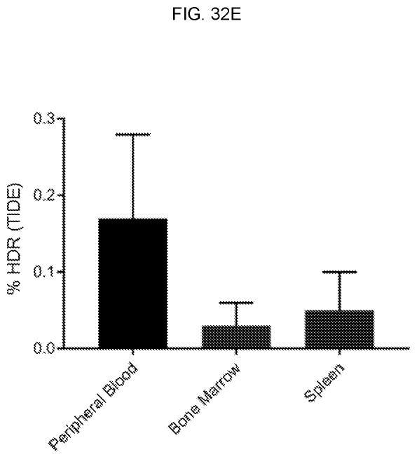

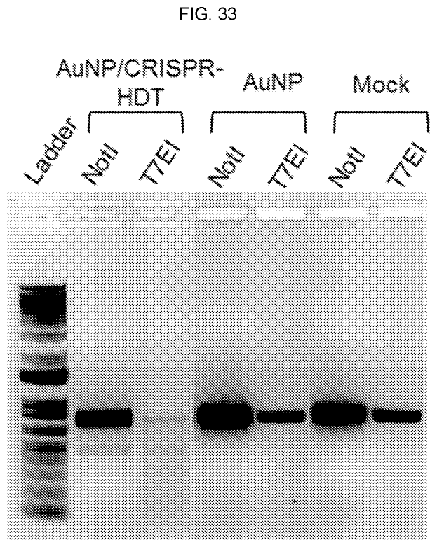

In terms of gene editing, 9.8% total editing and 9.3% of HDR were observed by TIDE analysis in HSPC at the time of transplant (FIGS. 32A, 33). Stable levels of total gene editing (5%) were observed in peripheral blood cells with one transiently high value of 17% observed at week 20 (FIG. 32B). Interestingly, the levels of NotI restriction enzyme incorporation were consistently lower than 1% across all time points (FIG. 32C). Analyzing the necropsy samples from different tissues showed that HDR was comparably low in blood, bone marrow and spleen (FIGS. 32D, 32E).

Gene editing is a promising approach for genetic screening to identifying unknown genes and understanding gene function and correcting defective genes in congenital or acquired genetic diseases (Xiong et al., Annual Review of Genomics and Human Genetics, 17(1): 131-154 (2016)). Gene-editing technology is moving rapidly from basic science to clinical application, however the current state of the clinical art for delivery of gene-editing components in HSPC requires electroporation, possibly with AAV transduction, which is far more complex than retrovirus-mediated gene transfer. Despite all achieved experience from RNA, DNA and protein delivery, there is no generalizable, simple approach for gene-editing component delivery which is both effective and safe, suggesting that various cell types and tissues may require different delivery strategies.

In this study Au was used to develop a widely applicable gene-editing delivery system. This multilayered NP was able to package all the required gene editing components with or without a DNA repair template on a single AuNP core with little impact on NP monodispersity. Stringent characterization at each component loading step was critical to the design. Optimal NP remained in a non-aggregated state and successfully penetrated into hard-to-transfect CD34+ hematopoietic cells. Data from other cell types has shown that Au/CRISPR NP are internalized through endocytosis inside small vesicles which then burst and release into the cytoplasm. A PEI-induced proton sponge effect could be facilitating escape from HSPC lysosomes (Benjaminsen et al., Molecular therapy: the journal of the American Society of Gene Therapy, 21(1): 149-157 (2013)). Additionally, PEI has been shown to play an active role in nuclear trafficking of the NP which in addition to nuclear localizatiom signals on nuclease proteins could facilitate payload delivery (Reza et al., Nanotechnology, 28(2): 025103 (2017)). The CCR5 and γ-globin promoter loci targeted here were very unique, encoding PAM sites for Cpf1 and Cas9 with the same guide recognition site, enabling unbiased comparison of these two nuclease platforms with this NP. Importantly, 10 μg/mL Au/CRISPR NP concentrations produced up to 17.6% total editing with 13.4% HDR at the CCR5 locus and 12.1% total editing with 8.8% HDR at the γ-globin promoter locus when Cpf1 nuclease was included in the NP. Total editing and HDR results were comparable to or higher than electroporation-mediated delivery, suggesting a HSPC biology more amenable to CRISPR gene editing when AuNPs are the delivery mode. Also, the higher levels of HDR observed with Cpf1 as opposed to Cas9 in the NP suggest that staggered nuclease cutting may favor HDR, at least at these therapeutically-relevant loci (Zetsche et al., Cell, 163(3): 759-771 (2015) and Nakade et al., Bioengineered, 8(3): 265-273 (2017)).

Colony assays results and xenoengraftment data demonstrate that Au/CRISPR-HDT-NP treatment did not have any adverse effect on HSPC fitness following ex vivo treatment and suggest that repopulating potential may even be increased.

Evidence is provided that Au/gene-editing NP produce surprisingly efficient and safe delivery of gene editing machinery to HSPCs. This study expands the available delivery toolkit for gene-editing component delivery.

Materials. Synthesis and characterization of NP. AuNP were synthesized by Turkevich's method with slight modification (Turkevich et al., Discussions of the Faraday Society, 11(0): 55-75 (1951) and Shahbazi et al., Nanomedicine (London, England), 12(16): 1961-1973 (2017)). 0.25 mM Chloroauric acid solution (Sigma-Aldrich, St. Louis, Mo.) was brought to the boiling point and reduced by adding 3.33% sodium citrate solution (Sigma-Aldrich, St. Louis, Mo.) and stirred vigorously under reflux system for 10 min. Synthesized NP were washed three times by centrifuging at 17000 for 15 min and re-dispersed in ultra-pure water (Invitrogen, Carlsbad, Calif.).

All oligonucleotides used in this study were purchased from Integrated DNA Technologies (IDT, Coralville, Iowa). Cas9 and Cpf1 enzymes were purchased from Aldevron, LLC (Fargo, N. Dak.). crRNAs with an 18 oligo ethylene glycol (OEG) spacer-thiol modification on the 3′ end for AsCpf1 and 5′ end for SpCas9 were used (sequence information can be found in FIG. 34). crRNA and tracrRNA duplex (gRNA) for Cas9 nuclease were made by mixing them in equimolar concentration in duplex buffer and incubating at 95° C. for 5 min and cooling on the bench top. AuNPs in 10 μg/mL concentration were added to crRNA or gRNA solution in AuNP/crRNA w/w ratio of 0.5. Citrate buffer (pH 3.0) was added to 10 mM and the resulting solution was mixed for 5 min. Prepared AuNP/crRNA nanoconjugates were centrifuged down and re-dispersed in 154 mM sodium chloride (NaCl) (Sigma-Aldrich, St. Louis, Mo.). Then, nuclease was added in AuNP/Cpf1 or AuNP/Cas9 w/w ratio of 0.6, and mixed by pipetting the solution up and down and incubating for 15 min. Following that, NP were centrifuged at 16000 g for 15 min and redispersed in NaCl solution. Polyethyleneimine (PEI) of 2000 MW (Polysciences, Philadelphia, Pa.) was added in 0.005% concentration, mixed thoroughly and after 10 min incubation NP were centrifuged at 15000 g for 15 min and redispersed in NaCl solution. In the final step, HDT was added in the AuNP/HDT w/w ratio of 2 and after 10 min incubation NP were centrifuged and redispersed in NaCl solution.

The size and shape of the prepared NP were characterized by transmission electron microscope (TEM) (JEOL JEM 1400, Akishima, Tokyo, JP). Samples were negatively stained first by glow-discharging carbon-coated grid, using the PELCO easiGlow Glow Discharge system (Ted Pella Inc., Redding, Calif.). A volume of 2 μL of the sample was dropped on the grid and after 30s it was blotted off, washed and stained in 0.75% uranyl formate solution (Polysciences, Philadelphia, Pa.). Finally, grids were dried inside the desiccator overnight and imaged by TEM (Booth et al., JoVE (58): 3227 (2011)).

The hydrodynamic size and polydispersity index of the NP were characterized by Zetasizer Nano S device (Malvern, UK). Measurements were carried out in triplicate and results were reported as mean±SD. Low volume disposable cuvettes (ZEN0040) (Malvern, UK) were used for the measurements.

The zeta potential of the NP was characterized by using Zetasizer Nano ZS (Malvern, UK). Disposable Folded Capillary Zeta Cell (Malvern, UK) was used for the measurements and results are reported as mean±SD.

Also, layer by layer conjugation of the CRISPR components was characterized by measuring the shifts in the localized surface plasmon resonance (LSPR) of AuNP using a nanodrop device (Thermo Fisher Scientific, Waltham, Mass.).

Isolation and culture of CD34+ cells. Primary human CD34+ cells were isolated from healthy donors mobilized with granulocyte colony stimulating factor (G-CSF; Filgrastim, Amgen, Thousand Oaks, Calif.). Whole leukapheresis products were obtained and CD34-expressing cells were purified by immunomagnetic bead-based separation on a CliniMACS™ Prodigy device using previously published protocols (Adair et al., Nat Commun, 7: 13173 (2016)). Resulting CD34+ cells were cultured in StemSpan Serum-Free Expansion Medium version II (SFEM II; Stem Cell Technologies) or Iscove's Modified Dulbecco's Medium (IMDM; Invitrogen Life Sciences, Carlsbad, Calif.) containing 10% fetal bovine serum (FBS; Gibco, Waltham, Mass.), and 100 ng/mL each of recombinant human stem cell factor (SCF), Flt-3 ligand (Flt3) and thrombopoietin (TPO), all from Cellgenix (Freiburg, Germany). Incubation conditions were 37° C., 85% relative humidity, 5% CO₂ and normoxia.

In vitro gene editing studies. CD34+ cells were thawed and pre-stimulated overnight in SFEM II media containing SCF, Flt3 and TPO. Following that, cells were seeded in a 96 well plate at 1×10⁶/mL and treated with Au/CRISPR NP at 10 μg/mL concentration of AuNPs. All in vitro experiments were carried out in triplicate. After 48 h incubation, cells were washed with Dulbecco's phosphate buffered saline (D-PBS) (Gibco, Waltham, Mass.) and harvested for gDNA extraction and gene editing analysis.

Electroporation of the CRISPR components was also carried out for comparison. To do so, 49 pmol crRNA or gRNA was mixed with the same amount of Cpf1 or Cas9 nucleases (8.5 pmol) and incubated for 15 min. Cells were dispersed in electroporation buffer and mixed with ribonucleoprotein (RNP) complex. The mixture was added to 1 mm electroporation cuvettes and electroporated under 250 V, and 5 ms pulse duration using a BTX electroporator device (BTX, Holliston, Mass.). After that, cells were put in culture and washed after 24 h followed by another 24 h incubation. After 48 h incubation, cells were washed with D-PBS and harvested for gDNA extraction and gene editing analysis.

Cell viability analysis. Cell viability after treatment with Au/CRISPR NP and electroporation was analyzed at different time points using Countess II FL Automated Cell Counter (ThermoFisher Scientific, Waltham, Mass.). 10 μL of the trypan blue stain (0.4%) (Invitrogen) was mixed with 10 μL of cell suspension, and 10 μL of the mixture was applied to a disposable cell counting chamber slide and inserted into the device. Percent cell viability of each sample was recorded and reported as mean±SD.

In order to confirm the results, cell viability was also analyzed using the LIVE/DEAD® assay kit (Invitrogen, Carlsbad, Calif.). Cells were washed in D-PBS and sedimented by centrifugation. Then, an aliquot of the cell suspension was transferred to a coverslip. Cells were allowed to settle to the surface of the glass coverslip at 37° C. in a covered 35 mm petri dish. Calcein AM (2 μM) and ethidium homodimer-1 (EthD-1) (4 μM) working solution was prepared and 150 μL of the combined LIVE/DEAD® assay reagents were added to the surface of a 22 mm square coverslip, so that all cells were covered with solution. Cells were incubated in a covered dish for 30 min at room temperature. Following incubation, 10 μL of D-PBS was added to a clean microscope slide and a coverslip was inverted and mounted on the microscope slide. Labeled cells were imaged under the fluorescence microscope (Nikon Ti Live, Japan) using excitation and emission values of 494/517 nm for Calcein AM, and 528/617 nm for EthD-1. Live and dead cells were counted using the cellomics vHSC software (v1.6.3.0, Thermo Fisher Scientific, Waltham, Mass.). Images were processed using ImageJ software (V 1.5i, National Institutes of Health, Rockville, Md.).

Colony Forming Cell (CFC) Assay. For CFC assays, cells were plated in methylcellulose (H4230: Stem Cell Technologies, Vancouver, Calif.) containing recombinant human growth factors according to the manufacturer's specifications and incubated for a period of 14 days. Resulting colonies were counted and scored for morphology on a stereo microscope (ZEISS Stemi 508, Germany) to determine the number of colony-forming cells for every 100,000 cells plated.

Genome editing detection by T7 Endonuclease I. To analyze the total gene editing percentage, genomic DNA was extracted using PureLink® (Thermo Fisher Scientific, Waltham, Mass.) Genomic DNA Mini Kit following the manufacturer's protocol and PCR amplified.

The genomic region flanking the CRISPR target site (755 bp) was PCR amplified (sequence information can be found in FIG. 34), and products were purified using PureLink® PCR Purification Kit following the manufacturer's protocol. 200 ng total of the purified PCR products were mixed with 2 μL 10×NEBuffer 2 (New England BioLabs, Ipswich, Mass.) and ultrapure water to a final volume of 19 μL and were subjected to a re-annealing process to enable heteroduplex formation: 95° C. for 5 min, 95° C. to 85° C. ramping at −2° C./s, 85° C. to 25° C. at −0.1° C./s, and 4° C. hold. After re-annealing, products were treated with 1 μL of T7EI nuclease (New England BioLabs, Ipswich, Mass.) and incubated for 15 min at 37° C. After incubation digested products were purified by PureLink® PCR Purification Kit and analyzed on 2% agarose gel. Gels were imaged with a Gel Doc gel imaging system (Bio-Rad, Hercules, Calif.). Quantification was based on relative band intensities. Indel percentage was determined by the formula, % gene modification=100×(1−(1− fraction cleaved)1/2).

NotI restriction enzyme digestion. Genomic regions flanking the CRISPR target site (755 bp) was PCR amplified and products were purified using PureLink® PCR Purification Kit following the manufacturer's protocol. 1000 ng total of the purified PCR products were mixed with 5 μL CutSmart® Buffer (New England BioLabs, Ipswich, Mass.), 1 μL of NotI enzyme (New England BioLabs, Ipswich, Mass.) and ultrapure water to a final volume of 50 μL. After incubation for 15 min at 37° C., digested products were purified by PureLink® PCR Purification Kit and analyzed on 2% agarose gel. Gels were imaged with a Gel Doc gel imaging system (Bio-Rad, Hercules, Calif.). Quantification was based on relative band intensities. Gene insertion percentage was determined by the formula, % gene modification=100× (1−(1−fraction cleaved)1/2).

Genome editing detection by TIDE assay. Genomic regions flanking the CRISPR target site (755 bp) were PCR amplified (sequence information can be found in FIG. 34). and products were purified using PureLink® PCR Purification Kit following the manufacturer's protocol. Sanger sequencing was carried out by mixing 20 ng of DNA sample with 4 μL of BigDye® Terminator (Thermo Fisher Scientific, Waltham, Mass.), and ultrapure water to a final volume of 10 μL. After cycle sequencing, samples were analyzed by 3730×1 DNA Analyzer (Applied Biosystems, Foster City, Calif.). Obtained sequences were run on TIDE software (https://tide.nki.nl/) and results were reported as percent gene modification (Brinkman et al., Nucleic Acids Research, 42(22): e168-e168 (2014)).

Miseq analysis. First PCR was carried out on the genomic region flanking the CRISPR target site (755 bp) (sequence information can be found in FIG. 34). and products were purified using PureLink® PCR Purification Kit following the manufacturer's protocol. A second PCR was carried out using primers with Miseq adapter sequences on the genomic region flanking the CRISPR target site (157 bp) and products were purified using PureLink® PCR Purification Kit. Specific bands were checked by running the 5 μL of the sample on 2% agarose gel. Following that, indexing of the DNA was carried out using the Nextera Index kit (96 indexes) (Illumina, San Diego, Calif.) with 8 cycles. Products were purified using PureLink® PCR Purification Kit. Finally, the prepared library was diluted to 4 nM, pooled and analyzed by Illumina HiSeq 2500 (Illumina, San Diego, Calif.). Sequencing reads were analyzed using an in-house bioinformatics pipeline. Paired High-throughput sequencing reads (Miseq) were combined with PAIR [PMID 24142950]. Combined reads were then filtered with a custom python script. Reads without perfect primer sequences were discarded. Primer sequences were trimmed from the reads and then identical sequences were grouped together. A Needleman-Wunsch aligner from the emboss suite was used to align the sequence reads to the reference amplicon [PMID 5420325, Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley]. The options used with this aligner were: -gapopen 10.0, -gapextend 0.5, and -aformat3 sam. The custom python script then reads the Concise Idiosyncratic Gap Alignment Report (CIGAR) string from the Sequence Alignment Map (SAM) output and uses this information to identify and quantify insertions and deletions. Each aligned sequence was also compared to the reference amplicon to identify substitution mutations. Any mutation found in only one read was removed from the analysis. A table containing mutation sequences, read count, and frequency for each mutation was then output for further analysis. In each sequencing run, a control sample consisting of electroporated cells from the same animal prior to transplantation determined the average frequency of mutation classes (insertion, deletion, substitution, insertion and substitution, etc.), and was used to perform a one-tailed binomial t-test on each mutation from the corresponding mutation class. Mutations from experimental samples were retained if they demonstrated a p-value <0.05. All custom scripts are available on request.

In vivo engraftment studies in NSG-mice. All experiments involving animals were conducted in accordance with the controlling institutional guidelines in accordance with the Office of Laboratory Animal Welfare (OLAW) Public Health Assurance (PHS) policy, United States Department of Agriculture (USDA) Animal Welfare Act and Regulations, the Guide for the Care and Use of Laboratory Animals and IACUC protocol No. 1864.

NOD.Cg-Prkdcscidll2rgtm1Wjl/Szj (NOD SCID gamma−/−; NSG) mice were obtained from The Jackson Laboratory and bred in-house in pathogen-free housing conditions. Adult mice (8-12 weeks old) received 175 cGy total body irradiation from a Cesium irradiator followed 3-4 hours later by a single, intrahepatic injection of 1×10⁶ primary human CD34+ hematopoietic cells resuspended in 30 μL of phosphate-buffered saline (PBS; Invitrogen Life Sciences) containing 1% heparin (APP Pharmaceuticals). Four weeks post-engraftment, blood was collected by retro-orbital puncture to determine the level of human blood cells by flow cytometry. Blood was collected every two weeks for the duration of follow-up. White blood cells were isolated and stained with anti-human CD45 antibody (Clone 2D1), CD3 (Clone UCHT1), CD4 (Clone RPA-T4), CD20 (Clone 2H7), and CD14 (Clone M5E2) (all from BD Biosciences, San Jose, Calif.) as previously reported (Haworth et al., Mol Ther Methods Clin Dev, 6: 17-30 (2017)). Stained cells were acquired on a FACS Canto II (BD Biosciences, San Jose, Calif.) and analyzed using FlowJo software v10.1 (Tree Star).

Confocal microscopy imaging. In order to track intracellular biodistribution, Cpf1 crRNA, and HDT were fluorescently tagged by Alexa 488, and Alexa 660 fluorophores on the 5′ end, respectively (IDT, Coralville, Iowa). Au/CRISPR NP were prepared and incubated with cells for 6 h. At the end of incubation cells were washed and dispersed in FluoroBrite™ DMEM media (Gibco, Waltham, Mass.) inside a FluoroDish. Two drops of NucBlue™ Live ReadyProbes™ Reagent (Ex/Em 360/460 nm) (Invitrogen, Carlsbad, Calif.) were added to the cells and incubated for 30 min at room temperature. Finally, cells were imaged on a Zeiss LSM 780 Confocal and Multi-Photon with Airyscan microscope (Zeiss, Germany). Images were analyzed using ZEN Lite software (Zeiss, Germany). Imaging was carried out using a 60× objective after background adjustments.

Statistical analysis. All data are reported as means±standard deviation, and statistical analysis was performed using the paired Student's t-test with GraphPad Prism software, version 7.03 for Windows, (GraphPad Software, USA). A p-value <0.05 was considered as statistically significant.

### Example 3. Targeting Efficiency In Vitro

The goal of this Example will be to show that NP can be targeted to specific blood cell types (HSPC or T cells) in mixed cell populations (unmanipulated blood or bone marrow products).

Currently clinical gene therapy in blood cells requires the target immune cells (e.g., HSPC or T cells) to be purified from other blood cell types. A NP that can specifically bind and deliver gene edits to immune cells without purification would dramatically simplify the current gene therapy manufacturing process, as it would negate the need to purify and culture cells ex vivo for patient-specific cellular therapy. Moreover, this would accelerate the potential for in vivo delivery of gene editing to blood cells, which represents the most globally portable gene therapy strategy. This highly simplified manufacturing strategy is referred to as a “minimal manipulation” approach.

The cell types to be tested in this Example include: 1) primary human HSPC (CD34+ cells and/or CD34+/CD45RA−/CD90+ cells), and 2) primary human T cells (CD3+ and CD4+ cells). Clinically relevant sources for HSPC include bone marrow, granulocyte colony stimulating factor (GCSF) mobilized peripheral blood, and AMD3100 (plerixafor) mobilized peripheral blood. A clinically relevant source for T cells include whole peripheral blood.

The genetic loci to be edited include: 1) the γ-globin promoter in HSPC, which has relevance in hemoglobinopathies such as Sickle Cell Disease; and 2) CCR5 in T cells, which has relevance in the setting of HIV infection.

The targeting molecules to be tested in HSPC include: a) Antibodies that bind: CD34, CD90, or CD133 (tested alone and in combinations of 2); b) Aptamer that binds: CD133 (tested alone and in combination with antibodies or ligands); and c) Ligands: human chorionic gonadotropin (HCG) and SR1 (Stem Regenin 1). The targeting molecules to be tested in T cells include: a) Antibodies that bind: CD3, CD4 (tested alone and in combination); and b) Aptamer: that binds CD3 (tested alone and in combination with antibodies). The chemistry required to add each of these molecule types to the existing NP will utilize amine-to-sulfhydryl, or sulfhydryl to sulfhydryl crosslinkers with various PEG spacers.

Unmanipulated blood cell products from a healthy donor will be divided into aliquots, one for each targeting molecule or combination or set thereof. Each targeting molecule will be tested as the surface displayed cargo of the NP. To track uptake, the guide RNA (innermost layer) will be tagged with a far-red fluorescent dye. Target and non-target cell populations will be tracked with fluorescently-labeled antibodies using different wavelength fluorophores below far-red. The experiment will be repeated across a minimum of 6 and a maximum of 10 unique donors (biological replicates) for each blood cell source noted above.

Confocal microscopy and flow cytometry will be used to assess uptake of the NP by target and non-target cells. For both assays, indications for selection of targeting molecule, cell type, and/or blood products for further testing can include: (i) a minimum of 50% and a maximum of 100% of target cells showing a red fluorescence phenotype, and (ii) a minimum of 0% and a maximum of 20% of non-target cells showing a red fluorescence phenotype. Criteria for selection of targeting molecule, cell type, and/or blood products for further testing can include: (i) a mean value of >50% target cell (HSPC or T cell) red fluorescence observed across donors for at least one experimental group in one clinically relevant cell type, and (ii) 520% red fluorescence observed across donors for any other non-target cell type.

Criteria for elimination of targeting molecule, cell type, and/or blood products from further testing can include: (i)<50% of target cell uptake observed in all experimental conditions tested, or (ii) >20% nontarget cell uptake.

This study will determine which tested targeting molecule best selectively associates NP with desired cell phenotypes in unmanipulated, clinically relevant blood cell products.

### Example 4. Preclinical Evaluation of Minimally Manipulated Cell Products In Vitro

This Example is to demonstrate that the disclosed NP are a clinically viable strategy to achieve “minimal manipulation” of blood cell products for gene therapy, negating the need for purification and culture of target cells ex vivo.

For clinical translation of the targeted NP, feasibility of manufacturing minimally manipulated blood cell products at clinical scale that meet current criteria for reinfusion into a human patient (see Table 3) will be demonstrated. The AuNP-based gene-editing delivery system of the present disclosure, with and without a targeting molecule (identified from Example 3), in unmanipulated human donor blood products at clinical scale will be tested to demonstrate feasibility of scale-up. This feasibility data will be critical for establishing the transformative manufacturing approach for patient-specific cell therapy that does not include purification, culture, electroporation, or engineered viruses.

The specific blood product and cell type associated with indications or criteria for further testing (from Example 3) will be the target for this Example. When more than one cell type and blood product meet criteria for further testing, the highest performing (i.e. highest level of gene editing and best targeting potential) ones will be further tested first, with lesser performing candidates tested thereafter.

The clinically relevant sources for HSPC and T cells are as described in Example 3: (i) bone marrow, GCSF mobilized peripheral blood, and AMD3100 (plerixafor) mobilized peripheral blood for HSPC; and (ii) whole peripheral blood for T cells.

The genetic loci to be edited are as described in Example 3: 1) the γ-globin promoter in HSPC; and 2) CCR5 in T cells.

Blood/bone marrow products from at least three individual donors will be collected. Each product from each donor will be divided into three equal aliquots: one for no treatment (mock control), one for treatment with the (untargeted) AuNP-based gene-editing delivery system of the present disclosure, and one for treatment with the AuNP-based gene-editing delivery system of the present disclosure+ selected targeting molecule.

Assays that will be used in this Example include: fluorescence-assisted cell sorting (FACS) or immunomagnetic bead-based sorting, gene editing analysis, trace element analysis by Inductively Coupled Plasma Mass Spectrometry (ICP-MS), viability assays, and release testing (i.e. suitability for reinfusion testing). For sorting cells by FACS or immunomagnetic beads, the minimum purity of the target cell pool needed to adequately assess all other parameters is >90%, with maximum purity being 100%. There are no threshold requirements for the non-target (negative) fraction purities. For gene editing analysis, the minimum threshold for the target cell phenotype is 20% total gene editing, with a maximum of 50% gene editing; the minimum threshold for the non-target cell phenotype is 0% gene editing and a maximum of 20% gene editing. Products must meet standard release criteria for reinfusion of autologous, gene modified cell products (see Table 3 below). Trace element analysis will be performed on final products formulated for infusion solely for the purpose of understanding what mass of Au is present. There is no minimum threshold and the maximum cannot exceed the total mass added for the initial treatment (maximum of 10 μg/mL of starting cell product). When selection criteria discussed below are met, this data will be used to evaluate biodistribution and clearance in vivo in Example 5.

Criteria for selection of a NP for further testing can include: (i) a mean value of >20% total gene editing observed in target cells only across donors, and (ii) >70% cell viability with all other release criteria met.

This Example can demonstrate that selected NP are suitable for a minimal manipulation approach with human blood cell products or which cell types or blood product components (serum, macrophages, etc.) present the largest hurdle to success.

TABLE 3
Standard release criteria for autologous, genetically
modified cell products to be re-infused.

| Test | Required Result |
|---|---|
| Gram Stain | Negative |
| 3 Day Sterility† | Negative |
| 14 Day Sterility† | Negative |
| Mycoplasma | Negative |
| Endotoxin ^{ε} | ≤0.5 EU/mL |
| Cell Viability by Trypan Blue | ≥70% |

†Final release sterility testing performed by LABS ™ includes bacterial, fungal and yeast testing over 14-day incubation under USP<71> guidelines in controlled cleanrooms.
^{ε} Testing performed by institution quality control using the limulus amebocyte lysate (LAL) test under USP<71> guidelines.

### Example 5. Preclinical Evaluation of Minimally Manipulated Human Cell Products In Vivo

This Example demonstrates preclinical safety and feasibility of a minimally manipulated human blood cell product in an immune-deficient mouse model.

An established model to demonstrate safety and efficacy of genetically modified human blood cells is the xenotransplant. In this model, human blood cells are transplanted into an irradiated immune-deficient mouse. This model permits the cells from one human donor to be transplanted across many individual mice. Parameters that can be studied in this model include blood cell performance in the animal, toxicity, biodistribution, and clearance. Importantly, it is anticipated that some AuNP can still be present in a minimally manipulated blood cell product at the time of reinfusion, and this study can aid in understanding the physiological impacts of NP administration. This information is important for clinical translation of the approach and will also be informative for direct in vivo administration studies. In this Example, the minimally manipulated human blood cell products selected for further study (from Example 4) will be injected into sub-lethally irradiated immune-deficient mice to monitor cell performance (engraftment), and biodistribution and clearance of any residual NP which are infused along with the blood cell product. This can be considered to be a “de-risking” experiment for the disclosed technology.

The specific blood product and cell type selected for further study from Example 3 will be the target for these studies.

The clinically relevant sources for HSPC and T cells are as described in Examples 3 and 4: (i) bone marrow, GCSF mobilized peripheral blood, and AMD3100 (plerixafor) mobilized peripheral blood for HSPC; and (ii) whole peripheral blood for T cells.

The genetic loci to be edited are as described in Examples 3 and 4: 1) the γ-globin promoter in HSPC; and 2) CCR5 in T cells.

The minimally-manipulated blood/bone marrow products from three individual donors in Example 4 will be infused into immune deficient mice within 12-24 hours after sub-lethal total body irradiation. Human cell engraftment will be monitored over time after transplant, as well as engraftment of gene edited cells and overall health and wellness of the animals. Imaging, urine, and feces can be obtained from these mice following infusion to determine biodistribution and clearance of NP which may be present in the infusion product.

Assays and experiments that will be conducted in the study include: Visual monitoring of health of the infused mice (grooming, weight and activity level); hematologic recovery after transplant; engraftment and persistence of gene edited cells; trace element analysis of the infused product by ICP-MS; and analysis of the urine and feces by ICP-MS for 72 hours after infusion to determine whether all NP have been cleared (mass balance). If bioaccumulation is indicated, micro computed tomography (CT) imaging of live mice can be performed to assess the location of accumulation. If accumulation is too low to visualize with micro CT, a necropsy and additional trace element analysis by ICP-MS can be performed to determine sites for bioaccumulation. The micro CT, necropsy, and/or trace element analysis can be combined with histopathology to assess potential toxicity. Readout thresholds for these various assays are described in the next few paragraphs.

Engraftment and persistence. Flow cytometry can be used to assess levels of human CD45-expressing cells in blood, bone marrow, and spleen. The minimum threshold is 0%, and the maximum threshold is 100%.

Gene editing analysis. The minimum threshold is 5% in human cells, and the maximum threshold is 100%. It is not anticipated that sufficient NP will remain in the formulation to edit mouse cells; however, assays will evaluate whether gene editing is detected in mouse CD45-expressing cells or any tissues displaying bioaccumulation as described below.

Health monitoring. Pain and distress evaluation (min PD1, max PD4) and body condition evaluation (min BC1, max BC5) will be performed for each mouse prior to administration of NP, then daily for 3 days after administration of NP, and weekly thereafter. Scoring is based on that published by Burkholder et al. Health Evaluation of Experimental Laboratory Mice. Current Protocols in Mouse Biology, 2012; 2:145-165. Any adverse effects will be recorded and summarized.

Trace element analysis. The minimum threshold in urine/feces over 72 hours is 0, and the maximum threshold cannot exceed total mass injected. The minimum threshold in tissues is 0, and the maximum threshold cannot exceed total mass injected.

Micro-CT imaging. The minimum threshold is no contrast enhancement, and the maximum threshold is to be determined.

Histopathology. The assay will assess notable organ toxicity relative to untreated controls from all donors. The minimum threshold is no toxicity, and the maximum threshold is graded using adverse event criteria as published for each target organ.

The study described in this Example will establish preclinical in vivo safety and efficacy of minimally-manipulated human blood products.

### (XIV) CLOSING PARAGRAPHS

The disclosed nucleic acid sequences are shown using standard letter abbreviations for nucleotide bases, as defined in 37 C.F.R. 1.822. Only one strand of each nucleic acid sequence is shown, but the complementary strand is understood as included.

Variants of protein and/or nucleic acid sequences disclosed herein can also be used. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein wherein the variant exhibits substantially similar or improved biological function.

“% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

In particular embodiments, variant proteins include conservative amino acid substitutions. In particular embodiments, a conservative amino acid substitution may not substantially change the structural characteristics of the reference sequence (e.g., a replacement amino acid should not tend to break a helix that occurs in the reference sequence or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105 (1991).

In particular embodiments, a “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (lie), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and lie. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, lie, Val, and Cys; and large aromatic residues: Phe, Tyr, and Trp. Additional information is found in Creighton (1984) Proteins, W.H. Freeman and Company.

In particular embodiments “affinity” refers to the strength of the sum total of noncovalent interactions between a single binding site of an antibody and its target marker. Unless indicated otherwise, “binding affinity” refers to intrinsic binding affinity which reflects a 1:1 interaction between members of a binding pair (i.e., antibody and target marker). The affinity of an antibody for its target marker can generally be represented by the dissociation constant (Kd) or the association constant (K_{A}). Affinity can be measured by common methods known in the art.

As is understood by one of ordinary skill in the art, there are a number of commercially available antibodies and targeting ligands that bind the cellular markers described herein.

In particular embodiments, binding affinities can be assessed in relevant in vitro conditions, such as a buffered salt solution approximating physiological pH (7.4) at room temperature or 37° C.

In particular embodiments, “bind” means that the antibody associates with its target marker with a dissociation constant (1(D) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷ M, in particular embodiments of from 10⁻⁸ M to 10⁻¹³ M, or in particular embodiments of from 10⁻⁹ M to 10⁻¹³ M. The term can be further used to indicate that the antibody does not bind to other biomolecules present, (e.g., it binds to other biomolecules with a dissociation constant (KD) of 10⁻⁴ M or more, in particular embodiments of from 10⁻⁴ M to 1 M).

In particular embodiments, “bind” means that the antibody associates with its target marker with an affinity constant (i.e., association constant, K_{A}) of 107 M⁻¹ or more, in particular embodiments of from 10⁵ M⁻¹ to 10¹³ M⁻¹, in particular embodiments of from 10⁵ M⁻¹ to 10¹⁰ M⁻¹, in particular embodiments of from 10⁵ M⁻¹ to 10⁸ M⁻¹, in particular embodiments of from 107 M⁻¹ to 10¹³ M⁻¹, or in particular embodiments of from 107 M⁻¹ to 10⁸ M⁻¹. The term can be further used to indicate that the antibody does not bind to other biomolecules present, (e.g., it binds to other biomolecules with an association constant (K_{A}) of 10⁴ M⁻¹ or less, in particular embodiments of from 10⁴ M⁻¹ to 1 M⁻¹).

As indicated particular embodiments can utilize variants of targeting ligand binding domains. Variants of targeting ligand binding domains can include those having one or more conservative amino acid substitutions or one or more non-conservative substitutions that do not adversely affect the binding of the antibody to the targeted epitope.

In particular embodiments, a V_{L} region can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared to an antibody produced and characterized according to methods disclosed herein. An insertion, deletion or substitution may be anywhere in the V_{L} region, including at the amino- or carboxy-terminus or both ends of this region, provided that each CDR includes zero changes or at most one, two, or three changes and provided an antibody including the modified V_{L} region can still specifically bind the targeted epitope with an affinity similar to the reference antibody.

In particular embodiments, a V_{H} region can be derived from or based on a disclosed V_{H} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with an antibody produced and characterized according to methods disclosed herein. An insertion, deletion or substitution may be anywhere in the V_{H} region, including at the amino- or carboxy-terminus or both ends of this region, provided that each CDR includes zero changes or at most one, two, or three changes and provided an antibody including the modified V_{H} region can still specifically bind its target epitope with an affinity similar to the reference antibody.

Reference to CD34, CD45RA, CD90, CD117, CD123, CD133, CD164 and other CDs described herein are understood by those of ordinary skill in the art. For other readers, CD (clusters of differentiation) antigens are proteins expressed on the surface of a cell that are detectable via specific antibodies. CD34 is a highly glycosylated type I transmembrane protein expressed on 1-4% of bone marrow cells. CD45RA is related to fibronectin type Ill, has a molecular weight of 205-220 kDa and is expressed on B cells, naïve T cells, and monocytes. CD90 is a GPI-cell anchored molecule found on prothymocyte cells in humans. CD117 is the c-kit ligand receptor found on 1-4% of bone marrow stem cells. CD123A is related to the cytokine receptor superfamily and the fibronectin type Ill superfamily, has a molecular weight of 70 kDa and is expressed on bone marrow stem cells granulocytes, monocytes and megakaryocytes. CD133 is a pentaspan transmembrane glycoprotein expressed on primitive hematopoietic progenitor cells and other stem cells. CD164 is a type I integral transmembrane sialomucin expressed by human hematopoietic progenitor cells and bone marrow stromal cells.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in the ability to selectively genetically modify an intended cell type within an ex vivo blood cell product that has been subject to minimal manipulation.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; 18% of the stated value; 17% of the stated value; 16% of the stated value; 15% of the stated value; 14% of the stated value; ±13% of the stated value; ±12% of the stated value; 11% of the stated value; 10% of the stated value; ±9% of the stated value; 8% of the stated value; 7% of the stated value; 6% of the stated value; 5% of the stated value; 4% of the stated value; 3% of the stated value; 2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein).

Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of genetically modifying a hematopoietic stem and progenitor cell (HSPC) population in a biological sample comprising adding a gold nanoparticle (AuNP) to the biological sample, wherein the AuNP comprises
a gold (Au) core that is less than 20 nm in diameter;
a Clustered Regularly Interspaced Short Palindromic Repeat (CRISPR) guide RNA (crRNA)-nuclease ribonucleoprotein (RNP) complex wherein the crRNA comprises a 3′ end and a 5′ end, wherein the 3′ end is conjugated to a spacer with a thiol modification, and the 5′ end is conjugated to the nuclease, and wherein the thiol modification is covalently linked to the surface of the Au core and wherein the crRNA has a sequence set forth in SEQ ID NO: 262; SEQ ID NO: 13; SEQ ID NO: 14; or SEQ ID NO: 241-261;
a positively-charged polyethyleneimine polymer coating wherein the positively-charged polyethyleneimine polymer has a molecular weight of less than 2500 daltons, surrounds the RNP complex, and contacts the surface of the Au core; and
a donor template comprising a homology-directed repair template (HDT) on the surface of the positively-charged polymer coating wherein the HDT template comprises a sequence set forth in SEQ ID NO: 48; SEQ ID NO: 4; SEQ ID NO: 15; SEQ ID NO: 33-41; SEQ ID NO: 44-47; or SEQ ID NO: 49-51; and
a CD133 targeting ligand comprising a binding domain of antibody clone REA820, REA753, REA816, 293C3, AC141, AC133, or 7
wherein the targeting ligand is linked to the nuclease through an amine-to-sulfhydryl crosslinker or a sulfhydryl-to-sulfhydryl crosslinker and
wherein the HSPC population has not been exposed to electroporation, a viral vector encoding an HDT, or a magnetic cell separation process, and wherein the method results in no more than 30% HSPC cellular toxicity and provides a gene-editing efficiency within the HSPC population of at least 10%.

2. The method of claim 1, wherein the crRNA targets a sequence set forth in SEQ ID NO: 25; SEQ ID NO: 3; SEQ ID NO: 24; SEQ ID NO: 26-32; SEQ ID NO: 42; SEQ ID NO: 43; or SEQ ID NO: 214-224.

3. The method of claim 1, wherein the crRNA has a sequence as set forth in SEQ ID NO: 262, SEQ ID NO: 261 or SEQ ID NO: 259.

4. The method of claim 1, wherein the nuclease comprises Cpf1 or Cas9.

5. The method of claim 1, wherein the positively-charged polymer coating comprises polyethyleneimine with a molecular weight of 2000 daltons.

6. The method of claim 1, wherein the weight/weight (w/w) ratio of Au core to nuclease is 0.6.

7. The method of claim 1, wherein the w/w ratio of Au core to HDT is 1.0.

8. A method of genetically modifying a selected cell population in a biological sample comprising adding a gold nanoparticle (AuNP) to the biological sample, wherein the AuNP comprises
a gold (Au) core that is less than 30 nm in diameter;
a guide RNA (gRNA)-nuclease ribonucleoprotein (RNP) complex wherein the gRNA comprises a 3′ end and a 5′ end, wherein the 3′ end is conjugated to a spacer with a chemical modification, and the 5′ end is conjugated to the nuclease, and wherein the chemical modification is covalently linked to the surface of the Au core;
a positively-charged polymer coating wherein the positively-charged polymer has a molecular weight of less than 2500 daltons, surrounds the RNP complex, and contacts the surface of the Au core; and
a donor template comprising a homology-directed repair template (HDT) on the surface of the positively-charged polymer coating
wherein the selected cell population has not been exposed to electroporation or a viral vector encoding an and wherein the method results in no more 30% cellular toxicity of the selected cell population and provides a gene-editing efficiency within the selected cell population of at least 10%.

9. The method of claim 8, wherein the weight/weight (w/w) ratio of Au core to nuclease is 0.6.

10. The method of claim 8, wherein the w/w ratio of Au core to HDT is 1.0.

11. The method of claim 8, wherein the AuNP is less than 70 nm in diameter.

12. The method of claim 8, wherein the AuNP has a polydispersity index (PDI) of less than 0.2.

13. The method of claim 8, wherein the gRNA comprises a Clustered Regularly Interspaced Short Palindromic Repeat (CRISPR) crRNA.

14. The method of claim 13, wherein the crRNA targets a sequence as set forth in SEQ ID NO: 1; SEQ ID NO: 3; SEQ ID NO: 20-32; SEQ ID NO: 42; SEQ ID NO: 43; SEQ ID NO: 84-97; or SEQ ID NO: 214-224.

15. The method of claim 13, wherein the crRNA comprises a sequence set forth in SEQ ID NO: 5; SEQ ID NO: 6; SEQ ID NO: 13; SEQ ID NO: 14; or SEQ ID NO: 225-264.

16. The method of claim 8, wherein the nuclease comprises Cpf1 or Cas9.

17. The method of claim 8, wherein the positively-charged polymer coating comprises polyethyleneimine (PEI), polyamidoamine (PAMAM); polylysine (PLL), polyarginine; cellulose, dextran, spermine, spermidine, or poly(vinylbenzyl trialkyl ammonium).

18. The method of claim 8, wherein the positively-charged polymer has a molecular weight of 1500-2500 daltons.

19. The method of claim 8, wherein the positively-charged polymer has a molecular weight of 2000 daltons.

20. The method of claim 8, wherein the chemical modification comprises a free thiol, amine, or carboxylate functional group.

21. The method of claim 8, wherein the spacer comprises an oligoethylene glycol spacer.

22. The method of claim 21, wherein the oligoethylene glycol spacer comprises an 18 atom oligoethylene glycol spacer.

23. The method of claim 8, wherein the HDT comprises sequences having homology to genomic sequences undergoing modification.

24. The method of claim 23, wherein the HDT comprises a sequence as set forth in SEQ ID NO: 2; SEQ ID NO: 4; SEQ ID NO: 8; SEQ ID NO: 15; SEQ ID NO: 33-41; or SEQ ID NO: 44-52.

25. The method of claim 8, wherein the HDT comprises single-stranded DNA (ssDNA).

26. The method of claim 8, wherein the donor template comprises a therapeutic gene.

27. The method of claim 26, wherein the therapeutic gene comprises or encodes skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72, α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, FancI, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT−1, YES, zac1, iduronidase, IDS, GNS, HGSNAT, SGSH, NAGLU, GUSB, GALNS, GLB1, ARSB, HYAL1, F8, F9, HBB, CYB5R3, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.

28. The method of claim 8, wherein the AuNP further comprises a targeting ligand linked to the nuclease.

29. The method of claim 28, wherein the AuNP with the linked targeting ligand is 60-150 nm in diameter.

30. The method of claim 28, wherein the targeting ligand comprises a binding molecule that binds CD3, CD4, CD34, CD46, CD90, CD133, CD164, a luteinizing hormone-releasing hormone (LHRH) receptor, or an aryl hydrocarbon receptor (AHR).

31. The method of claim 28, wherein the targeting ligand comprises an anti-human CD3 antibody or antigen binding fragment thereof, an anti-human CD4 antibody or antigen binding fragment thereof, an anti-human CD34 antibody or antigen binding fragment thereof, an anti-human CD46 antibody or antigen binding fragment thereof, an anti-human CD90 antibody or antigen binding fragment thereof, an anti-human CD133 antibody or antigen binding fragment thereof, an anti-human CD164 antibody or antigen binding fragment thereof, an anti-human CD133 aptamer, a human luteinizing hormone, a human chorionic gonadotropin, degerelix acetate, or StemRegenin 1.

32. The method of claim 28, wherein the targeting ligand comprises antibody clone: 581; antibody clone: 561; antibody clone: REA 164; antibody clone: AC136; antibody clone: 5E10; antibody clone: DG3; antibody clone: REA897; antibody clone: REA820; antibody clone: REA753; antibody clone: REA816; antibody clone: 293C3; antibody clone: AC141; antibody clone: AC133; antibody clone: 7; aptamer A15; aptamer B19; HCG (Protein/Ligand); or Luteinizing hormone (LH Protein/Ligand).

33. The method of claim 28, wherein the nuclease and targeting ligand are linked through an amino acid linker.

34. The method of claim 33, wherein the amino acid linker comprises a direct amino acid linker, a flexible amino acid linker, or a tag-based amino acid linker.

35. The method of claim 28, wherein the nuclease and targeting ligand are linked through polyethylene glycol (PEG).

36. The method of claim 28, wherein the nuclease and targeting ligand are linked through an amine-to-sulfhydryl crosslinker or a or sulfhydryl to sulfhydryl crosslinker.

37. The method of claim 28, wherein the nuclease and targeting ligand are linked through PEG and an amine-to-sulfhydryl crosslinker or are linked through PEG and a sulfhydryl to sulfhydryl crosslinker.

38. The method of claim 28, wherein the selected cell population has not undergone a magnetic separation process to remove the selected cells from the biological sample.

39. The method of claim 8, wherein the selected cell population comprises a blood cell selected from a hematopoietic stem cell (HSC), a hematopoietic progenitor cell (HPC), a hematopoietic stem and progenitor cell (HSPC), a T cell, a natural killer (NK) cell, a B cell, a macrophage, a monocyte, a mesenchymal stem cell (MSC), a white blood cell (WBC), a mononuclear cell (MNC), an endothelial cell (EC), a stromal cell, and/or a bone marrow fibroblast.

40. The method of claim 39, wherein the blood cell comprises a CD34⁺CD45RA⁻CD90⁺ HSC; a CD34⁺/CD133⁺ HSC; an LH⁺ HSC; a CD34⁺CD90⁺ HSPC; a CD34⁺CD90⁺ CD133⁺ HSPC; and/or an AHR⁺ HSPC.

41. The method of claim 39, wherein the blood cell comprises a CD3⁺ T cell and/or a CD4⁺ T cell.

42. The method of claim 8, wherein the biological sample comprises peripheral blood, bone marrow, granulocyte colony stimulating factor (GCSF) mobilized peripheral blood, and/or plerixafor mobilized peripheral blood.

43. The method of claim 8, wherein the adding is in an amount of 1, 2, 3, 4, 5, 8, 10, 12, 15, or 20 μg of AuNP per milliliter (mL) of biological sample.

44. The method of claim 42, wherein the biological sample and the added AuNP are incubated for 1-48 hours.

45. The method of claim 42, wherein the biological sample and the added AuNP are incubated until testing confirms the uptake of the AuNP into cells.

46. The method of claim 45, wherein the testing comprises confocal microscopy imaging, inductively coupled plasma (ICP)-mass spectrometry (ICP-MS), ICP-atomic emission spectroscopy (ICP-AES), or ICP-optical emission spectroscopy (ICP-OES).

47. A cell modified according to a method of claim 8.

48. A therapeutic formulation comprising a cell of claim 47.

49. A method of providing a therapeutic nucleic acid sequence to a subject in need thereof comprising administering a cell of claim 47 or a therapeutic formulation of claim 48 to the subject thereby providing a therapeutic nucleic acid sequence to the subject.

50. A gold nanoparticle (AuNP) comprising
a gold (Au) core that is less than 30 nm in diameter;
a guide RNA-nuclease ribonucleoprotein (RNP) complex wherein the gRNA comprises a 3′ end and a 5′ end, wherein the 3′ end is conjugated to a spacer with a chemical modification, and the 5′ end is conjugated to the nuclease, and wherein the chemical modification is covalently linked to the surface of the Au core;
a positively-charged polymer coating wherein the positively-charged polymer has a molecular weight of less than 2500 daltons, surrounds the RNP complex, and contacts the surface of the Au core; and
a donor template comprising a homology-directed repair template (HDT) on the surface of the positively-charged polymer coating.

51. The AuNP of claim 50, wherein the weight/weight (w/w) ratio of Au core to nuclease is 0.6.

52. The AuNP of claim 50, wherein the w/w ratio of Au core to HDT is 1.0.

53. The AuNP of claim 50, wherein the AuNP is less than 70 nm in diameter.

54. The AuNP of claim 50, wherein the AuNP has a polydispersity index (PDI) of less than 0.2.

55. The AuNP of claim 50, wherein the gRNA comprises a Clustered Regularly Interspaced Short Palindromic Repeat (CRISPR) crRNA.

56. The AuNP of claim 55, wherein the crRNA targets a sequence as set forth in SEQ ID NO: 1; SEQ ID NO: 3; SEQ ID NO: 20-32; SEQ ID NO: 42; SEQ ID NO: 43; SEQ ID NO: 84-97; or SEQ ID NO: 214-224.

57. The AuNP of claim 55, wherein the crRNA comprises a sequence as set forth in SEQ ID NO: 5; SEQ ID NO: 6; SEQ ID NO: 13; SEQ ID NO: 14; or SEQ ID NO: 225-264.

58. The AuNP of claim 50, wherein the nuclease comprises Cpf1 or Cas9.

59. The AuNP of claim 50, wherein the positively-charged polymer coating comprises polyethyleneimine (PEI), polyamidoamine (PAMAM); polylysine (PLL), polyarginine; cellulose, dextran, spermine, spermidine, or poly(vinylbenzyl trialkyl ammonium).

60. The AuNP of claim 50, wherein the positively-charged polymer has a molecular weight of 1500-2500 daltons.

61. The AuNP of claim 50, wherein the positively-charged polymer has a molecular weight of 2000 daltons.

62. The AuNP of claim 50, wherein the chemical modification comprises a free thiol, amine, or carboxylate functional group.

63. The AuNP of claim 50, wherein the spacer comprises an oligoethylene glycol spacer.

64. The AuNP of claim 63, wherein the oligoethylene glycol spacer comprises an 18 atom oligoethylene glycol spacer.

65. The AuNP of claim 50, wherein the HDT comprises sequences having homology to genomic sequences undergoing modification.

66. The AuNP of claim 65, wherein the HDT comprises a sequence set forth in SEQ ID NO: 2; SEQ ID NO: 4; SEQ ID NO: 8; SEQ ID NO: 15; SEQ ID NO: 33-41; or SEQ ID NO: 44-52.

67. The AuNP of claim 50, wherein the HDT comprises single-stranded DNA (ssDNA).

68. The AuNP of claim 50, wherein the donor template comprises a therapeutic gene.

69. The AuNP of claim 68, wherein the therapeutic gene encodes skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72, α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, FancI, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT−1, YES, zac1, iduronidase, IDS, GNS, HGSNAT, SGSH, NAGLU, GUSB, GALNS, GLB1, ARSB, HYAL1, F8, F9, HBB, CYB5R3, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.

70. The AuNP of claim 50, wherein the AuNP further comprises a targeting ligand linked to the nuclease.

71. The AuNP of claim 70, wherein the targeting ligand comprises a binding molecule that binds CD3, CD4, CD34, CD46, CD90, CD133, CD164, a luteinizing hormone-releasing hormone (LHRH) receptor, or an aryl hydrocarbon receptor (AHR).

72. The AuNP of claim 70, wherein the targeting ligand comprises an anti-human CD3 antibody or antigen binding fragment thereof, an anti-human CD4 antibody or antigen binding fragment thereof, an anti-human CD34 antibody or antigen binding fragment thereof, an anti-human CD46 antibody or antigen binding fragment thereof, an anti-human CD90 antibody or antigen binding fragment thereof, an anti-human CD133 antibody or antigen binding fragment thereof, an anti-human CD164 antibody or antigen binding fragment thereof, an anti-human CD133 aptamer, a human luteinizing hormone, a human chorionic gonadotropin, degerelix acetate, or StemRegenin 1.

73. The AuNP of claim 70, wherein the targeting ligand comprises antibody clone: 581; antibody clone: 561; antibody clone: REA1164; antibody clone: AC136; antibody clone: 5E10; antibody clone: DG3; antibody clone: REA897; antibody clone: REA820; antibody clone: REA753; antibody clone: REA816; antibody clone: 293C3; antibody clone: AC141; antibody clone: AC133; antibody clone: 7; aptamer A15; aptamer B19; HCG (Protein/Ligand); Luteinizing hormone (LH Protein/Ligand); or a binding fragment derived from any of the foregoing.

74. The AuNP of claim 70, wherein the nuclease and targeting ligand are linked through an amino acid linker.

75. The AuNP of claim 74, wherein the amino acid linker comprises a direct amino acid linker, a flexible amino acid linker, or a tag-based amino acid linker.

76. The AuNP of claim 70, wherein the nuclease and targeting ligand are linked through polyethylene glycol (PEG).

77. The AuNP of claim 70, wherein the nuclease and targeting ligand are linked through an amine-to-sulfhydryl crosslinker.

78. A composition comprising the AuNP of claim 8 and a biological sample comprising a selected cell population.

79. The composition of claim 78, wherein the biological sample comprises a selected cell population comprising a blood cell selected from a hematopoietic stem cell (HSC), a hematopoietic progenitor cell (HPC), a hematopoietic stem and progenitor cell (HSPC), a T cell, a natural killer (NK) cell, a B cell, a macrophage, a monocyte, a mesenchymal stem cell (MSC), a white blood cell (WBC), a mononuclear cell (MNC), an endothelial cell (EC), a stromal cell, and/or a bone marrow fibroblast.

80. The composition of claim 79, wherein the blood cell comprises a CD34⁺CD45RA−CD90⁺ HSC; a CD34⁺/CD133⁺ HSC; an LH⁺ HSC; a CD34⁺CD90⁺ HSPC; a CD34⁺CD90⁺CD133⁺ HSPC; and/or an AHR⁺ HSPC.

81. The composition of claim 79, wherein the blood cell comprises a CD3⁺ T cell and/or a CD4⁺ T cell.

82. The composition of claim 78, wherein the biological sample comprises peripheral blood, bone marrow, granulocyte colony stimulating factor (GCSF) mobilized peripheral blood, and/or plerixafor mobilized peripheral blood.

83. The composition of claim 78, wherein AuNP is within the biological sample in an amount of 1, 2, 3, 4, 5, 8, 10, 12, 15, or 20 μg of AuNP per milliliter (mL) of biological sample.


SEQUENCE LISTING (264 sequences)

SEQ ID NO: 1 - DNA, 43 bp, Artificial Sequence
  Target locus on CCR5 gene
aagctcagtttacacccgatccactggggagcaggaaatatct

SEQ ID NO: 2 - DNA, 88 bp, Artificial Sequence
  Homology template
  misc_feature (1)..(1): Optional Alexa660N at 5′ end of sequence
ccacttgagtccgtgtcacaagcccacagatatttcctgcgcggccgctccccagtggatcgggtgtaaactgagcttgctcgctcgg

SEQ ID NO: 3 - DNA, 44 bp, Artificial Sequence
  Target locus within gamma-globin gene promoter
tggtcaagtttgccttgtcaaggctattggtcaaggcaaggctg

SEQ ID NO: 4 - DNA, 60 bp, Artificial Sequence
  Homology template
tactctaagactattggtcaagttcgccttgtcaaggcaaggctggccaacccatgggtg

SEQ ID NO: 5 - RNA, 41 nt, Artificial Sequence
  crRNA
  misc_feature (1)..(1): Optional Alexa488N at the 5′ end of the sequence
  misc_feature (1)..(1): Optional AltR1 at 5′ end of the sequence
  misc_feature (41)..(41): Optional 18-atom hexa-ethyleneglycol spacer (iSp18) and a thiol modifier C3 S-S (thioMC3-D) at the 3′ end of the sequence
uaauuucuacucuuguagaucacccgauccacuggggagca

SEQ ID NO: 6 - RNA, 36 nt, Artificial Sequence
  Cas9 crRNA
  misc_feature (1)..(1): A thiol modifier C6 S-S (thioMC6-D) and an 18-atom hexa-ethyleneglycol spacer (iSp18) are located at 5′ end of sequence
  misc_feature (36)..(36): An optional AltR2 at the 3′ end of the sequence
cacccgauccacuggggagcguuuuagagcuaugcu

SEQ ID NO: 7 - RNA, 67 nt, Artificial Sequence
  Cas9tracrRNA
agcauagcaaguuaaaauaaggcuaguccguuaucaacuugaaaaaguggcaccgagucggugcuuu

SEQ ID NO: 8 - DNA, 88 bp, Artificial Sequence
  CCR5 HDT templatefor target strand
ccgagcgagcaagctcagtttacacccgatccactggggagcggccgcgcaggaaatatctgtgggcttgtgacacggactcaagtgg

SEQ ID NO: 9 - DNA, 21 bp, Artificial Sequence
  CCR5 Forward primer
agatagtcatcttggggctgg

SEQ ID NO: 10 - DNA, 21 bp, Artificial Sequence
  CCR5 Reverse primer
ggagtgaagggagagtttgtc

SEQ ID NO: 11 - DNA, 53 bp, Artificial Sequence
  CCR5 Forward primer
tcgtcggcagcgtcagatgtgtataagagacagacattgccaaacgcttctgc

SEQ ID NO: 12 - DNA, 54 bp, Artificial Sequence
  CCR5 Reverse primer
gtctcgtgggctcggagatgtgtataagagacagtgcacaactctgactgggtc

SEQ ID NO: 13 - RNA, 41 nt, Artificial Sequence
  gamma-globin Cpf1 crRNA
  misc_feature (41)..(41): An 18-atom hexa-ethyleneglycol spacer (iSp18) and a thiol modifier C3 S-S (thioMC3-D) at the 3′ end of the sequence
uaauuucuacucuuguagauccuugucaaggcuauugguca

SEQ ID NO: 14 - RNA, 36 nt, Artificial Sequence
  gamma-globin Cas9 crRNA
  misc_feature (1)..(1): A thiol modifier C6 S-S (thioMC6-D) and an 18-atom hexa-ethyleneglycol spacer (iSp18) are located at 5′ end of sequence
cuugucaaggcuauuggucaguuuuagagcuaugcu

SEQ ID NO: 15 - DNA, 60 bp, Artificial Sequence
  gamma-globin HDT templatefor non-target strand
cacccatgggttggccagccttgccttgacaaggcgaacttgaccaatagtcttagagta

SEQ ID NO: 16 - DNA, 20 bp, Artificial Sequence
  gamma-globin forward primer
ccttcttgccatgtgccttg

SEQ ID NO: 17 - DNA, 25 bp, Artificial Sequence
  gamma-globin reverse primer
tctatggtgggagaagaaaactagc

SEQ ID NO: 18 - DNA, 49 bp, Artificial Sequence
  gamma-globin forward primer
tcgtcggcagcgtcagatgtgtataagagacagggcccctggcctcact

SEQ ID NO: 19 - DNA, 59 bp, Artificial Sequence
  gamma-globin reverse primer
gtctcgtgggctcggagatgtgtataagagacagtcaatgcaaatatctgtctgaaacg

SEQ ID NO: 20 - DNA, 25 bp, Artificial Sequence
  CCR5 Cpf1 crRNA
  misc_feature (4)..(4): n is a, c, g, or t
tttncacccgatccactggggagca

SEQ ID NO: 21 - DNA, 25 bp, Artificial Sequence
  target site
  misc_feature (1)..(3): PAM site
tttacacccgatccactggggagca

SEQ ID NO: 22 - DNA, 23 bp, Artificial Sequence
  CCR5 Cas9 crRNA
  misc_feature (21)..(21): n is a, c, g, or t
cacccgatccactggggagcngg

SEQ ID NO: 23 - DNA, 23 bp, Artificial Sequence
  CCR5 Cas9 DNA
cacccgatccactggggagcagg

SEQ ID NO: 24 - DNA, 25 bp, Artificial Sequence
  gamma-globinCpf1 crRNA
  misc_feature (4)..(4): n is a, c, g, or t
tttnccttgtcaaggctattggtca

SEQ ID NO: 25 - DNA, 25 bp, Artificial Sequence
  Cpf1 Guide
tttgccttgtcaaggctattggtca

SEQ ID NO: 26 - DNA, 24 bp, Artificial Sequence
  gamma-globin Cas9 crRNA
  misc_feature (22)..(22): n is a, c, g, or t
ccttgtcaaggctattggtcangg

SEQ ID NO: 27 - DNA, 24 bp, Artificial Sequence
  gamma-globin Cas9 DNA
ccttgtcagggctgttggtcgagg

SEQ ID NO: 28 - DNA, 23 bp, Artificial Sequence
  Cas9 Guide
gtggggaaggggcccccaagagg

SEQ ID NO: 29 - DNA, 23 bp, Artificial Sequence
  Cas9 Guide
attgagatagtgtggggaagggg

SEQ ID NO: 30 - DNA, 23 bp, Artificial Sequence
  Cas9 Guide
cattgagatagtgtggggaaggg

SEQ ID NO: 31 - DNA, 23 bp, Artificial Sequence
  Cas9 Guide
gcattgagatagtgtggggaagg

SEQ ID NO: 32 - DNA, 23 bp, Artificial Sequence
  Cas9 Guide
atttgcattgagatagtgtgggg

SEQ ID NO: 33 - DNA, 23 bp, Artificial Sequence
  Cas9 HDR Template
  misc_feature (1)..(1): A homology arm is located at the 5′ end of the sequence
  misc_feature (23)..(23): A homology arm is located at the 3′ end of the sequence
gtggggaaggcgcccccaagagg

SEQ ID NO: 34 - DNA, 23 bp, Artificial Sequence
  Cas9 HDR Template
  misc_feature (1)..(1): A homology arm is located at the 5′ end of the sequence
  misc_feature (23)..(23): A homology arm is located at the 3′ end of the sequence
gtggagaaggggcccccaagagg

SEQ ID NO: 35 - DNA, 23 bp, Artificial Sequence
  Cas9 HDR Template
  misc_feature (1)..(1): A homology arm is located at the 5′ end of the sequence
  misc_feature (23)..(23): A homology arm is located at the 3′ end of the sequence
gtggagaaggcgcccccaagagg

SEQ ID NO: 36 - DNA, 23 bp, Artificial Sequence
  Cas9 HDR Template
  misc_feature (1)..(1): A homology arm is located at the 5′ end of the sequence
  misc_feature (23)..(23): A homology arm is located at the 3′ end of the sequence
gtttgcattgagatagtgtgggg

SEQ ID NO: 37 - DNA, 23 bp, Artificial Sequence
  Cas9 HDR Template
  misc_feature (1)..(1): A homology arm is located at the 5′ end of the sequence
  misc_feature (23)..(23): A homology arm is located at the 3′ end of the sequence
gctattggttaaggcaaggctgg

SEQ ID NO: 38 - DNA, 23 bp, Artificial Sequence
  Cas9 HDR Template
  misc_feature (1)..(1): A homology arm is located at the 5′ end of the sequence
  misc_feature (23)..(23): A homology arm is located at the 3′ end of the sequence
gctattagtcaaggcaaggctgg

SEQ ID NO: 39 - DNA, 23 bp, Artificial Sequence
  Cas9 HDR Template
  misc_feature (1)..(1): A homology arm is located at the 5′ end of the sequence
  misc_feature (23)..(23): A homology arm is located at the 3′ end of the sequence
gctattagttaaggcaaggctgg

SEQ ID NO: 40 - DNA, 10 bp, Artificial Sequence
  Cas9 HDR Template
  misc_feature (1)..(1): A homology arm is located at the 5′ end of the sequence
  misc_feature (10)..(10): A homology arm is located at the 3′ end of the sequence
gtttgccttg

SEQ ID NO: 41 - DNA, 25 bp, Artificial Sequence
  Cas9 HDR Template
  misc_feature (1)..(1): A homology arm is located at the 5′ end of the sequence
  misc_feature (25)..(25): A homology arm is located at the 3′ end of the sequence
tttgccttagttaaggcaaggctgg

SEQ ID NO: 42 - DNA, 25 bp, Artificial Sequence
  Cpf1 Guide
tttgcattgagatagtgtggggaag

SEQ ID NO: 43 - DNA, 25 bp, Artificial Sequence
  Cpf1 Guide
tttagccagggaccgtttcagacag

SEQ ID NO: 44 - DNA, 33 bp, Artificial Sequence
  Cpf1 HDR Template
  misc_feature (1)..(1): A homology arm is located at the 5′ end of the sequence
  misc_feature (33)..(33): A homology arm is located at the 3′ end of the sequence
tttgcattgagatagtgtggggaaggcgccccc

SEQ ID NO: 45 - DNA, 33 bp, Artificial Sequence
  Cpf1 HDR Template
  misc_feature (1)..(1): A homology arm is located at the 5′ end of the sequence
  misc_feature (33)..(33): A homology arm is located at the 3′ end of the sequence
tttgcattgagatagtgtggagaaggggccccc

SEQ ID NO: 46 - DNA, 33 bp, Artificial Sequence
  Cpf1 HDR Template
  misc_feature (1)..(1): A homology arm is located at the 5′ end of the sequence
  misc_feature (33)..(33): A homology arm is located at the 3′ end of the sequence
tttgcattgagatagtgtggagaaggcgccccc

SEQ ID NO: 47 - DNA, 34 bp, Artificial Sequence
  Cpf1 HDR Template
  misc_feature (1)..(1): A homology arm is located at the 5′ end of the sequence
  misc_feature (34)..(34): A homology arm is located at the 3′ end of the sequence
tttagccagggaccgtttcagacagatgtttgca

SEQ ID NO: 48 - DNA, 25 bp, Artificial Sequence
  Cpf1 HDR Template
  misc_feature (1)..(1): A homology arm is located at the 5′ end of the sequence
  misc_feature (25)..(25): A homology arm is located at the 3′ end of the sequence
tttgccttgtcaaggctattggtta

SEQ ID NO: 49 - DNA, 25 bp, Artificial Sequence
  Cpf1 HDR Template
  misc_feature (1)..(1): A homology arm is located at the 5′ end of the sequence
  misc_feature (25)..(25): A homology arm is located at the 3′ end of the sequence
tttgccttgtcaaggctattagtca

SEQ ID NO: 50 - DNA, 25 bp, Artificial Sequence
  Cpf1 HDR Template
  misc_feature (1)..(1): A homology arm is located at the 5′ end of the sequence
  misc_feature (25)..(25): A homology arm is located at the 3′ end of the sequence
tttgccttgtcaaggctattagtta

SEQ ID NO: 51 - DNA, 12 bp, Artificial Sequence
  Cpf1 HDR Template
  misc_feature (1)..(1): A homology arm is located at the 5′ end of the sequence
  misc_feature (12)..(12): A homology arm is located at the 3′ end of the sequence
tttgccttgtca

SEQ ID NO: 52 - DNA, 13 bp, Artificial Sequence
  Cpf1 HDR Template
  misc_feature (1)..(1): A homology arm is located at the 5′ end of the sequence
  misc_feature (13)..(13): A homology arm is located at the 3′ end of the sequence
tttgccttagtta

SEQ ID NO: 53 - protein, 87 aa, Homo sapiens
DCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 54 - protein, 87 aa, Mus musculus
GCPECKLKENKYFSKLGAPIYQCMGCCFSRAYPTPARSKKTMLVPKNITSEATCCVAKAFTKATVMGNARVENHTECHCSTCYYHKS

SEQ ID NO: 55 - protein, 121 aa, Homo sapiens
SREPLRPWCHPINAILAVEKEGCPVCITVNTTICAGYCPTMMRVLQAVLPPLPQVVCTYRDVRFESIRLPGCPRGVDPVVSFPVALSCRCGPCRRSTSDCGGPKDHPLTCDHPQLSGLLFL

SEQ ID NO: 56 - protein, 121 aa, Mus musculus
SRGPLRPLCRPVNATLAAENEFCPVCITFTTSICAGYCPSMVRVLPAALPPVPQPVCTYRELRFASVRLPGCPPGVDPIVSFPVALSCRCGPCRLSSSDCGGPRTQPMACDLPHLPGLLLL

SEQ ID NO: 57 - protein, 9 aa, Artificial Sequence
  CDRH1 anti-LHR binding agent
GYSITSGYG

SEQ ID NO: 58 - protein, 7 aa, Artificial Sequence
  CDRH2 anti-LHR binding agent
IHYSGST

SEQ ID NO: 59 - protein, 6 aa, Artificial Sequence
  CDRH3 anti-LHR binding agent
ARSLRY

SEQ ID NO: 60 - protein, 5 aa, Artificial Sequence
  CDRL1 anti-LHR binding agent
SSVNY

SEQ ID NO: 61 - protein, 9 aa, Artificial Sequence
  CDRL3 anti-LHR binding agent
HQWSSYPYT

SEQ ID NO: 62 - protein, 8 aa, Artificial Sequence
  CDRH1 anti-LHR binding agent
GFSLTTYG

SEQ ID NO: 63 - protein, 7 aa, Artificial Sequence
  CDRH2 anti-LHR binding agent
IWGDGST

SEQ ID NO: 64 - protein, 9 aa, Artificial Sequence
  CDRH3 anti-LHR binding agent
AEGSSLFAY

SEQ ID NO: 65 - protein, 12 aa, Artificial Sequence
  CDRL1 anti-LHR binding agent
QSLLNSGNQKNY

SEQ ID NO: 66 - protein, 9 aa, Artificial Sequence
  CDRL3 anti-LHR binding agent
QNDYSYPLT

SEQ ID NO: 67 - protein, 8 aa, Artificial Sequence
  CDRH1 anti-LHR binding agent
GYSFTGYY

SEQ ID NO: 68 - protein, 8 aa, Artificial Sequence
  CDRH2 anti-LHR binding agent
IYPYNGVS

SEQ ID NO: 69 - protein, 14 aa, Artificial Sequence
  CDRH3 anti-LHR binding agent
ARERGLYQLRAMDY

SEQ ID NO: 70 - protein, 6 aa, Artificial Sequence
  CDRL1 anti-LHR binding agent
QSISNN

SEQ ID NO: 71 - protein, 9 aa, Artificial Sequence
  CDRL3 anti-LHR binding agent
QQSNSWPYT

SEQ ID NO: 72 - protein, 111 aa, Artificial Sequence
  anti-LHR binding agent heavy chain
EVQLQESGPDLVKPSQSLSLTCTVTGYSITSGYGWHRQFPGNKLEWMGYIHYSGSTTYNPSLKSRISISRDTSKNQFFLQLNSVTTEDTATYYCARSLRYWGQGTTLTVSS

SEQ ID NO: 73 - protein, 106 aa, Artificial Sequence
  anti-LHR binding agent light chain
DIVMTQTPAIMSASPGQKVTITCSASSSVNYMHWYQQKLGSSPKLWIYDTSKLAPGVPARFSGSGSGTSYSLTISSMEAEDAASYFCHQWSSYPYTFGSGTKLEIK

SEQ ID NO: 74 - protein, 115 aa, Artificial Sequence
  anti-LHR binding agent heavy chain
QVQLKESGPGLVAPSQSLSRRCTVSGFSLTTYGVSWVRQPPGKGLEWLGVIWGDGSTYYHSALISRLSISKDNSKSQVFLKLNSLQTDDTATYYCAEGSSLFAYWGQGTLVTVSA

SEQ ID NO: 75 - protein, 113 aa, Artificial Sequence
  anti-LHR binding agent light chain
  misc_feature (89)..(89): Xaa can be any naturally occurring amino acid
DIVMTQSPSSLTVTAGEKVTMSCKSSQSLLNSGNQKNYLTWYQQKPGQPPKLLIYWASTRQSGVPDRFTGSGSGTDFTLTISSVQAEDXAVYYCQNDYSYPLTFGSGTKLEIK

SEQ ID NO: 76 - protein, 103 aa, Artificial Sequence
  anti-LHR binding agent heavy chain
EVQLEQSGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRLL

SEQ ID NO: 77 - protein, 107 aa, Artificial Sequence
  anti-LHR binding agent light chain
DIVLTQTPSSLSASLGDTITITCHASQNINVWLFWYQQKPGNIPKLLIYKASNLLTGVPSRFSGSGSGTGFTLTISSLQPEDIATYYCQQGQSFPWTFGGGTKLEIK

SEQ ID NO: 78 - protein, 121 aa, Artificial Sequence
  anti-LHR binding agent heavy chain
QVKLQQSGPELVKPGASVKISCKASGYSFTGYYMHWVKQSHGNILDWIGYIYPYNGVSSYNQKFKGKATLTVDKSSSTAYMELRSLTSEDSAVYYCARERGLYQLRAMDYWGQGTSVTVSS

SEQ ID NO: 79 - protein, 107 aa, Artificial Sequence
  anti-LHR binding agent light chain
DIVLTQTPATLSVTPGDSVSLSCRASQSISNNLHWYQQKSHESPRLLIKNASQSISGIPSKFSGSGSGTDFTLRINSVETEDFGMYFCQQSNSWPYTFGSGTKLEIK

SEQ ID NO: 80 - RNA, 41 nt, Artificial Sequence
  crRNA
uaauuucuacucuuguagauuucggacccgugcuacaacuu

SEQ ID NO: 81 - RNA, 41 nt, Artificial Sequence
  crRNA
uaauuucuacucuuguagauauagaauagccucauauuuua

SEQ ID NO: 82 - RNA, 43 nt, Artificial Sequence
  crRNA
uaauuucuacucuuguagaugagcuguuggcaucauguuccug

SEQ ID NO: 83 - RNA, 41 nt, Artificial Sequence
  crRNA
uaauuucuacucuuguagauuccaaaccuccuaaaugauac

SEQ ID NO: 84 - DNA, 27 bp, Artificial Sequence
  target site
  misc_feature (1)..(3): PAM site
tttgtgtccccgttttggttggtaaac

SEQ ID NO: 85 - DNA, 27 bp, Artificial Sequence
  target site
  misc_feature (1)..(3): PAM site
tttaaaaatcaataccgataataatga

SEQ ID NO: 86 - DNA, 27 bp, Artificial Sequence
  target site
  misc_feature (1)..(3): PAM site
tttcttaatatgaatattaatatcggt

SEQ ID NO: 87 - DNA, 27 bp, Artificial Sequence
  target site
  misc_feature (1)..(3): PAM site
tttccgtatctggaaggggcatcttgg

SEQ ID NO: 88 - DNA, 27 bp, Artificial Sequence
  target site
  misc_feature (1)..(3): PAM site
tttccttaggaccggaaggattacagc

SEQ ID NO: 89 - DNA, 27 bp, Artificial Sequence
  target site
  misc_feature (1)..(3): PAM site
tttgcctaaaaggcactatgtcaaatg

SEQ ID NO: 90 - DNA, 27 bp, Artificial Sequence
  target site
  misc_feature (1)..(3): PAM site
tttggagctgttggcatcatgttcctg

SEQ ID NO: 91 - DNA, 27 bp, Artificial Sequence
  target site
  misc_feature (1)..(3): PAM site
tttgattcttttctatctcaggacaga

SEQ ID NO: 92 - DNA, 27 bp, Artificial Sequence
  target site
  misc_feature (1)..(3): PAM site
tttatagacatcccacactgtagttct

SEQ ID NO: 93 - DNA, 27 bp, Artificial Sequence
  target site
  misc_feature (1)..(3): PAM site
tttattaatttgagaaccaacataagg

SEQ ID NO: 94 - DNA, 27 bp, Artificial Sequence
  target site
  misc_feature (1)..(3): PAM site
tttattttctttttggtaagaaggaac

SEQ ID NO: 95 - DNA, 27 bp, Artificial Sequence
  target site
  misc_feature (1)..(3): PAM site
tttcacacacacacacacacacacaca

SEQ ID NO: 96 - DNA, 25 bp, Artificial Sequence
  target site
  misc_feature (1)..(3): PAM site
tttatccaaacctcctaaatgatac

SEQ ID NO: 97 - DNA, 27 bp, Artificial Sequence
  target site
  misc_feature (1)..(3): PAM site
tttttgattcttttctatctcaggaca

SEQ ID NO: 98 - protein, 15 aa, Artificial Sequence
  Gly Ser linker
GGGGSGGGGSGGGGS

SEQ ID NO: 99 - protein, 8 aa, Artificial Sequence
  Gly Ser linker
GGGSGGGS

SEQ ID NO: 100 - protein, 11 aa, Artificial Sequence
  Gly Ser linker
GGGSGGGSGGS

SEQ ID NO: 101 - protein, 14 aa, Artificial Sequence
  anti-4-1BB CDRH2
KIYPGDSYTNYSPS

SEQ ID NO: 102 - protein, 7 aa, Artificial Sequence
  anti-4-1BB CDRH3
GYGIFDY

SEQ ID NO: 103 - protein, 11 aa, Artificial Sequence
  anti-CD8 CDRL1
RTSRSISQYLA

SEQ ID NO: 104 - protein, 7 aa, Artificial Sequence
  anti-CD8 CDRL2
SGSTLQS

SEQ ID NO: 105 - protein, 9 aa, Artificial Sequence
  anti-CD8 CDRL3
QQHNENPLT

SEQ ID NO: 106 - protein, 6 aa, Artificial Sequence
  anti-CD8 CDRH1
GFNIKD

SEQ ID NO: 107 - protein, 9 aa, Artificial Sequence
  anti-CD8 CDRH2
RIDPANDNT

SEQ ID NO: 108 - protein, 9 aa, Artificial Sequence
  anti-CD8 CDRH3
GYGYYVFDH

SEQ ID NO: 109 - protein, 109 aa, Artificial Sequence
  anti-KIR2DL1 and anti-KIR2DL2/3 variable light chain
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT

SEQ ID NO: 110 - protein, 123 aa, Artificial Sequence
  anti-KIR2DL1 and anti-KIR2DL2/3 variable heavy chain
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSS

SEQ ID NO: 111 - RNA, 35 nt, Artificial Sequence
  RNA aptamer consensus sequence binding CD133
gcucaacccacccuccuacauagggaggaacgagu

SEQ ID NO: 112 - DNA, 4107 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 113 - DNA, 3903 bp, Francisella tularensis
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 114 - DNA, 3921 bp, Acidaminococcus sp. BV3L6
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 115 - DNA, 3699 bp, Lachnospiraceae bacterium
atggattacggcaacggccagtttgagcggagagcccccctgaccaagacaatcaccctgcgcctgaagcctatcggcgagacacgggagacaatccgcgagcagaagctgctggagcaggacgccgccttcagaaagctggtggagacagtgacccctatcgtggacgattgtatcaggaagatcgccgataacgccctgtgccactttggcaccgagtatgacttcagctgtctgggcaacgccatctctaagaatgacagcaaggccatcaagaaggagacagagaaggtggagaagctgctggccaaggtgctgaccgagaatctgccagatggcctgcgcaaggtgaacgacatcaattccgccgcctttatccaggatacactgacctctttcgtgcaggacgatgccgacaagcgggtgctgatccaggagctgaagggcaagaccgtgctgatgcagcggttcctgaccacacggatcacagccctgaccgtgtggctgcccgacagagtgttcgagaactttaatatcttcatcgagaacgccgagaagatgagaatcctgctggactcccctctgaatgagaagatcatgaagtttgacccagatgccgagcagtacgcctctctggagttctatggccagtgcctgtctcagaaggacatcgatagctacaacctgatcatctccggcatctatgccgacgatgaggtgaagaaccctggcatcaatgagatcgtgaaggagtacaatcagcagatccggggcgacaaggatgagtccccactgcccaagctgaagaagctgcacaagcagatcctgatgccagtggagaaggccttctttgtgcgcgtgctgtctaacgacagcgatgcccggagcatcctggagaagatcctgaaggacacagagatgctgccctccaagatcatcgaggccatgaaggaggcagatgcaggcgacatcgccgtgtacggcagccggctgcacgagctgagccacgtgatctacggcgatcacggcaagctgtcccagatcatctatgacaaggagtccaagaggatctctgagctgatggagacactgtctccaaaggagcgcaaggagagcaagaagcggctggagggcctggaggagcacatcagaaagtctacatacaccttcgacgagctgaacaggtatgccgagaagaatgtgatggcagcatacatcgcagcagtggaggagtcttgtgccgagatcatgagaaaggagaaggatctgaggaccctgctgagcaaggaggacgtgaagatccggggcaacagacacaatacactgatcgtgaagaactactttaatgcctggaccgtgttccggaacctgatcagaatcctgaggcgcaagtccgaggccgagatcgactctgacttctacgatgtgctggacgattccgtggaggtgctgtctctgacatacaagggcgagaatctgtgccgcagctatatcaccaagaagatcggctccgacctgaagcccgagatcgccacatacggcagcgccctgaggcctaacagccgctggtggtccccaggagagaagtttaatgtgaagttccacaccatcgtgcggagagatggccggctgtactatttcatcctgcccaagggcgccaagcctgtggagctggaggacatggatggcgacatcgagtgtctgcagatgagaaagatccctaacccaacaatctttctgcccaagctggtgttcaaggaccctgaggccttctttagggataatccagaggccgacgagttcgtgtttctgagcggcatgaaggcccccgtgacaatcaccagagagacatacgaggcctacaggtataagctgtataccgtgggcaagctgcgcgatggcgaggtgtccgaagaggagtacaagcgggccctgctgcaggtgctgaccgcctacaaggagtttctggagaacagaatgatctatgccgacctgaatttcggctttaaggatctggaggagtataaggacagctccgagtttatcaagcaggtggagacacacaacaccttcatgtgctgggccaaggtgtctagctcccagctggacgatctggtgaagtctggcaacggcctgctgttcgagatctggagcgagcgcctggagtcctactataagtacggcaatgagaaggtgctgcggggctatgagggcgtgctgctgagcatcctgaaggatgagaacctggtgtccatgcggaccctgctgaacagccggcccatgctggtgtaccggccaaaggagtctagcaagcctatggtggtgcaccgggatggcagcagagtggtggacaggtttgataaggacggcaagtacatcccccctgaggtgcacgacgagctgtatcgcttctttaacaatctgctgatcaaggagaagctgggcgagaaggcccggaagatcctggacaacaagaaggtgaaggtgaaggtgctggagagcgagagagtgaagtggtccaagttctacgatgagcagtttgccgtgaccttcagcgtgaagaagaacgccgattgtctggacaccacaaaggacctgaatgccgaagtgatggagcagtatagcgagtccaacagactgatcctgatcaggaataccacagatatcctgtactatctggtgctggacaagaatggcaaggtgctgaagcagagatccctgaacatcatcaatgacggcgccagggatgtggactggaaggagaggttccgccaggtgacaaaggatagaaacgagggctacaatgagtgggattattccaggacctctaacgacctgaaggaggtgtacctgaattatgccctgaaggagatcgccgaggccgtgatcgagtacaacgccatcctgatcatcgagaagatgtctaatgcctttaaggacaagtatagcttcctggacgacgtgaccttcaagggcttcgagacaaagctgctggccaagctgagcgatctgcactttaggggcatcaaggacggcgagccatgttccttcacaaaccccctgcagctgtgccagaacgattctaataagatcctgcaggacggcgtgatctttatggtgccaaattctatgacacggagcctggaccccgacaccggcttcatctttgccatcaacgaccacaatatcaggaccaagaaggccaagctgaactttctgagcaagttcgatcagctgaaggtgtcctctgagggctgcctgatcatgaagtacagcggcgattccctgcctacacacaacaccgacaatcgcgtgtggaactgctgttgcaatcacccaatcacaaactatgaccgggagacaaagaaggtggagttcatcgaggagcccgtggaggagctgtcccgcgtgctggaggagaatggcatcgagacagacaccgagctgaacaagctgaatgagcgggagaacgtgcctggcaaggtggtggatgccatctactctctggtgctgaattatctgcgcggcacagtgagcggagtggcaggacagagggccgtgtactatagccctgtgaccggcaagaagtacgatatctcctttatccaggccatgaacctgaataggaagtgtgactactataggatcggctccaaggagaggggagagtggaccgatttcgtggcccagctgatcaac

SEQ ID NO: 116 - DNA, 3684 bp, Lachnospiraceae bacterium
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 117 - DNA, 3900 bp, Francisella tularensis
atgagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataac

SEQ ID NO: 118 - DNA, 4431 bp, Peregrinibacteria
atgtccaacttctttaagaatttcaccaacctgtatgagctgtccaagacactgaggtttgagctgaagcccgtgggcgacaccctgacaaacatgaaggaccacctggagtacgatgagaagctgcagaccttcctgaaggatcagaatatcgacgatgcctatcaggccctgaagcctcagttcgacgagatccacgaggagtttatcacagattctctggagagcaagaaggccaaggagatcgacttctccgagtacctggatctgtttcaggagaagaaggagctgaacgactctgagaagaagctgcgcaacaagatcggcgagacattcaacaaggccggcgagaagtggaagaaggagaagtaccctcagtatgagtggaagaagggctccaagatcgccaatggcgccgacatcctgtcttgccaggatatgctgcagtttatcaagtataagaacccagaggatgagaagatcaagaattacatcgacgatacactgaagggcttctttacctatttcggcggctttaatcagaacagggccaactactatgagacaaagaaggaggcctccaccgcagtggcaacaaggatcgtgcacgagaacctgccaaagttctgtgacaatgtgatccagtttaagcacatcatcaagcggaagaaggatggcaccgtggagaaaaccgagagaaagaccgagtacctgaacgcctaccagtatctgaagaacaataacaagatcacacagatcaaggacgccgagacagagaagatgatcgagtctacacccatcgccgagaagatcttcgacgtgtactacttcagcagctgcctgagccagaagcagatcgaggagtacaaccggatcatcggccactataatctgctgatcaacctgtataaccaggccaagagatctgagggcaagcacctgagcgccaacgagaagaagtataaggacctgcctaagttcaagaccctgtataagcagatcggctgcggcaagaagaaggacctgttttacacaatcaagtgtgataccgaggaggaggccaataagtcccggaacgagggcaaggagtcccactctgtggaggagatcatcaacaaggcccaggaggccatcaataagtacttcaagtctaataacgactgtgagaatatcaacaccgtgcccgacttcatcaactatatcctgacaaaggagaattacgagggcgtgtattggagcaaggccgccatgaacaccatctccgacaagtacttcgccaattatcacgacctgcaggatagactgaaggaggccaaggtgtttcagaaggccgataagaagtccgaggacgatatcaagatcccagaggccatcgagctgtctggcctgttcggcgtgctggacagcctggccgattggcagaccacactgtttaagtctagcatcctgagcaacgaggacaagctgaagatcatcacagattcccagaccccctctgaggccctgctgaagatgatcttcaatgacatcgagaagaacatggagtcctttctgaaggagacaaacgatatcatcaccctgaagaagtataagggcaataaggagggcaccgagaagatcaagcagtggttcgactatacactggccatcaaccggatgctgaagtactttctggtgaaggagaataagatcaagggcaactccctggataccaatatctctgaggccctgaaaaccctgatctacagcgacgatgccgagtggttcaagtggtacgacgccctgagaaactatctgacccagaagcctcaggatgaggccaaggagaataagctgaagctgaatttcgacaacccatctctggccggcggctgggatgtgaacaaggagtgcagcaatttttgcgtgatcctgaaggacaagaacgagaagaagtacctggccatcatgaagaagggcgagaataccctgttccagaaggagtggacagagggccggggcaagaacctgacaaagaagtctaatccactgttcgagatcaataactgcgagatcctgagcaagatggagtatgacttttgggccgacgtgagcaagatgatccccaagtgtagcacccagctgaaggccgtggtgaaccacttcaagcagtccgacaatgagttcatctttcctatcggctacaaggtgacaagcggcgagaagtttagggaggagtgcaagatctccaagcaggacttcgagctgaataacaaggtgtttaataagaacgagctgagcgtgaccgccatgcgctacgatctgtcctctacacaggagaagcagtatatcaaggccttccagaaggagtactgggagctgctgtttaagcaggagaagcgggacaccaagctgacaaataacgagatcttcaacgagtggatcaatttttgcaacaagaagtatagcgagctgctgtcctgggagagaaagtacaaggatgccctgaccaattggatcaacttctgtaagtactttctgagcaagtatcccaagaccacactgttcaactactcttttaaggagagcgagaattataactccctggacgagttctaccgggacgtggatatctgttcttacaagctgaatatcaacaccacaatcaataagagcatcctggatagactggtggaggagggcaagctgtacctgtttgagatcaagaatcaggacagcaacgatggcaagtccatcggccacaagaataacctgcacaccatctactggaacgccatcttcgagaattttgacaacaggcctaagctgaatggcgaggccgagatcttctatcgcaaggccatctccaaggataagctgggcatcgtgaagggcaagaaaaccaagaacggcaccgagatcatcaagaattacagattcagcaaggagaagtttatcctgcacgtgccaatcaccctgaacttctgctccaataacgagtatgtgaatgacatcgtgaacacaaagttctacaatttttccaacctgcactttctgggcatcgataggggcgagaagcacctggcctactattctctggtgaataagaacggcgagatcgtggaccagggcacactgaacctgcctttcaccgacaaggatggcaatcagcgcagcatcaagaaggagaagtacttttataacaagcaggaggacaagtgggaggccaaggaggtggattgttggaattataacgacctgctggatgccatggcctctaaccgggacatggccagaaagaattggcagaggatcggcaccatcaaggaggccaagaacggctacgtgagcctggtcatcaggaagatcgccgatctggccgtgaataacgagcgccccgccttcatcgtgctggaggacctgaatacaggctttaagcggtccagacagaagatcgataagagcgtgtaccagaagttcgagctggccctggccaagaagctgaactttctggtggacaagaatgccaagcgcgatgagatcggctcccctacaaaggccctgcagctgaccccccctgtgaataactacggcgacattgagaacaagaagcaggccggcatcatgctgtatacccgggccaattatacctctcagacagatccagccacaggctggagaaagaccatctatctgaaggccggccccgaggagacaacatacaagaaggacggcaagatcaagaacaagagcgtgaaggaccagatcatcgagacattcaccgatatcggctttgacggcaaggattactatttcgagtacgacaagggcgagtttgtggatgagaaaaccggcgagatcaagcccaagaagtggcggctgtactccggcgagaatggcaagtccctggacaggttccgcggagagagggagaaggataagtatgagtggaagatcgacaagatcgatatcgtgaagatcctggacgatctgttcgtgaattttgacaagaacatcagcctgctgaagcagctgaaggagggcgtggagctgacccggaataacgagcacggcacaggcgagtccctgagattcgccatcaacctgatccagcagatccggaataccggcaataacgagagagacaacgatttcatcctgtccccagtgagggacgagaatggcaagcactttgactctcgcgagtactgggataaggagacaaagggcgagaagatcagcatgcccagctccggcgatgccaatggcgccttcaacatcgcccggaagggcatcatcatgaacgcccacatcctggccaatagcgactccaaggatctgtccctgttcgtgtctgacgaggagtgggatctgcacctgaataacaagaccgagtggaagaagcagctgaacatcttttctagcaggaaggccatggccaagcgcaagaag

SEQ ID NO: 119 - DNA, 4056 bp, Parcubacteria
atggagaacatcttcgaccagtttatcggcaagtacagcctgtccaagaccctgagattcgagctgaagcccgtgggcaagacagaggacttcctgaagatcaacaaggtgtttgagaaggatcagaccatcgacgatagctacaatcaggccaagttctattttgattccctgcaccagaagtttatcgacgccgccctggcctccgataagacatccgagctgtctttccagaactttgccgacgtgctggagaagcagaataagatcatcctggataagaagagagagatgggcgccctgaggaagcgcgacaagaacgccgtgggcatcgataggctgcagaaggagatcaatgacgccgaggatatcatccagaaggagaaggagaagatctacaaggacgtgcgcaccctgttcgataacgaggccgagtcttggaaaacctactatcaggagcgggaggtggacggcaagaagatcaccttcagcaaggccgacctgaagcagaagggcgccgattttctgacagccgccggcatcctgaaggtgctgaagtatgagttccccgaggagaaggagaaggagtttcaggccaagaaccagccctccctgttcgtggaggagaaggagaatcctggccagaagaggtacatcttcgactcttttgataagttcgccggctatctgaccaagtttcagcagacaaagaagaatctgtacgcagcagacggcaccagcacagcagtggccacccgcatcgccgataactttatcatcttccaccagaataccaaggtgttccgggacaagtacaagaacaatcacacagacctgggcttcgatgaggagaacatctttgagatcgagaggtataagaattgcctgctgcagcgcgagatcgagcacatcaagaatgagaatagctacaacaagatcatcggccggatcaataagaagatcaaggagtatcgggaccagaaggccaaggataccaagctgacaaagtccgacttccctttctttaagaacctggataagcagatcctgggcgaggtggagaaggagaagcagctgatcgagaaaacccgggagaaaaccgaggaggacgtgctgatcgagcggttcaaggagttcatcgagaacaatgaggagaggttcaccgccgccaagaagctgatgaatgccttctgtaacggcgagtttgagtccgagtacgagggcatctatctgaagaataaggccatcaacacaatctcccggagatggttcgtgtctgacagagattttgagctgaagctgcctcagcagaagtccaagaacaagtctgagaagaatgagccaaaggtgaagaagttcatctccatcgccgagatcaagaacgccgtggaggagctggacggcgatatctttaaggccgtgttctacgacaagaagatcatcgcccagggcggctctaagctggagcagttcctggtcatctggaagtacgagtttgagtatctgttccgggacatcgagagagagaacggcgagaagctgctgggctatgatagctgcctgaagatcgccaagcagctgggcatcttcccacaggagaaggaggcccgcgagaaggcaaccgccgtgatcaagaattacgccgacgccggcctgggcatcttccagatgatgaagtatttttctctggacgataaggatcggaagaacacccccggccagctgagcacaaatttctacgccgagtatgacggctactacaaggatttcgagtttatcaagtactacaacgagtttaggaacttcatcaccaagaagcctttcgacgaggataagatcaagctgaactttgagaatggcgccctgctgaagggctgggacgagaacaaggagtacgatttcatgggcgtgatcctgaagaaggagggccgcctgtatctgggcatcatgcacaagaaccaccggaagctgtttcagtccatgggcaatgccaagggcgacaacgccaatagataccagaagatgatctataagcagatcgccgacgcctctaaggatgtgcccaggctgctgctgaccagcaagaaggccatggagaagttcaagccttcccaggagatcctgagaatcaagaaggagaaaaccttcaagcgggagagcaagaacttttccctgagagatctgcacgccctgatcgagtactataggaactgcatccctcagtacagcaattggtccttttatgacttccagtttcaggataccggcaagtaccagaatatcaaggagttcacagacgatgtgcagaagtacggctataagatctcctttcgcgacatcgacgatgagtatatcaatcaggccctgaacgagggcaagatgtacctgttcgaggtggtgaacaaggatatctataacaccaagaatggctccaagaatctgcacacactgtactttgagcacatcctgtctgccgagaacctgaatgacccagtgttcaagctgtctggcatggccgagatctttcagcggcagcccagcgtgaacgaaagagagaagatcaccacacagaagaatcagtgtatcctggacaagggcgatagagcctacaagtataggcgctacaccgagaagaagatcatgttccacatgagcctggtgctgaacacaggcaagggcgagatcaagcaggtgcagtttaataagatcatcaaccagaggatcagctcctctgacaacgagatgagggtgaatgtgatcggcatcgatcgcggcgagaagaacctgctgtactatagcgtggtgaagcagaatggcgagatcatcgagcaggcctccctgaacgagatcaatggcgtgaactaccgggacaagctgatcgagagggagaaggagcgcctgaagaaccggcagagctggaagcctgtggtgaagatcaaggatctgaagaagggctacatctcccacgtgatccacaagatctgccagctgatcgagaagtattctgccatcgtggtgctggaggacctgaatatgagattcaagcagatcaggggaggaatcgagcggagcgtgtaccagcagttcgagaaggccctgatcgataagctgggctatctggtgtttaaggacaacagggatctgagggcaccaggaggcgtgctgaatggctaccagctgtctgccccctttgtgagcttcgagaagatgcgcaagcagaccggcatcctgttctacacacaggccgagtataccagcaagacagacccaatcaccggctttcggaagaacgtgtatatctctaatagcgcctccctggataagatcaaggaggccgtgaagaagttcgacgccatcggctgggatggcaaggagcagtcttacttctttaagtacaacccttacaacctggccgacgagaagtataagaactctaccgtgagcaaggagtgggccatctttgccagcgccccaagaatccggagacagaagggcgaggacggctactggaagtatgatagggtgaaagtgaatgaggagttcgagaagctgctgaaggtctggaattttgtgaacccaaaggccacagatatcaagcaggagatcatcaagaaggagaaggcaggcgacctgcagggagagaaggagctggatggccggctgagaaacttttggcactctttcatctacctgtttaacctggtgctggagctgcgcaattctttcagcctgcagatcaagatcaaggcaggagaagtgatcgcagtggacgagggcgtggacttcatcgccagcccagtgaagcccttctttaccacacccaacccttacatcccctccaacctgtgctggctggccgtggagaatgcagacgcaaacggagcctataatatcgccaggaagggcgtgatgatcctgaagaagatccgcgagcacgccaagaaggaccccgagttcaagaagctgccaaacctgtttatcagcaatgcagagtgggacgaggcagcccgggattggggcaagtacgcaggcaccacagccctgaacctggaccac

SEQ ID NO: 120 - DNA, 3618 bp, Lachnospiraceae bacterium
atgtactatgagtccctgaccaagcagtaccccgtgtctaagacaatccggaatgagctgatccctatcggcaagacactggataacatccgccagaacaatatcctggagagcgacgtgaagcggaagcagaactacgagcacgtgaagggcatcctggatgagtatcacaagcagctgatcaacgaggccctggacaattgcaccctgccatccctgaagatcgccgccgagatctacctgaagaatcagaaggaggtgtctgacagagaggatttcaacaagacacaggacctgctgaggaaggaggtggtggagaagctgaaggcccacgagaactttaccaagatcggcaagaaggacatcctggatctgctggagaagctgccttccatctctgaggacgattacaatgccctggagagcttccgcaacttttacacctatttcacatcctacaacaaggtgcgggagaatctgtattctgataaggagaagagctccacagtggcctacagactgatcaacgagaatttcccaaagtttctggacaatgtgaagagctataggtttgtgaaaaccgcaggcatcctggcagatggcctgggagaggaggagcaggactccctgttcatcgtggagacattcaacaagaccctgacacaggacggcatcgatacctacaattctcaagtgggcaagatcaactctagcatcaatctgtataaccagaagaatcagaaggccaatggcttcagaaagatccccaagatgaagatgctgtataagcagatcctgtccgatagggaggagtctttcatcgacgagtttcagagcgatgaggtgctgatcgacaacgtggagtcttatggcagcgtgctgatcgagtctctgaagtcctctaaggtgagcgccttctttgatgccctgagagagtctaagggcaagaacgtgtacgtgaagaatgacctggccaagacagccatgagcaacatcgtgttcgagaattggaggacctttgacgatctgctgaaccaggagtacgacctggccaacgagaacaagaagaaggacgataagtatttcgagaagcgccagaaggagctgaagaagaataagagctactccctggagcacctgtgcaacctgtccgaggattcttgtaacctgatcgagaattatatccaccagatctccgacgatatcgagaatatcatcatcaacaatgagacattcctgcgcatcgtgatcaatgagcacgacaggtcccgcaagctggccaagaaccggaaggccgtgaaggccatcaaggactttctggattctatcaaggtgctggagcgggagctgaagctgatcaacagctccggccaggagctggagaaggatctgatcgtgtactctgcccacgaggagctgctggtggagctgaagcaggtggacagcctgtataacatgaccagaaattatctgacaaagaagcctttctctaccgagaaggtgaagctgaactttaatcgcagcacactgctgaacggctgggatcggaataaggagacagacaacctgggcgtgctgctgctgaaggacggcaagtactatctgggcatcatgaacacaagcgccaataaggccttcgtgaatccccctgtggccaagaccgagaaggtgtttaagaaggtggattacaagctgctgccagtgcccaaccagatgctgccaaaggtgttctttgccaagagcaatatcgacttctataacccctctagcgagatctactccaattataagaagggcacccacaagaagggcaatatgttttccctggaggattgtcacaacctgatcgacttctttaaggagtctatcagcaagcacgaggactggagcaagttcggctttaagttcagcgatacagcctcctacaacgacatctccgagttctatcgcgaggtggagaagcagggctacaagctgacctatacagacatcgatgagacatacatcaatgatctgatcgagcggaacgagctgtacctgttccagatctataataaggactttagcatgtactccaagggcaagctgaacctgcacacactgtatttcatgatgctgtttgatcagcgcaatatcgacgacgtggtgtataagctgaacggagaggcagaggtgttctataggccagcctccatctctgaggacgagctgatcatccacaaggccggcgaggagatcaagaacaagaatcctaaccgggccagaaccaaggagacaagcaccttcagctacgacatcgtgaaggataagcggtatagcaaggataagtttaccctgcacatccccatcacaatgaacttcggcgtggatgaggtgaagcggttcaacgacgccgtgaacagcgccatccggatcgatgagaatgtgaacgtgatcggcatcgaccggggcgagagaaatctgctgtacgtggtggtcatcgactctaagggcaacatcctggagcagatctccctgaactctatcatcaataaggagtacgacatcgagacagattatcacgcactgctggatgagagggagggcggcagagataaggcccggaaggactggaacaccgtggagaatatcagggacctgaaggccggctacctgagccaggtggtgaacgtggtggccaagctggtgctgaagtataatgccatcatctgcctggaggacctgaactttggcttcaagaggggccgccagaaggtggagaagcaggtgtaccagaagttcgagaagatgctgatcgataagctgaattacctggtcatcgacaagagccgcgagcagacatcccctaaggagctgggaggcgccctgaacgcactgcagctgacctctaagttcaagagctttaaggagctgggcaagcagtccggcgtgatctactatgtgcctgcctacctgacctctaagatcgatccaaccacaggcttcgccaatctgttttatatgaagtgtgagaacgtggagaagtccaagagattctttgacggctttgatttcatcaggttcaacgccctggagaacgtgttcgagttcggctttgactaccggagcttcacccagagggcctgcggcatcaattccaagtggaccgtgtgcaccaacggcgagcgcatcatcaagtatcggaatccagataagaacaatatgttcgacgagaaggtggtggtggtgaccgatgagatgaagaacctgtttgagcagtacaagatcccctatgaggatggcagaaatgtgaaggacatgatcatcagcaacgaggaggccgagttctaccggagactgtataggctgctgcagcagaccctgcagatgagaaacagcacctccgacggcacaagggattacatcatctcccctgtgaagaataagagagaggcctacttcaacagcgagctgtccgacggctctgtgccaaaggacgccgatgccaacggcgcctacaatatcgccagaaagggcctgtgggtgctggagcagatcaggcagaagagcgagggcgagaagatcaatctggccatgaccaacgccgagtggctggagtatgcccagacacacctgctg

SEQ ID NO: 121 - DNA, 3714 bp, Candidatus Methanoplasma termitum
atgaacaattacgacgagttcaccaagctgtatcctatccagaaaaccatccggtttgagctgaagccacagggcagaaccatggagcacctggagacattcaacttctttgaggaggaccgggatagagccgagaagtataagatcctgaaggaggccatcgacgagtaccacaagaagtttatcgatgagcacctgaccaatatgtccctggattggaactctctgaagcagatcagcgagaagtactataagagcagggaggagaaggacaagaaggtgttcctgtccgagcagaagaggatgcgccaggagatcgtgtctgagtttaagaaggacgatcgcttcaaggacctgttttccaagaagctgttctctgagctgctgaaggaggagatctacaagaagggcaaccaccaggagatcgacgccctgaagagcttcgataagttttccggctatttcatcggcctgcacgagaataggaagaacatgtactccgacggcgatgagatcaccgccatctccaatcgcatcgtgaatgagaacttccccaagtttctggataacctgcagaagtaccaggaggccaggaagaagtatcctgagtggatcatcaaggccgagagcgccctggtggcccacaatatcaagatggacgaggtgttctccctggagtactttaataaggtgctgaaccaggagggcatccagcggtacaacctggccctgggcggctatgtgaccaagagcggcgagaagatgatgggcctgaatgatgccctgaacctggcccaccagtccgagaagagctccaagggcagaatccacatgacccccctgttcaagcagatcctgtccgagaaggagtccttctcttacatccccgacgtgtttacagaggattctcagctgctgcctagcatcggcggcttctttgcccagatcgagaatgacaaggatggcaacatcttcgaccgggccctggagctgatctctagctacgccgagtatgataccgagcggatctatatcagacaggccgacatcaatagagtgtccaacgtgatctttggagagtggggcaccctgggaggcctgatgagggagtacaaggccgactctatcaatgatatcaacctggagcgcacatgcaagaaggtggacaagtggctggattctaaggagtttgccctgagcgatgtgctggaggccatcaagaggaccggcaacaatgacgccttcaacgagtatatctccaagatgcggacagccagagagaagatcgatgccgcccgcaaggagatgaagttcatcagcgagaagatctccggcgatgaggagtctatccacatcatcaagaccctgctggacagcgtgcagcagttcctgcacttctttaatctgtttaaggcaaggcaggacatcccactggatggagccttctacgccgagtttgacgaggtgcacagcaagctgtttgccatcgtgcccctgtataacaaggtgcggaactatctgaccaagaacaatctgaacacaaagaagatcaagctgaatttcaagaaccctacactggccaatggctgggaccagaacaaggtgtacgattatgcctccctgatctttctgcgggacggcaattactatctgggcatcatcaatcctaagagaaagaagaacatcaagttcgagcagggctctggcaacggccccttctaccggaagatggtgtataagcagatccccggccctaataagaacctgccaagagtgttcctgacctccacaaagggcaagaaggagtataagccctctaaggagatcatcgagggctacgaggccgacaagcacatcaggggcgataagttcgacctggatttttgtcacaagctgatcgatttctttaaggagtccatcgagaagcacaaggactggtctaagttcaacttctacttcagcccaaccgagagctatggcgacatctctgagttctacctggatgtggagaagcagggctatcgcatgcactttgagaatatcagcgccgagacaatcgacgagtatgtggagaagggcgatctgtttctgttccagatctacaacaaggattttgtgaaggccgccaccggcaagaaggacatgcacacaatctactggaatgccgccttcagccccgagaacctgcaggacgtggtggtgaagctgaacggcgaggccgagctgttttatagggacaagtccgatatcaaggagatcgtgcaccgcgagggcgagatcctggtgaataggacctacaacggccgcacaccagtgcccgacaagatccacaagaagctgaccgattatcacaatggccggacaaaggacctgggcgaggccaaggagtacctggataaggtgagatacttcaaggcccactatgacatcaccaaggatcggagatacctgaacgacaagatctatttccacgtgcctctgaccctgaacttcaaggccaacggcaagaagaatctgaacaagatggtcatcgagaagttcctgtccgatgagaaggcccacatcatcggcatcgacaggggcgagcgcaatctgctgtactattccatcatcgacaggtctggcaagatcatcgatcagcagagcctgaatgtgatcgacggctttgattatcgggagaagctgaaccagagagagatcgagatgaaggatgcccgccagtcttggaacgccatcggcaagatcaaggacctgaaggagggctacctgagcaaggccgtgcacgagatcaccaagatggccatccagtataatgccatcgtggtcatggaggagctgaactacggcttcaagcggggccggttcaaggtggagaagcagatctatcagaagttcgagaatatgctgatcgataagatgaactacctggtgtttaaggacgcacctgatgagtccccaggaggcgtgctgaatgcctaccagctgacaaacccactggagtctttcgccaagctgggcaagcagaccggcatcctgttttacgtgccagccgcctatacatccaagatcgaccccaccacaggcttcgtgaatctgtttaacacctcctctaagacaaacgcccaggagcggaaggagttcctgcagaagtttgagagcatctcctattctgccaaggatggcggcatctttgccttcgcctttgactacagaaagttcggcaccagcaagacagatcacaagaacgtgtggaccgcctatacaaacggcgagaggatgcgctacatcaaggagaagaagcggaatgagctgtttgacccttctaaggagatcaaggaggccctgaccagctccggcatcaagtacgatggcggccagaacatcctgccagacatcctgaggagcaacaataacggcctgatctacacaatgtattctagcttcatcgccgccatccagatgcgcgtgtacgacggcaaggaggattatatcatcagccccatcaagaactccaagggcgagttctttaggaccgaccccaagaggcgcgagctgcctatcgacgccgatgccaatggcgcctacaacatcgccctgaggggagagctgacaatgagggcaatcgcagagaagttcgaccctgatagcgagaagatggccaagctggagctgaagcacaaggattggttcgagtttatgcagaccagaggcgac

SEQ ID NO: 122 - DNA, 3846 bp, Eubacterium eligens
atgaacggcaataggtccatcgtgtaccgcgagttcgtgggcgtgatccccgtggccaagaccctgaggaatgagctgcgccctgtgggccacacacaggagcacatcatccagaacggcctgatccaggaggacgagctgcggcaggagaagagcaccgagctgaagaacatcatggacgattactatagagagtacatcgataagtctctgagcggcgtgaccgacctggacttcaccctgctgttcgagctgatgaacctggtgcagagctccccctccaaggacaataagaaggccctggagaaggagcagtctaagatgagggagcagatctgcacccacctgcagtccgactctaactacaagaatatctttaacgccaagctgctgaaggagatcctgcctgatttcatcaagaactacaatcagtatgacgtgaaggataaggccggcaagctggagacactggccctgtttaatggcttcagcacatactttaccgacttctttgagaagaggaagaacgtgttcaccaaggaggccgtgagcacatccatcgcctaccgcatcgtgcacgagaactccctgatcttcctggccaatatgacctcttataagaagatcagcgagaaggccctggatgagatcgaagtgatcgagaagaacaatcaggacaagatgggcgattgggagctgaatcagatctttaaccctgacttctacaatatggtgctgatccagtccggcatcgacttctacaacgagatctgcggcgtggtgaatgcccacatgaacctgtactgtcagcagaccaagaacaattataacctgttcaagatgcggaagctgcacaagcagatcctggcctacaccagcaccagcttcgaggtgcccaagatgttcgaggacgatatgagcgtgtataacgccgtgaacgccttcatcgacgagacagagaagggcaacatcatcggcaagctgaaggatatcgtgaataagtacgacgagctggatgagaagagaatctatatcagcaaggacttttacgagacactgagctgcttcatgtccggcaactggaatctgatcacaggctgcgtggagaacttctacgatgagaacatccacgccaagggcaagtccaaggaggagaaggtgaagaaggccgtgaaggaggacaagtacaagtctatcaatgacgtgaacgatctggtggagaagtatatcgatgagaaggagaggaatgagttcaagaacagcaatgccaagcagtacatccgcgagatctccaacatcatcaccgacacagagacagcccacctggagtatgacgatcacatctctctgatcgagagcgaggagaaggccgacgagatgaagaagcggctggatatgtatatgaacatgtaccactgggccaaggcctttatcgtggacgaggtgctggacagagatgagatgttctacagcgatatcgacgatatctataatatcctggagaacatcgtgccactgtataatcgggtgagaaactacgtgacccagaagccctacaactctaagaagatcaagctgaatttccagagccctacactggccaatggctggtcccagtctaaggagttcgacaacaatgccatcatcctgatcagagataacaagtactatctggccatcttcaatgccaagaacaagccagacaagaagatcatccagggcaactccgataagaagaacgacaacgattacaagaagatggtgtataacctgctgccaggcgccaacaagatgctgcccaaggtgtttctgtctaagaagggcatcgagacattcaagccctccgactatatcatctctggctacaacgcccacaagcacatcaagacaagcgagaattttgatatctccttctgtcgggacctgatcgattacttcaagaacagcatcgagaagcacgccgagtggagaaagtatgagttcaagttttccgccaccgacagctactccgatatctctgagttctatcgggaggtggagatgcagggctacagaatcgactggacatatatcagcgaggccgacatcaacaagctggatgaggagggcaagatctatctgtttcagatctacaataaggatttcgccgagaacagcaccggcaaggagaatctgcacacaatgtactttaagaacatcttctccgaggagaatctgaaggacatcatcatcaagctgaacggccaggccgagctgttttatcggagagcctctgtgaagaatcccgtgaagcacaagaaggatagcgtgctggtgaacaagacctacaagaatcagctggacaacggcgacgtggtgagaatccccatccctgacgatatctataacgagatctacaagatgtataatggctacatcaaggagtccgacctgtctgaggccgccaaggagtacctggataaggtggaggtgaggaccgcccagaaggacatcgtgaaggattaccgctatacagtggacaagtacttcatccacacacctatcaccatcaactataaggtgaccgcccgcaacaatgtgaatgatatggtggtgaagtacatcgcccagaacgacgatatccacgtgatcggcatcgaccggggcgagagaaacctgatctacatctccgtgatcgattctcacggcaacatcgtgaagcagaaatcctacaacatcctgaacaactacgactacaagaagaagctggtggagaaggagaaaacccgggagtacgccagaaagaactggaagagcatcggcaatatcaaggagctgaaggagggctatatctccggcgtggtgcacgagatcgccatgctgatcgtggagtacaacgccatcatcgccatggaggacctgaattatggctttaagaggggccgcttcaaggtggagcggcaggtgtaccagaagtttgagagcatgctgatcaataagctgaactatttcgccagcaaggagaagtccgtggacgagccaggaggcctgctgaagggctatcagctgacctacgtgcccgataatatcaagaacctgggcaagcagtgcggcgtgatcttttacgtgcctgccgccttcaccagcaagatcgacccatccacaggctttatctctgccttcaactttaagtctatcagcacaaatgcctctcggaagcagttctttatgcagtttgacgagatcagatactgtgccgagaaggatatgttcagctttggcttcgactacaacaacttcgatacctacaacatcacaatgggcaagacacagtggaccgtgtatacaaacggcgagagactgcagtctgagttcaacaatgccaggcgcaccggcaagacaaagagcatcaatctgacagagacaatcaagctgctgctggaggacaatgagatcaactacgccgacggccacgatatcaggatcgatatggagaagatggacgaggataagaagagcgagttctttgcccagctgctgagcctgtataagctgaccgtgcagatgcgcaattcctatacagaggccgaggagcaggagaacggcatctcttacgacaagatcatcagccctgtgatcaatgatgagggcgagttctttgactccgataactataaggagtctgacgataaggagtgcaagatgccaaaggacgccgatgccaacggcgcctactgtatcgccctgaagggcctgtatgaggtgctgaagatcaagagcgagtggaccgaggacggctttgataggaattgcctgaagctgccacacgcagagtggctggacttcatccagaacaagcggtacgag

SEQ ID NO: 123 - DNA, 4119 bp, Moraxella bovoculi
atgctgttccaggactttacccacctgtatccactgtccaagacagtgagatttgagctgaagcccatcgataggaccctggagcacatccacgccaagaacttcctgtctcaggacgagacaatggccgatatgcaccagaaggtgaaagtgatcctggacgattaccaccgcgacttcatcgccgatatgatgggcgaggtgaagctgaccaagctggccgagttctatgacgtgtacctgaagtttcggaagaacccaaaggacgatgagctgcagaagcagctgaaggatctgcaggccgtgctgagaaaggagatcgtgaagcccatcggcaatggcggcaagtataaggccggctacgacaggctgttcggcgccaagctgtttaaggacggcaaggagctgggcgatctggccaagttcgtgatcgcacaggagggagagagctccccaaagctggcccacctggcccacttcgagaagttttccacctatttcacaggctttcacgataaccggaagaatatgtattctgacgaggataagcacaccgccatcgcctaccgcctgatccacgagaacctgccccggtttatcgacaatctgcagatcctgaccacaatcaagcagaagcactctgccctgtacgatcagatcatcaacgagctgaccgccagcggcctggacgtgtctctggccagccacctggatggctatcacaagctgctgacacaggagggcatcaccgcctacaatacactgctgggaggaatctccggagaggcaggctctcctaagatccagggcatcaacgagctgatcaattctcaccacaaccagcactgccacaagagcgagagaatcgccaagctgaggccactgcacaagcagatcctgtccgacggcatgagcgtgtccttcctgccctctaagtttgccgacgatagcgagatgtgccaggccgtgaacgagttctatcgccactacgccgacgtgttcgccaaggtgcagagcctgttcgacggctttgacgatcaccagaaggatggcatctacgtggagcacaagaacctgaatgagctgtccaagcaggccttcggcgactttgcactgctgggacgcgtgctggacggatactatgtggatgtggtgaatccagagttcaacgagcggtttgccaaggccaagaccgacaatgccaaggccaagctgacaaaggagaaggataagttcatcaagggcgtgcactccctggcctctctggagcaggccatcgagcactataccgcaaggcacgacgatgagagcgtgcaggcaggcaagctgggacagtacttcaagcacggcctggccggagtggacaaccccatccagaagatccacaacaatcacagcaccatcaagggctttctggagagggagcgccctgcaggagagagagccctgccaaagatcaagtccggcaagaatcctgagatgacacagctgaggcagctgaaggagctgctggataacgccctgaatgtggcccacttcgccaagctgctgaccacaaagaccacactggacaatcaggatggcaacttctatggcgagtttggcgtgctgtacgacgagctggccaagatccccaccctgtataacaaggtgagagattacctgagccagaagcctttctccaccgagaagtacaagctgaactttggcaatccaacactgctgaatggctgggacctgaacaaggagaaggataatttcggcgtgatcctgcagaaggacggctgctactatctggccctgctggacaaggcccacaagaaggtgtttgataacgcccctaatacaggcaagagcatctatcagaagatgatctataagtacctggaggtgaggaagcagttccccaaggtgttcttttccaaggaggccatcgccatcaactaccacccttctaaggagctggtggagatcaaggacaagggccggcagagatccgacgatgagcgcctgaagctgtatcggtttatcctggagtgtctgaagatccaccctaagtacgataagaagttcgagggcgccatcggcgacatccagctgtttaagaaggataagaagggcagagaggtgccaatcagcgagaaggacctgttcgataagatcaacggcatcttttctagcaagcctaagctggagatggaggacttctttatcggcgagttcaagaggtataacccaagccaggacctggtggatcagtataatatctacaagaagatcgactccaacgataatcgcaagaaggagaatttctacaacaatcaccccaagtttaagaaggatctggtgcggtactattacgagtctatgtgcaagcacgaggagtgggaggagagcttcgagttttccaagaagctgcaggacatcggctgttacgtggatgtgaacgagctgtttaccgagatcgagacacggagactgaattataagatctccttctgcaacatcaatgccgactacatcgatgagctggtggagcagggccagctgtatctgttccagatctacaacaaggacttttccccaaaggcccacggcaagcccaatctgcacaccctgtacttcaaggccctgttttctgaggacaacctggccgatcctatctataagctgaatggcgaggcccagatcttctacagaaaggcctccctggacatgaacgagacaacaatccacagggccggcgaggtgctggagaacaagaatcccgataatcctaagaagagacagttcgtgtacgacatcatcaaggataagaggtacacacaggacaagttcatgctgcacgtgccaatcaccatgaactttggcgtgcagggcatgacaatcaaggagttcaataagaaggtgaaccagtctatccagcagtatgacgaggtgaacgtgatcggcatcgatcggggcgagagacacctgctgtacctgaccgtgatcaatagcaagggcgagatcctggagcagtgttccctgaacgacatcaccacagcctctgccaatggcacacagatgaccacaccttaccacaagatcctggataagagggagatcgagcgcctgaacgcccgggtgggatggggcgagatcgagacaatcaaggagctgaagtctggctatctgagccacgtggtgcaccagatcagccagctgatgctgaagtacaacgccatcgtggtgctggaggacctgaatttcggctttaagaggggccgctttaaggtggagaagcagatctatcagaacttcgagaatgccctgatcaagaagctgaaccacctggtgctgaaggacaaggccgacgatgagatcggctcttacaagaatgccctgcagctgaccaacaatttcacagatctgaagagcatcggcaagcagaccggcttcctgttttatgtgcccgcctggaacacctctaagatcgaccctgagacaggctttgtggatctgctgaagccaagatacgagaacatcgcccagagccaggccttctttggcaagttcgacaagatctgctataatgccgacaaggattacttcgagtttcacatcgactacgccaagtttaccgataaggccaagaatagccgccagatctggacaatctgttcccacggcgacaagcggtacgtgtacgataagacagccaaccagaataagggcgccgccaagggcatcaacgtgaatgatgagctgaagtccctgttcgcccgccaccacatcaacgagaagcagcccaacctggtcatggacatctgccagaacaatgataaggagtttcacaagtctctgatgtacctgctgaaaaccctgctggccctgcggtacagcaacgcctcctctgacgaggatttcatcctgtcccccgtggcaaacgacgagggcgtgttctttaatagcgccctggccgacgatacacagcctcagaatgccgatgccaacggcgcctaccacatcgccctgaagggcctgtggctgctgaatgagctgaagaactccgacgatctgaacaaggtgaagctggccatcgacaatcagacctggctgaatttcgcccagaacagg

SEQ ID NO: 124 - DNA, 3969 bp, Prevotella disiens
atggagaactatcaggagttcaccaacctgtttcagctgaataagacactgagattcgagctgaagcccatcggcaagacctgcgagctgctggaggagggcaagatcttcgccagcggctcctttctggagaaggacaaggtgagggccgataacgtgagctacgtgaagaaggagatcgacaagaagcacaagatctttatcgaggagacactgagctccttctctatcagcaacgatctgctgaagcagtactttgactgctataatgagctgaaggccttcaagaaggactgtaagagcgatgaggaggaggtgaagaaaaccgccctgcgcaacaagtgtacctccatccagagggccatgcgcgaggccatctctcaggcctttctgaagagcccccagaagaagctgctggccatcaagaacctgatcgagaacgtgttcaaggccgacgagaatgtgcagcacttctccgagtttaccagctatttctccggctttgagacaaacagagagaatttctactctgacgaggagaagtccacatctatcgcctataggctggtgcacgataacctgcctatcttcatcaagaacatctacatcttcgagaagctgaaggagcagttcgacgccaagaccctgagcgagatcttcgagaactacaagctgtatgtggccggctctagcctggatgaggtgttctccctggagtactttaacaataccctgacacagaagggcatcgacaactataatgccgtgatcggcaagatcgtgaaggaggataagcaggagatccagggcctgaacgagcacatcaacctgtataatcagaagcacaaggaccggagactgcccttctttatctccctgaagaagcagatcctgtccgatcgggaggccctgtcttggctgcctgacatgttcaagaatgattctgaagtgatcaaggccctgaagggcttctacatcgaggacggctttgagaacaatgtgctgacacctctggccaccctgctgtcctctctggataagtacaacctgaatggcatctttatccgcaacaatgaggccctgagctccctgtcccagaacgtgtatcggaatttttctatcgacgaggccatcgatgccaacgccgagctgcagaccttcaacaattacgagctgatcgccaatgccctgcgcgccaagatcaagaaggagacaaagcagggccggaagtctttcgagaagtacgaggagtatatcgataagaaggtgaaggccatcgacagcctgtccatccaggagatcaacgagctggtggagaattacgtgagcgagtttaactctaatagcggcaacatgccaagaaaggtggaggactacttcagcctgatgaggaagggcgacttcggctccaacgatctgatcgaaaatatcaagaccaagctgagcgccgcagagaagctgctgggcacaaagtaccaggagacagccaaggacatcttcaagaaggatgagaactccaagctgatcaaggagctgctggacgccaccaagcagttccagcactttatcaagccactgctgggcacaggcgaggaggcagatcgggacctggtgttctacggcgattttctgcccctgtatgagaagtttgaggagctgaccctgctgtataacaaggtgcggaatagactgacacagaagccctattccaaggacaagatccgcctgtgcttcaacaagcctaagctgatgacaggctgggtggattccaagaccgagaagtctgacaacggcacacagtacggcggctatctgtttcggaagaagaatgagatcggcgagtacgattattttctgggcatctctagcaaggcccagctgttcagaaagaacgaggccgtgatcggcgactacgagaggctggattactatcagccaaaggccaataccatctacggctctgcctatgagggcgagaacagctacaaggaggacaagaagcggctgaacaaagtgatcatcgcctatatcgagcagatcaagcagacaaacatcaagaagtctatcatcgagtccatctctaagtatcctaatatcagcgacgatgacaaggtgaccccatcctctctgctggagaagatcaagaaggtgtctatcgacagctacaacggcatcctgtccttcaagtcttttcagagcgtgaacaaggaagtgatcgataacctgctgaaaaccatcagccccctgaagaacaaggccgagtttctggacctgatcaataaggattatcagatcttcaccgaggtgcaggccgtgatcgacgagatctgcaagcagaaaaccttcatctactttccaatctccaacgtggagctggagaaggagatgggcgataaggacaagcccctgtgcctgttccagatcagcaataaggatctgtccttcgccaagacctttagcgccaacctgcggaagaagagaggcgccgagaatctgcacacaatgctgtttaaggccctgatggagggcaaccaggataatctggacctgggctctggcgccatcttctacagagccaagagcctggacggcaacaagcccacacaccctgccaatgaggccatcaagtgtaggaacgtggccaataaggataaggtgtccctgttcacctacgacatctataagaacaggcgctacatggagaataagttcctgtttcacctgagcatcgtgcagaactataaggccgccaatgactccgcccagctgaacagctccgccaccgagtatatcagaaaggccgatgacctgcacatcatcggcatcgataggggcgagcgcaatctgctgtactattccgtgatcgatatgaagggcaacatcgtggagcaggactctctgaatatcatcaggaacaatgacctggagacagattaccacgacctgctggataagagggagaaggagcgcaaggccaaccggcagaattgggaggccgtggagggcatcaaggacctgaagaagggctacctgagccaggccgtgcaccagatcgcccagctgatgctgaagtataacgccatcatcgccctggaggatctgggccagatgtttgtgacccgcggccagaagatcgagaaggccgtgtaccagcagttcgagaagagcctggtggataagctgtcctacctggtggacaagaagcggccttataatgagctgggcggcatcctgaaggcctaccagctggcctctagcatcaccaagaacaattctgacaagcagaacggcttcctgttttatgtgccagcctggaatacaagcaagatcgatcccgtgaccggctttacagacctgctgcggcccaaggccatgaccatcaaggaggcccaggacttctttggcgccttcgataacatctcttacaatgacaagggctatttcgagtttgagacaaactacgacaagtttaagatcagaatgaagagcgcccagaccaggtggacaatctgcaccttcggcaatcggatcaagagaaagaaggataagaactactggaattatgaggaggtggagctgaccgaggagttcaagaagctgtttaaggacagcaacatcgattacgagaactgtaatctgaaggaggagatccagaacaaggacaatcgcaagttctttgatgacctgatcaagctgctgcagctgacactgcagatgcggaactccgatgacaagggcaatgattatatcatctctcctgtggccaacgccgagggccagttctttgactcccgcaatggcgataagaagctgccactggatgcagacgcaaacggagcctacaatatcgcccgcaagggcctgtggaacatccggcagatcaagcagaccaagaacgacaagaagctgaatctgagcatctcctctacagagtggctggatttcgtgcgggagaagccttacctgaag

SEQ ID NO: 125 - protein, 1368 aa, Streptococcus pyogenes [sequence omitted]

SEQ ID NO: 126 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 127 - protein, 1307 aa, Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 128 - protein, 1233 aa, Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKLLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 129 - protein, 1246 aa, Lachnospiraceae bacterium
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 130 - protein, 1228 aa, Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 131 - protein, 1300 aa, Francisella tularensis; identical to SEQ ID NO: 126

SEQ ID NO: 132 - protein, 1477 aa, Peregrinibacteria
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 133 - protein, 1352 aa, Parcubacteria
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 134 - protein, 1206 aa, Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 135 - protein, 1238 aa, Candidatus Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 136 - protein, 1282 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 137 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 138 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 139 - protein, 16 aa, Artificial Sequence
  anti-CD34 CDRL1
RSSQTIVHSNGNTYLE

SEQ ID NO: 140 - protein, 7 aa, Artificial Sequence
  anti-CD34 CDRL2
QVSNRFS

SEQ ID NO: 141 - protein, 9 aa, Artificial Sequence
  anti-CD34 CDRL3
FQGSHVPRT

SEQ ID NO: 142 - protein, 10 aa, Artificial Sequence
  anti-CD34 CDRH1
GYTFTNYGMN

SEQ ID NO: 143 - protein, 17 aa, Artificial Sequence
  anti-CD34 CDRH2
WINTNTGEPKYAEEFKG

SEQ ID NO: 144 - protein, 13 aa, Artificial Sequence
  anti-CD34 CDRH3
GYGNYARGAWLAY

SEQ ID NO: 145 - protein, 260 aa, Artificial Sequence
  anti-cD90 scFv
CMASASQVQLVQSGAEVKKPGASVKVSCKASGYTFTGYYVHWVRQAPGQGLEWMGWVNPNSGDTNYAQKFQGRVTMTRDTSISTAYMELSGLRSDDTAVYYCARDGDEDWYFDLWGRGTPVTVSSGILGSGGGGSGGGGSGGGGSDIRLTQSPSSLSASIGDRVTITCRASQGISRSLVWYQQKPGKAPRLLIYAASTLQSGVPSRFSGSGSGTDFTLTISSLQPEDFATYYCLQHNTYPFTFGPGTKVDIKSGIPEQKL

SEQ ID NO: 146 - protein, 108 aa, Artificial Sequence
  anti-CD133 variable light chain
NIVMTQSPKSMSMSLGERVTLSCKASENVDTYVSWYQQKPEQSPKVLIYGASNRYTGVPDRFTGSGSATDFSLTISNVQAEDLADYHCGQSYRYPLTFGAGTKLELKR

SEQ ID NO: 147 - protein, 120 aa, Artificial Sequence
  anti-CD133 variable heavy chain
EIQLQQSGPDLMKPGASVKISCKASGYSFTNYYVHWVKQSLDKSLEWIGYVDPFNGDFNYNQKFKDKATLTVDKSSSTAYMHLSSLTSEDSAVYYCARGGLDWYDTSYWYFDVWGAGTAV

SEQ ID NO: 148 - protein, 13 aa, Artificial Sequence
  anti-CD133 CDRL1
QSSQSVYNNNYLA

SEQ ID NO: 149 - protein, 7 aa, Artificial Sequence
  anti-CD133 CDRL2
RASTLAS

SEQ ID NO: 150 - protein, 13 aa, Artificial Sequence
  anti-CD133 CDRL3
QGEFSCDSADCAA

SEQ ID NO: 151 - protein, 7 aa, Artificial Sequence
  anti-CD133 CDRH1
GIDLNNY

SEQ ID NO: 152 - protein, 5 aa, Artificial Sequence
  anti-CD133 CDRH2
FGSDS

SEQ ID NO: 153 - RNA, 15 nt, Artificial Sequence
  RNA aptamer consensus sequence binding CD133
cccuccuacauaggg

SEQ ID NO: 154 - RNA, 81 nt, Artificial Sequence
  RNA aptamer consensus sequence binding CD133
gagacaagaauaaacgcucaacccacccuccuacauagggaggaacgaguuacuauagagcuucgacaggaggcucacaac

SEQ ID NO: 155 - RNA, 58 nt, Artificial Sequence
  RNA aptamer consensus sequence binding CD133
gagacaagaauaaacgcucaacccacccuccuacauagggaggaacgaguuacuauag

SEQ ID NO: 156 - RNA, 21 nt, Artificial Sequence
  RNA aptamer consensus sequence binding CD133
ccacccuccuacauagggugg

SEQ ID NO: 157 - RNA, 19 nt, Artificial Sequence
  RNA aptamer consensus sequence binding CD133
cagaacguauacuauucug

SEQ ID NO: 158 - RNA, 15 nt, Artificial Sequence
  RNA aptamer consensus sequence binding CD133
agaacguauacuauu

SEQ ID NO: 159 - RNA, 85 nt, Artificial Sequence
  RNA aptamer consensus sequence binding CD133
gagacaagaauaaacgcucaaggaaagcgcuuauuguuugcuauguuagaacguauacuauuucgacaggaggcucacaacaggc

SEQ ID NO: 160 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 161 - protein, 108 aa, Artificial Sequence [sequence omitted]

SEQ ID NO: 162 - protein, 118 aa, Artificial Sequence [sequence omitted]

SEQ ID NO: 163 - protein, 118 aa, Artificial Sequence
  anti-CD3 variable heavy chain
QVQLVQSGGGVVQSGRSLRLSCAASGFKFSGYGMHWVRQAPGKGLEWVAVIWYDGSKKYYVDSVKGRFTISRDNSKNTLYLQMNSLRGEDTAVYYCARQMGYWHFDLWGRGTLVTVSS

SEQ ID NO: 164 - protein, 10 aa, Artificial Sequence
  anti-CD3 CDRL1
SASSSVSYMN

SEQ ID NO: 165 - protein, 11 aa, Artificial Sequence
  anti-CD3 CDRL2
RWIYDTSKLAS

SEQ ID NO: 166 - protein, 9 aa, Artificial Sequence
  anti-CD3 CDRL3
QQWSSNPFT

SEQ ID NO: 167 - protein, 13 aa, Artificial Sequence
  anti-CD3 CDRH1
KASGYTFTRYTMH

SEQ ID NO: 168 - protein, 16 aa, Artificial Sequence
  anti-CD3 CDRH2
INPSRGYTNYNQKFKD

SEQ ID NO: 169 - protein, 10 aa, Artificial Sequence
  anti-CD3 CDRH3
YYDDHYCLDY

SEQ ID NO: 170 - protein, 11 aa, Artificial Sequence
  anti-CD3 CDRL1
QSLVHNNGNTY

SEQ ID NO: 171 - protein, 9 aa, Artificial Sequence
  anti-CD3 CDRL3
GQGTQYPFT

SEQ ID NO: 172 - protein, 8 aa, Artificial Sequence
  anti-CD3 CDRH1
GFTFTKAW

SEQ ID NO: 173 - protein, 10 aa, Artificial Sequence
  anti-CD3 CDRH2
IKDKSNSYAT

SEQ ID NO: 174 - protein, 12 aa, Artificial Sequence
  anti-CD3 CDRH3
RGVYYALSPFDY

SEQ ID NO: 175 - protein, 11 aa, Artificial Sequence
  anti-CD3 CDRL1
QSLVHDNGNTY

SEQ ID NO: 176 - protein, 8 aa, Artificial Sequence
  anti-CD3 CDRH1
GFTFSNAW

SEQ ID NO: 177 - protein, 10 aa, Artificial Sequence
  anti-CD3 CDRH2
IKARSNNYAT

SEQ ID NO: 178 - protein, 12 aa, Artificial Sequence
  anti-CD3 CDRH3
RGTYYASKPFDY

SEQ ID NO: 179 - protein, 11 aa, Artificial Sequence
  anti-CD3 CDRL1
QSLEHNNGNTY

SEQ ID NO: 180 - protein, 10 aa, Artificial Sequence
  anti-CD3 CDRH2
IKDKSNNYAT

SEQ ID NO: 181 - protein, 13 aa, Artificial Sequence
  anti-CD3 CDRH3
RYVHYGIGYAMDA

SEQ ID NO: 182 - protein, 11 aa, Artificial Sequence
  anti-CD3 CDRL1
QSLVHTNGNTY

SEQ ID NO: 183 - protein, 9 aa, Artificial Sequence
  anti-CD3 CDRL3
GQGTHYPFT

SEQ ID NO: 184 - protein, 8 aa, Artificial Sequence
  anti-CD3 CDRH1
GFTFTNAW

SEQ ID NO: 185 - protein, 9 aa, Artificial Sequence
  anti-CD3 CDRH2
KDKSNNYAT

SEQ ID NO: 186 - protein, 13 aa, Artificial Sequence
  anti-CD3 CDRH3
RYVHYRFAYALDA

SEQ ID NO: 187 - protein, 112 aa, Artificial Sequence
  anti-CD4 variable light chain
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIK

SEQ ID NO: 188 - protein, 122 aa, Artificial Sequence
  anti-CD4 variable heavy chain
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSS

SEQ ID NO: 189 - protein, 17 aa, Artificial Sequence
  anti-CD4 CDRL1
KSSQSLLYSTNQKNYLA

SEQ ID NO: 190 - protein, 7 aa, Artificial Sequence
  anti-CD4 CDRL2
WASTRES

SEQ ID NO: 191 - protein, 8 aa, Artificial Sequence
  anti-CD4 CDRL3
QQYYSYRT

SEQ ID NO: 192 - protein, 10 aa, Artificial Sequence
  anti-CD4 CDRH1
GYTFTSYVIH

SEQ ID NO: 193 - protein, 17 aa, Artificial Sequence
  anti-CD4 CDRH2
YINPYNDGTDYDEKFKG

SEQ ID NO: 194 - protein, 13 aa, Artificial Sequence
  anti-CD4 CDRH3
EKDNYATGAWFAY

SEQ ID NO: 195 - protein, 120 aa, Artificial Sequence
  anti-CD28 variable heavy chain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 196 - protein, 107 aa, Artificial Sequence
  anti-CD28 variable light chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 197 - protein, 11 aa, Artificial Sequence
  anti-CD28 CDRL1
HASQNIYVWLN

SEQ ID NO: 198 - protein, 7 aa, Artificial Sequence
  anti-CD28 CDRL2
KASNLHT

SEQ ID NO: 199 - protein, 9 aa, Artificial Sequence
  anti-CD28 CDRL3
QQGQTYPYT

SEQ ID NO: 200 - protein, 10 aa, Artificial Sequence
  anti-CD28 CDRH1
GYTFTSYYIH

SEQ ID NO: 201 - protein, 14 aa, Artificial Sequence
  anti-CD28 CDRH2
CIYPGNVNTNYNEK

SEQ ID NO: 202 - protein, 11 aa, Artificial Sequence
  anti-CD28 CDRH3
SHYGLDWNFDV

SEQ ID NO: 203 - protein, 5 aa, Artificial Sequence
  anti-CD28 CDRH1
SYYIH

SEQ ID NO: 204 - protein, 17 aa, Artificial Sequence
  anti-CD28 CDRH2
CIYPGNVNTNYNEKFKD

SEQ ID NO: 205 - protein, 7 aa, Artificial Sequence
  anti-4-1BB CDRL1
RASQSVS

SEQ ID NO: 206 - protein, 6 aa, Artificial Sequence
  anti-4-1BB CDRL2
ASNRAT

SEQ ID NO: 207 - protein, 10 aa, Artificial Sequence
  anti-4-1BB CDRL3
QRSNWPPALT

SEQ ID NO: 208 - protein, 4 aa, Artificial Sequence
  anti-4-1BB CDRH1
YYWS

SEQ ID NO: 209 - protein, 12 aa, Artificial Sequence
  anti-4-1BB CDRH3
YGPGNYDWYFDL

SEQ ID NO: 210 - protein, 11 aa, Artificial Sequence
  anti-4-1BB CDRL1
SGDNIGDQYAH

SEQ ID NO: 211 - protein, 7 aa, Artificial Sequence
  anti-4-1BB CDRL2
QDKNRPS

SEQ ID NO: 212 - protein, 11 aa, Artificial Sequence
  anti-4-1BB CDRL3
ATYTGFGSLAV

SEQ ID NO: 213 - protein, 10 aa, Artificial Sequence
  anti-4-1BB CDRH1
GYSFSTYWIS

SEQ ID NO: 214 - DNA, 23 bp, Artificial Sequence
  Cas9 guide
tatttgcattgagatagtgtggg

SEQ ID NO: 215 - DNA, 23 bp, Artificial Sequence
  Cas9 guide
atatttgcattgagatagtgtgg

SEQ ID NO: 216 - DNA, 23 bp, Artificial Sequence
  Cas9 guide
atgcaaatatctgtctgaaacgg

SEQ ID NO: 217 - DNA, 23 bp, Artificial Sequence
  Cas9 guide
tatctgtctgaaacggtccctgg

SEQ ID NO: 218 - DNA, 23 bp, Artificial Sequence
  Cas9 guide
gctattggtcaaggcaaggctgg

SEQ ID NO: 219 - DNA, 23 bp, Artificial Sequence
  Cas9 guide
caaggctattggtcaaggcaagg

SEQ ID NO: 220 - DNA, 23 bp, Artificial Sequence
  Cas9 guide
cttgtcaaggctattggtcaagg

SEQ ID NO: 221 - DNA, 23 bp, Artificial Sequence
  Cas9 guide
cttgaccaatagccttgacaagg

SEQ ID NO: 222 - DNA, 23 bp, Artificial Sequence
  Cas9 guide
gtttgccttgtcaaggctattgg

SEQ ID NO: 223 - DNA, 23 bp, Artificial Sequence
  Cas9 guide
tggtcaagtttgccttgtcaagg

SEQ ID NO: 224 - DNA, 25 bp, Artificial Sequence
  Cpf1 guide
tttcagacagatatttgcattgaga

SEQ ID NO: 225 - RNA, 24 nt, Artificial Sequence
  transcription of DNA target site
guguccccguuuugguugguaaac

SEQ ID NO: 226 - RNA, 24 nt, Artificial Sequence
  transcription of DNA target site
aaaaaucaauaccgauaauaauga

SEQ ID NO: 227 - RNA, 24 nt, Artificial Sequence
  transcription of DNA target site
cuuaauaugaauauuaauaucggu

SEQ ID NO: 228 - RNA, 24 nt, Artificial Sequence
  transcription of DNA target site
ccguaucuggaaggggcaucuugg

SEQ ID NO: 229 - RNA, 24 nt, Artificial Sequence
  transcription of DNA target site
ccuuaggaccggaaggauuacagc

SEQ ID NO: 230 - RNA, 24 nt, Artificial Sequence
  transcription of DNA target site
gccuaaaaggcacuaugucaaaug

SEQ ID NO: 231 - RNA, 24 nt, Artificial Sequence
  transcription of DNA target site
ggagcuguuggcaucauguuccug

SEQ ID NO: 232 - RNA, 24 nt, Artificial Sequence
  transcription of DNA target site
gauucuuuucuaucucaggacaga

SEQ ID NO: 233 - RNA, 24 nt, Artificial Sequence
  transcription of DNA target site
auagacaucccacacuguaguucu

SEQ ID NO: 234 - RNA, 24 nt, Artificial Sequence
  transcription of DNA target site
auuaauuugagaaccaacauaagg

SEQ ID NO: 235 - RNA, 24 nt, Artificial Sequence
  transcription of DNA target site
auuuucuuuuugguaagaaggaac

SEQ ID NO: 236 - RNA, 24 nt, Artificial Sequence
  transcription of DNA target site
cacacacacacacacacacacaca

SEQ ID NO: 237 - RNA, 22 nt, Artificial Sequence
  transcription of DNA target site
auccaaaccuccuaaaugauac

SEQ ID NO: 238 - RNA, 22 nt, Artificial Sequence
  transcription of DNA target site
acacccgauccacuggggagca

SEQ ID NO: 239 - RNA, 24 nt, Artificial Sequence
  transcription of DNA target site
uugauucuuuucuaucucaggaca

SEQ ID NO: 240 - RNA, 22 nt, Artificial Sequence
  transcription of DNA target site
  misc_feature (1)..(1): n is a, c, g, or u
ncacccgauccacuggggagca

SEQ ID NO: 241 - RNA, 20 nt, Artificial Sequence
  transcription of DNA target site
cacccgauccacuggggagc

SEQ ID NO: 242 - RNA, 22 nt, Artificial Sequence
  transcription of DNA target site
  misc_feature (1)..(1): n is a, c, g, or u
nccuugucaaggcuauugguca

SEQ ID NO: 243 - RNA, 21 nt, Artificial Sequence
  transcription of DNA target site
ccuugucaaggcuauugguca

SEQ ID NO: 244 - RNA, 20 nt, Artificial Sequence
  transcription of DNA target site
guggggaaggggcccccaag

SEQ ID NO: 245 - RNA, 20 nt, Artificial Sequence
  transcription of DNA target site
auugagauaguguggggaag

SEQ ID NO: 246 - RNA, 20 nt, Artificial Sequence
  transcription of DNA target site
cauugagauaguguggggaa

SEQ ID NO: 247 - RNA, 20 nt, Artificial Sequence
  transcription of DNA target site
gcauugagauagugugggga

SEQ ID NO: 248 - RNA, 20 nt, Artificial Sequence
  transcription of DNA target site
auuugcauugagauagugug

SEQ ID NO: 249 - RNA, 20 nt, Artificial Sequence
  transcription of DNA target site
uauuugcauugagauagugu

SEQ ID NO: 250 - RNA, 20 nt, Artificial Sequence
  transcription of DNA target site
auauuugcauugagauagug

SEQ ID NO: 251 - RNA, 20 nt, Artificial Sequence
  transcription of DNA target site
augcaaauaucugucugaaa

SEQ ID NO: 252 - RNA, 20 nt, Artificial Sequence
  transcription of DNA target site
uaucugucugaaacgguccc

SEQ ID NO: 253 - RNA, 20 nt, Artificial Sequence
  transcription of DNA target site
gcuauuggucaaggcaaggc

SEQ ID NO: 254 - RNA, 20 nt, Artificial Sequence
  transcription of DNA target site
caaggcuauuggucaaggca

SEQ ID NO: 255 - RNA, 20 nt, Artificial Sequence
  transcription of DNA target site
cuugucaaggcuauugguca

SEQ ID NO: 256 - RNA, 20 nt, Artificial Sequence
  transcription of DNA target site
cuugaccaauagccuugaca

SEQ ID NO: 257 - RNA, 20 nt, Artificial Sequence
  transcription of DNA target site
guuugccuugucaaggcuau

SEQ ID NO: 258 - RNA, 20 nt, Artificial Sequence
  transcription of DNA target site
uggucaaguuugccuuguca

SEQ ID NO: 259 - RNA, 22 nt, Artificial Sequence
  transcription of DNA target site
gcauugagauaguguggggaag

SEQ ID NO: 260 - RNA, 22 nt, Artificial Sequence
  transcription of DNA target site
cagacagauauuugcauugaga

SEQ ID NO: 261 - RNA, 22 nt, Artificial Sequence
  transcription of DNA target site
agccagggaccguuucagacag

SEQ ID NO: 262 - RNA, 22 nt, Artificial Sequence
  transcription of DNA target site
gccuugucaaggcuauugguca

SEQ ID NO: 263 - RNA, 20 nt, Artificial Sequence
  RNA transcribed from DNA target site
cacccgauccacuggggagc

SEQ ID NO: 264 - RNA, 21 nt, Artificial Sequence
  RNA transcribed from DNA target site
ccuugucagggcuguuggucg